(19) United States
(12) Patent Application Publication
(10) Pub. No.: US 2021/0171948 A1
(43) Pub. Date: Jun. 10, 2021

(54) COMPOSITIONS AND METHODS FOR TARGETING GLYPICAN-2 IN THE TREATMENT OF CANCER

(71) Applicant: Shepherd Therapeutics, Inc., Natick, MA (US)

(72) Inventors: William M. SIDERS, Franklin, MA (US); Rajesha RUPAIMOOLE, Natick, MA (US)

(21) Appl. No.: 17/116,214

(22) Filed: Dec. 9, 2020

Related U.S. Application Data
(60) Provisional application No. 62/945,436, filed on Dec. 9, 2019.

(51) Int. Cl. C12N 15/113; A61K 33/243; A61K 31/535; A61K 31/704; A61K 31/4745; A61K 31/357; A61K 47/69

(52) U.S. Cl. CPC C12N 15/113; A61K 33/243; A61K 31/535; A61K 31/704; C12N 2310/344; A61K 31/357; A61K 47/6937; C12N 2310/14; C12N 2310/3231; A61K 31/4745

(57) ABSTRACT

The disclosure relates to dsRNAs and siRNAs targeting glypican-2 (GPC2) and nanoparticles comprising same, for the treatment of cancer.

### RELATED APPLICATIONS

This application claims benefit of, and priority to, U.S. Application No. 62/945,436, filed on Dec. 9, 2019, the entire contents of which are incorporated herein by reference.

### TECHNICAL FIELD

The disclosure relates to the use of siRNA based therapeutics for the treatment of cancer.

### INCORPORATION-BY-REFERENCE OF SEQUENCE LISTING

The contents of the text file named “SHEP_003_001US_SeqList_ST25”, which was created on Dec. 8, 2020 and is 845 KB in size, are hereby incorporated by reference in their entirety.

### BACKGROUND

Cancer is a proliferative disease in which the cells of a subject grow abnormally and in an uncontrolled way, in some cases leading to the death of the subject. There are many independent events and causes which can lead to cancer, and many different cell types and tissues that can give rise to cancers. As such, treatments developed for one type of cancer may not work on another type of cancer. Despite many years of research, and a plethora of treatments available to cancer sufferers, there is still a long felt need in the art for additional cancer therapies. Glypican-2 (Glypican 2, GPC2, or GPC-2) is a cell surface protein that belongs to a family of six proteoglycans. These proteins play diverse roles in signaling and cancer cell growth. Although GPC2 was initially thought to be solely expressed during nervous system development, GPC2 is also expressed on the surface of some types of cancer cells, such as neuroblastoma cells. The disclosure provides additional methods for the treatment of cancer by targeting GPC2 mRNA for degradation via RNA interference.

### SUMMARY

The disclosure provides nanoparticles comprising small interfering RNAs (siRNAs), wherein the siRNA comprises a sense region and anti-sense region complementary to said sense region such that the sense region and the anti-sense region together form an RNA duplex, and wherein the sense region comprises a sequence at least 70% identical to a glypican-2 (GPC2) mRNA sequence.

In some embodiments of the nanoparticles of the disclosure, the sense region comprises a sequence that is identical to the GPC2 mRNA sequence.

In some embodiments of the nanoparticles of the disclosure, the siRNA is capable of inducing RNAi-mediated degradation of the GPC2 mRNA.

In some embodiments of the nanoparticles of the disclosure, the sense region is encoded by a first single stranded RNA molecule and the anti-sense region is encoded by a second single stranded RNA molecule. In some embodiments, the first single stranded RNA molecule comprises a first 3′ overhang. In some embodiments, the second single stranded RNA molecule comprises a second 3′ overhang. In some embodiments, the first and second 3′ overhangs comprise a dinucleotide. In some embodiments, the dinucleotide comprises thymidine-thymidine (dT-dT) or Uracil-Uracil (UU). In some embodiments, the RNA duplex is between 17 and 24 nucleotides in length. In some embodiments, the RNA duplex is 19 nucleotides in length. In some embodiments, the GPC2 mRNA sequence comprises SEQ ID NO: 1 or SEQ ID NO: 2. In some embodiments, the sense region comprises a sequence selected from the group listed in Table 1 and Table 2. In some embodiments, the siRNA comprises a sequence selected from the group consisting of SEQ ID NOs: 3-232. In some embodiments, anti-sense region comprises a sequence selected from the group listed in Table 1 and Table 2. In some embodiments, the sense region comprises a sequence of CCUGCUUGGACCUCGAUAA (SEQ ID NO: 3), CUCAGUAGCCCAGCACUCU (SEQ ID NO: 4) or CUCCUUUCUGGUUCACACA (SEQ ID NO: 5). In some embodiments, the sense region comprises a sequence of CCUGCUUGGACCUCGAUAA (SEQ ID NO: 3) and the anti-sense region comprises a sequence of UUAUCGAGGUCCAAGCAGG (SEQ ID NO: 6). In some embodiments, the sense region comprises a sequence of CUCAGUAGCCCAGCACUCU (SEQ ID NO: 4) and the anti-sense region comprises a sequence of AGAGUGCUGGGCUACUGAG (SEQ ID NO: 7). In some embodiments, the sense region comprises a sequence of CUCCUUUCUGGUUCACACA (SEQ ID NO: 5) and the anti-sense region comprises a sequence of UGUGUGAACCAGAAAGGAG (SEQ ID NO: 8).

In some embodiments of the nanoparticles of the disclosure, the siRNA comprises at least one modified nucleotide. In some embodiments, the at least one modified nucleotide increases stability of the RNA duplex. In some embodiments, the at least one modified nucleotide comprises a locked nucleic acid (LNA).

In some embodiments of the nanoparticles of the disclosure, the nanoparticle comprises a liposome, a micelle, a polymer-based nanoparticle, a lipid-polymer based nanoparticle, a nanocrystal, a carbon nanotube based nanoparticle or a polymeric micelle. In some embodiments, the polymer-based nanoparticle comprises a multiblock copolymer, a diblock copolymer, a polymeric micelle or a hyperbranched macromolecule. In some embodiments, the polymer-based nanoparticle comprises a multiblock copolymer a diblock copolymer. In some embodiments, the polymer-based nanoparticle comprises a poly(lactic-co-glycolic acid) PLGA polymer.

In some embodiments of the nanoparticles of the disclosure, the nanoparticle comprises a targeting agent. In some embodiments, the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment. In some embodiments, the polysaccharide ligand comprises hyaluronic acid. In some embodiments, the targeting agent binds to the surface of a cell of the cancer of the subject.

In some embodiments of the nanoparticles of the disclosure, the nanoparticle further comprises a chemotherapeutic agent. In some embodiments, the chemotherapeutic agent comprises a platinum based antineoplastic agent, a DNA alkylating agent, a DNA intercalating agent, or a topoisomerase inhibitor. In some embodiments, the platinum based antineoplastic agent is Cisplatin or Carboplatin. In some embodiments, the DNA alkylating agent is Cyclophosphamide. In some embodiments, the DNA intercalating agent is Doxorubicin. In some embodiments, the topoisomerase inhibitor is Etoposide or Topotecan.

The disclosure provides pharmaceutical compositions comprising the nanoparticles of the disclosure, and a pharmaceutically acceptable carrier, diluent or excipient. In some embodiments, the pharmaceutical composition further comprises a chemotherapeutic agent. In some embodiments the nanoparticle further comprises a chemotherapeutic agent. In some embodiments, the chemotherapeutic agent comprises a platinum based antineoplastic agent, a DNA alkylating agent, a DNA intercalating agent, or a topoisomerase inhibitor. In some embodiments, the platinum based antineoplastic agent is Cisplatin or Carboplatin. In some embodiments, the DNA alkylating agent is Cyclophosphamide. In some embodiments, the DNA intercalating agent is Doxorubicin. In some embodiments, the topoisomerase inhibitor is Etoposide or Topotecan. In some embodiments, the platinum based antineoplastic agent is Cisplatin or Carboplatin. In some embodiments, the DNA alkylating agent is Cyclophosphamide. In some embodiments, the DNA intercalating agent is Doxorubicin. In some embodiments, the topoisomerase inhibitor is Etoposide or Topotecan.

The disclosure provides kits comprising the nanoparticles or pharmaceutical compositions of the disclosure. In some embodiments, the kits further comprise instructions for administrating the nanoparticles or pharmaceutical compositions to a subject.

The disclosure provides methods of treating a cancer in a subject, comprising administering to the subject a therapeutically effective amount of the nanoparticles of the disclosure. In some embodiments, the methods comprise administering a chemotherapeutic agent to the subject.

The disclosure provides methods of treating a cancer in a subject, comprising administering to the subject a therapeutically effective amount of the pharmaceutical compositions of the disclosure to the subject. In some embodiments, the methods comprise administering a chemotherapeutic agent to the subject.

In some embodiments of the methods of the disclosure, the chemotherapeutic agent comprises a platinum based antineoplastic agent, a DNA alkylating agent, a DNA intercalating agent, or a topoisomerase inhibitor.

In some embodiments of the methods of the disclosure, the nanoparticles or the pharmaceutical composition is administered parenterally.

The disclosure provides methods of treating a cancer in a subject, comprising administering to the subject a therapeutically effective amount of a composition comprising a nanoparticle, the nanoparticle comprising a small interfering RNA (siRNA), wherein the siRNA comprises a sense region and anti-sense region complementary to the sense region that together form an RNA duplex, wherein the sense region comprises a sequence at least 70% identical to a glypican-2 (GPC2) mRNA sequence of SEQ ID NO: 1 or SEQ ID NO: 2.

In some embodiments of the methods of the disclosure, the sense region comprises a sequence that is identical to the GPC2 mRNA sequence. In some embodiments, the siRNA is capable of inducing RNAi-mediated degradation of a GPC2 mRNA in a cell of the cancer. In some embodiments, the sense region is encoded by a first single stranded RNA molecule and the anti-sense region is encoded by a second single stranded RNA molecule. In some embodiments, the first and second single stranded RNA molecules comprise 3′ overhangs. In some embodiments, the 3′ overhangs comprise thymidine-thymidine (dT-dT) or Uracil-Uracil (UU). In some embodiments, the RNA duplex is between 17 and 24 nucleotides in length. In some embodiments, the RNA duplex is 19 nucleotides in length. In some embodiments, the sense region comprises a sequence selected from the group listed in Table 1 or Table 2. In some embodiments, the siRNA comprises a sequence selected from the group consisting of SEQ ID NOs: 3-232. In some embodiments, the anti-sense region comprises a sequence selected from the group listed in Table 1 or Table 2. In some embodiments, the sense region comprises a sequence of CCUGCUUGGACCUCGAUAA (SEQ ID NO: 3), CUCAGUAGCCCAGCACUCU (SEQ ID NO: 4) or CUCCUUUCUGGUUCACACA (SEQ ID NO: 5). In some embodiments, the sense region comprises a sequence of CCUGCUUGGACCUCGAUAA (SEQ ID NO: 3) and the anti-sense region comprises a sequence of UUAUCGAGGUCCAAGCAGG (SEQ ID NO: 6). In some embodiments, the sense region comprises a sequence of CUCAGUAGCCCAGCACUCU (SEQ ID NO: 4) and the anti-sense region comprises a sequence of AGAGUGCUGGGCUACUGAG (SEQ ID NO: 7). In some embodiments, the sense region comprises a sequence of CUCCUUUCUGGUUCACACA (SEQ ID NO: 5) and the anti-sense region comprises a sequence of UGUGUGAACCAGAAAGGAG SEQ ID NO: 8).

In some embodiments of the methods of the disclosure, the siRNA comprises at least one modified nucleotide. In some embodiments, the at least one modified nucleotide increases stability of the RNA duplex. In some embodiments, the at least one modified nucleotide comprises a locked nucleic acid (LNA).

In some embodiments of the methods of the disclosure, the methods comprise administering a chemotherapeutic agent. In some embodiments, the chemotherapeutic agent comprises a platinum based antineoplastic agent, a DNA alkylating agent, a DNA intercalating agent, or a topoisomerase inhibitor. In some embodiments, the platinum based antineoplastic agent is Cisplatin or Carboplatin. In some embodiments, the DNA alkylating agent is Cyclophosphamide. In some embodiments, the DNA intercalating agent is Doxorubicin. In some embodiments, the topoisomerase inhibitor is Etoposide or Topotecan. In some embodiments, the platinum based antineoplastic agent is Cisplatin or Carboplatin. In some embodiments, the DNA alkylating agent is Cyclophosphamide. In some embodiments, the DNA intercalating agent is Doxorubicin. In some embodiments, the topoisomerase inhibitor is Etoposide or Topotecan.

In some embodiments of the methods of the disclosure, the pharmaceutical composition is administered at the same time as the chemotherapeutic agent. In some embodiments, the chemotherapeutic agent is formulated in the composition comprising the nanoparticle. In some embodiments, the chemotherapeutic and the siRNA are formulated in the same nanoparticle. In some embodiments, the pharmaceutical composition is administered in temporal proximity to the chemotherapeutic agent.

In some embodiments of the methods of the disclosure, the pharmaceutical composition is administered parenterally. In some embodiments, the parenteral administration is intravenous, subcutaneous, intraperitoneal or intramuscular. In some embodiments, the parenteral administration comprises an intravenous injection or infusion. In some embodiments, the methods further comprise a standard of care for the cancer. In some embodiments of the methods of the disclosure, the cancer expresses GPC2 on a surface of a cell of the cancer.

In some embodiments, the cancer is selected from the group consisting of astrocytoma, breast cancer, colorectal cancer, Ewing's sarcoma, gastric cancer, leiomyosarcoma, liver cancer, lung cancer, mesothelioma, ovarian cancer, pancreatic cancer, renal cancer, rhabdomyosarcoma and neuroblastoma. In some embodiments, the cancer is neuroblastoma.

In some embodiments of the methods of the disclosure, administration of the nanoparticle or the pharmaceutical composition decreases viability of a cell of the cancer. In some embodiments, administration of the composition increases apoptosis of cancer cells. In some embodiments, administration of the composition increases sensitivity of the cancer to the chemotherapeutic agent. In some embodiments, administration of the composition increases the effectiveness of the chemotherapeutic agent. In some embodiments, administration of the composition decreases the IC50 of the chemotherapeutic agent. In some embodiments, administration of the composition reduces a side effect of the chemotherapeutic agent. In some embodiments, administration of the composition reduces the therapeutically effective dose of a chemotherapeutic agent. In some embodiments, administration of the composition reduces a sign or a symptom of the cancer.

### BRIEF DESCRIPTION OF THE DRAWINGS

FIG. 1 is a graph showing percentages of cells stained for GPC2 in neuroblastoma (NB) tumor samples and normal tissues (NORMAL). Increased GPC2 protein expression is observed in neuroblastoma tumors when compared to normal tissue.

FIG. 2A is a plot showing knockdown of GPC2 mRNA using siRNA sequences 1-5 from Table 7 in the Ewing's Sarcoma TC-32 cell line. Expression is compared to a scrambled control siRNA.

FIG. 2B is a plot showing that GPC2 mRNA knockdown in the Ewing's Sarcoma TC-32 cell line was sustained for at least 96 hours following transfection with siRNAs sequence 3 or 5 from Table 7. Expression is compared to a scrambled control siRNA.

FIG. 3A is a plot showing relative GPC2 mRNA expression following siRNA mediated knockdown of GPC2 in the CHP212, SKNAS and SHSY5Y neuroblastoma cell lines using GPC2 siRNA sequences 3, 5, 12, 13, 14, and 15 from Table 7. Expression is compared to a scrambled control siRNA.

FIG. 3B is a plot showing percent viability of cells following siRNA mediated knockdown of GPC2 in the CHP212, SKNAS and SHSY5Y neuroblastoma cell lines using GPC2 siRNA sequences 3, 5, 12, 13, 14, and 15 from Table 7. Viability is compared to a scrambled control siRNA.

FIG. 4A is a plot showing relative GPC2 mRNA expression following GPC2 mRNA knockdown in the CHP212 neuroblastoma cell line following transfection with GPC2 siRNA sequences 3, 5, 6, 7, 8, 9, 10 and 11 from Table 7 and a scrambled control siRNA.

FIG. 4B is a plot cell viability following GPC2 mRNA knockdown in the CHP212 neuroblastoma cell line following transfection with GPC2 siRNAs 3, 5, 6, 7, 8, 9, 10 and 11 from Table 7 and a scrambled control siRNA.

FIG. 5A is a plot showing relative GPC2 mRNA expression in CHP212 neuroblastoma cells following transfection with GPC2 siRNA sequences 5 and 7 from Table 7 and a scrambled control siRNA. Reduced GPC2 mRNA expression seen with siRNA sequences 5 and 7 was sustained for 72 hours.

FIG. 5B is a plot showing relative GPC2 mRNA expression in SH-SY5Y neuroblastoma cells following transfection with GPC2 siRNA sequences 5 and 7 from Table 7 and a scrambled control siRNA. Reduced GPC2 mRNA expression seen with siRNA sequences 5 and 7 was sustained for 72 hours.

FIG. 5C is a plot showing relative GPC2 mRNA expression in CHLA90 neuroblastoma cells following transfection with GPC2 siRNA sequences 5 and 7 from Table 7 and a scrambled control siRNA. Reduced GPC2 mRNA expression seen with siRNA sequences 5 and 7 was sustained for 72 hours.

FIG. 5D is a plot showing GPC2 protein expression levels in neuroblastoma CHP212 cells following transfection with GPC2 siRNA sequences 5 and 7 from Table 7 and a scrambled control siRNA. Protein expression was measured via flow cytometry of cells fixed and immunostained with a GPC2 antibody, and is reported as Mean Fluorescence (FL1) Intensity. Reduced GPC2 protein expression with siRNA sequences 5 and 7 can be seen at 48 and 72 hours.

FIG. 5E is a plot showing GPC2 protein expression levels in SH-SY5Y neuroblastoma cells following transfection with GPC2 siRNA sequences 5 and 7 from Table 7 and a scrambled control siRNA. Protein expression was measured via flow cytometry of cells fixed and immunostained with a GPC2 antibody, and is reported as Mean Fluorescence (FL1) Intensity. Reduced GPC2 protein expression with siRNA sequences 5 and 7 can be seen at 48 and 72 hours.

FIG. 5F is a plot showing GPC2 protein expression levels in CHLA90 neuroblastoma cells following transfection with GPC2 siRNA sequences 5 and 7 from Table 7 and a scrambled control siRNA. Protein expression was measured via flow cytometry of cells fixed and immunostained with a GPC2 antibody, and is reported as Mean Fluorescence (FL1) Intensity. Reduced GPC2 protein expression with siRNA sequences 5 and 7 can be seen at 72 hours.

FIG. 6 is as series of 12 images showing the effect of siRNA-mediated reduction in GPC2 expression on tumor spheroid formation. Columns, from left to right, show tumor spheroids from CHP212, SKNAS, SKNBE2 and CHLA90 neuroblastoma cells. At bottom, the percent knockdown efficiencies of GPC2 mRNA with the siRNA of sequence 5 from Table 1 are indicated. Top row: no treatment; middle row: cells transfected with a scrambled control siRNA; bottom row: cells were transfected with GPC2 siRNA of sequence 5 from Table 7.

FIG. 7A is a series of four plots showing the relative levels of GPC2 mRNA following siRNA mediated GPC2 mRNA knockdown in established spheroid cultures. From left to right, spheroids from CHP212, SKNAS and SK—N-BE2 and CHLA90 neuroblastoma cells were transfected with either a scrambled siRNA control (siControl), or siRNAs of sequence 5 or sequence 7 from Table 7.

FIG. 7B is a series of four plots showing the effect on cell viability of siRNA mediated GPC2 mRNA knockdown in established spheroid cultures. From left to right, spheroids from CHP212, SKNAS and SK—N-BE2 and CHLA90 neuroblastoma cells were transfected with either a scrambled siRNA control (siControl), or siRNAs of sequence 5 or sequence 7 from Table 7.

### DETAILED DESCRIPTION

Glypican-2 (Glypican 2, GPC2, or GPC-2) is a cell surface protein that belongs to a family of six proteoglycans. These proteins are Glycosylphosphatidylinositol (GPI) anchored to the cell membrane, and play diverse roles in signaling and cancer cell growth. Although GPC2 was initially thought to be solely expressed during nervous system development, GPC2 is also expressed in neuroblastoma and other cancers. Further, GPC2 is regulated by the MYC oncogene via direct transcriptional activation. Treating GPC2 positive neuroblastoma cells with a GPC2 antibody conjugated to pyrrolobenzodiazepine, a cytotoxic DNA crosslinking agent, reduced cell proliferation and increased apoptosis in neuroblastoma cell lines and increased survival in neuroblast patient derived xenograft (PDX) mouse models. Higher levels of GPC2 expression can result in enhanced tumor cell growth, while decreasing GPC2 expression can decrease neuroblastoma cell viability.

Without wishing to be bound by theory, the inventors have found that use of a small interfering RNA (siRNA) can decrease GPC2 messenger RNA expression, in turn leading to a decrease in the degree of GPC-2 protein expression, and inhibiting proliferation of cancer cells expressing GPC2. Affecting GPC2 protein expression leads to a decrease in GPC2 function which results in an anti-cancer activity, for example by increasing apoptosis.

The inventors have shown that knocking down GPC2 mRNA resulted in a decrease in cell viability in multiple cancer cell lines. In addition, when combined with a chemotherapeutic agent, knocking down GPC2 mRNA resulted in a decrease in the IC₅₀ of the chemotherapeutic agent in treating cancer. Thus, knocking down GPC2 mRNA can enhance the activity of chemotherapeutic or therapeutic agents. When chemotherapeutic agents are administered in combination with GPC2 siRNAs, this combination can (a) reduce the therapeutically effective dose of the chemotherapeutic agent, (b) reduce side effects of the chemotherapeutic agent, and (c) increase the effectiveness of the chemotherapeutic agent.

In some embodiments, the siRNA targeting GPC2 is encapsulated in a nanoparticle that contains a tumor targeting agent or moiety. Exemplary tumor targeting agents include hyaluronic acid (HA). Tumor targeting agents on the surface of the nanoparticle can specifically target the nanoparticle to tumor cells that express a binding partner or receptor for the tumor targeting agent. For example, in the case of HA, HA on the nanoparticle can bind to CD44 molecules (CD44) on surface of cancer cells expressing CD44. Encapsulating GPC2 targeting siRNAs in a nanoparticle comprising a tumor targeting agent such as HA can thus increase the delivery of the siRNA to tumor cells.

GPC2 targeting siRNAs, and nanoparticles comprising same siRNAs, have utility for the treatment of any cancer that expresses GPC2. For example, neuroblastomas frequently express GPC2. GPC2 targeting siRNAs, and nanoparticles comprising siRNAs targeting GPC2, can be thus be used to treat neuroblastomas.

Accordingly, the disclosure provides nanoparticles comprising siRNAs, wherein the siRNA comprises a sense region and anti-sense region complementary to the sense region that together form an RNA duplex, and wherein the sense region comprises a sequence at least 70% to 100% identical to a glypican-2 (GPC2) mRNA sequence.

The disclosure provides pharmaceutical compositions comprising the nanoparticles comprising siRNAs targeting a GPC2 mRNA sequence described herein, and a pharmaceutically acceptable carrier, diluent or excipient.

The disclosure provides methods of treating a cancer in a subject, comprising administering to the subject a therapeutically effective amount of a composition comprising nanoparticles comprising siRNAs targeting a GPC2 mRNA described herein. In some embodiments, the methods further comprise combining the nanoparticles with a chemotherapeutic agent.

### Definitions

“RNAi” or “RNA interference” refers to the process of sequence-specific post-transcriptional gene silencing, mediated by double-stranded RNA (dsRNA). Duplex RNA siRNA (small interfering RNA), miRNA (micro RNA), shRNA (short hairpin RNA), ddRNA (DNA-directed RNA), piRNA (Piwi-interacting RNA), or rasiRNA (repeat associated siRNA) and modified forms thereof are all capable of mediating RNA interference. These dsRNA molecules may be commercially available or may be designed and prepared based on known sequence information, etc. The anti-sense strand of these molecules can include RNA, DNA, PNA, or a combination thereof. These DNA/RNA chimera polynucleotide includes, but is not limited to, a double-strand polynucleotide composed of DNA and RNA that inhibits the expression of a target gene. These dsRNA molecules can also include one or more modified nucleotides, as described herein, which can be incorporated on either strand.

In the RNAi gene silencing or knockdown process, dsRNA comprising a first (anti-sense) strand that is complementary to a portion of a target gene and a second (sense) strand that is fully or partially complementary to the first anti-sense strand is introduced into an organism. After introduction into the organism, the target gene-specific dsRNA is processed into relatively small fragments (siRNAs) and can subsequently become distributed throughout the organism, decrease messenger RNA of target gene, leading to a phenotype that may come to closely resemble the phenotype arising from a complete or partial deletion of the target gene.

Certain dsRNAs in cells can undergo the action of Dicer enzyme, a ribonuclease III enzyme. Dicer can process the dsRNA into shorter pieces of dsRNA, i.e. siRNAs. RNAi also involves an endonuclease complex known as the RNA induced silencing complex (RISC). Following cleavage by Dicer, siRNAs enter the RISC complex and direct cleavage of a single stranded RNA target having a sequence complementary to the anti-sense strand of the siRNA duplex. The other strand of the siRNA is the passenger strand. Cleavage of the target RNA takes place in the middle of the region complementary to the anti-sense strand of the siRNA duplex. siRNAs can thus down regulate or knock down gene expression by mediating RNA interference in a sequence-specific manner.

As used herein, “target gene” or “target sequence” refers to a gene or gene sequence whose corresponding RNA is targeted for degradation through the RNAi pathway using dsRNAs or siRNAs as described herein. To target a gene, for example using an siRNA, the siRNA comprises an anti-sense region complementary to, or substantially complementary to, at least a portion of the target gene or sequence, and sense strand complementary to the anti-sense strand. Once introduced into a cell, the siRNA directs the RISC complex to cleave an RNA comprising a target sequence, thereby degrading the RNA.

As used herein, “nucleic acid,” “nucleotide sequence,” and “polynucleotide” are used interchangeably and encompass both RNA and DNA, including cDNA, genomic DNA, mRNA, synthetic (e.g., chemically synthesized) DNA or RNA and chimeras of RNA and DNA. The term polynucleotide, nucleotide sequence, or nucleic acid refers to a chain of nucleotides without regard to length of the chain. The nucleic acid can be double-stranded or single-stranded. Where single-stranded, the nucleic acid can be a sense strand or an anti-sense strand. The nucleic acid can be synthesized using oligonucleotide analogs or derivatives (e.g., inosine or phosphorothioate nucleotides). Such oligonucleotides can be used, for example, to prepare nucleic acids that have altered base-pairing abilities or increased resistance to nucleases. The present invention further provides a nucleic acid that is the complement (which can be either a full complement or a partial complement) of a nucleic acid, nucleotide sequence, or polynucleotide of this invention. When dsRNA is produced synthetically, less common bases, such as inosine, 5-methylcytosine, 6-methyladenine, hypoxanthine and others can also be used for anti-sense, dsRNA, and ribozyme pairing. Other modifications, such as modification to the phosphodiester backbone, or the 2′-hydroxy or 2′O-methyl in the ribose sugar group of the RNA can also be made.

The term “isolated” can refer to a nucleic acid, nucleotide sequence or polypeptide that is substantially free of cellular material, viral material, and/or culture medium (when produced by recombinant DNA techniques), or chemical precursors or other chemicals (when chemically synthesized). Moreover, an “isolated fragment” is a fragment of a nucleic acid, nucleotide sequence or polypeptide that is not naturally occurring as a fragment and would not be found in the natural state. “Isolated” does not mean that the preparation is technically pure (homogeneous), but it is sufficiently pure to provide the polypeptide or nucleic acid in a form in which it can be used for the intended purpose.

The term “fragment,” as applied to a polynucleotide, will be understood to mean a nucleotide sequence of reduced length relative to a reference nucleic acid or nucleotide sequence and comprising, consisting essentially of, and/or consisting of a nucleotide sequence of contiguous nucleotides identical or almost identical (e.g., 60%, 70%, 80%, 90%, 92%, 95%, 98% or 99% identical) to the reference nucleic acid or nucleotide sequence. Such a nucleic acid fragment according to the invention may be, where appropriate, included in a larger polynucleotide of which it is a constituent. In some embodiments, such fragments can comprise, consist essentially of, and/or consist of oligonucleotides having a length of at least about 8, 10, 12, 15, 20, 25, 30, 35, 40, 45, 50, 75, 100, 150, 200, or more consecutive nucleotides of a nucleic acid or nucleotide sequence according to the invention.

As used herein, “complementary” polynucleotides are those that are capable of base pairing according to the standard Watson-Crick complementarity rules. Specifically, purines will base pair with pyrimidines to form a combination of guanine paired with cytosine (G:C) and adenine paired with either thymine (A:T) in the case of DNA, or adenine paired with uracil (A:U) in the case of RNA. For example, the sequence “A-G-T” binds to the complementary sequence “T-C-A.” It is understood that two polynucleotides may hybridize to each other even if they are not completely complementary to each other, provided that each has at least one region that is substantially complementary to the other.

As used herein, the terms “substantially complementary” or “partially complementary” mean that two nucleic acid sequences are complementary at least at about 50%, 60%, 70%, 80% or 90% of their nucleotides.

In some embodiments, the two nucleic acid sequences can be complementary at least at 85%, 90%, 95%, 96%, 97%, 98%, 99% or more of their nucleotides. In some embodiments, the two nucleic acid sequences can be between 60% to 100% complementary, between 70% to 100% complementary, between 80% and 100% complementary, between 90% and 100% complementary, between 60% to 90% complementary, between 60% to 80% complementary, between 60% and 70% complementary, between 70% and 90% complementary, between 70% and 80% complementary, between 80% and 100% complementary, or between 80% and 90% complementary.

The terms “substantially complementary” and “partially complementary” can also mean that two nucleic acid sequences can hybridize under high stringency conditions, and such conditions are well known in the art.

As used herein, the term “identity” means that sequences are compared with one another as follows. In order to determine the percentage identity of two nucleic acid sequences, the sequences can first be aligned with respect to one another in order subsequently to make a comparison of these sequences possible. For this e.g. gaps can be inserted into the sequence of the first nucleic acid sequence and the nucleotides can be compared with the corresponding position of the second nucleic acid sequence. If a position in the first nucleic acid sequence is occupied by the same nucleotide as is the case at a position in the second sequence, the two sequences are identical at this position. The percentage identity between two sequences is a function of the number of identical positions divided by the number of all the positions compared in the sequences investigated.

A “percent identity” for aligned segments of a test sequence and a reference sequence is the percent of identical components which are shared by the two aligned sequences divided by the total number of components in reference sequence segment, i.e., the entire reference sequence or a smaller defined part of the reference sequence.

The percentage identity of two sequences can be determined with the aid of a mathematical algorithm. A preferred, but not limiting, example of a mathematical algorithm which can be used for comparison of two sequences is the algorithm of Karlin et al. (1993), PNAS USA, 90:5873-5877. Such an algorithm is integrated in the NBLAST program, with which sequences which have a desired identity to the sequences of the present invention can be identified. In order to obtain a gapped alignment, as described here, the “Gapped BLAST” program can be used, as is described in Altschul et al. (1997), Nucleic Acids Res, 25:3389-3402. If BLAST and Gapped BLAST programs are used, the preset parameters of the particular program (e.g. NBLAST) can be used. The sequences can be aligned further using version 9 of GAP (global alignment program) of the “Genetic Computing Group” using the preset (BLOSUM62) matrix (values −4 to +11) with a gap open penalty of −12 (for the first zero of a gap) and a gap extension penalty of −4 (for each additional successive zero in the gap). After the alignment, the percentage identity is calculated by expressing the number of agreements as a percentage content of the nucleic acids in the sequence claimed. The methods described for determination of the percentage identity of two nucleic acid sequences can also be used correspondingly, if necessary, on the coded amino acid sequences.

Useful methods for determining sequence identity are also disclosed in Guide to Huge Computers (Martin J. Bishop, ed., Academic Press, San Diego (1994)), and Carillo, H., and Lipton, D., (Applied Math 48:1073(1988)). More particularly, preferred computer programs for determining sequence identity include but are not limited to the Basic Local Alignment Search Tool (BLAST) programs which are publicly available from National Center Biotechnology Information (NCBI) at the National Library of Medicine, National Institute of Health, Bethesda, Md. 20894; see BLAST Manual, Altschul et al., NCBI, NLM, NIH; (Altschul et al., J. Mol. Biol. 215:403-410 (1990)); version 2.0 or higher of BLAST programs allows the introduction of gaps (deletions and insertions) into alignments; for peptide sequence BLASTX can be used to determine sequence identity; and, for polynucleotide sequence BLASTN can be used to determine sequence identity. Percent identity can be 70% identity or greater, e.g., at least 70% identity, at least 75% identity, at least 80% identity, at least 85% identity, at least 90% identity, at least 95% identity, at least 98% identity, at least 99% identity or 100% identity.

As used herein, “heterologous” refers to a nucleic acid sequence that either originates from another species or is from the same species or organism but is modified from either its original form or the form primarily expressed in the cell. Thus, a nucleotide sequence derived from an organism or species different from that of the cell into which the nucleotide sequence is introduced, is heterologous with respect to that cell and the cell's descendants. In addition, a heterologous nucleotide sequence includes a nucleotide sequence derived from and inserted into the same natural, original cell type, but which is present in a non-natural state, e.g., a different copy number, and/or under the control of different regulatory sequences than that found in nature.

### Double Stranded RNAs Targeting Glypican-2

The disclosure provides double stranded RNAs (dsRNAs) which target a glypican-2 (GPC2) mRNA sequence for degradation. The double stranded RNA molecule of the invention may be in the form of any type of RNA interference molecule known in the art. In some embodiments, the double stranded RNA molecule is a small interfering RNA (siRNA). In other embodiments, the double stranded RNA molecule is a short hairpin RNA (shRNA) molecule. In other embodiments, the double stranded RNA molecule is a Dicer substrate that is processed in a cell to produce an siRNA. In other embodiments the double stranded RNA molecule is part of a microRNA precursor molecule.

In some embodiments, the dsRNA is a small interfering RNA (siRNA) which targets a glypican-2 (GPC2) mRNA sequence for degradation. In some embodiments, the siRNA targeting GPC2 is packaged in a nanoparticle.

An exemplary Glypican-2 sequence is described in NM_152742.3, the contents of which are incorporated by reference in their entirety herein. In some embodiments, a human GPC2 mRNA comprises a sequence of:

| (SEQ ID NO: 1) | |
|---|---|
| 1 | GCTCCCATTG TCTCGGCAGA TGCCGCCTGG TCCAGCTATC GTGCTCGGTA TTCAGTTTTC |
| ` | |
| 61 | CGGAGCAGCG CTCTTTCTCT GGCCCGCGGA GCGGTCCCGC GGCCGAGTAC CGGATTCCCG |
| 121 | AGTTTGGGAG GCTCTGCTTT CCTCCTTAGG ACCCACTTTG CCGTCCTGGG GTGGCTGCAG |
| 181 | TTATGTCCGC GCTGCGACCT CTCCTGCTTC TGCTGCTGCC TCTGTGTCCC GGTCCTGGTC |
| 241 | CCGGACCCGG GAGCGAGGCA AAGGTCACCC GGAGTTGTGC AGAGACCCGG CAGGTGCTGG |
| 301 | GGGCCCGGGG ATATAGCTTA AACCTAATCC CTCCCGCCCT GATCTCAGGT GAGCACCTCC |
| 361 | GGGTCTGTCC CCAGGAGTAC ACCTGCTGTT CCAGTGAGAC AGAGCAGAGG CTGATCAGGG |
| 421 | AGACTGAGGC CACCTTCCGA GGCCTGGTGG AGGACAGCGG CTCCTTTCTG GTTCACACAC |
| 481 | TGGCTGCCAG GCACAGAAAA TTTGATGAGT TTTTTCTGGA GATGCTCTCA GTAGCCCAGC |
| 541 | ACTCTCTGAC CCAGCTCTTC TCCCACTCCT ACGGCCGCCT GTATGCCCAG CACGCCCTCA |
| 601 | TATTCAATGG CCTGTTCTCT CGGCTGCGAG ACTTCTATGG GGAATCTGGT GAGGGGTTGG |
| 661 | ATGACACCCT GGCGGATTTC TGGGCACAGC TCCTGGAGAG AGTGTTCCCG CTGCTGCACC |
| 721 | CACAGTACAG CTTCCCCCCT GACTACCTGC TCTGCCTCTC ACGCTTGGCC TCATCTACCG |
| 781 | ATGGCTCTCT GCAGCCCTTT GGGGACTCAC CCCGCCGCCT CCGCCTGCAG ATAACCCGGA |
| 841 | CCCTGGTGGC TGCCCGAGCC TTTGTGCAGG GCCTGGAGAC TGGAAGAAAT GTGGTCAGCG |
| 901 | AAGCGCTTAA GGTGCCGGTG TCTGAAGGCT GCAGCCAGGC TCTGATGCGT CTCATCGGCT |
| 961 | GTCCCCTGTG CCGGGGGGTC CCCTCACTTA TGCCCTGCCA GGGCTTCTGC CTCAACGTGG |
| 1021 | TTCGTGGCTG TCTCAGCAGC AGGGGACTGG AGCCTGACTG GGGCAACTAT CTGGATGGTC |
| 1081 | TCCTGATCCT GGCTGATAAG CTCCAGGGCC CCTTTTCCTT TGAGCTGACG GCCGAGTCCA |
| 1141 | TTGGGGTGAA GATCTCGGAG GGTTTGATGT ACCTGCAGGA AAACAGTGCG AAGGTGTCCG |
| 1201 | CCCAGGTGTT TCAGGAGTGC GGCCCCCCCG ACCCGGTGCC TGCCCGCAAC CGTCGAGCCC |
| 1261 | CGCCGCCCCG GGAAGAGGCG GGCCGGCTGT GGTCGATGGT GACCGAGGAG GAGCGGCCCA |
| 1321 | CGACGGCCGC AGGCACCAAC CTGCACCGGC TGGTGTGGGA GCTCCGCGAG CGTCTGGCCC |
| 1381 | GGATGCGGGG CTTCTGGGCC CGGCTGTCCC TGACGGTGTG CGGAGACTCT CGCATGGCAG |
| 1441 | CGGACGCCTC GCTGGAGGCG GCGCCCTGCT GGACCGGAGC CGGGCGGGGC CGGTACTTGC |
| 1501 | CGCCAGTGGT CGGGGGCTCC CCGGCCGAGC AGGTCAACAA CCCCGAGCTC AAGGTGGACG |
| 1561 | CCTCGGGCCC CGATGTCCCG ACACGGCGGC GTCGGCTACA GCTCCGGGCG GCCACGGCCA |
| 1621 | GAATGAAAAC GGCCGCACTG GGACACGACC TGGACGGGCA GGACGCGGAT GAGGATGCCA |
| 1681 | GCGGCTCTGG AGGGGGACAG CAGTATGCAG ATGACTGGAT GGCTGGGGCT GTGGCTCCCC |
| 1741 | CAGCCCGGCC TCCTCGGCCT CCATACCCTC CTAGAAGGGA TGGTTCTGGG GGCAAAGGAG |
| 1801 | GAGGTGGCAG TGCCCGCTAC AACCAGGGCC GGAGCAGGAG TGGGGGGGCA TCTATTGGTT |
| 1861 | TTCACACCCA AACCATCCTC ATTCTCTCCC TCTCAGCCCT GGCCCTGCTT GGACCTCGAT |
| 1921 | AACGGGGGAG GGGTGCCCTA GCATCAGAAG GGTTCATGGC CCTTTCCCCT CCTCCCCCCT |
| 1981 | CAGCTGGGCC TGGGGAGGAG TCGAAGGGGG CTGCAGAGAG GGTAGAGAAG GGACTTTGCA |
| 2041 | GGTGAATGGC TGGGGCCCCA AATCCAGGAG ATTTTCATCA GAGGTGGGTG GGTGTTCACA |
| 2101 | ATATTTATTT TTTCATTTGG TAATGGGAGG GGGGCCTGGG GGTATTTATT TAGGAGGGAG |
| 2161 | TGTGGTTTCC TTAGAAGGTA TAGTCTCTAG CCCTCTAAGG CTGGGGCTGG TGATCAGCCC |
| 2221 | CAACAGAGAA AATGAGGAGT TTAGAGTTGC AGCTGGGGAA GGGGTTTGAA GGAAGTTGGA |
| 2281 | AGTGGGGAGG GGTGGGGGCA TCTGGTCTCA GAAATGGACC AGCTGGATGC AGGGCAGGGG |
| 2341 | ACTGAGGGTG CTTGAGTAGG ATGTGAGACT TCATGGGCCT GGGTTCTGTT GAGTTTTTTC |
| 2401 | AGTATCAATT TCTTAAACCA AATTTTAAAA AAAACAAGGT GGGGGGGTGC TCATCTCGTG |
| 2461 | ACCTCTGCCA CCCACATCCT TCACAAACTC CATGTTTCAG TGTTTGAGTC CATGTTTATT |
| 2521 | CTGCAAATAA ATGGTAATGT ATTGGA. |

A further example of a Glypican-2 sequence is described in NM_152742.2, the contents of which are incorporated by reference in their entirety herein. In some embodiments, a human GPC2 mRNA comprises a sequence of:

| (SEQ ID NO: 2) | |
|---|---|
| 1 | ATTGGCGGGG CCCCGCCTCG GGCCCCGCCC CCTGTCCGGC TCCCCGCTCC CATTGTCTCG |
| 61 | GCAGATGCCG CCTGGTCCAG CTATCGTGCT CGGTATTCAG TTTTCCGGAG CAGCGCTCTT |
| 121 | TCTCTGGCCC GCGGAGCGGT CCCGCGGCCG AGTACCGGAT TCCCGAGTTT GGGAGGCTCT |
| 181 | GCTTTCCTCC TTAGGACCCA CTTTGCCGTC CTGGGGTGGC TGCAGTTATG TCCGCGCTGC |
| 241 | GACCTCTCCT GCTTCTGCTG CTGCCTCTGT GTCCCGGTCC TGGTCCCGGA CCCGGGAGCG |
| 301 | AGGCAAAGGT CACCCGGAGT TGTGCAGAGA CCCGGCAGGT GCTGGGGGCC CGGGGATATA |
| 361 | GCTTAAACCT AATCCCTCCC GCCCTGATCT CAGGTGAGCA CCTCCGGGTC TGTCCCCAGG |
| 421 | AGTACACCTG CTGTTCCAGT GAGACAGAGC AGAGGCTGAT CAGGGAGACT GAGGCCACCT |
| 481 | TCCGAGGCCT GGTGGAGGAC AGCGGCTCCT TTCTGGTTCA CACACTGGCT GCCAGGCACA |
| 541 | GAAAATTTGA TGAGTTTTTT CTGGAGATGC TCTCAGTAGC CCAGCACTCT CTGACCCAGC |
| 601 | TCTTCTCCCA CTCCTACGGC CGCCTGTATG CCCAGCACGC CCTCATATTC AATGGCCTGT |
| 661 | TCTCTCGGCT GCGAGACTTC TATGGGGAAT CTGGTGAGGG GTTGGATGAC ACCCTGGCGG |
| 721 | ATTTCTGGGC ACAGCTCCTG GAGAGAGTGT TCCCGCTGCT GCACCCACAG TACAGCTTCC |
| 781 | CCCCTGACTA CCTGCTCTGC CTCTCACGCT TGGCCTCATC TACCGATGGC TCTCTGCAGC |
| 841 | CCTTTGGGGA CTCACCCCGC CGCCTCCGCC TGCAGATAAC CCGGACCCTG GTGGCTGCCC |
| 901 | GAGCCTTTGT GCAGGGCCTG GAGACTGGAA GAAATGTGGT CAGCGAAGCG CTTAAGGTGC |
| 961 | CGGTGTCTGA AGGCTGCAGC CAGGCTCTGA TGCGTCTCAT CGGCTGTCCC CTGTGCCGGG |
| 1021 | GGGTCCCCTC ACTTATGCCC TGCCAGGGCT TCTGCCTCAA CGTGGTTCGT GGCTGTCTCA |
| 1081 | GCAGCAGGGG ACTGGAGCCT GACTGGGGCA ACTATCTGGA TGGTCTCCTG ATCCTGGCTG |
| 1141 | ATAAGCTCCA GGGCCCCTTT TCCTTTGAGC TGACGGCCGA GTCCATTGGG GTGAAGATCT |
| 1201 | CGGAGGGTTT GATGTACCTG CAGGAAAACA GTGCGAAGGT GTCCGCCCAG GTGTTTCAGG |
| 1261 | AGTGCGGCCC CCCCGACCCG GTGCCTGCCC GCAACCGTCG AGCCCCGCCG CCCCGGGAAG |
| 1321 | AGGCGGGCCG GCTGTGGTCG ATGGTGACCG AGGAGGAGCG GCCCACGACG GCCGCAGGCA |
| 1381 | CCAACCTGCA CCGGCTGGTG TGGGAGCTCC GCGAGCGTCT GGCCCGGATG CGGGGCTTCT |
| 1441 | GGGCCCGGCT GTCCCTGACG GTGTGCGGAG ACTCTCGCAT GGCAGCGGAC GCCTCGCTGG |
| 1501 | AGGCGGCGCC CTGCTGGACC GGAGCCGGGC GGGGCCGGTA CTTGCCGCCA GTGGTCGGGG |
| 1561 | GCTCCCCGGC CGAGCAGGTC AACAACCCCG AGCTCAAGGT GGACGCCTCG GGCCCCGATG |
| 1621 | TCCCGACACG GCGGCGTCGG CTACAGCTCC GGGCGGCCAC GGCCAGAATG AAAACGGCCG |
| 1681 | CACTGGGACA CGACCTGGAC GGGCAGGACG CGGATGAGGA TGCCAGCGGC TCTGGAGGGG |
| 1741 | GACAGCAGTA TGCAGATGAC TGGATGGCTG GGGCTGTGGC TCCCCCAGCC CGGCCTCCTC |
| 1801 | GGCCTCCATA CCCTCCTAGA AGGGATGGTT CTGGGGGCAA AGGAGGAGGT GGCAGTGCCC |
| 1861 | GCTACAACCA GGGCCGGAGC AGGAGTGGGG GGGCATCTAT TGGTTTTCAC ACCCAAACCA |
| 1921 | TCCTCATTCT CTCCCTCTCA GCCCTGGCCC TGCTTGGACC TCGATAACGG GGGAGGGGTG |
| 1981 | CCCTAGCATC AGAAGGGTTC ATGGCCCTTT CCCCTCCTCC CCCCTCAGCT GGGCCTGGGG |
| 2041 | AGGAGTCGAA GGGGGCTGCA GAGAGGGTAG AGAAGGGACT TTGCAGGTGA ATGGCTGGGG |
| 2101 | CCCCAAATCC AGGAGATTTT CATCAGAGGT GGGTGGGTGT TCACAATATT TATTTTTTCA |
| 2161 | TTTGGTAATG GGAGGGGGGC CTGGGGGTAT TTATTTAGGA GGGAGTGTGG TTTCCTTAGA |
| 2221 | AGGTATAGTC TCTAGCCCTC TAAGGCTGGG GCTGGTGATC AGCCCCAACA GAGAAAATGA |
| 2281 | GGAGTTTAGA GTTGCAGCTG GGGAAGGGGT TTGAAGGAAG TTGGAAGTGG GGAGGGGTGG |
| 2341 | GGGCATCTGG TCTCAGAAAT GGACCAGCTG GATGCAGGGC AGGGGACTGA GGGTGCTTGA |
| 2401 | GTAGGATGTG AGACTTCATG GGCCTGGGTT CTGTTGAGTT TTTTCAGTAT CAATTTCTTA |
| 2461 | AACCAAATTT TAAAAAAAAC AAGGTGGGGG GGTGCTCATC TCGTGACCTC TGCCACCCAC |
| 2521 | ATCCTTCACA AACTCCATGT TTCAGTGTTT GAGTCCATGT TTATTCTGCA AATAAATGGT |
| 2581 | AATGTATTGG AAAAAAAAAA AAAAAAAAAA AAAAA. |

siRNAs targeting GPC2 for degradation can comprise a sense strand at least 70% identical to any fragment of a GPC2 mRNA, for example the GPC2 mRNA of SEQ ID NO: 1 or SEQ ID NO: 2. In some embodiments, the sense strand comprises or consists essentially of a sequence at least 70%, at least 80%, at least 90%, at least 95% or is 100% identical to any fragment of SEQ ID NO: 1 or SEQ ID NO: 2. siRNAs targeting GPC2 for degradation can comprise an anti-sense strand at least 70% identical to a sequence complementary to any fragment of a GPC2 mRNA, for example the GPC2 mRNA of SEQ ID NO: 1 or SEQ ID NO: 2. In some embodiments, the anti-sense strand comprises or consists essentially of a sequence at least 70%, at least 80%, at least 90%, at least 95% or is 100% identical to a sequence complementary to any fragment of SEQ ID NO: 1 or SEQ ID NO: 2. In some embodiments, the sense region and anti-sense regions are complementary, and basepair to form an RNA duplex structure. The fragment of the GPC2 mRNA that has percent identity to the sense region of the siRNA, and which is complementary to the anti-sense region of the siRNA, can be protein coding sequence of the mRNA, an untranslated region (UTR) of the mRNA (5′ UTR or 3′ UTR), or both.

In some embodiments, the siRNA comprises a sense region and anti-sense region complementary to the sense region that together form an RNA duplex, and the sense region comprises a sequence at least 70% identical to a glypican-2 (GPC2) mRNA sequence. In some embodiments, the sense region is identical to a GPC2 mRNA sequence.

As used herein, the term “sense strand” or “sense region” refers to a nucleotide sequence of an siRNA molecule that is partially or fully complementary to at least a portion of a corresponding anti-sense strand or anti-sense region of the siRNA molecule. The sense strand of an siRNA molecule can include a nucleic acid sequence having some percentage identity with a target nucleic acid sequence such as a GPC2 mRNA sequence. In some cases, the sense region may have 100% identity, i.e. complete identity or homology, to the target nucleic acid sequence. In other cases, there may be one or more mismatches between the sense region and the target nucleic acid sequence. For example, there may be 1, 2, 3, 4, 5, 6, or 7 mismatches between the sense region and the target nucleic acid sequence.

As used herein, the term “anti-sense strand” or “anti-sense region” refers to a nucleotide sequence of an siRNA molecule that is partially or fully complementary to at least a portion of a target nucleic acid sequence. The anti-sense strand of an siRNA molecule can include a nucleic acid sequence that is complementary to at least a portion of a corresponding sense strand of the siRNA molecule.

In some embodiments, the sense region comprises a sequence that is at least 70% identical, at least 75% identical, at least 80% identical, at least 85% identical, at least 90% identical, at least 95% identical, at least 97% identical or 100% identical to a sequence of SEQ ID NO: 1 or SEQ ID NO: 2. In some embodiments, the sense region consists essentially of a sequence that is at least 70% identical, at least 75% identical, at least 80% identical, at least 85% identical, at least 90% identical, at least 95% identical, at least 97% identical or 100% identical to a sequence of SEQ ID NO: 1 or SEQ ID NO: 2. In some embodiments, the sense region comprises a sequence that is identical to a sequence of SEQ ID NO: 1 or SEQ ID NO: 2. In some embodiments, the sense region consists essentially of a sequence that is identical to a sequence of SEQ ID NO: 1 or SEQ ID NO: 2.

In some embodiments, the sense region of the siRNA targeting GPC2 has one or more mismatches between the sequence of the siRNA and the GPC2 sequence. For example, the sequence of the sense region may have 1, 2, 3, 4 or 5 mismatches between the sequence of the sense region of the siRNA and the GPC2 sequence. In some embodiments, the GPC2 sequence is a GPC2 3′ untranslated region sequence (3′ UTR). Without wishing to be bound by theory, it is thought that siRNAs targeting the 3′ UTR have elevated mismatch tolerance when compared to mismatches in siRNAs targeting coding regions of a gene. Further, siRNAs may be tolerant of mismatches outside the siRNA seed region. As used herein, the “seed region” of the siRNA refers to base pairs 2-8 of the anti-sense region of the siRNA, i.e. the strand of the siRNA that is complementary to and hybridizes to the target mRNA.

In some embodiments, the anti-sense region comprises a sequence that is at least 70% identical, at least 75% identical, at least 80% identical, at least 85% identical, at least 90% identical, at least 95% identical, at least 97% identical or 100% identical to a sequence complementary to a sequence of SEQ ID NO: 1 or SEQ ID NO: 2. In some embodiments, the anti-sense region consists essentially of a sequence that is at least 70% identical, at least 75% identical, at least 80% identical, at least 85% identical, at least 90% identical, at least 95% identical, at least 97% identical or 100% identical to a sequence complementary to a sequence of SEQ ID NO: 1 or SEQ ID NO: 2. In some embodiments, the anti-sense region comprises a sequence that is identical to a sequence complementary to a sequence of SEQ ID NO: 1 or SEQ ID NO: 2. In some embodiments, the sense region consists essentially of a sequence that is complementary to a sequence of SEQ ID NO: 1 or SEQ ID NO: 2.

The anti-sense region of the GPC2 targeting siRNA is complementary to the sense region. In some embodiments, the sense region and the anti-sense region are fully complementary (no mismatches). In some embodiments the anti-sense region is partially complementary to the sense region, i.e., there are 1, 2, 3, 4 or 5 mismatches between the sense region and the anti-sense region.

In general, siRNAs comprise an RNA duplex that is about 16 to about 25 nucleotides in length. In some embodiments, the RNA duplex is between 17 and 24 nucleotides in length, between about 18 and 23 nucleotides in length, or between about 19 and 22 nucleotides in length. In some embodiments, the RNA duplex is 19 nucleotides in length.

In some embodiments, the sense region is encoded by a first single stranded RNA molecule, and the anti-sense region is encoded by a second single stranded RNA molecule. In some embodiments, the siRNA targeting GPC2 comprises two different single stranded RNAs, the first comprising the sense region and the second comprising the anti-sense region, which hybridize to form an RNA duplex.

siRNAs of the disclosure can have one or more overhangs from the duplex region. The overhangs, which are non-base-paired, single strand regions, can be from one to eight nucleotides in length, or longer. An overhang can be a 3′ overhang, wherein the 3′-end of a strand has a single strand region of from one to eight nucleotides. An overhang can be a 5′ overhang, wherein the 5′-end of a strand has a single strand region of from one to eight nucleotides.

The overhangs of the siRNAs can be the same length, or can be different lengths.

siRNAs can have one or more blunt ends, in which the duplex region ends with no overhang, and the strands are base paired to the end of the duplex region. siRNAs of the disclosure can have one or more blunt ends, or can have one or more overhangs, or can have a combination of a blunt end and an overhang end. For example, the 5′ end of the siRNA can be blunt and the 3′ end of the same siRNA comprise an overhang, or vice versa.

In some embodiments, both ends of the siRNA are blunt ends.

In additional embodiments, both ends of siRNA have an overhang. In some embodiments, the overhang is a 3′ overhang, for example a 3′ dinucleotide overhang on each end. The overhangs at the 5′- and 3′-ends may be of different lengths, or be the same length.

An overhang of an siRNA can contain one or more deoxyribonucleotides, one or more ribonucleotides, or a combination of deoxyribonucleotides and ribonucleotides. In some embodiments, one, or both, of the overhang nucleotides of an siRNA may be 2′-deoxyribonucleotides.

In some embodiments, the first single stranded RNA molecule comprises a first 3′ overhang. In some embodiments, the second single stranded RNA molecule comprises a second 3′ overhang. In some embodiments, the first and second 3′ overhangs comprise a dinucleotide. In some embodiments, the dinucleotide comprises thymidine-thymidine (dT-dT) or Uracil-Uracil (UU). Without wishing to be bound by theory, it is thought that 3′ overhangs, such as dinucleotide overhangs, enhance siRNA mediated mRNA degradation by enhancing siRNA-RISC complex formation, and/or rate of cleavage of the target mRNA by the siRNA-RISC complex.

In some embodiments, the sense region comprises a sequence selected from the group listed in Table 1 and Table 2. In some embodiments, the anti-sense region comprises a sequence selected from the group listed in Table 1 or Table 2. In some embodiments, the sense and anti-sense regions comprise complementary sequences selected from the group listed in Table 1 and Table 2.

TABLE 1
Sense and anti-sense regions of
representative GPC2 siRNAs

| Sense Region | Anti-sense Region |
|---|---|
| CUCCUGAUCCUGGCUGAUA | UAUCAGCCAGGAUCAGGAG |
| (SEQ ID NO: 9) | (SEQ ID NO: 10) |
| CUCAUCUACCGAUGGCUCU | AGAGCCAUCGGUAGAUGAG |
| (SEQ ID NO: 11) | (SEQ ID NO: 12) |
| CCUGCUUGGACCUCGAUAA | UUAUCGAGGUCCAAGCAGG |
| (SEQ ID NO: 3) | (SEQ ID NO: 6) |
| GUGGUUCGUGGCUGUCUCA | UGAGACAGCCACGAACCAC |
| (SEQ ID NO: 13) | (SEQ ID NO: 14) |
| CUCAGUAGCCCAGCACUCU | AGAGUGCUGGGCUACUGAG |
| (SEQ ID NO: 4) | (SEQ ID NO: 7) |
| CUGCUGUUCCAGUGAGACA | UGUCUCACUGGAACAGCAG |
| (SEQ ID NO: 15) | (SEQ ID NO: 16) |
| CUCCUUUCUGGUUCACACA | UGUGUGAACCAGAAAGGAG |
| (SEQ ID NO: 5) | (SEQ ID NO: 8) |
| GAGUGUGGUUUCCUUAGAA | UUCUAAGGAAACCACACUC |
| (SEQ ID NO: 17) | (SEQ ID NO: 18) |
| GAGUACACCUGCUGUUCCA | UGGAACAGCAGGUGUACUC |
| (SEQ ID NO: 19) | (SEQ ID NO: 20) |
| GACACGACCUGGACGGGCA | UGCCCGUCCAGGUCGUGUC |
| (SEQ ID NO: 21) | (SEQ ID NO: 22) |
| CUGACUACCUGCUCUGCCU | AGGCAGAGCAGGUAGUCAG |
| (SEQ ID NO: 23) | (SEQ ID NO: 24) |
| GCGCUUAAGGUGCCGGUGU | ACACCGGCACCUUAAGCGC |
| (SEQ ID NO: 25) | (SEQ ID NO: 26) |
| CCUUUGAGCUGACGGCCGA | UCGGCCGUCAGCUCAAAGG |
| (SEQ ID NO: 27) | (SEQ ID NO: 28) |
| CCUGCUUCUGCUGCUGCCU | AGGCAGCAGCAGAAGCAGG |
| (SEQ ID NO: 29) | (SEQ ID NO: 30) |
| GAAGAAAUGUGGUCAGCGA | UCGCUGACCACAUUUCUUC |
| (SEQ ID NO: 31) | (SEQ ID NO: 32) |

In some embodiments, the sense region comprises a sequence of CCUGCUUGGACCUCGAUAA (SEQ ID NO: 3), CUCAGUAGCCCAGCACUCU (SEQ ID NO: 4) or CUCCUUUCUGGUUCACACA (SEQ ID NO: 5). In some embodiments, the anti-sense region comprises a sequence complementary to the sense region.

In some embodiments, the sense region comprises a sequence of CUCCUGAUCCUGGCUGAUA (SEQ ID NO: 9) and the anti-sense region comprises a sequence of UAUCAGCCAGGAUCAGGAG (SEQ ID NO: 10).

In some embodiments, the sense region comprises a sequence of CUCAUCUACCGAUGGCUCU (SEQ ID NO: 11) and the anti-sense region comprises a sequence of AGAGCCAUCGGUAGAUGAG (SEQ ID NO: 12).

In some embodiments, the sense region comprises a sequence of GUGGUUCGUGGCUGUCUCA (SEQ ID NO: 13) and the anti-sense region comprises a sequence of UGAGACAGCCACGAACCAC (SEQ ID NO: 14).

In some embodiments, the sense region comprises a sequence of CUGCUGUUCCAGUGAGACA (SEQ ID NO: 15) and the anti-sense region comprises a sequence of UGUCUCACUGGAACAGCAG (SEQ ID NO: 16).

In some embodiments, the sense region comprises a sequence of GAGUGUGGUUUCCUUAGAA (SEQ ID NO: 17) and the anti-sense region comprises a sequence of UUCUAAGGAAACCACACUC (SEQ ID NO: 18).

In some embodiments, the sense region comprises a sequence of GAGUACACCUGCUGUUCCA (SEQ ID NO: 19) and the anti-sense region comprises a sequence of UGGAACAGCAGGUGUACUC (SEQ ID NO: 20).

In some embodiments, the sense region comprises a sequence of GACACGACCUGGACGGGCA (SEQ ID NO: 21) and the anti-sense region comprises a sequence of UGCCCGUCCAGGUCGUGUC (SEQ ID NO: 22).

In some embodiments, the sense region comprises a sequence of CUGACUACCUGCUCUGCCU (SEQ ID NO: 23) and the anti-sense region comprises a sequence of AGGCAGAGCAGGUAGUCAG (SEQ ID NO: 24).

In some embodiments, the sense region comprises a sequence of GCGCUUAAGGUGCCGGUGU (SEQ ID NO: 25) and the anti-sense region comprises a sequence of ACACCGGCACCUUAAGCGC (SEQ ID NO: 26).

In some embodiments, the sense region comprises a sequence of CCUUUGAGCUGACGGCCGA (SEQ ID NO: 27) and the anti-sense region comprises a sequence of UCGGCCGUCAGCUCAAAGG (SEQ ID NO: 28).

In some embodiments, the sense region comprises a sequence of CCUGCUUCUGCUGCUGCCU (SEQ ID NO: 29) and the anti-sense region comprises a sequence of AGGCAGCAGCAGAAGCAGG (SEQ ID NO: 30).

In some embodiments, the sense region comprises a sequence of GAAGAAAUGUGGUCAGCGA (SEQ ID NO: 31) and the anti-sense region comprises a sequence of UCGCUGACCACAUUUCUUC (SEQ ID NO: 32).

In some embodiments, the sense region comprises a sequence of CCUGCUUGGACCUCGAUAA (SEQ ID NO: 3) and the anti-sense region comprises a sequence of UUAUCGAGGUCCAAGCAGG (SEQ ID NO: 6).

In some embodiments, the sense region comprises a sequence of CUCAGUAGCCCAGCACUCU (SEQ ID NO: 4) and the anti-sense region comprises a sequence of AGAGUGCUGGGCUACUGAG (SEQ ID NO: 7).

In some embodiments, the sense region comprises a sequence of CUCCUUUCUGGUUCACACA (SEQ ID NO: 5) and the anti-sense region comprises a sequence of UGUGUGAACCAGAAAGGAG (SEQ ID NO: 8).

TABLE 2
Sense and anti-sense regions of
representative GPC2 siRNAs

| Sense Region | Anti-sense Region |
|---|---|
| GGCUCCUUUCUGGUUCACA | UGUGAACCAGAAAGGAGCC |
| (SEQ ID NO: 33) | (SEQ ID NO: 34) |
| GCCUGGAGACUGGAAGAAA | UUUCUUCCAGUCUCCAGGC |
| (SEQ ID NO: 35) | (SEQ ID NO: 36) |
| GCAACUAUCUGGAUGGUCU | AGACCAUCCAGAUAGUUGC |
| (SEQ ID NO: 37) | (SEQ ID NO: 38) |
| GACUGAGGGUGCUUGAGUA | UACUCAAGCACCCUCAGUC |
| (SEQ ID NO: 39) | (SEQ ID NO: 40) |
| GUGCUUGAGUAGGAUGUGA | UCACAUCCUACUCAAGCAC |
| (SEQ ID NO: 41) | (SEQ ID NO: 42) |
| GCUUGAGUAGGAUGUGAGA | UCUCACAUCCUACUCAAGC |
| (SEQ ID NO: 43) | (SEQ ID NO: 44) |
| GUAGGAUGUGAGACUUCAU | AUGAAGUCUCACAUCCUAC |
| (SEQ ID NO: 45) | (SEQ ID NO: 46) |
| CCUCCUUAGGACCCACUUU | AAAGUGGGUCCUAAGGAGG |
| (SEQ ID NO: 47) | (SEQ ID NO: 48) |
| GCUGCGACCUCUCCUGCUU | AAGCAGGAGAGGUCGCAGC |
| (SEQ ID NO: 49) | (SEQ ID NO: 50) |
| GCACGCCCUCAUAUUCAAU | AUUGAAUAUGAGGGCGUGC |
| (SEQ ID NO: 51) | (SEQ ID NO: 52) |
| GGAUGACACCCUGGCGGAU | AUCCGCCAGGGUGUCAUCC |
| (SEQ ID NO: 53) | (SEQ ID NO: 54) |
| CCUGGAGACUGGAAGAAAU | AUUUCUUCCAGUCUCCAGG |
| (SEQ ID NO: 55) | (SEQ ID NO: 56) |
| GCCAGGGCUUCUGCCUCAA | UUGAGGCAGAAGCCCUGGC |
| (SEQ ID NO: 57) | (SEQ ID NO: 58) |
| GCUGACGGCCGAGUCCAUU | AAUGGACUCGGCCGUCAGC |
| (SEQ ID NO: 59) | (SEQ ID NO: 60) |
| CCCUAGCAUCAGAAGGGUU | AACCCUUCUGAUGCUAGGG |
| (SEQ ID NO: 61) | (SEQ ID NO: 62) |
| CCACCCACAUCCUUCACAA | UUGUGAAGGAUGUGGGUGG |
| (SEQ ID NO: 63) | (SEQ ID NO: 64) |
| CCAUGUUUAUUCUGCAAAU | AUUUGCAGAAUAAACAUGG |
| (SEQ ID NO: 65) | (SEQ ID NO: 66) |
| GCUAUCGUGCUCGGUAUUCA | UGAAUACCGAGCACGAUAGC |
| (SEQ ID NO: 67) | (SEQ ID NO: 68) |
| AUCGUGCUCGGUAUUCAGUU | AACUGAAUACCGAGCACGAU |
| (SEQ ID NO: 69) | (SEQ ID NO: 70) |
| GCUCUGCUUUCCUCCUUAGG | CCUAAGGAGGAAAGCAGAGC |
| (SEQ ID NO: 71) | (SEQ ID NO: 72) |
| GCUUCUGCUGCUGCCUCUGU | ACAGAGGCAGCAGCAGAAGC |
| (SEQ ID NO: 73) | (SEQ ID NO: 74) |
| GGGAGCGAGGCAAAGGUCAC | GUGACCUUUGCCUCGCUCCC |
| (SEQ ID NO: 75) | (SEQ ID NO: 76) |
| GCAAAGGUCACCCGGAGUUG | CAACUCCGGGUGACCUUUGC |
| (SEQ ID NO: 77) | (SEQ ID NO: 78) |
| GGGAUAUAGCUUAAACCUAA | UUAGGUUUAAGCUAUAUCCC |
| (SEQ ID NO: 79) | (SEQ ID NO: 80) |
| GGAUAUAGCUUAAACCUAAU | AUUAGGUUUAAGCUAUAUCC |
| (SEQ ID NO: 81) | (SEQ ID NO: 82) |
| GUGAGCACCUCCGGGUCUGU | ACAGACCCGGAGGUGCUCAC |
| (SEQ ID NO: 83) | (SEQ ID NO: 84) |
| GAGCACCUCCGGGUCUGUCC | GGACAGACCCGGAGGUGCUC |
| (SEQ ID NO: 85) | (SEQ ID NO: 86) |
| GGGUCUGUCCCCAGGAGUAC | GUACUCCUGGGGACAGACCC |
| (SEQ ID NO: 87) | (SEQ ID NO: 88) |
| GGUCUGUCCCCAGGAGUACA | UGUACUCCUGGGGACAGACC |
| (SEQ ID NO: 89) | (SEQ ID NO: 90) |
| GUCUGUCCCCAGGAGUACAC | GUGUACUCCUGGGGACAGAC |
| (SEQ ID NO: 91) | (SEQ ID NO: 92) |
| GCGGCUCCUUUCUGGUUCAC | GUGAACCAGAAAGGAGCCGC |
| (SEQ ID NO: 93) | (SEQ ID NO: 94) |
| GGCUCCUUUCUGGUUCACAC | GUGUGAACCAGAAAGGAGCC |
| (SEQ ID NO: 95) | (SEQ ID NO: 96) |
| GCUCUUCUCCCACUCCUACG | CGUAGGAGUGGGAGAAGAGC |
| (SEQ ID NO: 97) | (SEQ ID NO: 98) |
| GCCCAGCACGCCCUCAUAUU | AAUAUGAGGGCGUGCUGGGC |
| (SEQ ID NO: 99) | (SEQ ID NO: 100) |
| GCACGCCCUCAUAUUCAAUG | CAUUGAAUAUGAGGGCGUGC |
| (SEQ ID NO: 101) | (SEQ ID NO: 102) |
| GCUGCUGCACCCACAGUACA | UGUACUGUGGGUGCAGCAGC |
| (SEQ ID NO: 103) | (SEQ ID NO: 104) |
| GGCCUGGAGACUGGAAGAAA | UUUCUUCCAGUCUCCAGGCC |
| (SEQ ID NO: 105) | (SEQ ID NO: 106) |
| GCCUGGAGACUGGAAGAAAU | AUUUCUUCCAGUCUCCAGGC |
| (SEQ ID NO: 107) | (SEQ ID NO: 108) |
| GCUUAAGGUGCCGGUGUCUG | CAGACACCGGCACCUUAAGC |
| (SEQ ID NO: 109) | (SEQ ID NO: 110) |
| GGCAACUAUCUGGAUGGUCU | AGACCAUCCAGAUAGUUGCC |
| (SEQ ID NO: 111) | (SEQ ID NO: 112) |
| GCAACUAUCUGGAUGGUCUC | GAGACCAUCCAGAUAGUUGC |
| (SEQ ID NO: 113) | (SEQ ID NO: 114) |
| GGAAAACAGUGCGAAGGUGU | ACACCUUCGCACUGUUUUCC |
| (SEQ ID NO: 115) | (SEQ ID NO: 116) |
| GAAAACAGUGCGAAGGUGUC | GACACCUUCGCACUGUUUUC |
| (SEQ ID NO: 117) | (SEQ ID NO: 118) |
| GGACAGCAGUAUGCAGAUGA | UCAUCUGCAUACUGCUGUCC |
| (SEQ ID NO: 119) | (SEQ ID NO: 120) |
| GACAGCAGUAUGCAGAUGAC | GUCAUCUGCAUACUGCUGUC |
| (SEQ ID NO: 121) | (SEQ ID NO: 122) |
| ACAGCAGUAUGCAGAUGACU | AGUCAUCUGCAUACUGCUGU |
| (SEQ ID NO: 123) | (SEQ ID NO: 124) |
| GCCUCCUCGGCCUCCAUACC | GGUAUGGAGGCCGAGGAGGC |
| (SEQ ID NO: 125) | (SEQ ID NO: 126) |
| GGCCUCCAUACCCUCCUAGA | UCUAGGAGGGUAUGGAGGCC |
| (SEQ ID NO: 127) | (SEQ ID NO: 128) |
| GCCUCCAUACCCUCCUAGAA | UUCUAGGAGGGUAUGGAGGC |
| (SEQ ID NO: 129) | (SEQ ID NO: 130) |
| ACCCAAACCAUCCUCAUUCU | AGAAUGAGGAUGGUUUGGGU |
| (SEQ ID NO: 131) | (SEQ ID NO: 132) |
| GGGUGCCCUAGCAUCAGAAG | UUCUGAUGCUAGGGCACCC |
| (SEQ ID NO: 133) | (SEQ ID NO: 134) |
| GGGCUGCAGAGAGGGUAGAG | CUCUACCCUCUCUGCAGCCC |
| (SEQ ID NO: 135) | (SEQ ID NO: 136) |
| GGCUGCAGAGAGGGUAGAGA | UCUCUACCCUCUCUGCAGCC |
| (SEQ ID NO: 137) | (SEQ ID NO: 138) |
| GCUGCAGAGAGGGUAGAGAA | UUCUCUACCCUCUCUGCAGC |
| (SEQ ID NO: 139) | (SEQ ID NO: 140) |
| GAGGGUAGAGAAGGGACUUU | AAAGUCCCUUCUCUACCCUC |
| (SEQ ID NO: 141) | (SEQ ID NO: 142) |
| AUUUAUUUAGGAGGGAGUGU | ACACUCCCUCCUAAAUAAAU |
| (SEQ ID NO: 143) | (SEQ ID NO: 144) |
| GGUGAUCAGCCCCAACAGAG | CUCUGUUGGGGCUGAUCACC |
| (SEQ ID NO: 145) | (SEQ ID NO: 146) |
| AGCCCCAACAGAGAAAAUGA | UCAUUUUCUCUGUUGGGGCU |
| (SEQ ID NO: 147) | (SEQ ID NO: 148) |
| GCCCCAACAGAGAAAAUGAG | CUCAUUUUCUCUGUUGGGGC |
| (SEQ ID NO: 149) | (SEQ ID NO: 150) |
| AACAGAGAAAAUGAGGAGUU | AACUCCUCAUUUUCUCUGUU |
| (SEQ ID NO: 151) | (SEQ ID NO: 152) |
| GAGAAAAUGAGGAGUUUAGA | UCUAAACUCCUCAUUUUCUC |
| (SEQ ID NO: 153) | (SEQ ID NO: 154) |
| GAAAAUGAGGAGUUUAGAGU | ACUCUAAACUCCUCAUUUUC |
| (SEQ ID NO: 155) | (SEQ ID NO: 156) |
| AAAAUGAGGAGUUUAGAGUU | AACUCUAAACUCCUCAUUUU |
| (SEQ ID NO: 157) | (SEQ ID NO: 158) |
| GGGACUGAGGGUGCUUGAGU | ACUCAAGCACCCUCAGUCCC |
| (SEQ ID NO: 159) | (SEQ ID NO: 160) |
| GGACUGAGGGUGCUUGAGUA | UACUCAAGCACCCUCAGUCC |
| (SEQ ID NO: 161) | (SEQ ID NO: 162) |
| GACUGAGGGUGCUUGAGUAG | CUACUCAAGCACCCUCAGUC |
| (SEQ ID NO: 163) | (SEQ ID NO: 164) |
| GGUGCUUGAGUAGGAUGUGA | UCACAUCCUACUCAAGCACC |
| (SEQ ID NO: 165) | (SEQ ID NO: 166) |
| GUGCUUGAGUAGGAUGUGAG | CUCACAUCCUACUCAAGCAC |
| (SEQ ID NO: 167) | (SEQ ID NO: 168) |
| GCUUGAGUAGGAUGUGAGAC | GUCUCACAUCCUACUCAAGC |
| (SEQ ID NO: 169) | (SEQ ID NO: 170) |
| GUAGGAUGUGAGACUUCAUG | CAUGAAGUCUCACAUCCUAC |
| (SEQ ID NO: 171) | (SEQ ID NO: 172) |
| GGGUGCUCAUCUCGUGACCU | AGGUCACGAGAUGAGCACCC |
| (SEQ ID NO: 173) | (SEQ ID NO: 174) |
| GGUGCUCAUCUCGUGACCUC | GAGGUCACGAGAUGAGCACC |
| (SEQ ID NO: 175) | (SEQ ID NO: 176) |
| GUGCUCAUCUCGUGACCUCU | AGAGGUCACGAGAUGAGCAC |
| (SEQ ID NO: 177) | (SEQ ID NO: 178) |
| GCCACCCACAUCCUUCACAA | UUGUGAAGGAUGUGGGUGGC |
| (SEQ ID NO: 179) | (SEQ ID NO: 180) |
| ACCCACAUCCUUCACAAACU | AGUUUGUGAAGGAUGUGGGU |
| (SEQ ID NO: 181) | (SEQ ID NO: 182) |
| GCAAAUAAAUGGUAAUGUAU | AUACAUUACCAUUUAUUUGC |
| (SEQ ID NO: 183) | (SEQ ID NO: 184) |
| GGGAUAUAGCUUAAACCUAAU | AUUAGGUUUAAGCUAUAUCCC |
| (SEQ ID NO: 185) | (SEQ ID NO: 186) |
| GGCCUGGAGACUGGAAGAAAU | AUUUCUUCCAGUCUCCAGGCC |
| (SEQ ID NO: 187) | (SEQ ID NO: 188) |
| GGGCAACUAUCUGGAUGGUCU | AGACCAUCCAGAUAGUUGCCC |
| (SEQ ID NO: 189) | (SEQ ID NO: 190) |
| GGGACAGCAGUAUGCAGAUGA | UCAUCUGCAUACUGCUGUCCC |
| (SEQ ID NO: 191) | (SEQ ID NO: 192) |
| GACAGCAGUAUGCAGAUGACU | AGUCAUCUGCAUACUGCUGUC |
| (SEQ ID NO: 193) | (SEQ ID NO: 194) |
| GGGUGCUUGAGUAGGAUGUGA | UCACAUCCUACUCAAGCACCC |
| (SEQ ID NO: 195) | (SEQ ID NO: 196) |
| GUGCUUGAGUAGGAUGUGAGA | UCUCACAUCCUACUCAAGCAC |
| (SEQ ID NO: 197) | (SEQ ID NO: 198) |
| GCUUGAGUAGGAUGUGAGACU | AGUCUCACAUCCUACUCAAGC |
| (SEQ ID NO: 199) | (SEQ ID NO: 200) |
| GCCACCCACAUCCUUCACAAA | UUUGUGAAGGAUGUGGGUGGC |
| (SEQ ID NO: 201) | (SEQ ID NO: 202) |

In some embodiments, the siRNA targeting GPC2 comprises a sense region encoded by a first single stranded RNA molecule and an anti-sense region is encoded by a second single stranded RNA molecule, the anti-sense region is complementary to the sense region, and the first and second single stranded RNA molecules further comprise 3′ overhangs. In some embodiments, the first single stranded RNA comprises or consists essentially of a sequence selected from the group listed in Table 3. In some embodiments, the second single stranded RNA comprises or consists essentially of a sequence selected from the group listed in Table 3. In some embodiments, the first and second single stranded RNAs are complementary sequences, exclusive of the 3′ overhangs, selected from the group listed in Table 3.

Exemplary sequences of first single stranded RNAs comprising the sense region and second single stranded RNAs comprising the anti-sense region, with dinucleotide 3′ overhangs are shown in table 3 below. Each row of the table shows pairs of first and second single stranded RNAs capable of hybridizing to form the mature duplex siRNA.

TABLE 3
Representative GPC2 siRNAs

| Sense | Anti-sense |
|---|---|
| CUCCUGAUCCUGGCUGAUA | UAUCAGCCAGGAUCAGGAG[dT][dT] |
| [dT][dT] | |
| (SEQ ID NO: 203) | (SEQ ID NO: 204) |
| CUCAUCUACCGAUGGCUCU | AGAGCCAUCGGUAGAUGAG[dT][dT] |
| [dT][dT] | |
| (SEQ ID NO: 205) | (SEQ ID NO: 206) |
| CCUGCUUGGACCUCGAUAA | UUAUCGAGGUCCAAGCAGG[dT][dT] |
| [dT][dT] | |
| (SEQ ID NO: 207) | (SEQ ID NO: 208) |
| GUGGUUCGUGGCUGUCUCA | UGAGACAGCCACGAACCAC[dT][dT] |
| [dT][dT] | |
| (SEQ ID NO: 209) | (SEQ ID NO: 210) |
| CUCAGUAGCCCAGCACUCU | AGAGUGCUGGGCUACUGAG[dT][dT] |
| [dT][dT] | |
| (SEQ ID NO: 211) | (SEQ ID NO: 212) |
| CUGCUGUUCCAGUGAGACA | UGUCUCACUGGAACAGCAG[dT][dT] |
| [dT][dT] | |
| (SEQ ID NO: 213) | (SEQ ID NO: 214) |
| CUCCUUUCUGGUUCACACA | UGUGUGAACCAGAAAGGAG[dT][dT] |
| [dT][dT] | |
| (SEQ ID NO: 215) | (SEQ ID NO: 216) |
| GAGUGUGGUUUCCUUAGAA | UUCUAAGGAAACCACACUC[dT][dT] |
| [dT][dT] | |
| (SEQ ID NO: 217) | (SEQ ID NO: 218) |
| GAGUACACCUGCUGUUCCA | UGGAACAGCAGGUGUACUC[dT][dT] |
| [dT][dT] | |
| (SEQ ID NO: 219) | (SEQ ID NO: 220) |
| GACACGACCUGGACGGGCA | UGCCCGUCCAGGUCGUGUC[dT][dT] |
| [dT][dT] | |
| (SEQ ID NO: 221) | (SEQ ID NO: 222) |
| CUGACUACCUGCUCUGCCU | AGGCAGAGCAGGUAGUCAG[dT][dT] |
| [dT][dT] | |
| (SEQ ID NO: 223) | (SEQ ID NO: 224) |
| GCGCUUAAGGUGCCGGUGU | ACACCGGCACCUUAAGCGC[dT][dT] |
| [dT][dT] | |
| (SEQ ID NO: 225) | (SEQ ID NO: 226) |
| CCUUUGAGCUGACGGCCGA | UCGGCCGUCAGCUCAAAGG[dT][dT] |
| [dT][dT] | |
| (SEQ ID NO: 227) | (SEQ ID NO: 228) |
| CCUGCUUCUGCUGCUGCCU | AGGCAGCAGCAGAAGCAGG[dT][dT] |
| [dT][dT] | |
| (SEQ ID NO: 229) | (SEQ ID NO: 230) |
| GAAGAAAUGUGGUCAGCGA | UCGCUGACCACAUUUCUUC[dT][dT] |
| [dT][dT] | |
| (SEQ ID NO: 231) | (SEQ ID NO: 232) |

Unless otherwise indicated, sequences in the Tables refer to ribonucleic acids (RNA). d[T] refers to deoxyribonucleic acids (DNA).

siRNAs can be designed using commercially available design tools and kits, such as those available from Ambion, Inc. (Austin, Tex.), the Whitehead Institute of Biomedical Research at MIT (Cambridge, Mass.), Invivogen (www.invivogen.com/sirnawizard/siRNA.php) and ThermoFisher (rnaidesigner.thermofisher.com/rnaiexpress/design.do) allow for the design and production of siRNA. siRNAs can also be designed using the Designer of Small Interfering RNA (DSIR) web-based tool available at biodev.extra.cea.fr/DSIR/DSIR.html. Using DSIR, siRNAs are designed and given a score based on the predicted efficacy of the siRNA based on a 19 or 21 nucleotide model.

In some embodiments, the siRNA comprises an RNA duplex that is 21 nucleotides in length. In some embodiments, the RNA duplex comprises a sense region and an antisense region that are selected from the group of sequences in Table 4.

TABLE 4
siRNA sense regions and anti-sense regions, 21 bp in length

| | SEQ | | SEQ | | | Corr. |
|---|---|---|---|---|---|---|
| Pos. | ID NO: | Sense Region | ID NO: | Antisense Region | Score | Score |
| 1 | 233 | UCCCAUUGUCUCGGCAGAUGC | 1477 | AUCUGCCGAGACAAUGGGAGC | 60.2 | 60.2 |
| 2 | 234 | CCCAUUGUCUCGGCAGAUGCC | 1478 | CAUCUGCCGAGACAAUGGGAG | 67.5 | 67.5 |
| 3 | 235 | CCAUUGUCUCGGCAGAUGCCG | 1479 | GCAUCUGCCGAGACAAUGGGA | 68.3 | 68.3 |
| 4 | 236 | CAUUGUCUCGGCAGAUGCCGC | 1480 | GGCAUCUGCCGAGACAAUGGG | 64.7 | 64.7 |
| 5 | 237 | AUUGUCUCGGCAGAUGCCGCC | 1481 | CGGCAUCUGCCGAGACAAUGG | 49.5 | 49.5 |
| 6 | 238 | UUGUCUCGGCAGAUGCCGCCU | 1482 | GCGGCAUCUGCCGAGACAAUG | 48.4 | 48.4 |
| 7 | 239 | UGUCUCGGCAGAUGCCGCCUG | 1483 | GGCGGCAUCUGCCGAGACAAU | 43.5 | 43.5 |
| 8 | 240 | GUCUCGGCAGAUGCCGCCUGG | 1484 | AGGCGGCAUCUGCCGAGACAA | 61.2 | 61.2 |
| 9 | 241 | UCUCGGCAGAUGCCGCCUGGU | 1485 | CAGGCGGCAUCUGCCGAGACA | 49.5 | 49.5 |
| 10 | 242 | CUCGGCAGAUGCCGCCUGGUC | 1486 | CCAGGCGGCAUCUGCCGAGAC | 47.8 | 47.8 |
| 11 | 243 | UCGGCAGAUGCCGCCUGGUCC | 1487 | ACCAGGCGGCAUCUGCCGAGA | 55 | 55 |
| 12 | 244 | CGGCAGAUGCCGCCUGGUCCA | 1488 | GACCAGGCGGCAUCUGCCGAG | 65.2 | 65.2 |
| 13 | 245 | GGCAGAUGCCGCCUGGUCCAG | 1489 | GGACCAGGCGGCAUCUGCCGA | 61.6 | 61.6 |
| 14 | 246 | GCAGAUGCCGCCUGGUCCAGC | 1490 | UGGACCAGGCGGCAUCUGCCG | 79.9 | 79.9 |
| 15 | 247 | CAGAUGCCGCCUGGUCCAGCU | 1491 | CUGGACCAGGCGGCAUCUGCC | 52 | 52 |
| 16 | 248 | AGAUGCCGCCUGGUCCAGCAU | 1492 | GCUGGACCAGGCGGCAUCUGC | 42.4 | 42.4 |
| 17 | 249 | GAUGCCGCCUGGUCCAGCUAU | 1493 | AGCUGGACCAGGCGGCAUCUG | 71.1 | 71.1 |
| 18 | 250 | AUGCCGCCUGGUCCAGCUAUC | 1494 | UAGCUGGACCAGGCGGCAUCU | 71.4 | 71.4 |
| 19 | 251 | UGCCGCCUGGUCCAGCUAUCG | 1495 | AUAGCUGGACCAGGCGGCAUC | 61.3 | 61.3 |
| 20 | 252 | GCCGCCUGGUCCAGCUAUCGU | 1496 | GAUAGCUGGACCAGGCGGCAU | 66.6 | 66.6 |
| 21 | 253 | CCGCCUGGUCCAGCUAUCGUG | 1497 | CGAUAGCUGGACCAGGCGGCA | 57.7 | 57.7 |
| 22 | 254 | CGCCUGGUCCAGCUAUCGUGC | 1498 | ACGAUAGCUGGACCAGGCGGC | 68.2 | 68.2 |
| 23 | 255 | GCCUGGUCCAGCUAUCGUGCU | 1499 | CACGAUAGCUGGACCAGGCGG | 73.4 | 73.4 |
| 24 | 256 | CCUGGUCCAGCUAUCGUGCUC | 1500 | GCACGAUAGCUGGACCAGGCG | 61.3 | 61.3 |
| 25 | 257 | CUGGUCCAGCUAUCGUGCUCG | 1501 | AGCACGAUAGCUGGACCAGGC | 61.9 | 61.9 |
| 26 | 258 | UGGUCCAGCUAUCGUGCUCGG | 1502 | GAGCACGAUAGCUGGACCAGG | 64 | 64 |
| 27 | 259 | GGUCCAGCUAUCGUGCUCGGU | 1503 | CGAGCACGAUAGCUGGACCAG | 64.4 | 64.4 |
| 28 | 260 | GUCCAGCUAUCGUGCUCGGUA | 1504 | CCGAGCACGAUAGCUGGACCA | 64 | 64 |
| 29 | 261 | UCCAGCUAUCGUGCUCGGUAU | 1505 | ACCGAGCACGAUAGCUGGACC | 49.2 | 49.2 |
| 30 | 262 | CCAGCUAUCGUGCUCGGUAUU | 1506 | UACCGAGCACGAUAGCUGGAC | 78.1 | 78.1 |
| 31 | 263 | CAGCUAUCGUGCUCGGUAUUC | 1507 | AUACCGAGCACGAUAGCUGGA | 87 | 87 |
| 32 | 264 | AGCUAUCGUGCUCGGUAUUCA | 1508 | AAUACCGAGCACGAUAGCUGG | 82.9 | 82.9 |
| 33 | 265 | GCUAUCGUGCUCGGUAUUCAG | 1509 | GAAUACCGAGCACGAUAGCUG | 65.9 | 65.9 |
| 34 | 266 | CUAUCGUGCUCGGUAUUCAGU | 1510 | UGAAUACCGAGCACGAUAGCU | 79 | 79 |
| 35 | 267 | UAUCGUGCUCGGUAUUCAGUU | 1511 | CUGAAUACCGAGCACGAUAGC | 60.2 | 60.2 |
| 36 | 268 | AUCGUGCUCGGUAUUCAGUUU | 1512 | ACUGAAUACCGAGCACGAUAG | 64.9 | 64.9 |
| 55 | 269 | UUUCCGGAGCAGCGCUCUUUC | 1513 | AAGAGCGCUGCUCCGGAAAAC | 59.4 | 59.4 |
| 56 | 270 | UUCCGGAGCAGCGCUCUUUCU | 1514 | AAAGAGCGCUGCUCCGGAAAA | 59.6 | 59.6 |
| 57 | 271 | UCCGGAGCAGCGCUCUUUCUC | 1515 | GAAAGAGCGCUGCUCCGGAAA | 56.3 | 56.3 |
| 58 | 272 | CCGGAGCAGCGCUCUUUCUCU | 1516 | AGAAAGAGCGCUGCUCCGGAA | 75.8 | 75.8 |
| 59 | 273 | CGGAGCAGCGCUCUUUCUCUG | 1517 | GAGAAAGAGCGCUGCUCCGGA | 66.5 | 66.5 |
| 60 | 274 | GGAGCAGCGCUCUUUCUCUGG | 1518 | AGAGAAAGAGCGCUGCUCCGG | 80.4 | 80.4 |
| 79 | 275 | GGCCCGCGGAGCGGUCCCGCG | 1519 | CGGGACCGCUCCGCGGGCCAG | 53.9 | 53.9 |
| 80 | 276 | GCCCGCGGAGCGGUCCCGCGG | 1520 | GCGGGACCGCUCCGCGGGCCA | 42.7 | 42.7 |
| 81 | 277 | CCCGCGGAGCGGUCCCGCGGC | 1521 | CGCGGGACCGCUCCGCGGGCC | 47.6 | 47.6 |
| 82 | 278 | CCGCGGAGCGGUCCCGCGGCC | 1522 | CCGCGGGACCGCUCCGCGGGC | 52.7 | 52.7 |
| 83 | 279 | CGCGGAGCGGUCCCGCGGCCG | 1523 | GCCGCGGGACCGCUCCGCGGG | 56.2 | 56.2 |
| 84 | 280 | GCGGAGCGGUCCCGCGGCCGA | 1524 | GGCCGCGGGACCGCUCCGCGG | 57.1 | 57.1 |
| 85 | 281 | CGGAGCGGUCCCGCGGCCGAG | 1525 | CGGCCGCGGGACCGCUCCGCG | 49.2 | 49.2 |
| 86 | 282 | GGAGCGGUCCCGCGGCCGAGU | 1526 | UCGGCCGCGGGACCGCUCCGC | 74.8 | 74.8 |
| 87 | 283 | GAGCGGUCCCGCGGCCGAGUA | 1527 | CUCGGCCGCGGGACCGCUCCG | 80.8 | 60.8 |
| 88 | 284 | AGCGGUCCCGCGGCCGAGUAC | 1528 | ACUCGGCCGCGGGACCGCUCC | 54.5 | 54.5 |
| 89 | 285 | GCGGUCCCGCGGCCGAGUACC | 1529 | UACUCGGCCGCGGGACCGCUC | 73.9 | 73.9 |
| 90 | 286 | CGGUCCCGCGGCCGAGUACCG | 1530 | GUACUCGGCCGCGGGACCGCU | 62.1 | 62.1 |
| 91 | 287 | GGUCCCGCGGCCGAGUACCGG | 1531 | GGUACUCGGCCGCGGGACCGC | 50.3 | 50.3 |
| 92 | 288 | GUCCCGCGGCCGAGUACCGGA | 1532 | CGGUACUCGGCCGCGGGACCG | 55.9 | 55.9 |
| 93 | 289 | UCCCGCGGCCGAGUACCGGAU | 1533 | CCGGUACUCGGCCGCGGGACC | 31.9 | 31.9 |
| 94 | 290 | CCCGCGGCCGAGUACCGGAUU | 1534 | UCCGGUACUCGGCCGCGGGAC | 68.6 | 68.6 |
| 95 | 291 | CCGCGGCCGAGUACCGGAUUC | 1535 | AUCCGGUACUCGGCCGCGGGA | 76.3 | 76.3 |
| 96 | 292 | CGCGGCCGAGUACCGGAUUCC | 1536 | AAUCCGGUACUCGGCCGCGGG | 77.9 | 77.9 |
| 97 | 293 | GCGGCCGAGUACCGGAUUCGC | 1537 | GAAUCCGGUACUCGGCCGCGG | 71.7 | 71.7 |
| 98 | 294 | CGGCCGAGUACCGGAUUCCCG | 1538 | GGAAUCCGGUACUCGGCCGCG | 61.2 | 61.2 |
| 99 | 295 | GGCCGAGUACCGGAUUCCCGA | 1539 | GGGAAUCCGGUACUCGGCCGC | 58.7 | 58.7 |
| 100 | 296 | GCCGAGUACCGGAUUCCCGAG | 1540 | CGGGAAUCCGGUACUCGGCCG | 64.7 | 63.7 |
| 101 | 297 | CCGAGUACCGGAUUCCCGAGU | 1541 | UCGGGAAUCCGGUACUCGGCC | 76.4 | 75.4 |
| 102 | 298 | CGAGUACCGGAUUCCCGAGUU | 1542 | CUCGGGAAUCCGGUACUCGGC | 68.9 | 67.9 |
| 103 | 299 | GAGUACCGGAUUCCCGAGUUU | 1543 | ACUCGGGAAUCCGGUACUCGG | 73.7 | 72.7 |
| 104 | 300 | AGUACCGGAUUCCCGAGUUUG | 1544 | AACUCGGGAAUCCGGUACUCG | 77.4 | 76.4 |
| 105 | 301 | GUACCGGAUUCCCGAGUUUGG | 1545 | AAACUCGGGAAUCCGGUACUC | 79.3 | 78.3 |
| 106 | 302 | UACCGGAUUCCCGAGUUUGGG | 1546 | CAAACUCGGGAAUCCGGUACU | 59.4 | 58.4 |
| 107 | 303 | ACCGGAUUCCCGAGUUUGGGA | 1547 | CCAAACUCGGGAAUCCGGUAC | 59.9 | 58.9 |
| 108 | 304 | CCGGAUUCCCGAGUUUGGGAG | 1548 | CCCAAACUCGGGAAUCCGGUA | 60.9 | 59.9 |
| 109 | 305 | CGGAUUCCCGAGUUUGGGAGG | 1549 | UCCCAAACUCGGGAAUCCGGU | 30.2 | 79.2 |
| 110 | 306 | GGAUUCCCGAGUUUGGGAGGG | 1550 | GUCCCAAACUCGGGAAUCCGG | 68.1 | 67.1 |
| 111 | 307 | GAUUCCCGAGUUUGGGAGGCU | 1551 | CCUCCCAAACUCGGGAAUCCG | 59.3 | 58.3 |
| 112 | 308 | AUUCCCGAGUUUGGGAGGCUC | 1552 | GCCUCCCAAACUCGGGAAUCC | 33.2 | 32.2 |
| 113 | 309 | UUCCCGAGUUUGGGAGGCUCU | 1553 | AGCCUCCCAAACUCGGGAAUC | 47.8 | 46.8 |
| 114 | 310 | UCCCGAGUUUGGGAGGCUCUG | 1554 | GAGCCUCCCAAACUCGGGAAU | 54.2 | 53.2 |
| 115 | 311 | CCCGAGUUUGGGAGGCUCUGC | 1555 | AGAGCCUCCCAAACUCGGGAA | 72.3 | 71.3 |
| 116 | 312 | CCGAGUUUGGGAGGCUCUGCU | 1556 | CAGAGCCUCCCAAACUCGGGA | 68.3 | 67.3 |
| 117 | 313 | CGAGUUUGGGAGGCUCUGCUU | 1557 | GCAGAGCCUCCCAAACUCGGG | 64 | 63 |
| 118 | 314 | GAGUUUGGGAGGCUCUGCUUU | 1558 | AGCAGAGCCUCCCAAACUCGG | 72.8 | 71.8 |
| 119 | 315 | AGUUUGGGAGGCUCUGCUUUC | 1559 | AAGCAGAGCCUCCCAAACUCG | 7.58 | 74.8 |
| 120 | 316 | GUUUGGGAGGCUCUGCUUUCC | 1560 | AAAGCAGAGCCUCCCAAACUC | 69 | 68 |
| 121 | 317 | UUUGGGAGGCUCUGCUUUCCU | 1561 | GAAAGCAGAGCCUCCCAAACU | 49 | 48 |
| 122 | 318 | UUGGGAGGCUCUGCUUUCCUC | 1562 | GGAAAGCAGAGCCUCCCAAAC | 37.8 | 36.8 |
| 123 | 319 | UGGGAGGCUCUGCUUUCCUCC | 1563 | AGGAAAGCAGAGCCUCCCAAA | 61.1 | 60.1 |
| 124 | 320 | GGGAGGCUCUGCUUUCCUCCU | 1564 | GAGGAAAGCAGAGCCUCCCAA | 69 | 68 |
| 125 | 321 | GGAGGCUCUGCUUUCCUCCUU | 1565 | GGAGGAAAGCAGAGCCUCCCA | 59.2 | 58.2 |
| 126 | 322 | GAGGCUCUGCUUUCCUCCUUA | 1566 | AGGAGGAAAGCAGAGCCUCCC | 66.8 | 65.8 |
| 127 | 323 | AGGCUCUGCUUUCCUCCUUAG | 1567 | AAGGAGGAAAGCAGAGCCUCC | 66.5 | 65.5 |
| 128 | 324 | GGCUCUGCUUUCCUCCUUAGG | 1568 | UAAGGAGGAAAGCAGAGCCUC | 87.2 | 86.2 |
| 129 | 325 | GCUCUGCUUUCCUCCUUAGGA | 1569 | CUAAGGAGGAAAGCAGAGCCU | 75.7 | 74.7 |
| 130 | 326 | CUCUGCUUUCCUCCUUAGGAC | 1570 | CCUAAGGAGGAAAGCAGAGCC | 52.3 | 51.3 |
| 131 | 327 | UCUGCUUUCCUCCUUAGGACC | 1571 | UCCUAAGGAGGAAAGCAGAGC | 64.3 | 53.3 |
| 132 | 328 | CUGCUUUCCUCCUUAGGACCC | 1572 | GUCCUAAGGAGGAAAGCAGAG | 66.2 | 65.2 |
| 133 | 329 | UGCUUUCCUCCUUAGGACCCA | 1573 | GGUCCUAAGGAGGAAAGCAGA | 55.9 | 54.9 |
| 134 | 330 | GCUUUCCUCCUUAGGACCCAC | 1574 | GGGUCCUAAGGAGGAAAGCAG | 58.5 | 57.5 |
| 135 | 331 | CUUUCCUCCUUAGGACCCACU | 1575 | UGGGUCCUAAGGAGGAAAGCA | 68.6 | 67.6 |
| 136 | 332 | UUUCCUCCUUAGGACCCACUU | 1576 | GUGGGUCCUAAGGAGGAAAGC | 46 | 45 |
| 137 | 333 | UUCCUCCUUAGGACCCACUUU | 1577 | AGUGGGUCCUAAGGAGGAAAG | 53.8 | 52.8 |
| 138 | 334 | UCCUCCUUAGGACCCACUUUG | 1578 | AAGUGGGUCCUAAGGAGGAAA | 66.4 | 65.4 |
| 139 | 335 | CCUCCUUAGGACCCACUUUGC | 1579 | AAAGUGGGUCCUAAGGAGGAA | 79.9 | 78.9 |
| 140 | 336 | CUCCUUAGGACCCACUUUGCC | 1580 | CAAAGUGGGUCCUAAGGAGGA | 66 | 65 |
| 141 | 337 | UCCUUAGGACCCACUUUGCCG | 1581 | GCAAAGUGGGUCCUAAGGAGG | 55.6 | 54.6 |
| 142 | 338 | CCUUAGGACCCACUUUGCCGU | 1582 | GGCAAAGUGGGUCCUAAGGAG | 58.2 | 57.2 |
| 143 | 339 | CUUAGGACCCACUUUGCCGUC | 1583 | CGGCAAAGUGGGUCCUAAGGA | 55.8 | 54.8 |
| 144 | 340 | UAGGACCCACUUUGCCGUCCU | 1584 | ACGGCAAAGUGGGUCCUAAGG | 54.6 | 53.6 |
| 145 | 341 | UAGGACCCACUUUGCCGUCCU | 1585 | GACGGCAAAGUGGGUCCUAAG | 47.2 | 46.2 |
| 146 | 342 | AGGACCCACUUUGCCGUCCUG | 1586 | GGACGGCAAAGUGGGUCCUAA | 47.4 | 46.4 |
| 147 | 348 | GGACCCACUUUGCCGUCCUGG | 1587 | AGGACGGCAAAGUGGGUCCUA | 74.4 | 73.4 |
| 148 | 344 | GACCCACUUUGCCGUCCUGGG | 1588 | CAGGACGGCAAAGUGGGUCCU | 66.1 | 55.1 |
| 171 | 345 | GGCUGCAGUUAUGUCCGCGCU | 1589 | CGCGGACAUAACUGCAGCCAC | 58 | 57 |
| 172 | 346 | GCUGCAGUUAUGUCCGCGCUG | 1590 | GCGCGGACAUAACUGCAGCCA | 64.4 | 53.4 |
| 173 | 347 | CUGCAGUUAUGUCCGCGCUGC | 1591 | AGCGCGGACAUAACUGCAGCC | 67.3 | 66.3 |
| 174 | 348 | UGCAGUUAUGUCCGCGCUGCG | 1592 | CAGCGCGGACAUAACUGCAGC | 59.4 | 58.4 |
| 175 | 349 | GCAGUUAUGUCCGCGCUGCGA | 1593 | GCAGCGCGGACAUAACUGCAG | 66.9 | 65.9 |
| 176 | 350 | CAGUUAUGUCCGCGCUGCGAC | 1594 | CGCAGCGCGGACAUAACUGCA | 65.7 | 64.7 |
| 177 | 351 | AGUUAUGUCCGCGCUGCGACC | 1595 | UCGCAGCGCGGACAUAACUGC | 76.5 | 75.5 |
| 178 | 352 | GUUAUGUCCGCGCUGCGACCU | 1596 | GUCGCAGCGCGGACAUAACUG | 68.4 | 67.4 |
| 179 | 353 | UUAUGUCCGCGCUGCGACCUC | 1597 | GGUCGCAGCGCGGACAUAACU | 49.4 | 48.4 |
| 180 | 354 | UAUGUCCGCGCUGCGACCUCU | 1598 | AGGUCGCAGCGCGGACAUAAC | 49.2 | 48.2 |
| 181 | 355 | AUGUCCGCGCUGCGACCUCUC | 1599 | GAGGUCGCAGCGCGGACAUAA | 56.7 | 55.7 |
| 182 | 356 | UGUCCGCGCUGCGACCUCUCC | 1600 | AGAGGUCGCAGCGCGGACAUA | 70.6 | 69.6 |
| 183 | 357 | GUCCGCGCUGCGACCUCUCCU | 1601 | GAGAGGUCGCAGCGCGGACAU | 63.1 | 62.1 |
| 184 | 358 | UCCGCGCUGCGACCUCUCCUG | 1602 | GGAGAGGUCGCAGCGCGGACA | 50.6 | 49.6 |
| 185 | 359 | CCGCGCUGCGACCUCUCCUGC | 1603 | AGGAGAGGUCGCAGCGCGGAC | 65.3 | 64.3 |
| 186 | 360 | CGCGCUGCGACCUCUCCUGCU | 1604 | CAGGAGAGGUCGCAGCGCGGA | 72.8 | 71.8 |
| 187 | 361 | GCGCUGCGACCUCUCCUGCUU | 1605 | GCAGGAGAGGUCGCAGCGCGG | 67.1 | 66.1 |
| 188 | 362 | CGCUGCGACCUCUCCUGCUUC | 1806 | AGCAGGAGAGGUCGCAGCGCG | 76.1 | 75.1 |
| 189 | 363 | GCUGCGACCUCUCCUGCUUCU | 1607 | AAGCAGGAGAGGUCGCAGCGC | 83.4 | 82.4 |
| 190 | 364 | CUGCGACCUCUCCUGCUUCUG | 1608 | GAAGCAGGAGAGGUCGCAGCG | 66 | 65 |
| 191 | 365 | UGCGACCUCUCCUGCUUCUGC | 1609 | AGAAGCAGGAGAGGUCGCAGC | 65.8 | 64.8 |
| 192 | 366 | GCGACCUCUCCUGCUUCUGCU | 1610 | CAGAAGCAGGAGAGGUCGCAG | 71.4 | 70.4 |
| 193 | 367 | CGACCUCUCCUGCUUCUGCUG | 1611 | GCAGAAGCAGGAGAGGUCGCA | 65.7 | 64.7 |
| 194 | 368 | GACCUCUCCUGCUUCUGCUGC | 1612 | AGCAGAAGCAGGAGAGGUCGC | 69.2 | 68.2 |
| 195 | 369 | ACCUCUCCUGCUUCUGCUGCU | 1613 | CAGCAGAAGCAGGAGAGGUCG | 65.1 | 64.1 |
| 196 | 370 | CCUCUCCUGCUUCUGCUGCUG | 1614 | GCAGCAGAAGCAGGAGAGGUC | 51.7 | 50.7 |
| 197 | 371 | CUCUCCUGCUUCUGCUGCUGC | 1615 | AGCAGCAGAAGCAGGAGAGGU | 72.4 | 71.4 |
| 198 | 372 | UCUCCUGCUUCUGCUGCUGCC | 1616 | CAGCAGCAGAAGCAGGAGAGG | 62.2 | 61.2 |
| 199 | 373 | CUCCUGCUUCUGCUGCUGCCU | 1617 | GCAGCAGCAGAAGCAGGAGAG | 58.2 | 57.2 |
| 200 | 374 | UCCUGCUUCUGCUGCUGCCUC | 1618 | GGCAGCAGCAGAAGCAGGAGA | 53.3 | 51.3 |
| 201 | 375 | CCUGCUUCUGCUGCUGCCUCU | 1619 | AGGCAGCAGCAGAAGCAGGAG | 71.7 | 69.7 |
| 202 | 376 | CUGCUUCUGCUGCUGCCUCUG | 1620 | GAGGCAGCAGCAGAAGCAGGA | 61.5 | 59.5 |
| 203 | 377 | UGCUUCUGCUGCUGCCUCUGU | 1621 | AGAGGCAGCAGCAGAAGCAGG | 70 | 68 |
| 204 | 378 | GCUUCUGCUGCUGCCUCUGUG | 1622 | CAGAGGCAGCAGCAGAAGCAG | 70.9 | 68.9 |
| 205 | 379 | CUUCUGCUGCUGCCUCUGUGU | 1623 | ACAGAGGCAGCAGCAGAAGCA | 68.7 | 66.7 |
| 206 | 380 | UUCUGCUGCUGCCUCUGUGUC | 1624 | CACAGAGGCAGCAGCAGAAGC | 50.1 | 48.1 |
| 207 | 381 | UCUGCUGCUGCCUCUGUGUCC | 1625 | ACACAGAGGCAGCAGCAGAAG | 69.2 | 67.2 |
| 208 | 382 | CUGCUGCUGCCUCUGUGUCCC | 1626 | GACACAGAGGCAGCAGCAGAA | 63.6 | 61.6 |
| 209 | 383 | UGCUGCUGCCUCUGUGUCCCG | 1627 | GGACACAGAGGCAGCAGCAGA | 56.7 | 54.7 |
| 210 | 384 | GCUGCUGCCUCUGUGUCCCGG | 1628 | GGGACACAGAGGCAGCAGCAG | 63.9 | 61.9 |
| 211 | 385 | CUGCUGCCUCUGUGUCCCGGU | 1629 | CGGGACACAGAGGCAGCAGCA | 57 | 55 |
| 212 | 386 | UGCUGCCUCUGUGUCCCGGUC | 1630 | CCGGGACACAGAGGCAGCAGC | 44.9 | 42.9 |
| 213 | 387 | GCUGCCUCUGUGUCCCGGUCC | 1631 | ACCGGGACACAGAGGCAGCAG | 72.3 | 70.3 |
| 214 | 388 | CUGCCUCUGUGUCCCGGUCCU | 1632 | GACCGGGACACAGAGGCAGCA | 61.9 | 59.9 |
| 215 | 389 | UGCCUCUGUGUCCCGGUCCUG | 1633 | GGACCGGGACACAGAGGCAGC | 42.8 | 40.8 |
| 216 | 390 | GCCUCUGUGUCCCGGUCCUGG | 1834 | AGGACCGGGACACAGAGGCAG | 80.1 | 78.1 |
| 217 | 391 | CCUCUGUGUCCCGGUCCUGGU | 1635 | CAGGACCGGGACACAGAGGCA | 64.8 | 62.8 |
| 218 | 392 | CUCUGUGUCCCGGUCCUGGUC | 1636 | CCAGGACCGGGACACAGAGGC | 52.1 | 50.1 |
| 219 | 393 | UCUGUGUCCCGGUCCUGGUCC | 1637 | ACCAGGACCGGGACACAGAGG | 68.4 | 66.4 |
| 220 | 394 | CUGUGUCCCGGUCCUGGUCCC | 1638 | GACCAGGACCGGGACACAGAG | 64.9 | 62.9 |
| 221 | 395 | UGUGUCCCGGUCCUGGUCCCG | 1639 | GGACCAGGACCGGGACACAGA | 47.6 | 45.6 |
| 222 | 396 | GUGUCCCGGUCCUGGUCCCGG | 1640 | GGGACCAGGACCGGGACACAG | 58.5 | 56.5 |
| 223 | 397 | UGUCCCGGUCCUGGUCCCGGA | 1641 | CGGGACCAGGACCGGGACACA | 44.1 | 42.1 |
| 224 | 398 | GUCCCGGUCCUGGUCCCGGAC | 1642 | CCGGGACCAGGACCGGGACAC | 43.7 | 41.7 |
| 225 | 399 | UCCCGGUCCUGGUCCCGGACC | 1643 | UCCGGGACCAGGACCGGGACA | 66.8 | 64.8 |
| 226 | 400 | CCCGGUCCUGGUCCCGGACGC | 1644 | GUCCGGGACCAGGACCGGGAC | 55.5 | 53.5 |
| 227 | 401 | CCGGUCCUGGUCCCGGACCCG | 1645 | GGUCCGGGACCAGGACCGGGA | 52.4 | 50.4 |
| 228 | 402 | CGGUCCUGGUCCCGGACCCGG | 1646 | GGGUCCGGGACCAGGACCGGG | 62.9 | 60.9 |
| 229 | 403 | GGUCCUGGUCCCGGACCCGGG | 1647 | CGGGUCCGGGACCAGGACCGG | 63.3 | 61.3 |
| 230 | 404 | GUCCUGGUCCCGGACCCGGGA | 1648 | CCGGGUCCGGGACCAGGACCG | 52.1 | 50.1 |
| 231 | 405 | UCCUGGUCCCGGACCCGGGAG | 1649 | CCCGGGUCCGGGACCAGGACC | 39 | 37 |
| 232 | 406 | CCUGGUCCCGGACCCGGGAGC | 1650 | UCCCGGGUCCGGGACCAGGAC | 65.8 | 63.8 |
| 233 | 407 | CUGGUCCCGGACCCGGGAGCG | 1651 | CUCCCGGGUCCGGGACCAGGA | 51.2 | 49.2 |
| 234 | 408 | UGGUCCCGGACCGGGGAGCGA | 1652 | GCUCCCGGGUCCGGGACCAGG | 44.1 | 42.1 |
| 235 | 409 | GGUCCCGGACCCGGGAGCGAG | 1653 | CGCUCCCGGGUCCGGGACCAG | 53.4 | 51.4 |
| 236 | 410 | GUCCCGGACCCGGGAGCGAGG | 1654 | UCGCUCCCGGGUCCGGGACCA | 68.1 | 66.1 |
| 237 | 411 | UCCCGGACCCGGGAGCGAGGC | 1655 | CUCGCUCCCGGGUCCGGGACG | 42.9 | 40.9 |
| 238 | 412 | CGCGGACCGGGGAGCGAGGCA | 1656 | CCUCGCUCCGGGGUCCGGGAC | 52 | 50 |
| 239 | 413 | CCGGACCCGGGAGCGAGGCAA | 1657 | GCCUCGCUCCCGGGUCCGGGA | 46.6 | 44.6 |
| 240 | 414 | CGGACCCGGGAGCGAGGCAAA | 1658 | UGCCUCGCUCCCGGGUCCGGG | 74.7 | 72.7 |
| 241 | 415 | GGACCCGGGAGCGAGGCAAAG | 1659 | UUGCCUCGCUCCCGGGUCCGG | 83.5 | 81.5 |
| 242 | 416 | GACCCGGGAGCGAGGCAAAGG | 1660 | UUUGCCUCGCUCCCGGGUCCG | 84.7 | 82.7 |
| 243 | 417 | ACCCGGGAGCGAGGCAAAGGU | 1661 | CUUUGCCUCGCUCCCGGGUCC | 51.8 | 49.8 |
| 244 | 418 | CCCGGGAGCGAGGCAAAGGUC | 1662 | CCUUUGCCUCGCUCCCGGGUC | 52.1 | 50.1 |
| 245 | 419 | CCGGGAGCGAGGCAAAGGUCA | 1663 | ACCUUUGCCUCGCUCCCGGGU | 73.1 | 71.1 |
| 246 | 420 | CGGGAGCGAGGCAAAGGUCAC | 1664 | GACCUUUGCCUCGCUCCCGGG | 71.1 | 69.1 |
| 247 | 421 | GGGAGCGAGGCAAAGGUCACC | 1665 | UGACCUUUGCCUCGCUCCCGG | 86.2 | 84.2 |
| 248 | 422 | GGAGCGAGGCAAAGGUCACCC | 1666 | GUGACCUUUGCCUCGCUCCCG | 78.9 | 76.9 |
| 249 | 423 | GAGCGAGGCAAAGGUCACCCG | 1667 | GGUGACCUUUGCCUCGCUCCC | 54.4 | 52.4 |
| 250 | 424 | AGCGAGGCAAAGGUCACCCGG | 1668 | GGGUGACCUUUGCCUCGCUCC | 46.1 | 44.1 |
| 251 | 425 | GCGAGGCAAAGGUCACCCGGA | 1669 | CGGGUGACCUUUGCCUCGCUC | 62.4 | 60.4 |
| 252 | 426 | CGAGGCAAAGGUCACCCGGAG | 1670 | CCGGGUGACCUUUGCCUCGCU | 63.5 | 61.5 |
| 253 | 427 | GAGGCAAAGGUCACGCGGAGU | 1671 | UCCGGGUGACCUUUGCCUCGC | 73.7 | 71.7 |
| 254 | 428 | AGGCAAAGGUCACCCGGAGUU | 1672 | CUCCGGGUGACCUUUGCCUCG | 66.8 | 64.8 |
| 255 | 429 | GGCAAAGGUCACCCGGAGUUG | 1673 | ACUCCGGGUGACCUUUGCCUC | 72.3 | 70.3 |
| 256 | 430 | GCAAAGGUCACCCGGAGUUGU | 1674 | AACUCCGGGUGACCUUUGCCU | 78.7 | 76.7 |
| 257 | 431 | CAAAGGUCACCCGGAGUUGUG | 1675 | CAACUCCGGGUGACCUUUGCC | 61.8 | 59.8 |
| 258 | 432 | AAAGGUCACCCGGAGUUGUGC | 1676 | ACAACUCCGGGUGACCUUUGC | 66.6 | 64.6 |
| 259 | 433 | AAGGUCACCCGGAGUUGUGCA | 1677 | CACAACUCCGGGUGACCUUUG | 66.7 | 64.7 |
| 260 | 434 | AGGUCACCCGGAGUUGUGCAG | 1678 | GCACAACUCCGGGUGACCUUU | 61 | 59 |
| 261 | 435 | GGUCACCCGGAGUUGUGCAGA | 1679 | UGCACAACUCCGGGUGACCUU | 84.8 | 82.8 |
| 262 | 436 | GUCACCCGGAGUUGUGCAGAG | 1680 | CUGCACAACUCCGGGUGACCU | 64.1 | 62.1 |
| 263 | 437 | UCACCCGGAGUUGUGCAGAGA | 1681 | UCUGCACAACUCCGGGUGACC | 58.4 | 56.4 |
| 264 | 438 | CACCCGGAGUUGUGCAGAGAC | 1682 | CUCUGCACAACUCCGGGUGAC | 68 | 66 |
| 265 | 439 | ACCGGGAGUUGUGCAGAGACC | 1683 | UCUCUGCACAACUCCGGGUGA | 82.6 | 80.6 |
| 266 | 440 | CCCGGAGUUGUGCAGAGACCC | 1684 | GUCUCUGCACAACUCCGGGUG | 76.4 | 74.4 |
| 267 | 441 | CCGGAGUUGUGCAGAGACCCG | 1685 | GGUCUCUGCACAACUCCGGGU | 74.4 | 72.4 |
| 268 | 442 | CGGAGUUGUGCAGAGACCCGG | 1686 | GGGUCUCUGCACAACUCCGGG | 71.9 | 69.9 |
| 269 | 443 | GGAGUUGUGCAGAGACCCGGC | 1687 | CGGGUCUCUGCACAACUCCGG | 78 | 76 |
| 270 | 444 | GAGUUGUGCAGAGACCCGGCA | 1688 | CCGGGUCUCUGCACAACUCCG | 68.1 | 66.1 |
| 271 | 445 | AGUUGUGCAGAGACCCGGCAG | 1689 | GCCGGGUCUCUGCACAACUCC | 56.7 | 54.7 |
| 272 | 446 | GUUGUGCAGAGACCCGGCAGG | 1690 | UGCCGGGUCUCUGCACAACUC | 74.1 | 72.1 |
| 273 | 447 | UUGUGCAGAGACCCGGCAGGU | 1691 | CUGCCGGGUCUCUGCACAACU | 52.6 | 50.6 |
| 274 | 448 | UGUGCAGAGACCCGGCAGGUG | 1692 | CCUGCCGGGUCUCUGCACAAC | 51 | 49 |
| 275 | 449 | GUGCAGAGACCCGGCAGGUGC | 1693 | ACCUGCCGGGUCUCUGCACAA | 66.8 | 64.8 |
| 276 | 450 | UGCAGAGACCCGGCAGGUGCU | 1694 | CACCUGCCGGGUCUCUGCACA | 54.8 | 52.8 |
| 277 | 451 | GCAGAGACCCGGCAGGUGCUG | 1695 | GCACCUGCCGGGUCUCUGCAC | 60 | 58 |
| 278 | 452 | CAGAGACCCGGCAGGUGCUGG | 1696 | AGCACCUGCCGGGUCUCUGCA | 71.4 | 69.4 |
| 279 | 453 | AGAGACCCGGCAGGUGCUGGG | 1697 | CAGCACCUGCCGGGUCUCUGC | 53.8 | 51.8 |
| 306 | 454 | GGGAUAUAGCUUAAACCUAAU | 1698 | UAGGUUUAAGCUAUAUCCCCG | 94.2 | 91.2 |
| 307 | 455 | GGAUAUAGCUUAAACCUAAUC | 1699 | UUAGGUUUAAGCUAUAUCCCC | 94.4 | 91.4 |
| 308 | 456 | GAUAUAGCUUAAACCUAAUCC | 1700 | AUUAGGUUUAAGCUAUAUCCC | 82.2 | 79.2 |
| 309 | 457 | AUAUAGCUUAAACCUAAUCCC | 1701 | GAUUAGGUUUAAGCUAUAUCC | 53.4 | 50.4 |
| 310 | 458 | UAUAGCUUAAACCUAAUCCCU | 1702 | GGAUUAGGUUUAAGCUAUAUC | 44.1 | 41.1 |
| 311 | 459 | AUAGCUUAAACCUAAUCCCUC | 1703 | GGGAUUAGGUUUAAGCUAUAU | 55.9 | 52.9 |
| 312 | 460 | UAGCUUAAACCUAAUCCCUCC | 1704 | AGGGAUUAGGUUUAAGCUAUA | 66.1 | 63.1 |
| 313 | 461 | AGCUUAAACCUAAUCCCUCCC | 1705 | GAGGGAUUAGGUUUAAGCUAU | 63.2 | 60.2 |
| 314 | 462 | GCUUAAACCUAAUCCCUCCCG | 1706 | GGAGGGAUUAGGUUUAAGCUA | 68.5 | 65.5 |
| 315 | 463 | CUUAAACCUAAUCCCUCCCGC | 1707 | GGGAGGGAUUAGGUUUAAGCU | 50.4 | 47.4 |
| 316 | 464 | UUAAACCUAAUCCCUCCCGCC | 1708 | CGGGAGGGAUUAGGUUUAAGC | 32.3 | 29.3 |
| 317 | 465 | UAAACCUAAUCCCUCCCGCCC | 1709 | GCGGGAGGGAUUAGGUUUAAG | 43.3 | 40.3 |
| 318 | 466 | AAACCUAAUCCCUCCCGCCCU | 1710 | GGCGGGAGGGAUUAGGUUUAA | 46.4 | 43.4 |
| 319 | 467 | AACCUAAUCCCUCCCGCCCUG | 1711 | GGGCGGGAGGGAUUAGGUUUA | 45.2 | 42.2 |
| 320 | 468 | ACCUAAUCCCUCCCGCCCUGA | 1712 | AGGGCGGGAGGGAUUAGGUUU | 60.8 | 57.8 |
| 321 | 469 | CCUAAUCCCUCCCGCCCUGAU | 1713 | CAGGGCGGGAGGGAUUAGGUU | 61.7 | 58.7 |
| 322 | 470 | CUAAUCCCUCCCGCCCUGAUC | 1714 | UCAGGGCGGGAGGGAUUAGGU | 60 | 57 |
| 323 | 471 | UAAUCCCUCCCGCCCUGAUCU | 1715 | AUCAGGGCGGGAGGGAUUAGG | 58.8 | 55.8 |
| 324 | 472 | AAUCCCUCCCGCCCUGAUCUC | 1716 | GAUCAGGGCGGGAGGGAUUAG | 50.2 | 47.2 |
| 325 | 473 | AUCCCUCCCGCCCUGAUCUCA | 1717 | AGAUCAGGGCGGGAGGGAUUA | 53.3 | 50.3 |
| 326 | 474 | UCCCUCCCGCCCUGAUCUCAG | 1718 | GAGAUCAGGGCGGGAGGGAUU | 45.6 | 42.6 |
| 327 | 475 | CCCUCCCGCCCUGAUCUCAGG | 1719 | UGAGAUCAGGGCGGGAGGGAU | 66.5 | 63.5 |
| 328 | 476 | CCUCCCGCCCUGAUCUCAGGU | 1720 | CUGAGAUCAGGGCGGGAGGGA | 60.3 | 57.3 |
| 329 | 477 | CUCCCGCCCUGAUCUCAGGUG | 1721 | CCUGAGAUCAGGGCGGGAGGG | 58.5 | 55.5 |
| 330 | 478 | UCCCGCCCUGAUCUCAGGUGA | 1722 | ACCUGAGAUCAGGGCGGGAGG | 52.3 | 49.3 |
| 331 | 479 | CCCGCCCUGAUCUCAGGUGAG | 1723 | CACCUGAGAUCAGGGCGGGAG | 56.8 | 53.8 |
| 332 | 480 | CCGCCCUGAUCUCAGGUGAGC | 1724 | UCACCUGAGAUCAGGGCGGGA | 79.4 | 76.4 |
| 333 | 481 | CGCCCUGAUCUCAGGUGAGCA | 1725 | CUCACCUGAGAUCAGGGCGGG | 71.1 | 65.1 |
| 334 | 482 | GCCCUGAUCUCAGGUGAGCAC | 1726 | GCUCACCUGAGAUCAGGGCGG | 61.6 | 58.6 |
| 335 | 483 | CCCUGAUCUCAGGUGAGCACC | 1727 | UGCUCACCUGAGAUCAGGGCG | 76.1 | 73.1 |
| 336 | 484 | CCUGAUCUCAGGUGAGCACCU | 1728 | GUGCUCACCUGAGAUCAGGGC | 64.3 | 61.3 |
| 337 | 485 | CUGAUCUCAGGUGAGCACCUC | 1729 | GGUGCUCACCUGAGAUCAGGG | 53.9 | 50.9 |
| 338 | 486 | UGAUCUCAGGUGAGCACCUCC | 1730 | AGGUGCUCACCUGAGAUCAGG | 70.7 | 67.7 |
| 339 | 487 | GAUCUCAGGUGAGCACCUCCG | 1731 | GAGGUGCUCACCUGAGAUCAG | 66.3 | 63.3 |
| 340 | 488 | AUCUCAGGUGAGCACCUCCGG | 1732 | GGAGGUGCUCACCUGAGAUCA | 52.6 | 49.6 |
| 341 | 489 | UCUCAGGUGAGCACCUCCGGG | 1733 | CGGAGGUGCUCACCUGAGAUC | 49.8 | 46.8 |
| 342 | 490 | CUCAGGUGAGCACCUCCGGGU | 1734 | CCGGAGGUGCUCACCUGAGAU | 59.5 | 56.5 |
| 343 | 491 | UCAGGUGAGCACCUCCGGGUC | 1735 | CCCGGAGGUGCUCACCUGAGA | 49.4 | 46.4 |
| 344 | 492 | CAGGUGAGCACCUCCGGGUCU | 1736 | ACCCGGAGGUGCUCACCUGAG | 67.3 | 64.3 |
| 345 | 493 | AGGUGAGCACCUCCGGGUCUG | 1737 | GACCCGGAGGUGCUCACCUGA | 62.4 | 59.4 |
| 346 | 494 | GGUGAGCACCUCCGGGUCUGU | 1738 | AGACCCGGAGGUGCUCACCUG | 77.1 | 74.1 |
| 347 | 495 | GUGAGCACCUCCGGGUCUGUC | 1739 | CAGACCCGGAGGUGCUCACCU | 57.5 | 54.5 |
| 348 | 496 | UGAGCACCUCCGGGUCUGUCC | 1740 | ACAGACCCGGAGGUGCUCACC | 54.7 | 51.7 |
| 349 | 497 | GAGCACCUCCGGGUCUGUCCC | 1741 | GACAGACCCGGAGGUGCUCAC | 51.7 | 48.7 |
| 368 | 498 | CCCAGGAGUACACCUGCUGUU | 1742 | CAGCAGGUGUACUCCUGGGGA | 69 | 66 |
| 369 | 499 | CCAGGAGUACACCUGCUGUUC | 1743 | ACAGCAGGUGUACUCCUGGGG | 80.8 | 77.8 |
| 370 | 500 | CAGGAGUACACCUGCUGUUCC | 1744 | AACAGCAGGUGUACUCCUGGG | 87.6 | 84.6 |
| 371 | 501 | AGGAGUACACCUGCUGUUCCA | 1745 | GAACAGCAGGUGUACUCCUGG | 71.7 | 68.7 |
| 372 | 502 | GGAGUACACCUGCUGUUCCAG | 1746 | GGAACAGCAGGUGUACUCCUG | 74.3 | 71.3 |
| 373 | 503 | GAGUACACCUGCUGUUCCAGU | 1747 | UGGAACAGCAGGUGUACUCCU | 87.7 | 84.7 |
| 374 | 504 | AGUACACCUGCUGUUCCAGUG | 1748 | CUGGAACAGCAGGUGUACUCC | 59.9 | 56.9 |
| 375 | 505 | GUACACCUGCUGUUCCAGUGA | 1749 | ACUGGAACAGCAGGUGUACUC | 67.8 | 64.8 |
| 376 | 506 | UACACCUGCUGUUCCAGUGAG | 1750 | CACUGGAACAGCAGGUGUACU | 80.5 | 57.5 |
| 377 | 507 | ACACCUGCUGUUCCAGUGAGA | 1751 | UCACUGGAACAGCAGGUGUAC | 75.8 | 72.8 |
| 378 | 508 | CACCUGCUGUUCCAGUGAGAC | 1752 | CUCACUGGAACAGCAGGUGUA | 75.7 | 72.7 |
| 379 | 509 | ACCUGCUGUUCCAGUGAGACA | 1753 | UCUCACUGGAACAGCAGGUGU | 84.6 | 81.6 |
| 380 | 510 | CCUGCUGUUCCAGUGAGACAG | 1754 | GUCUCACUGGAACAGCAGGUG | 72.2 | 69.2 |
| 381 | 511 | CUGCUGUUCCAGUGAGACAGA | 1755 | UGUCUCACUGGAACAGCAGGU | 87.7 | 84.7 |
| 382 | 512 | UGCUGUUCCAGUGAGACAGAG | 1756 | CUGUCUCACUGGAACAGCAGG | 71.6 | 68.6 |
| 383 | 513 | GCUGUUCCAGUGAGACAGAGC | 1757 | UCUGUCUCACUGGAACAGCAG | 93.3 | 90.3 |
| 384 | 514 | CUGUUCCAGUGAGACAGAGCA | 1758 | CUCUGUCUCACUGGAACAGCA | 67.6 | 64.6 |
| 385 | 515 | UGUUCCAGUGAGACAGAGCAG | 1759 | GCUCUGUCUCACUGGAACAGC | 56 | 53 |
| 386 | 516 | GUUCCAGUGAGACAGAGCAGA | 1760 | UGCUCUGUCUCACUGGAACAG | 86.6 | 83.6 |
| 387 | 517 | UUCCAGUGAGACAGAGCAGAG | 1761 | CUGCUCUGUCUCACUGGAACA | 59.5 | 56.5 |
| 388 | 518 | UCCAGUGAGACAGAGCAGAGG | 1762 | UCUGCUCUGUCUCACUGGAAC | 68.1 | 65.1 |
| 389 | 519 | CCAGUGAGACAGAGCAGAGGC | 1763 | CUCUGCUCUGUCUCACUGGAA | 76.1 | 73.1 |
| 390 | 520 | CAGUGAGACAGAGCAGAGGCU | 1764 | CCUCUGCUCUGUCUCACUGGA | 68.6 | 65.6 |
| 391 | 521 | AGUGAGACAGAGCAGAGGCUG | 1765 | GCCUCUGCUCUGUCUCACUGG | 66.3 | 63.3 |
| 392 | 522 | GUGAGACAGAGCAGAGGCUGA | 1766 | AGCCUCUGCUCUGUCUCACUG | 76.9 | 73.9 |
| 393 | 523 | UGAGACAGAGCAGAGGCUGAU | 1767 | CAGCCUCUGCUCUGUCUCACU | 57.3 | 54.3 |
| 394 | 524 | GAGACAGAGCAGAGGCUGAUC | 1768 | UCAGCCUCUGCUCUGUCUCAC | 82.3 | 79.3 |
| 395 | 525 | AGACAGAGCAGAGGCUGAUCA | 1769 | AUCAGCCUCUGCUCUGUCUCA | 78.1 | 75.1 |
| 396 | 526 | GAGAGAGGAGAGGCUGAUCAG | 1770 | GAUCAGCCUCUGCUCUGUCUC | 63.4 | 60.4 |
| 397 | 527 | ACAGAGCAGAGGCUGAUCAGG | 1771 | UGAUCAGCCUCUGCUCUGUCU | 76.2 | 73.2 |
| 398 | 528 | CAGAGCAGAGGCUGAUCAGGG | 1772 | CUGAUCAGCCUCUGCUCUGUC | 67.3 | 64.3 |
| 399 | 529 | AGAGCAGAGGCUGAUCAGGGA | 1773 | CCUGAUCAGCCUCUGCUCUGU | 63.1 | 60.1 |
| 400 | 530 | GAGCAGAGGCUGAUCAGGGAG | 1774 | CCCUGAUCAGCCUCUGCUCUG | 63.2 | 59.2 |
| 401 | 531 | AGCAGAGGCUGAUCAGGGAGA | 1775 | UCCCUGAUCAGCCUCUGCUCU | 78.1 | 74.1 |
| 402 | 532 | GCAGAGGCUGAUCAGGGAGAC | 1776 | CUCCCUGAUCAGCCUCUGCUC | 64.2 | 60.2 |
| 403 | 533 | CAGAGGCUGAUCAGGGAGACU | 1777 | UCUCCCUGAUCAGCCUCUGCU | 76.1 | 72.1 |
| 404 | 534 | AGAGGCUGAUCAGGGAGACUG | 1778 | GUCUCCCUGAUCAGCCUCUGC | 57.2 | 53.2 |
| 405 | 538 | GAGGCUGAUCAGGGAGACUGA | 1779 | AGUCUCCCUGAUCAGCCUCUG | 75 | 71 |
| 406 | 536 | AGGCUGAUCAGGGAGACUGAG | 1780 | CAGUCUCCCUGAUCAGCCUCU | 61.4 | 57.4 |
| 407 | 537 | GGCUGAUCAGGGAGACUGAGG | 1781 | UCAGUCUCCCUGAUCAGCCUC | 85.3 | 81.3 |
| 408 | 538 | GCUGAUCAGGGAGACUGAGGC | 1782 | CUCAGUCUCCCUGAUCAGCCU | 68.8 | 64.8 |
| 409 | 539 | CUGAUCAGGGAGACUGAGGCC | 1783 | CCUCAGUCUCCCUGAUCAGCC | 47.5 | 43.5 |
| 410 | 540 | UGAUCAGGGAGACUGAGGCCA | 1784 | GCCUCAGUCUCCCUGAUCAGC | 45.1 | 41.1 |
| 411 | 541 | GAUCAGGGAGACUGAGGCCAC | 1785 | GGCCUCAGUCUCCCUGAUCAG | 57.9 | 53.9 |
| 430 | 542 | ACCUUCCGAGGCCUGGUGGAG | 1786 | CCACCAGGCCUCGGAAGGUGG | 51 | 47 |
| 431 | 543 | CCUUCCGAGGCCUGGUGGAGG | 1787 | UCCACCAGGCCUCGGAAGGUG | 76.9 | 72.9 |
| 432 | 544 | CUUCCGAGGCCUGGUGGAGGA | 1788 | CUCCACCAGGCCUCGGAAGGU | 57.9 | 53.9 |
| 433 | 545 | UUCCGAGGCCUGGUGGAGGAC | 1789 | CCUCCACCAGGCCUCGGAAGG | 41.3 | 37.3 |
| 434 | 546 | UCCGAGGCCUGGUGGAGGACA | 1790 | UCCUCCACCAGGCCUCGGAAG | 69.1 | 65.1 |
| 435 | 547 | CCGAGGCCUGGUGGAGGACAG | 1791 | GUCCUCCACCAGGCCUCGGAA | 58.7 | 54.7 |
| 436 | 548 | CGAGGCCUGGUGGAGGACAGC | 1792 | UGUCCUCCACCAGGCCUCGGA | 75.4 | 71.4 |
| 437 | 549 | GAGGCCUGGUGGAGGACAGCG | 1793 | CUGUCCUCCACCAGGCCUCGG | 70.8 | 66.8 |
| 438 | 550 | AGGCCUGGUGGAGGACAGCGG | 1794 | GCUGUCCUCCACCAGGCCUCG | 53.9 | 49.9 |
| 439 | 551 | GGCCUGGUGGAGGACAGCGGC | 1795 | CGCUGUCCUCCACCAGGCCUC | 55.2 | 51.2 |
| 440 | 552 | GCCUGGUGGAGGACAGCGGCU | 1796 | CCGCUGUCCUCCACCAGGCCU | 64.3 | 60.3 |
| 441 | 553 | CCUGGUGGAGGACAGCGGCUC | 1797 | GCCGCUGUCCUCCACCAGGCC | 53.3 | 49.3 |
| 442 | 554 | CUGGUGGAGGACAGCGGCUCC | 1798 | AGCCGCUGUCCUCCACCAGGC | 62 | 58 |
| 443 | 555 | UGGUGGAGGACAGCGGCUCCU | 1799 | GAGCCGCUGUCCUCCACCAGG | 58.5 | 54.5 |
| 444 | 556 | GGUGGAGGACAGCGGCUCCUU | 1800 | GGAGCCGCUGUCCUCCACCAG | 69.3 | 65.3 |
| 445 | 557 | GUGGAGGACAGCGGCUCCUUU | 1801 | AGGAGCCGCUGUCCUCCACCA | 65.8 | 61.8 |
| 446 | 558 | UGGAGGACAGCGGCUCCUUUC | 1802 | AAGGAGCCGCUGUCCUCCACC | 60.1 | 56.1 |
| 447 | 559 | GGAGGACAGCGGCUCCUUUCU | 1803 | AAAGGAGCCGCUGUCCUCCAC | 78.3 | 74.3 |
| 448 | 560 | GAGGACAGCGGCUCCUUUCUG | 1804 | GAAAGGAGCCGCUGUCCUCCA | 64.8 | 60.8 |
| 449 | 561 | AGGACAGCGGCUCCUUUCUGG | 1805 | AGAAAGGAGCCGCUGUCCUCC | 65.2 | 61.2 |
| 450 | 562 | GGACAGCGGCUCCUUUCUGGU | 1806 | CAGAAAGGAGCCGCUGUCCUC | 68.1 | 64.1 |
| 451 | 563 | GACAGCGGCUCCUUUCUGGUU | 1807 | CCAGAAAGGAGCCGCUGUCCU | 60 | 56 |
| 452 | 564 | ACAGCGGCUCCUUUCUGGUUC | 1808 | ACCAGAAAGGAGCCGCUGUCC | 59.5 | 55.5 |
| 453 | 565 | CAGCGGCUCCUUUCUGGUUCA | 1809 | AACCAGAAAGGAGCCGCUGUC | 73.3 | 69.3 |
| 454 | 566 | AGCGGCUCCUUUCUGGUUCAC | 1810 | GAACCAGAAAGGAGCCGCUGU | 64 | 60 |
| 455 | 567 | GCGGCUCCUUUCUGGUUCACA | 1811 | UGAACCAGAAAGGAGCCGCUG | 91.7 | 87.7 |
| 456 | 568 | CGGCUCCUUUCUGGUUCACAC | 1812 | GUGAACCAGAAAGGAGCCGCU | 65.8 | 61.8 |
| 457 | 569 | GGCUCCUUUCUGGUUCACACA | 1813 | UGUGAACCAGAAAGGAGCCGC | 74.9 | 70.9 |
| 458 | 570 | GCUCCUUUCUGGUUCACACAC | 1814 | GUGUGAACCAGAAAGGAGCCG | 77.6 | 73.6 |
| 511 | 571 | UUUCUGGAGAUGCUCUCAGUA | 1815 | CUGAGAGCAUCUCCAGAAAAA | 56 | 51 |
| 512 | 572 | UUCUGGAGAUGCUCUCAGUAG | 1816 | ACUGAGAGCAUCUCCAGAAAA | 69.8 | 64.8 |
| 513 | 573 | UCUGGAGAUGCUCUCAGUAGC | 1817 | UACUGAGAGCAUCUCCAGAAA | 80.1 | 75.1 |
| 514 | 574 | CUGGAGAUGCUCUCAGUAGCC | 1818 | CUACUGAGAGCAUCUCCAGAA | 73.2 | 65.2 |
| 515 | 575 | UGGAGAUGCUCUCAGUAGCCC | 1819 | GCUACUGAGAGCAUCUCCAGA | 68.1 | 63.1 |
| 516 | 576 | GGAGAUGCUCUCAGUAGCCCA | 1820 | GGCUACUGAGAGCAUCUCCAG | 70.2 | 65.2 |
| 517 | 577 | GAGAUGCUCUCAGUAGCCCAG | 1821 | GGGCUACUGAGAGCAUCUCCA | 58.2 | 53.2 |
| 518 | 578 | AGAUGCUCUCAGUAGCCCAGC | 1822 | UGGGCUACUGAGAGCAUCUCC | 74.7 | 69.7 |
| 519 | 579 | GAUGeUCUCAGUAGCCCAGCA | 1823 | CUGGGCUACUGAGAGCAUCUC | 67.4 | 62.4 |
| 520 | 580 | AUGCUCUCAGUAGCCCAGCAC | 1824 | GCUGGGCUACUGAGAGCAUCU | 42.6 | 37.6 |
| 521 | 581 | UGCUCUCAGUAGCCCAGCACU | 1825 | UGCUGGGCUACUGAGAGCAUC | 71.1 | 66.1 |
| 522 | 582 | GCUCUCAGUAGCCCAGCACUC | 1826 | GUGCUGGGCUACUGAGAGCAU | 65.2 | 60.2 |
| 523 | 583 | CUCUCAGUAGCCCAGCACUCU | 1827 | AGUGCUGGGCUACUGAGAGCA | 69.1 | 64.1 |
| 524 | 584 | UCUCAGUAGCCCAGCACUCUC | 1828 | GAGUGCUGGGCUACUGAGAGC | 53.9 | 48.9 |
| 525 | 585 | CUCAGUAGCCCAGCACUCUCU | 1829 | AGAGUGCUGGGCUACUGAGAG | 68.6 | 63.6 |
| 526 | 586 | UCAGUAGCCCAGCACUCUCUG | 1830 | GAGAGUGCUGGGCUACUGAGA | 60.6 | 55.6 |
| 527 | 587 | CAGUAGCCCAGCACUCUCUGA | 1831 | AGAGAGUGCUGGGCUACUGAG | 74 | 69 |
| 528 | 588 | AGUAGCCCAGCACUCUCUGAC | 1832 | CAGAGAGUGCUGGGCUACUGA | 62.6 | 57.6 |
| 529 | 589 | GUAGaCCAGCACUCUCUGACC | 1833 | UCAGAGAGUGCUGGGCUACUG | 77.9 | 72.9 |
| 530 | 590 | UAGCCCAGCACUCUCUGACCC | 1834 | GUCAGAGAGUGCUGGGCUACU | 47.4 | 42.4 |
| 531 | 591 | AGCCCAGCACUCUCUGACCCA | 1835 | GGUCAGAGAGUGCUGGGCUAC | 51.1 | 46.1 |
| 532 | 592 | GCCCAGCACUCUCUGACCCAG | 1836 | GGGUCAGAGAGUGCUGGGCUA | 59.9 | 54.9 |
| 533 | 593 | CeCAGCACUCUCUGACCCAGC | 1837 | UGGGUCAGAGAGUGCUGGGCU | 72.5 | 67.5 |
| 534 | 594 | CCAGCACUCUCUGACCCAGCU | 1838 | CUGGGUCAGAGAGUGCUGGGC | 63.9 | 58.9 |
| 535 | 595 | CAGCACUCUCUGACCCAGCUC | 1839 | GCUGGGUCAGAGAGUGCUGGG | 60.7 | 55.7 |
| 536 | 596 | AGCACUCUCUGACCCAGCUCU | 1840 | AGCUGGGUCAGAGAGUGCUGG | 75.2 | 70.2 |
| 537 | 597 | GCACUCUCUGACCCAGCUCUU | 1841 | GAGCUGGGUCAGAGAGUGCUG | 68.6 | 63.6 |
| 538 | 598 | CACUCUCUGACCCAGCUCUUC | 1842 | AGAGCUGGGUCAGAGAGUGCU | 71.8 | 66.8 |
| 539 | 599 | ACUCUCUGACCCAGCUCUUCU | 1843 | AAGAGCUGGGUCAGAGAGUGC | 68.7 | 63.7 |
| 540 | 600 | CUCUCUGACCCAGCUCUUCUC | 1844 | GAAGAGCUGGGUCAGAGAGUG | 64.2 | 59.2 |
| 541 | 601 | UCUCUGACCCAGCUCUUCUCC | 1845 | AGAAGAGCUGGGUCAGAGAGU | 64.9 | 59.9 |
| 542 | 602 | CUCUGACCCAGCUCUUCUCCC | 1846 | GAGAAGAGCUGGGUCAGAGAG | 66.2 | 61.2 |
| 543 | 603 | UCUGACCCAGCUCUUCUCCCA | 1847 | GGAGAAGAGCUGGGUCAGAGA | 45.5 | 40.5 |
| 544 | 604 | CUGACCCAGCUCUUCUCCCAC | 1848 | GGGAGAAGAGCUGGGUCAGAG | 51.4 | 46.4 |
| 545 | 605 | UGACCCAGCUCUUCUCCCACU | 1849 | UGGGAGAAGAGCUGGGUCAGA | 71.4 | 66.4 |
| 546 | 606 | GACCCAGCUCUUCUCCCACUC | 1850 | GUGGGAGAAGAGCUGGGUCAG | 65.7 | 60.7 |
| 547 | 607 | ACCCAGCUCUUCUCCCACUCC | 1851 | AGUGGGAGAAGAGCUGGGUCA | 65.4 | 60.4 |
| 548 | 608 | CCCAGCUCUUCUCCCACUCCU | 1852 | GAGUGGGAGAAGAGCUGGGUC | 59.1 | 54.1 |
| 549 | 609 | CCAGCUCUUCUCCCACUCCUA | 1853 | GGAGUGGGAGAAGAGCUGGGU | 61.4 | 56.4 |
| 550 | 610 | CAGCUCUUCUCCCACUCCUAC | 1854 | AGGAGUGGGAGAAGAGCUGGG | 75.9 | 70.9 |
| 551 | 611 | AGCUCUUCUCCCACUCCUACG | 1855 | UAGGAGUGGGAGAAGAGCUGG | 90.4 | 85.4 |
| 552 | 612 | GCUCUUCUCCCACUCCUACGG | 1856 | GUAGGAGUGGGAGAAGAGCUG | 73 | 68 |
| 553 | 613 | CUCUUCUCCCACUCCUACGGC | 1857 | CGUAGGAGUGGGAGAAGAGCU | 57 | 52 |
| 554 | 614 | UCUUCUCCCACUCCUACGGCC | 1858 | CCGUAGGAGUGGGAGAAGAGC | 47.7 | 42.7 |
| 555 | 615 | CUUCUCCCACUCCUACGGCCG | 1859 | GCCGUAGGAGUGGGAGAAGAG | 45.1 | 43.1 |
| 556 | 616 | UUCUCCCACUCCUACGGCCGC | 1860 | GGCCGUAGGAGUGGGAGAAGA | 41 | 36 |
| 557 | 617 | UCUCCCACUCCUACGGCCGCC | 1861 | CGGCCGUAGGAGUGGGAGAAG | 46.2 | 41.2 |
| 558 | 618 | CUCCCACUCCUACGGCCGCCU | 1862 | GCGGCCGUAGGAGUGGGAGAA | 49.9 | 44.9 |
| 559 | 619 | UCCCACUCCUACGGCCGCCUG | 1863 | GGCGGCCGUAGGAGUGGGAGA | 36.1 | 31.1 |
| 560 | 620 | CCCACUCCUACGGCCGCCUGU | 1864 | AGGCGGCCGUAGGAGUGGGAG | 59.3 | 54.3 |
| 561 | 621 | CCACUCCUACGGCCGCCUGUA | 1865 | CAGGCGGCCGUAGGAGUGGGA | 58.7 | 53.7 |
| 562 | 622 | CACUCCUACGGCCGCCUGUAU | 1866 | ACAGGCGGCCGUAGGAGUGGG | 68.9 | 63.9 |
| 563 | 623 | ACUCCUACGGCCGCCUGUAUG | 1867 | UACAGGCGGCCGUAGGAGUGG | 75.5 | 70.5 |
| 564 | 624 | CUCCUACGGCCGCCUGUAUGC | 1868 | AUACAGGCGGCCGUAGGAGUG | 74.2 | 69.2 |
| 565 | 625 | UCCUACGGCCGCCUGUAUGCC | 1869 | CAUACAGGCGGCCGUAGGAGU | 51.9 | 46.9 |
| 566 | 626 | CCUACGGCCGCCUGUAUGCCC | 1870 | GCAUACAGGCGGCCGUAGGAG | 58.8 | 53.8 |
| 567 | 627 | CUACGGCCGCCUGUAUGCCCA | 1871 | GGCAUACAGGCGGCCGUAGGA | 46.7 | 41.7 |
| 568 | 628 | UACGGCCGCCUGUAUGCCCAG | 1872 | GGGCAUACAGGCGGCCGUAGG | 40.1 | 35.1 |
| 569 | 629 | ACGGCCGCCUGUAUGCCCAGC | 1873 | UGGGCAUACAGGCGGCCGUAG | 64.8 | 59.8 |
| 570 | 630 | CGGCCGCCUGUAUGCCCAGCA | 1874 | CUGGGCAUACAGGCGGCCGUA | 63.9 | 58.9 |
| 571 | 631 | GGCCGCCUGUAUGCCCAGCAC | 1875 | GCUGGGCAUACAGGCGGCCGU | 52.4 | 47.4 |
| 572 | 632 | GCCGCCUGUAUGCCCAGCACG | 1876 | UGCUGGGCAUACAGGCGGCCG | 69 | 64 |
| 573 | 633 | CCGCCUGUAUGCCCAGCACGC | 1877 | GUGCUGGGCAUACAGGCGGCC | 58.6 | 53.6 |
| 574 | 634 | CGCCUGUAUGCCCAGCACGCC | 1878 | CGUGCUGGGCAUACAGGCGGC | 57.2 | 52.2 |
| 575 | 635 | GCCUGUAUGCCCAGCACGCCC | 1879 | GCGUGCUGGGCAUACAGGCGG | 63.6 | 58.6 |
| 576 | 636 | CCUGUAUGCCCAGCACGCCCU | 1880 | GGCGUGCUGGGCAUACAGGCG | 51.2 | 46.2 |
| 577 | 637 | CUGUAUGCCCAGCACGCCCUC | 1881 | GGGCGUGCUGGGCAUACAGGC | 47.4 | 42.4 |
| 578 | 638 | UGUAUGCCCAGCACGCCCUCA | 1882 | AGGGCGUGCUGGGCAUACAGG | 58.9 | 53.9 |
| 579 | 639 | GUAUGCCCAGCACGCCCUCAU | 1883 | GAGGGCGUGCUGGGCAUACAG | 54.8 | 49.8 |
| 580 | 640 | UAUGCCCAGCACGCCCUCAUA | 1884 | UGAGGGCGUGCUGGGCAUACA | 53.9 | 48.9 |
| 581 | 641 | AUGCCCAGCACGCCCUCAUAU | 1885 | AUGAGGGCGUGCUGGGCAUAC | 54 | 49 |
| 582 | 642 | UGCCCAGCACGCCCUCAUAUU | 1886 | UAUGAGGGCGUGCUGGGCAUA | 74.3 | 69.3 |
| 583 | 643 | GCCCAGCACGCCCUCAUAUUC | 1887 | AUAUGAGGGCGUGCUGGGCAU | 72.6 | 67.6 |
| 584 | 644 | CCCAGCACGCCCUCAUAUUCA | 1888 | AAUAUGAGGGCGUGCUGGGCA | 70.8 | 65.8 |
| 585 | 645 | CCAGCACGCCCUCAUAUUCAA | 1889 | GAAUAUGAGGGCGUGCUGGGC | 61.5 | 56.5 |
| 586 | 646 | CAGCACGCCCUCAUAUUCAAU | 1890 | UGAAUAUGAGGGCGUGCUGGG | 77.4 | 72.4 |
| 587 | 647 | AGCACGCCCUCAUAUUCAAUG | 1891 | UUGAAUAUGAGGGCGUGCUGG | 91.7 | 86.7 |
| 588 | 648 | GCACGCCCUCAUAUUCAAUGG | 1892 | AUUGAAUAUGAGGGCGUGCUG | 81.4 | 76.4 |
| 607 | 649 | GGCCUGUUCUCUCGGCUGCGA | 1893 | GCAGCCGAGAGAACAGGCCAU | 66.3 | 60.3 |
| 608 | 650 | GCCUGUUCUCUCGGCUGCGAG | 1894 | CGCAGCCGAGAGAACAGGCCA | 61.2 | 55.2 |
| 609 | 651 | CCUGUUCUCUCGGCUGCGAGA | 1895 | UCGCAGCCGAGAGAACAGGCC | 74.9 | 68.9 |
| 610 | 652 | CUGUUCUCUCGGCUGCGAGAC | 1896 | CUCGCAGCCGAGAGAACAGGC | 59.5 | 53.5 |
| 611 | 653 | UGUUCUCUCGGCUGCGAGACU | 1897 | UCUCGCAGCCGAGAGAACAGG | 12.9 | 76.9 |
| 612 | 654 | GUUCUCUCGGCUGCGAGACUU | 1898 | GUCUCGCAGCCGAGAGAACAG | 61.6 | 55.6 |
| 613 | 655 | UUCUCUCGGCUGCGAGACUUC | 1899 | AGUCUCGCAGCCGAGAGAACA | 60.5 | 54.5 |
| 614 | 656 | UCUCUCGGCUGCGAGACUUCU | 1900 | AAGUCUCGCAGCCGAGAGAAC | 61.9 | 55.9 |
| 615 | 657 | CUCUCGGCUGCGAGACUUCUA | 1901 | GAAGUCUCGCAGCCGAGAGAA | 67.9 | 61.9 |
| 616 | 658 | UCUCGGCUGCGAGACUUCUAU | 1902 | AGAAGUCUCGCAGCCGAGAGA | 69.2 | 63.2 |
| 617 | 659 | CUCGGCUGCGAGACUUCUAUG | 1903 | UAGAAGUCUCGCAGCCGAGAG | 88.9 | 82.9 |
| 618 | 660 | UCGGCUGCGAGACUUCUAUGG | 1904 | AUAGAAGUCUCGCAGCCGAGA | 76.5 | 70.5 |
| 619 | 661 | CGGCUCCGAGACUUCUAUGGG | 1905 | CAUAGAAGUCUCGCAGCCGAG | 79.7 | 73.7 |
| 669 | 662 | GGCGGAUUUCUGGGCACAGCU | 1906 | CUGUGCCCAGAAAUCCGCCAG | 69 | 63 |
| 670 | 663 | GCGGAUUUCUGGGCACAGCUC | 1007 | GCUGUGCCCAGAAAUCCGCCA | 60 | 54 |
| 671 | 664 | CGGAUUUCUGGGCACAGCUCC | 1908 | AGCUGUGCCCAGAAAUCCGCC | 66.1 | 60.1 |
| 672 | 665 | GGAUUUCUGGGCACAGCUCCU | 1909 | GAGCUGUGCCCAGAAAUCCGC | 64.6 | 58.6 |
| 673 | 666 | GAUUUCUGGGCACAGCUCCUG | 1910 | GGAGCUGUGCCCAGAAAUCCG | 57.3 | 51.3 |
| 674 | 667 | AUUUCUGGGCACAGCUCCUGG | 1911 | AGGAGCUGUGCCCAGAAAUCC | 56 | 50 |
| 675 | 668 | UUUCUGGGCACAGCUCCUGGA | 1912 | CAGGAGCUGUGCCCAGAAAUC | 40.1 | 34.1 |
| 676 | 669 | UUCUGGGCACAGCUCCUGGAG | 1913 | CCAGGAGCUGUGCCCAGAAAU | 45.7 | 39.7 |
| 677 | 670 | UCUGGGCACAGCUCCUGGAGA | 1914 | UCCAGGAGCUGUGCCCAGAAA | 67.2 | 61.2 |
| 678 | 671 | CUGGGCACAGCUCCUGGAGAG | 1915 | CUCCAGGAGCUGUGCCCAGAA | 56.6 | 50.6 |
| 679 | 672 | UGGGCACAGCUCCUGGAGAGA | 1916 | UCUCCAGGAGCUGUGCCCAGA | 70.5 | 64.5 |
| 680 | 673 | GGGCACAGCUCCUGGAGAGAG | 1917 | CUCUCCAGGAGCUGUGCCCAG | 71 | 65 |
| 681 | 674 | GGCACAGCUCCUGGAGAGAGU | 1918 | UCUCUCCAGGAGCUGUGCCCA | 81.7 | 75.7 |
| 682 | 675 | GCACAGCUCCUGGAGAGAGUG | 1919 | CUCUCUCCAGGAGCUGUGCCC | 62.6 | 56.6 |
| 683 | 676 | CACAGCUCCUGGAGAGAGUGU | 1920 | ACUCUCUCCAGGAGCUGUGCC | 69.9 | 63.9 |
| 684 | 677 | ACAGCUCCUGGAGAGAGUGUU | 1921 | CACUCUCUCCAGGAGCUGUGC | 59.9 | 53.9 |
| 685 | 678 | CAGCUCCUGGAGAGAGUGUUC | 1922 | ACACUCUCUCCAGGAGCUGUG | 78 | 72 |
| 686 | 679 | AGCUCCUGGAGAGAGUGUUCC | 1923 | AACACUCUCUCCAGGAGCUGU | 80 | 74 |
| 687 | 680 | GCUCCUGGAGAGAGUGUUCCC | 1924 | GAACACUCUCUCCAGGAGCUG | 81.5 | 75.5 |
| 688 | 681 | CUCCUGGAGAGAGUGUUCCCG | 1925 | GGAACACUCUCUCCAGGAGCU | 56.5 | 50.5 |
| 689 | 682 | UCCUGGAGAGAGUGUUCCCGC | 1926 | GGGAACACUCUCUCCAGGAGC | 54 | 48 |
| 690 | 683 | CCUGGAGAGAGUGUUCCCGCU | 1927 | CGGGAACACUCUCUCCAGGAG | 63.8 | 57.8 |
| 691 | 684 | CUGGAGAGAGUGUUCCCGCUG | 1928 | GCGGGAACACUCUCUCCAGGA | 59.5 | 53.5 |
| 692 | 685 | UGGAGAGAGUGUUCCCGCUGC | 1929 | AGCGGGAACACUCUCUCCAGG | 74.6 | 68.6 |
| 693 | 686 | GGAGAGAGUGUUCCCGCUGCU | 1930 | CAGCGGGAACACUCUCUCCAG | 77.3 | 71.3 |
| 694 | 687 | GAGAGAGUGUUCCCGCUGCUG | 1931 | GCAGCGGGAACACUCUCUCCA | 67.2 | 61.2 |
| 695 | 688 | AGAGAGUGUUCCCGCUGCUGC | 1932 | AGCAGCGGGAACACUCUCUCC | 72.2 | 66.2 |
| 696 | 689 | GAGAGUGUUCCCGCUGCUGCA | 1933 | CAGCAGCGGGAACACUCUCUC | 65.7 | 59.7 |
| 697 | 690 | AGAGUGUUCCCGCUGCUGCAC | 1934 | GCAGCAGCGGGAACACUCUCU | 61.3 | 55.3 |
| 698 | 691 | GAGUGUUCCCGCUGCUGCACC | 1935 | UGCAGCAGCGGGAACACUCUC | 83.8 | 77.8 |
| 699 | 692 | AGUGUUCCCGCUGCUGCACCC | 1936 | GUGCAGCAGCGGGAACACUCU | 61.3 | 66.3 |
| 700 | 693 | GUGUUCCCGCUGCUGCACCCA | 1937 | GGUGCAGCAGCGGGAACACUC | 49.4 | 42.4 |
| 701 | 694 | UGUUCCCGCUGCUGCACCCAC | 1938 | GGGUGCAGCAGCGGGAACACU | 51.7 | 44.7 |
| 702 | 695 | GUUCCCGCUGCUGCACCCACA | 1939 | UGGGUGCAGCAGCGGGAACAC | 61.8 | 54.8 |
| 703 | 696 | UUCCCGCUGCUGCACCCACAG | 1940 | GUGGGUGCAGCAGCGGGAACA | 47.4 | 40.4 |
| 704 | 697 | UCCCGCUGCUGCACCCACAGU | 1941 | UGUGGGUGCAGCAGCGGGAAC | 61.9 | 54.9 |
| 705 | 698 | CCCGCUGCUGCACCCACAGUA | 1942 | CUGUGGGUGCAGCAGCGGGAA | 66.2 | 59.2 |
| 706 | 699 | CCGCUGCUGCACCCACAGUAC | 1943 | ACUGUGGGUGCAGCAGCGGGA | 74.1 | 67.1 |
| 707 | 700 | CGCUGCUGCACCCACAGUACA | 1944 | UACUGUGGGUGCAGCAGCGGG | 89.1 | 82.1 |
| 708 | 701 | GCUGCUGCACCCACAGUACAG | 1945 | GUACUGUGGGUGCAGCAGCGG | 77.2 | 70.2 |
| 709 | 702 | CUGCUGCACCCACAGUACAGC | 1946 | UGUACUGUGGGUGCAGCAGCG | 81.2 | 74.2 |
| 710 | 703 | UGCUGCACCCACAGUACAGCU | 1947 | CUGUACUGUGGGUGCAGCAGC | 58.9 | 51.9 |
| 711 | 704 | GCUGCACCCACAGUACAGCUU | 1948 | GCUGUACUGUGGGUGCAGCAG | 61.8 | 54.8 |
| 712 | 705 | CUGCACCCACAGUACAGCUUC | 1949 | AGCUGUACUGUGGGUGCAGCA | 67.4 | 60.4 |
| 713 | 706 | UGCACCCACAGUACAGCUUCC | 1950 | AAGCUGUACUGUGGGUGCAGC | 64.4 | 57.4 |
| 714 | 707 | GCACCCACAGUACAGCUUCCC | 1951 | GAAGCUGUACUGUGGGUGCAG | 71.1 | 64.1 |
| 735 | 708 | CCCUGACUACCUGCUCUGCCU | 1952 | GCAGAGCAGGUAGUCAGGGGG | 64.2 | 57.2 |
| 736 | 709 | CCUGACUACCUGCUCUGCCUC | 1953 | GGCAGAGCAGGUAGUCAGGGG | 59.7 | 52.7 |
| 737 | 710 | CUGACUACCUGCUCUGCCUCU | 1954 | AGGCAGAGCAGGUAGUCAGGG | 76.6 | 69.8 |
| 738 | 711 | UGACUACCUGCUCUGCCUCUC | 1955 | GAGGCAGAGCAGGUAGUCAGG | 60.3 | 53.3 |
| 739 | 712 | GACUACCUGCUCUGCCUCUCA | 1956 | AGAGGCAGAGCAGGUAGUCAG | 74.4 | 67.4 |
| 740 | 713 | ACUACCUGCUCUGCCUCUCAC | 1957 | GAGAGGCAGAGCAGGUAGUCA | 57.9 | 50.9 |
| 741 | 714 | CUACCUGCUCUGCCUCUCACG | 1958 | UGAGAGGCAGAGCAGGUAGUC | 74.9 | 67.9 |
| 742 | 715 | UACCUGCUCUGCCUCUCACGC | 1959 | GUGAGAGGCAGAGCAGGUAGU | 58.7 | 51.7 |
| 743 | 715 | UACCUGCUCUGCCUCUCACGC | 1960 | CGUGAGAGGCAGAGCAGGUAG | 57.9 | 50.9 |
| 744 | 717 | CCUGCUCUGCCUCUCACGCUU | 1961 | GCGUGAGAGGCAGAGCAGGUA | 59.9 | 52.9 |
| 745 | 718 | CUGCUCUGCCUCUCACGCUUG | 1962 | AGCGUGAGAGGCAGAGCAGGU | 64.7 | 57.7 |
| 746 | 719 | UGCUCUGCCUCUCACGCUUGG | 1963 | AAGCGUGAGAGGCAGAGCAGG | 77.1 | 70.1 |
| 781 | 720 | GGCUCUCUGCAGCCCUUUGGG | 1964 | CAAAGGGCUGCAGAGAGCCAU | 72.2 | 65.2 |
| 809 | 721 | CCCGCCGCCUCCGCCUGCAGA | 1965 | UGCAGGCGGAGGCGGCGGGGU | 60.6 | 52.6 |
| 810 | 722 | CCGCCGCCUCCGCCUGCAGAU | 1966 | CUGCAGGCGGAGGCGGCGGGG | 59.5 | 51.5 |
| 811 | 723 | CGCCGCGUCCGCCUGCAGAUA | 1967 | UCUGCAGGCGGAGGCGGCGGG | 71.1 | 63.1 |
| 812 | 724 | GCCGCCUCCGCCUGCAGAUAA | 1968 | AUCUGCAGGCGGAGGCGGCGG | 72.4 | 64.4 |
| 813 | 725 | CCGCCUCCGCCUGCAGAUAAC | 1969 | UAUCUGCAGGCGGAGGCGGCG | 77.6 | 69.6 |
| 814 | 726 | CGCCUCCGCCUGCAGAUAACC | 1970 | UUAUCUGCAGGCGGAGGCGGC | 77 | 69 |
| 815 | 727 | GCCUCCGCCUGCAGAUAACCC | 1971 | GUUAUCUGCAGGCGGAGGCGG | 70.4 | 62.4 |
| 816 | 728 | CCUCCGCCUGCAGAUAACCCG | 1972 | GGUUAUCUGCAGGCGGAGGCG | 55.9 | 47.9 |
| 817 | 729 | CUCCGCCUGCAGAUAACCCGG | 1973 | GGGUUAUCUGCAGGCGGAGGC | 44.6 | 36.6 |
| 818 | 730 | UCCGCCUGCAGAUAACCCGGA | 1974 | CGGGUUAUCUGCAGGCGGAGG | 50.1 | 42.1 |
| 819 | 731 | CCGCCUGCAGAUAACCCGGAC | 1975 | CCGGGUUAUCUGCAGGCGGAG | 62.3 | 54.3 |
| 820 | 732 | CGCCUGCAGAUAACCCGGACC | 1976 | UCCGGGUUAUCUGCAGGCGGA | 76.9 | 58.9 |
| 821 | 733 | GCCUGCAGAUAACCCGGACCC | 1977 | GUCCGGGUUAUCUGCAGGCGG | 70 | 62 |
| 822 | 734 | CCUGCAGAUAACCCGGACCCU | 1978 | GGUCCGGGUUAUCUGCAGGCG | 60.4 | 52.4 |
| 823 | 735 | CUGCAGAUAACCCGGACCCUG | 1979 | GGGUCCGGGUUAUCUGCAGGC | 50.1 | 42.1 |
| 824 | 736 | UGCAGAUAACCCGGACCCUGG | 1980 | AGGGUCCGGGUUAUCUGCAGG | 65.3 | 57.3 |
| 825 | 737 | GCAGAUAACCGGGACCCUGGU | 1981 | CAGGGUCCGGGUUAUCUGCAG | 69.3 | 61.3 |
| 826 | 738 | CAGAUAACCCGGACCCUGGUG | 1982 | CCAGGGUCCGGGUUAUCUGCA | 60.1 | 52.1 |
| 827 | 739 | AGAUAACCGGGACCCUGGUGG | 1983 | ACCAGGGUCGGGGUUAUCUGC | 65.2 | 57.2 |
| 846 | 740 | GGCUGCCCGAGCCUUUGUGCA | 1984 | CACAAAGGCUCGGGCAGCCAC | 57.7 | 49.7 |
| 847 | 741 | GCUGCCCGAGCCUUUGUGCAG | 1985 | GCACAAAGGCUCGGGCAGCCA | 55.2 | 47.2 |
| 848 | 742 | CUGCCCGAGCCUUUGUGCAGG | 1986 | UGCACAAAGGCUCGGGCAGCC | 62.1 | 54.1 |
| 849 | 743 | UGCCCGAGCCUUUGUGCAGGG | 1987 | CUGCACAAAGGCUCGGGCAGC | 55.2 | 47.2 |
| 850 | 744 | GCCCGAGCCUUUGUGCAGGGC | 1988 | CCUGCACAAAGGCUCGGGCAG | 60.4 | 52.4 |
| 851 | 745 | CCCGAGCCUUUGUGCAGGGCC | 1989 | CCCUGCACAAAGGCUCGGGCA | 57.7 | 49.7 |
| 852 | 745 | CCGAGCCUUUGUGCAGGGCCU | 1990 | GCCCUGCACAAAGGCUCGGGC | 45.9 | 37.9 |
| 853 | 747 | CGAGCCUUUGUGCAGGGCCUG | 1991 | GGCCCUGCACAAAGGCUCGGG | 56.3 | 48.3 |
| 854 | 748 | GAGCCUUUGUGCAGGGCCUGG | 1992 | AGGCCCUGCACAAAGGCUCGG | 73.6 | 65.6 |
| 855 | 749 | AGCCUUUGUGCAGGGCCUGGA | 1993 | CAGGCCCUGCACAAAGGCUCG | 55 | 47 |
| 856 | 750 | GCCUUUGUGCAGGGCCUGGAG | 1994 | CCAGGCCCUGCACAAAGGCUC | 50.3 | 42.3 |
| 857 | 751 | CCUUUGUGCAGGGCCUGGAGA | 1995 | UCCAGGCCCUGCACAAAGGCU | 69.2 | 61.2 |
| 858 | 752 | CUUUGUGCAGGGCCUGGAGAC | 1996 | CUCCAGGCCCUGCACAAAGGC | 54.5 | 46.5 |
| 859 | 753 | UUUGUGCAGGGCCUGGAGACU | 1997 | UCUCCAGGCCCUGCACAAAGG | 60.9 | 52.9 |
| 860 | 754 | UUGUGCAGGGCCUGGAGACUG | 1998 | GUCUCCAGGCCCUGCACAAAG | 47.9 | 39.9 |
| 861 | 755 | UGUGCAGGGCCUGGAGACUGG | 1999 | AGUCUCCAGGCCCUGCACAAA | 60.6 | 52.6 |
| 862 | 756 | GUGCAGGGCCUGGAGACUGGA | 2000 | CAGUCUCCAGGCCCUGCACAA | 59.1 | 51.1 |
| 863 | 757 | UGCAGGGCCUGGAGACUGGAA | 2001 | CCAGUCUCCAGGCCCUGCACA | 53.4 | 53.4 |
| 864 | 758 | GCAGGGCCUGGAGACUGGAAG | 2002 | UCCAGUCUCCAGGCCCUGCAC | 70.4 | 62.4 |
| 865 | 759 | CAGGGCCUGGAGACUGGAAGA | 2003 | UUCCAGUCUCCAGGCCCUGCA | 75.6 | 67.6 |
| 866 | 760 | AGGGCCUGGAGACUGGAAGAA | 2004 | CUUCCAGUCUCCAGGCCCUGC | 54.1 | 46.1 |
| 867 | 761 | GGGCCUGGAGACUGGAAGAAA | 2005 | UCUUCCAGUCUCCAGGCCCUG | 87.6 | 79.6 |
| 868 | 762 | GGCCUGGAGACUGGAAGAAAU | 2006 | UUCUUCCAGUCUCCAGGCCCU | 83.3 | 75.3 |
| 869 | 763 | GCCUGGAGACUGGAAGAAAUG | 2007 | UUUCUUCCAGUCUCCAGGCCC | 82.9 | 74.9 |
| 870 | 764 | CCUGGAGACUGGAAGAAAUGU | 2008 | AUUUCUUCCAGUCUCCAGGCC | 83.3 | 75.3 |
| 871 | 765 | CUGGAGACUGGAAGAAAUGUG | 2009 | CAUUUCUUCCAGUCUCCAGGC | 67.1 | 59.1 |
| 872 | 766 | UGGAGACUGGAAGAAAUGUGG | 2010 | ACAUUUCUUCCAGUCUCCAGG | 74.6 | 66.6 |
| 873 | 767 | GGAGACUGGAAGAAAUGUGGU | 2011 | CACAUUUCUUCCAGUCUCCAG | 80.5 | 72.5 |
| 874 | 768 | GAGACUGGAAGAAAUGUGGUC | 2012 | CCACAUUUCUUCCAGUCUCCA | 71.8 | 63.8 |
| 875 | 769 | AGACUGGAAGAAAUGUGGUCA | 2013 | ACCACAUUUCUUCCAGUCUCC | 70 | 62 |
| 876 | 770 | GACUGGAAGAAAUGUGGUCAG | 2014 | GACCACAUUUCUUCCAGUCUC | 69.5 | 61.5 |
| 877 | 771 | ACUGGAAGAAAUGUGGUCAGC | 2015 | UGACCACAUUUCUUCCAGUCU | 75.8 | 67.8 |
| 878 | 772 | CUGGAAGAAAUGUGGUCAGCG | 2016 | CUGACCACAUUUCUUCCAGUC | 68.1 | 60.1 |
| 879 | 773 | UGGAAGAAAUGUGGUCAGCGA | 2017 | GCUGACCACAUUUCUUCCAGU | 60.4 | 52.4 |
| 880 | 774 | GGAAGAAAUGUGGUCAGCGAA | 2018 | CGCUGACCACAUUUCUUCCAG | 68.6 | 60.6 |
| 881 | 775 | GAAGAAAUGUGGUCAGCGAAG | 2019 | UCGCUGACCACAUUUCUUCCA | 93 | 85 |
| 882 | 776 | AAGAAAUGUGGUCAGCGAAGC | 2020 | UUCGCUGACCACAUUUCUUCC | 82 | 74 |
| 883 | 777 | AGAAAUGUGGUCAGCGAAGCG | 2021 | CUUCGCUGACCACAUUUCUUC | 70.4 | 62.4 |
| 884 | 778 | GAAAUGUGGUCAGCGAAGCGC | 2022 | GCUUCGCUGACCACAUUUCUU | 69.5 | 61.5 |
| 885 | 779 | AAAUGUGGUCAGCGAAGCGCU | 2023 | CGCUUCGCUGACCACAUUUCU | 63.9 | 55.9 |
| 886 | 780 | AAUGUGGUCAGCGAAGCGCUU | 2024 | GCGCUUCGCUGACCACAUUUC | 52 | 44 |
| 887 | 781 | AUGUGGUCAGCGAAGCGCUUA | 2025 | AGCGCUUCGCUGACCACAUUU | 73.7 | 65.7 |
| 888 | 782 | UGUGGUCAGCGAAGCGCUUAA | 2026 | AAGCGCUUCGCUGACCACAUU | 81.9 | 73.9 |
| 889 | 783 | GUGGUCAGCGAAGCGCUUAAG | 2027 | UAAGCGCUUCGCUGACCACAU | 81.6 | 73.6 |
| 890 | 784 | UGGUCAGCGAAGCGCUUAAGG | 2028 | UUAAGCGCUUCGCUGACCACA | 85.8 | 77.8 |
| 891 | 785 | GGUCAGCGAAGCGCUUAAGGU | 2029 | CUUAAGCGCUUCGCUGACCAC | 70.6 | 62.6 |
| 892 | 786 | GUCAGCGAAGCGCUUAAGGUG | 2030 | CCUUAAGCGCUUCGCUGACCA | 58.6 | 50.6 |
| 893 | 787 | UCAGCGAAGCGCUUAAGGUGC | 2031 | ACCUUAAGCGCUUCGCUGACC | 60 | 52 |
| 894 | 788 | CAGCGAAGCGCUUAAGGUGCC | 2032 | CACCUUAAGCGCUUCGCUGAC | 67.2 | 59.2 |
| 895 | 789 | AGCGAAGCGCUUAAGGUGCCG | 2033 | GCACCUUAAGCGCUUCGCUGA | 65.1 | 57.1 |
| 896 | 790 | GCGAAGCGCUUAAGGUGCCGG | 2034 | GGCACCUUAAGCGCUUCGCUG | 68.3 | 60.3 |
| 897 | 791 | CGAAGCGCUUAAGGUGCCGGU | 2035 | CGGCACCUUAAGCGCUUCGCU | 60.4 | 52.4 |
| 898 | 792 | GAAGCGCUUAAGGUGCCGGUG | 2036 | CCGGCACCUUAAGCGCUUCGC | 51.1 | 43.1 |
| 899 | 793 | AAGCGCUUAAGGUGCCGGUGU | 2037 | ACCGGCACCUUAAGCGCUUCG | 62.4 | 54.4 |
| 900 | 794 | AGCGCUUAAGGUGCCGGUGUC | 2038 | CACCGGCACCUUAAGCGCUUC | 56.3 | 47.3 |
| 901 | 795 | GCGCUUAAGGUGCCGGUGUCU | 2039 | ACACCGGCACCUUAAGCGCUU | 76.2 | 67.2 |
| 902 | 796 | CGCUUAAGGUGCCGGUGUCUG | 2040 | GACACCGGCACCUUAAGCGCU | 70.2 | 61.2 |
| 903 | 797 | GCUUAAGGUGCCGGUGUCUGA | 2041 | AGACACCGGCACCUUAAGCGC | 69 | 60 |
| 904 | 798 | CUUAAGGUGCCGGUGUCUGAA | 2042 | CAGACACCGGCACCUUAAGCG | 60.1 | 51.1 |
| 905 | 799 | UUAAGGUGCCGGUGUCUGAAG | 2043 | UCAGACACCGGCACCUUAAGC | 65.2 | 56.2 |
| 906 | 800 | UAAGGUGCCGGUGUCUGAAGG | 2044 | UUCAGACACCGGCACCUUAAG | 73.2 | 64.2 |
| 907 | 801 | AAGGUGCCGGUGUCUGAAGGC | 2045 | CUUCAGACACCGGCACCUUAA | 62.8 | 53.8 |
| 908 | 802 | AGGUGCCGGUGUCUGAAGGCU | 2046 | CCUUCAGACACCGGCACCUUA | 62.1 | 53.1 |
| 909 | 803 | GGUGCCGGUGUCUGAAGGCUG | 2047 | GCCUUCAGACACCGGCACCUU | 60.9 | 51.9 |
| 910 | 804 | GUGCCGGUGUCUGAAGGCUGC | 2048 | AGCCUUCAGACACCGGCACCU | 66.8 | 57.8 |
| 911 | 805 | UGCCGGUGUCUGAAGGCUGCA | 2049 | CAGCCUUCAGACACCGGCACC | 55.2 | 46.2 |
| 912 | 806 | GCCGGUGUCUGAAGGCUGCAG | 2050 | GCAGCCUUCAGACACCGGCAC | 61.4 | 52.4 |
| 913 | 807 | CCGGUGUCUGAAGGCUGCAGC | 2051 | UGCAGCCUUCAGACACCGGCA | 76.5 | 67.5 |
| 914 | 808 | CGGUGUCUGAAGGCUGCAGCC | 2052 | CUGCAGCCUUCAGACACCGGC | 66.5 | 57.5 |
| 915 | 809 | GGUGUCUGAAGGCUGCAGCCA | 2053 | GCUGCAGCCUUCAGACACCGG | 65.3 | 56.3 |
| 916 | 810 | GUGUCUGAAGGCUGCAGCCAG | 2054 | GGCUGCAGCCUUCAGACACCG | 58.8 | 49.8 |
| 935 | 811 | AGGCUCUGAUGCGUCUCAUCG | 2055 | AUGAGACGCAUCAGAGCCUGG | 77 | 68 |
| 936 | 812 | GGCUCUGAUGCGUCUCAUCGG | 2056 | GAUGAGACGCAUCAGAGCCUG | 76.5 | 67.5 |
| 937 | 813 | GCUCUGAUGCGUCUCAUCGGC | 2057 | CGAUGAGACGCAUCAGAGCCU | 65 | 56 |
| 938 | 814 | CUCUGAUGCGUCUCAUCGGCU | 2058 | CCGAUGAGACGCAUCAGAGCC | 55.3 | 46.3 |
| 939 | 815 | UCUGAUGCGUCUCAUCGGCUG | 2059 | GCCGAUGAGACGCAUCAGAGC | 46.4 | 37.4 |
| 940 | 816 | CUGAUGCGUCUCAUCGGCUGU | 2060 | AGCCGAUGAGACGCAUCAGAG | 64.6 | 55.6 |
| 941 | 817 | UGAUGCGUCUCAUCGGCUGUC | 2061 | CAGCCGAUGAGACGCAUCAGA | 62.1 | 53.1 |
| 942 | 818 | GAUGCGUCUCAUCGGCUGUCC | 2062 | ACAGCCGAUGAGACGCAUCAG | 79.4 | 70.4 |
| 943 | 819 | AUGCGUCUCAUCGGCUGUCCC | 2063 | GACAGCCGAUGAGACGCAUCA | 50.9 | 41.9 |
| 979 | 820 | CCCUCACUUAUGCCCUGCCAG | 2064 | GGCAGGGCAUAAGUGAGGGGA | 54.5 | 45.5 |
| 998 | 821 | AGGGCUUCUGCCUCAACGUGG | 2065 | ACGUUGAGGCAGAAGCCCUGG | 71 | 62 |
| 999 | 822 | GGGCUUCUGCCUCAACGUGGU | 2066 | CACGUUGAGGCAGAAGCCCUG | 71 | 62 |
| 1000 | 823 | GGCUUCUGCCUCAACGUGGUU | 2067 | CCACGUUGAGGCAGAAGCCCU | 62 | 52 |
| 1001 | 824 | GCUUCUGCCUCAACGUGGUUC | 2068 | ACCACGUUGAGGCAGAAGCCC | 71.6 | 61.6 |
| 1002 | 825 | CUUCUGCCUCAACGUGGUUCG | 2069 | AACCACGUUGAGGCAGAAGCC | 66.5 | 56.5 |
| 1003 | 826 | UUCUGCCUCAACGUGGUUCGU | 2070 | GAACCACGUUGAGGCAGAAGC | 38.9 | 28.9 |
| 1004 | 827 | UCUGCCUCAACGUGGUUCGUG | 2071 | CGAACCACGUUGAGGCAGAAG | 54.1 | 44.1 |
| 1005 | 828 | CUGCCUCAACGUGGUUCGUGG | 2072 | ACGAACCACGUUGAGGCAGAA | 68.2 | 58.2 |
| 1059 | 829 | GGGCAACUAUCUGGAUGGUCU | 2073 | ACCAUCCAGAUAGUUGCCCCA | 75 | 65 |
| 1060 | 830 | GGCAACUAUCUGGAUGGUCUC | 2074 | GACCAUCCAGAUAGUUGCCCC | 58 | 48 |
| 1061 | 831 | GCAACUAUCUGGAUGGUCUCC | 2075 | AGACCAUCCAGAUAGUUGCCC | 74.9 | 64.9 |
| 1062 | 832 | CAACUAUCUGGAUGGUCUCCU | 2076 | GAGACCAUCCAGAUAGUUGCC | 65.7 | 55.7 |
| 1063 | 833 | AACUAUCUGGAUGGUCUCCUG | 2077 | GGAGACCAUCCAGAUAGUUGC | 48.8 | 38.8 |
| 1064 | 834 | ACUAUCUGGAUGGUCUCCUGA | 2078 | AGGAGACCAUCCAGAUAGUUG | 64.4 | 54.4 |
| 1065 | 835 | CUAUCUGGAUGGUCUCCUGAU | 2079 | CAGGAGACCAUCCAGAUAGUU | 74.1 | 64.1 |
| 1066 | 836 | UAUCUGGAUGGUCUCCUGAUC | 2080 | UCAGGAGACCAUCCAGAUAGU | 70.1 | 60.1 |
| 1067 | 837 | AUCUGGAUGGUCUCCUGAUCC | 2081 | AUCAGGAGACCAUCCAGAUAG | 73.9 | 63.9 |
| 1068 | 838 | UCUGGAUGGUCUCCUGAUCCU | 2082 | GAUCAGGAGACCAUCCAGAUA | 64.5 | 54.5 |
| 1069 | 839 | CUGGAUGGUCUCCUGAUCCUG | 2083 | GGAUCAGGAGACCAUCCAGAU | 57 | 47 |
| 1070 | 840 | UGGAUGGUCUCCUGAUCCUGG | 2084 | AGGAUCAGGAGACCAUCCAGA | 71.6 | 61.6 |
| 1089 | 841 | GGCUGAUAAGCUCCAGGGCCC | 2085 | GCCCUGGAGCUUAUCAGCCAG | 63.4 | 53.4 |
| 1112 | 842 | UUUCCUUUGAGCUGACGGCCG | 2086 | GCCGUCAGCUCAAAGGAAAAG | 45.3 | 34.3 |
| 1113 | 843 | UUCCUUUGAGCUGACGGCCGA | 2087 | GGCCGUCAGCUCAAAGGAAAA | 36.6 | 25.6 |
| 1114 | 844 | UCCUUUGAGCUGACGGCCGAG | 2088 | CGGCCGUCAGCUCAAAGGAAA | 47.2 | 36.2 |
| 1115 | 845 | CCUUUGAGCUGACGGCCGAGU | 2089 | UCGGCCGUCAGCUCAAAGGAA | 81.2 | 70.2 |
| 1116 | 846 | CUUUGAGCUGACGGCCGAGUC | 2090 | CUCGGCCGUCAGCUCAAAGGA | 59.7 | 48.7 |
| 1117 | 847 | UUUGAGCUGACGGCCGAGUCC | 2091 | ACUCGGCCGUCAGCUCAAAGG | 54.1 | 43.1 |
| 1118 | 848 | UUGAGCUGACGGCCGAGUCCA | 2092 | GACUCGGCCGUCAGCUCAAAG | 50.2 | 39.2 |
| 1119 | 849 | UGAGCUGACGGCCGAGUCCAU | 2093 | GGACUCGGCCGUCAGCUCAAA | 54 | 43 |
| 1120 | 850 | GAGCUGACGGCCGAGUCCAUU | 2094 | UGGACUCGGCCGUCAGCUCAA | 77.8 | 66.8 |
| 1121 | 851 | AGCUGACGGCCGAGUCCAUUG | 2095 | AUGGACUCGGCCGUCAGCUCA | 77.7 | 66.7 |
| 1122 | 852 | GCUGACGGCCGAGUCCAUUGG | 2096 | AAUGGACUCGGCCGUCAGCUC | 68.2 | 57.2 |
| 1123 | 853 | CUGACGGCCGAGUCCAUUGGG | 2097 | CAAUGGACUCGGCCGUCAGCU | 62.8 | 51.8 |
| 1142 | 854 | GGGUGAAGAUCUCGGAGGGUU | 2098 | CCCUCCGAGAUCUUCACCCCA | 65.6 | 54.6 |
| 1143 | 855 | GGUGAAGAUCUCGGAGGGUUU | 2099 | ACCCUCCGAGAUCUUCACCCC | 64.3 | 53.3 |
| 1144 | 856 | GUGAAGAUCUCGGAGGGUUUG | 2100 | AACCCUCCGAGAUCUUCACCC | 67.2 | 56.2 |
| 1145 | 857 | UGAAGAUCUCGGAGGGUUUGA | 2101 | AAACCCUCCGAGAUCUUCACC | 67.7 | 56.7 |
| 1146 | 858 | GAAGAUCUCGGAGGGUUUGAU | 2102 | CAAACCCUCCGAGAUCUUCAC | 62.6 | 51.6 |
| 1147 | 859 | AAGAUCUCGGAGGGUUUGAUG | 2103 | UCAAACCCUCCGAGAUCUUCA | 67.3 | 56.3 |
| 1148 | 860 | AGAUCUCGGAGGGUUUGAUGU | 2104 | AUCAAACCCUCCGAGAUCUUC | 69.5 | 58.5 |
| 1149 | 861 | GAUCUCGGAGGGUUUGAUGUA | 2105 | CAUCAAACCCUCCGAGAUCUU | 65.6 | 54.6 |
| 1150 | 862 | AUCUCGGAGGGUUUGAUGUAC | 2106 | ACAUCAAACCCUCCGAGAUCU | 62.6 | 51.6 |
| 1151 | 863 | UCUCGGAGGGUUUGAUGUACC | 2107 | UACAUCAAACCCUCCGAGAUC | 73.3 | 62.3 |
| 1152 | 864 | CUCGGAGGGUUUGAUGUACCU | 2108 | GUACAUCAAACCCUCCGAGAU | 65 | 54 |
| 1153 | 865 | UCGGAGGGUUUGAUGUACCUG | 2109 | GGUACAUCAAACCCUCCGAGA | 54.9 | 43.9 |
| 1154 | 866 | CGGAGGGUUUGAUGUACCUGC | 2110 | AGGUACAUCAAACCCUCCGAG | 84 | 73 |
| 1155 | 867 | GGAGGGUUUGAUGUACCUGCA | 2111 | CAGGUACAUCAAACCCUCCGA | 72.8 | 61.8 |
| 1156 | 865 | GAGGGUUUGAUGUACCUGCAG | 2112 | GCAGGUACAUCAAACCCUCCG | 67 | 56 |
| 1157 | 869 | AGGGUUUGAUGUACCUGCAGG | 2113 | UGCAGGUACAUCAAACCCUCC | 77.7 | 66 |
| 1158 | 870 | GGGUUUGAUGUACCUGCAGGA | 2114 | CUGCAGGUACAUCAAACCCUC | 72.3 | 61.3 |
| 1159 | 871 | GGUUUGAUGUACCUGCAGGAA | 2115 | CCUGCAGGUACAUCAAACCCU | 72.4 | 61.4 |
| 1160 | 872 | GUUUGAUGUACCUGCAGGAAA | 2116 | UCCUGCAGGUACAUCAAACCC | 76.7 | 65.7 |
| 1179 | 873 | AAACAGUGCGAAGGUGUCCGC | 2117 | GGACACCUUCGCACUGUUUUC | 54.4 | 43.4 |
| 1180 | 874 | AACAGUGCGAAGGUGUCCGCC | 2118 | CGGACACCUUCGCACUGUUUU | 59 | 48 |
| 1181 | 875 | ACAGUGCGAAGGUGUCCGCCC | 2119 | GCGGACACCUUCGCACUGUUU | 62.6 | 51.6 |
| 1182 | 876 | CAGUGCGAAGGUGUCCGCCCA | 2120 | GGCGGACACCUUCGCACUGUU | 54.8 | 43.8 |
| 1183 | 877 | AGUGCGAAGGUGUCCGCCCAG | 2121 | GGGCGGACACCUUCGCACUGU | 60.1 | 49.1 |
| 1184 | 878 | GUGCGAAGGUGUCCGCCCAGG | 2122 | UGGGCGGACACCUUCGCACUG | 84.3 | 73.3 |
| 1185 | 879 | UGCGAAGGUGUCCGCCCAGGU | 2123 | CUGGGCGGACACCUUCGCACU | 57.9 | 46.9 |
| 1186 | 880 | GCGAAGGUGUCCGCCCAGGUG | 2124 | CCUGGGCGGACACCUUCGCAC | 55.7 | 44.7 |
| 1187 | 881 | CGAAGGUGUCCGCCCAGGUGU | 2125 | ACCUGGGCGGACACCUUCGCA | 73.5 | 62.5 |
| 1188 | 882 | GAAGGUGUCCGCCCAGGUGUU | 2126 | CACCUGGGCGGACACCUUCGC | 61.5 | 50.5 |
| 1189 | 883 | AAGGUGUCCGCCCAGGUGUUU | 2127 | ACACCUGGGCGGACACCUUCG | 66.1 | 55.1 |
| 1190 | 884 | AGGUGUCCGCCCAGGUGUUUC | 2128 | AACACCUGGGCGGACACCUUC | 74.3 | 63.3 |
| 1191 | 885 | GGUGUCCGCCCAGGUGUUUCA | 2129 | AAACACCUGGGCGGACACCUU | 77.3 | 66.3 |
| 1192 | 886 | GUGUCCGCCCAGGUGUUUCAG | 2130 | GAAACACCUGGGCGGACACCU | 56.6 | 45.6 |
| 1193 | 887 | UGUCCGCCCAGGUGUUUCAGG | 2131 | UGAAACACCUGGGCGGACACC | 67.9 | 56.9 |
| 1194 | 888 | GUCCGCCCAGGUGUUUCAGGA | 2132 | CUGAAACACCUGGGCGGACAC | 52.1 | 41.1 |
| 1195 | 889 | UCCGCCCAGGUGUUUCAGGAG | 2133 | CCUGAAACACCUGGGCGGACA | 43.3 | 32.3 |
| 1196 | 890 | CCGCCCAGGUGUUUCAGGAGU | 2134 | UCCUGAAACACCUGGGCGGAC | 70.4 | 59.4 |
| 1197 | 891 | CGCCCAGGUGUUUCAGGAGUG | 2135 | CUCCUGAAACACCUGGGCGGA | 69.7 | 58.7 |
| 1198 | 892 | GCCCAGGUGUUUCAGGAGUGC | 2136 | ACUCCUGAAACACCUGGGCGG | 80.8 | 69.8 |
| 1199 | 893 | CCGAGGUGUUUCAGGAGUGCG | 2137 | CACUCCUGAAACACCUGGGCG | 72.9 | 61.9 |
| 1200 | 894 | CCAGGUGUUUCAGGAGUGCGG | 2138 | GCACUCCUGAAACACCUGGGC | 62.5 | 50.5 |
| 1201 | 895 | CAGGUGUUUCAGGAGUGCGGC | 2139 | CGCACUCCUGAAACACCUGGG | 69 | 57 |
| 1202 | 896 | AGGUGUUUCAGGAGUGCGGCC | 2140 | CCGCACUCCUGAAACACCUGG | 71.9 | 59.9 |
| 1203 | 897 | GGUGUUUCAGGAGUGCGGCCC | 2141 | GCCGCACUCCUGAAACACCUG | 66.1 | 54.1 |
| 1225 | 898 | CCCGACCCGGUGCCUGCCCGC | 2142 | GGGCAGGCACCGGGUCGGGGG | 49 | 37 |
| 1226 | 899 | CCGACCCGGUGCCUGCCCGCA | 2143 | CGGGCAGGCACCGGGUCGGGG | 55.5 | 43.5 |
| 1227 | 900 | CGACCCGGUGCCUGCCCGCAA | 2144 | GCGGGCAGGCACCGGGUCGGG | 56 | 44 |
| 1228 | 901 | GACCCGGUGCCUGCCCGCAAC | 2145 | UGCGGGCAGGCACCGGGUCGG | 69.1 | 57.1 |
| 1229 | 902 | ACCCGGUGCCUGCCCGCAACC | 2146 | UUGCGGGCAGGCACCGGGUCG | 74.1 | 62.1 |
| 1230 | 903 | CCCGGUGCCUGCCCGCAACCG | 2147 | GUUGCGGGCAGGCACCGGGUC | 57.1 | 45.1 |
| 1231 | 904 | CCGGUGCCUGCCCGCAACCGU | 2148 | GGUUGCGGGCAGGCACCGGGU | 52.7 | 40.7 |
| 1232 | 905 | CGGUGCCUGCCCGCAACCGUC | 2149 | CGGUUGCGGGCAGGCACCGGG | 55.2 | 43.2 |
| 1233 | 906 | GGUGCCUGCCCGCAACCGUCG | 2150 | ACGGUUGCGGGCAGGCACCGG | 71.6 | 59.6 |
| 1234 | 907 | GUGCCUGCCCGCAACCGUCGA | 2151 | GACGGUUGCGGGCAGGCACCG | 57.2 | 45.2 |
| 1235 | 908 | UGCCUGCCCGCAACCGUCGAG | 2152 | CGACGGUUGCGGGCAGGCACC | 38.1 | 26.1 |
| 1236 | 909 | GCCUGCCCGCAACCGUCGAGC | 2153 | UCGACGGUUGCGGGCAGGCAC | 68.9 | 56.9 |
| 1237 | 910 | CCUGCCCGCAACCGUCGAGCC | 2154 | CUCGACGGUUGCGGGCAGGCA | 56.1 | 44.1 |
| 1238 | 911 | CUGCCCGCAACCGUCGAGCCC | 2155 | GCUCGACGGUUGCGGGCAGGC | 37.4 | 25.4 |
| 1265 | 912 | CCCGGGAAGAGGCGGGCCGGC | 2156 | CGGCCCGCCUCUUCCCGGGGC | 44.2 | 32.2 |
| 1266 | 913 | CCGGGAAGAGGCGGGCCGGCU | 2157 | CCGGCCCGCCUCUUCCCGGGG | 51.1 | 39.1 |
| 1267 | 914 | CGGGAAGAGGCGGGCCGGCUG | 2158 | GCCGGCCCGCCUCUUCCCGGG | 47.8 | 35.8 |
| 1268 | 915 | GGGAAGAGGCGGGCCGGCUGU | 2159 | AGCCGGCCCGCCUCUUCCCGG | 62.6 | 50.6 |
| 1269 | 916 | GGAAGAGGCGGGCCGGCUGUG | 2160 | CAGCCGGCCCGCCUCUUCCCG | 63.4 | 51.4 |
| 1270 | 917 | GAAGAGGCGGGCCGGCUGUGG | 2161 | ACAGCCGGCCCGCCUCUUCCC | 59.1 | 47.1 |
| 1271 | 918 | AAGAGGCGGGCCGGCUGUGGU | 2162 | CACAGCCGGCCCGCCUCUUCC | 42.4 | 30.4 |
| 1272 | 919 | AGAGGCGGGCCGGCUGUGGUC | 2163 | CCACAGCCGGCCCGCCUCUUC | 42.9 | 30.9 |
| 1273 | 920 | GAGGCGGGCCGGCUGUGGUCG | 2164 | ACCACAGCCGGCCCGCCUCUU | 65.8 | 53.8 |
| 1274 | 921 | AGGCGGGCCGGCUGUGGUCGA | 2165 | GACCACAGCCGGCCCGCCUCU | 53.8 | 41.8 |
| 1275 | 922 | GGCGGGCCGGCUGUGGUCGAU | 2166 | CGACCACAGCCGGCCCGCCUC | 48.2 | 36.2 |
| 1276 | 923 | GCGGGCCGGCUGUGGUCGAUG | 2167 | UCGACCACAGCCGGCCCGCCU | 74.9 | 62.9 |
| 1277 | 924 | CGGGCCGGCUGUGGUCGAUGG | 2168 | AUCGACCACAGCCGGCCCGCC | 65.7 | 53.7 |
| 1278 | 925 | GGGCCGGCUGUGGUCGAUGGU | 2169 | CAUCGACCACAGCCGGCCCGC | 59 | 47 |
| 1279 | 926 | GGCCGGCUGUGGUCGAUGGUG | 2170 | CCAUCGACCACAGCCGGCCCG | 71.2 | 59.2 |
| 1280 | 927 | GCCGGCUGUGGUCGAUGGUGA | 2171 | ACCAUCGACCACAGCCGGCCC | 67.3 | 55.3 |
| 1281 | 928 | CCGGCUGUGGUCGAUGGUGAC | 2172 | CACCAUCGACCACAGCCGGCC | 59.6 | 47.6 |
| 1282 | 929 | CGCCUGUGGUCGAUGGUGACC | 2173 | UCACCAUCGACCACAGCCGGC | 85.3 | 73.3 |
| 1283 | 930 | GGCUGUGGUCGAUGGUGACCG | 2174 | GUCACCAUCGACCACAGCCGG | 82.9 | 70.9 |
| 1284 | 931 | GCUGUGGUCCAUGGUGACCGA | 2175 | GGUCACCAUCGACCACAGCCG | 64.5 | 52.5 |
| 1285 | 932 | CUGUGGUaGAUGGUGACCGAG | 2176 | CGGUCACCAUCGACCACAGCC | 52.2 | 40.2 |
| 1286 | 933 | UGUGGUCGAUGGUGACCGAGG | 2177 | UCGGUCACCAUCGACCACAGC | 81.5 | 69.5 |
| 1287 | 934 | GUGGUCGAUGGUGACCGAGGA | 2178 | CUCGGUCACCAUCGACCACAG | 65.9 | 53.9 |
| 1288 | 935 | UGGUCGAUGGUGACCGAGGAG | 2179 | CCUCGGUCACCAUCGACCACA | 60.2 | 48.2 |
| 1289 | 936 | GGUCGAUGGUGACCGAGGAGG | 2180 | UCCUCGGUCACCAUCGACCAC | 88.9 | 76.9 |
| 1290 | 937 | GUCGAUGGUGACCGAGGAGGA | 2181 | CUCCUCGGUCACCAUCGACCA | 68.1 | 56.1 |
| 1291 | 938 | UCGAUGGUGACCGAGGAGGAG | 2182 | CCUCCUCGGUCACCAUCGACC | 44 | 32 |
| 1292 | 939 | CGAUGGUGACCGAGGAGGAGC | 2183 | UCCUCCUCGGUCACCAUCGAC | 84.9 | 72.9 |
| 1293 | 940 | GAUGGUGACCGAGGAGGAGCG | 2184 | CUCCUCCUCGGUCACCAUCGA | 70.6 | 58.6 |
| 1294 | 941 | AUGGUGACCGAGGAGGAGCGG | 2185 | GCUCCUCCUCGGUCACCAUCG | 49.1 | 37.1 |
| 1295 | 942 | UGGUGACCGAGGAGGAGCGGC | 2186 | CGCUCCUCCUCGGUCACCAUC | 52.5 | 40.5 |
| 1296 | 943 | GGUGACCGAGGAGGAGCGGCC | 2187 | CCGCUCCUCCUCGGUCACCAU | 61.4 | 49.4 |
| 1297 | 944 | GUGACCGAGGAGGAGCGGCCC | 2188 | GCCGCUCCUCCUCGGUCACCA | 47.4 | 35.4 |
| 1298 | 945 | UGACCGAGGAGGAGCGGCCCA | 2189 | GGCCGCUCCUCCUCGGUCACC | 41 | 29 |
| 1299 | 946 | GACCGAGGAGGAGCGGCCCAC | 2190 | GGGCCGCUCCUCCUCGGUCAC | 47.4 | 35.4 |
| 1300 | 947 | ACCGAGGAGGAGCGGCCCACG | 2191 | UGGGCCGCUCCUCCUCGGUCA | 66.6 | 53.6 |
| 1301 | 948 | CCGAGGAGGAGCGGCCCACGA | 2192 | GUGGGCCGCUCCUCCUCGGUC | 53 | 40 |
| 1302 | 949 | CGAGGAGGAGCGGCCCACGAC | 2193 | CGUGGGCCGCUCCUCCUCGGU | 59.1 | 46.1 |
| 1303 | 950 | GAGGAGGAGCGGCCCACGACG | 2194 | UCGUGGGCCGCUCCUCCUCGG | 80.4 | 67.4 |
| 1304 | 951 | AGGAGGAGCGGCCCACGACGG | 2195 | GUCGUGGGCCGCUCCUCCUCG | 80.4 | 47.4 |
| 1305 | 952 | GGAGGAGCGGCCCACGACGGC | 2196 | CGUCGUGGGCCGCUCCUCCUC | 61.2 | 48.2 |
| 1306 | 953 | GAGGAGCGGCCCACGACGGCC | 2197 | CCGUCGUGGGCCGCUCCUCCU | 58.8 | 45.8 |
| 1307 | 954 | AGGAGCGGCCCACGACGGCCG | 2198 | GCCGUCGUGGGCCGCUCCUCC | 41.9 | 28.9 |
| 1308 | 955 | GGAGCGGCCCACGACGGCCGC | 2199 | GGCCGUCGUGGGCCGCUCCUC | 49.4 | 36.4 |
| 1309 | 956 | GAGCGGCCCACGACGGCCGCA | 2200 | CGGCCGUCGUGGGCCGCUCCU | 53.6 | 40.6 |
| 1310 | 957 | AGCGGCCCACGACGGCCGCAG | 2201 | GCGGCCGUCGUGGGCCGCUCC | 39.8 | 26.8 |
| 1311 | 958 | GCGGCCCACGACGGCCGCAGG | 2202 | UGCGGCCGUCGUGGGCCGCUC | 60 | 47 |
| 1312 | 959 | CGGCCCACGACGGCCGCAGGC | 2203 | CUGCGGCCGUCGUGGGCCGCU | 53.4 | 40.4 |
| 1313 | 960 | GGCCCACGACGGCCGCAGGCA | 2204 | CCUGCGGCCGUCGUGGGCCGC | 54.9 | 41.9 |
| 1314 | 961 | GCCCACGACGGCCGCAGGCAC | 2205 | GCCUGCGGCCGUCGUGGGCCG | 48.1 | 35.1 |
| 1315 | 962 | CCCACGACGGCCGCAGGCACC | 2206 | UGCCUGCGGCCGUCGUGGGCC | 58.7 | 45.7 |
| 1316 | 963 | CCACGACGGCCGCAGGCACCA | 2207 | GUGCCUGCGGCCGUCGUGGGC | 59.6 | 46.6 |
| 1317 | 964 | CACGACGGCCGCAGGCACCAA | 2208 | GGUGCCUGCGGCCGUCGUGGG | 54.3 | 41.3 |
| 1318 | 965 | ACGACGGCCGCAGGCACCAAU | 2209 | UGGUGCCUGCGGCCGUCGUGG | 68.6 | 55.6 |
| 1319 | 966 | CGACGGCCGCAGGCACCAACC | 2210 | UUGGUGCCUGCGGCCGUCGUG | 85.8 | 73.8 |
| 1320 | 967 | GACGGCCGCAGGCACCAACCU | 2211 | GUUGGUGCCUGCGGCCGUCGU | 59.9 | 46.9 |
| 1321 | 968 | ACGGCCGCAGGCACCAACCUG | 2212 | GGUUGGUGCCUGCGGCCGUCG | 45.5 | 32.5 |
| 1322 | 969 | CGGCCGCAGGCACCAACCUGC | 2213 | AGGUUGGUGCCUGCGGCCGUC | 64.6 | 51.6 |
| 1323 | 970 | GGCCGCAGGCACCAACCUGCA | 2214 | CAGGUUGGUGCCUGCGGCCGU | 65.5 | 52.5 |
| 1324 | 971 | GCCGCAGGCACCAACCUGCAC | 2215 | GCAGGUUGGUGCCUGCGGCCG | 58.7 | 45.7 |
| 1325 | 972 | CCGCAGGCACCAACCUGCACC | 2216 | UGCAGGUUGGUGCCUGCGGCC | 70.5 | 57.5 |
| 1326 | 973 | CGCAGGCACCAACCUGCACCG | 2217 | GUGCAGGUUGGUGCCUGCGGC | 63.2 | 50.2 |
| 1327 | 974 | GCAGGCACCAACCUGCACCGG | 2218 | GGUGCAGGUUGGUGCCUGCGG | 58.4 | 45.4 |
| 1328 | 975 | CAGGCACCAACCUGCACCGGC | 2219 | CGGUGCAGGUUGGUGCCUGCG | 60 | 47 |
| 1329 | 976 | AGGCACCAACCUGCACCGGCU | 2220 | CCGGUGCAGGUUGGUGCCUGC | 46.5 | 33.5 |
| 1330 | 977 | GGCACCAACCUGCACCGGCUG | 2221 | GCCGGUGCAGGUUGGUGCCUG | 59.2 | 46.2 |
| 1331 | 978 | GCACCAACCUGCACCGGCUGG | 2222 | AGCCGGUGCAGGUUGGUGCCU | 74.1 | 61.1 |
| 1332 | 979 | CACCAACCUGCACCGGCUGGU | 2223 | CAGCCGGUGCAGGUUGGUGCC | 56.7 | 43.7 |
| 1333 | 980 | ACCAACCUGCACCGGCUGGUG | 2224 | CCAGCCGGUGCAGGUUGGUGC | 46.3 | 33.3 |
| 1334 | 981 | CCAACCUGCACCGGCUGGUGU | 2225 | ACCAGCCGGUGCAGGUUGGUG | 65.5 | 52.5 |
| 1335 | 982 | CAACCUGCACCGGCUGGUGUG | 2226 | CACCAGCCGGUGCAGGUUGGU | 63 | 50 |
| 1336 | 983 | AACCUGCACCGGCUGGUGUGG | 2227 | ACACCAGCCGGUGCAGGUUGG | 66.8 | 53.8 |
| 1337 | 984 | ACCUGCACCGGCUGGUGUGGG | 2228 | CACACCAGCCGGUGCAGGUUG | 64.3 | 51.3 |
| 1338 | 955 | CCUGCACCGGCUGGUGUGGGA | 2229 | CCACACCAGCCGGUGCAGGUU | 60.7 | 47.7 |
| 1339 | 986 | CUGCACCGGCUGGUGUGGGAG | 2230 | CCCACACCAGCCGGUGCAGGU | 47 | 34 |
| 1340 | 987 | UGCACCGGCUGGUGUGGGAGC | 2231 | UCCCACACCAGCCGGUGCAGG | 70.3 | 57.3 |
| 1341 | 988 | GCACCGGCUGGUGUGGGAGCU | 2232 | CUCCCACACCAGCCGGUGCAG | 62.5 | 49.5 |
| 1342 | 989 | CACCGGCUGGUGUGGGAGCUC | 2233 | GCUCCCACACCAGCCGGUGCA | 54.6 | 41.6 |
| 1343 | 990 | ACCGGCUGGUGUGGGAGCUCC | 2234 | AGCUCCCACACCAGCCGGUGC | 55.9 | 42.9 |
| 1344 | 991 | CCGGCUGGUGUGGGAGCUCCG | 2235 | GAGCUCCCACACCAGCCGGUG | 63.1 | 50.1 |
| 1345 | 992 | CGGCUGGUGUGGGAGCUCCGC | 2236 | GGAGCUCCCACACCAGCCGGU | 62.9 | 49.9 |
| 1346 | 993 | GGCUGGUGUGGGAGCUCCGCG | 2237 | CGGAGCUCCCACACCAGCCGG | 72.1 | 59.1 |
| 1347 | 994 | GCUGGUGUGGGAGCUCCGCGA | 2238 | GCGGAGCUCCCACACCAGCCG | 58.7 | 45.7 |
| 1348 | 995 | CUGGUGUGGGAGCUCCGCGAG | 2239 | CGCGGAGCUCCCACACCAGCC | 45.9 | 32.9 |
| 1349 | 996 | UGGUGUGGGAGCUCCGCGAGC | 2240 | UCGCGGAGCUCCCACACCAGC | 69.7 | 56.7 |
| 1350 | 997 | GGUGUGGGAGCUCCGCGAGCG | 2241 | CUCGCGGAGCUCCCACACCAG | 72 | 59 |
| 1351 | 998 | GUGUGGGAGCUCCGCGAGCGU | 2242 | GCUCGCGGAGCUCCCACACCA | 57.8 | 44.8 |
| 1352 | 999 | UGUGGGAGCUCCGCGAGCGUC | 2243 | CGCUCGCGGAGCUCCCACACC | 45.4 | 32.4 |
| 1353 | 1000 | GUGGGAGCUCCGCGAGCGUCU | 2244 | ACGCUCGCGGAGCUCCCACAC | 63.8 | 50.8 |
| 1354 | 1001 | UGGGAGCUCCGCGAGCGUCUG | 2245 | GACGCUCGCGGAGCUCCCACA | 50.5 | 37.5 |
| 1355 | 1002 | GGGAGCUCCGCGAGCGUCUGG | 2246 | AGACGCUCGCGGAGCUCCCAC | 67.3 | 54.3 |
| 1386 | 1003 | GGGCUUCUGGGCCCGGCUGUC | 2247 | CAGCCGGGCCCAGAAGCCCCG | 57 | 44 |
| 1387 | 1004 | GGCUUCUGGGCCCGGCUGUCC | 2248 | ACAGCCGGGCCCAGAAGCCCC | 56.8 | 43.8 |
| 1388 | 1005 | GCUUCUGGGCCCGGCUGUCCC | 2249 | GACAGCCGGGCCCAGAAGCCC | 50.8 | 37.6 |
| 1389 | 1006 | CUUCUGGGCCCGGCUGUCCCU | 2250 | GGACAGCCGGGCCCAGAAGCC | 36.1 | 23.1 |
| 1390 | 1007 | UUCUGGGCCCGGCUGUCCCUG | 2251 | GGGACAGCCGGGCCCAGAAGC | 31.3 | 18.3 |
| 1391 | 1008 | UCUGGGCCCGGCUGUCCCUGA | 2252 | AGGGACAGCCGGGCCCAGAAG | 54.2 | 41.2 |
| 1392 | 1009 | CUGGGCCCGGCUGUCCCUGAC | 2253 | CAGGGACAGCCGGGCCCAGAA | 44 | 31 |
| 1393 | 1010 | UGGGCCCGGCUGUCCCUGACG | 2254 | UCAGGGACAGCCGGGCCCAGA | 80.9 | 47.9 |
| 1394 | 1011 | GGGCCCGGCUGUCCCUGACGG | 2255 | GUCAGGGACAGCCGGGCCCAG | 63.1 | 50.1 |
| 1395 | 1012 | GGCCCGGCUGUCCCUGACGGU | 2256 | CGUCAGGGACAGCCGGGCCCA | 57.4 | 44.4 |
| 1396 | 1013 | GCCCGGCUGUCCCUGACGGUG | 2257 | CCGUCAGGGACAGCCGGGCCC | 52.9 | 39.9 |
| 1397 | 1014 | CCCGGCUGUCCCUGACGGUGU | 2258 | ACCGUCAGGGACAGCCGGGCC | 59.9 | 46.9 |
| 1398 | 1015 | CCGGCUGUCCCUGACGGUGUG | 2259 | CACCGUCAGGGACAGCCGGGC | 58.9 | 45.9 |
| 1399 | 1016 | CGGCUGUCCCUGACGGUGUGC | 2260 | ACACCGUCAGGGACAGCCGGG | 80.9 | 67.9 |
| 1400 | 1017 | GGCUGUCCCUGACGGUGUGCG | 2261 | CACACCGUCAGGGACAGCCGG | 81.1 | 67.1 |
| 1401 | 1018 | GCUGUCCCUGACGGUGUGCGG | 2262 | GCACACCGUCAGGGACAGCCG | 58.4 | 44.4 |
| 1402 | 1019 | CUGUCCCUGACGGUGUGCGGA | 2263 | CGCACACCGUCAGGGACAGCC | 44.7 | 30.7 |
| 1403 | 1020 | UGUCCCUGACGGUGUGCGGAG | 2264 | CCGCACACCGUCAGGGACAGC | 53.2 | 39.2 |
| 1404 | 1021 | GUCCCUGACGGUGUGCGGAGA | 2265 | UCCGCACACCGUCAGGGACAG | 73.6 | 59.6 |
| 1405 | 1022 | UCCCUGACGGUGUGCGGAGAC | 2266 | CUCCGCACACCGUCAGGGACA | 54.6 | 40.6 |
| 1406 | 1023 | CCCUGACGGUGUGCGGAGACU | 2267 | UCUCCGCACACCGUCAGGGAC | 76.6 | 62.6 |
| 1407 | 1024 | CCUGACGGUGUGCGGAGACUC | 2268 | GUCUCCGCACACCGUCAGGGA | 66.8 | 52.8 |
| 1408 | 1025 | CUGACGGUGUGCGGAGACUCU | 2269 | AGUCUCCGCACACCGUCAGGG | 76.3 | 62.3 |
| 1409 | 1026 | UGACGGUGUGCGGAGACUCUC | 2270 | GAGUCUCCGCACACCGUCAGG | 66.8 | 52.1 |
| 1410 | 1027 | GACGGUGUGCGGAGACUCUCG | 2271 | AGAGUCUCCGCACACCGUCAG | 55.1 | 71.1 |
| 1411 | 1028 | ACGGUGUGCGGAGACUCUCGC | 2272 | GAGAGUCUCCGCACACCGUCA | 65.2 | 51.2 |
| 1412 | 1029 | CGGUGUGCGGAGACUCUCGCA | 2273 | CGAGAGUCUCCGCACACCGUC | 69.5 | 55.5 |
| 1413 | 1030 | GGUGUGCGGAGACUCUCGCAU | 2274 | GCGAGAGUCUCCGCACACCGU | 68.6 | 54.6 |
| 1414 | 1031 | GUGUGCGGAGACUCUCGCAUG | 2275 | UGCGAGAGUCUCCGCACACCG | 78.1 | 64.1 |
| 1415 | 1032 | UGUGCGGAGACUCUCGCAUGG | 2276 | AUGCGAGAGUCUCCGCACACC | 65.2 | 51.2 |
| 1434 | 1033 | GGCAGCGGACGCCUCGCUGGA | 2277 | CAGCGAGGCGUCCGCUGCCAU | 62.5 | 48.5 |
| 1435 | 1034 | GCAGCGGACGCCUCGCUGGAG | 2278 | CCAGCGAGGCGUCCGCUGCCA | 60.6 | 46.6 |
| 1436 | 1035 | CAGCGGACGCCUCGCUGGAGG | 2279 | UCCAGCGAGGCGUCCGCUGCC | 69.5 | 55.5 |
| 1437 | 1036 | AGCGGACGCCUCGCUGGAGGC | 2280 | CUCCAGCGAGGCGUCCGCUGC | 52.1 | 38.1 |
| 1438 | 1037 | GCGGACGCGUCGCUGGAGGCG | 2281 | CCUCCAGCGAGGCGUCCGCUG | 59.8 | 45.8 |
| 1439 | 1038 | CGGACGCCUCGCUGGAGGCGG | 2282 | GCCUCCAGCGAGGCGUCCGCU | 54.2 | 40.2 |
| 1440 | 1039 | GGACGCCUCGCUGGAGGCGGC | 2283 | CGCCUCCAGCGAGGCGUCCGC | 46.6 | 32.6 |
| 1441 | 1040 | GACGCCUCGCUGGAGGCGGCG | 2284 | CCGCCUCCAGCGAGGCGUCCG | 50 | 36 |
| 1442 | 1041 | ACGCCUCGCUGGAGGCGGCGC | 2285 | GCCGCCUCCAGCGAGGCGUCC | 40.9 | 26.9 |
| 1443 | 1042 | CGCCUCGCUGGAGGCGGCGCC | 2286 | CGCCGCCUCCAGCGAGGCGUC | 41.2 | 27.2 |
| 1444 | 1043 | GCCUCGCUGGAGGCGGCGCCC | 2287 | GCGCCGCCUCCAGCGAGGCGU | 49.9 | 35.9 |
| 1445 | 1044 | CCUCGCUGGAGGCGGCGCCCU | 2288 | GGCGCCGCCUCCAGCGAGGCG | 45.7 | 31.7 |
| 1446 | 1045 | CUCGCUGGAGGCGGCGCCCUG | 2289 | GGGCGCCGCCUCCAGCGAGGC | 36.1 | 22.1 |
| 1447 | 1046 | UCGCUGGAGGCGGCGCCCUGC | 2290 | AGGGCGCCGCCUCCAGCGAGG | 48.8 | 34.8 |
| 1448 | 1047 | CGCUGGAGGCGGCGCCCUGCU | 2291 | CAGGGCGCCGCCUCCAGCGAG | 64.2 | 50.2 |
| 1449 | 1048 | GCUGGAGGCGGCGCCCUGCUG | 2292 | GCAGGGCGCCGCCUCCAGCGA | 52.8 | 38.8 |
| 1450 | 1049 | CUGGAGGCGGGGCCCUGCUGG | 2293 | AGCAGGGCGCCGCCUCCAGCG | 58.7 | 44.7 |
| 1451 | 1050 | UGGAGGCGGGGCCCUGCUGGA | 2294 | CAGCAGGGCGCCGCCUCCAGC | 47.6 | 33.6 |
| 1452 | 1051 | GGAGGCGGCGCCCUGCUGGAC | 2295 | CCAGCAGGGCGCCGCCUCCAG | 55.2 | 41.2 |
| 1453 | 1052 | GAGGCGGCGCCCUGCUGGACC | 2296 | UCCAGCAGGGCGCCGCCUCCA | 71.9 | 57.9 |
| 1454 | 1053 | AGGCGGCGCCCUGCUGGACCG | 2297 | GUCCAGCAGGGCGCCGCCUCC | 45.4 | 31.4 |
| 1455 | 1054 | GGCGGCGCCCUGCUGGACCGG | 2298 | GGUCCAGCAGGGCGCCGCCUC | 47.1 | 33.1 |
| 1456 | 1055 | GCGGCGCCCUGCUGGACCGGA | 2299 | CGGUCCAGCAGGGCGCCGCCU | 60.4 | 46.4 |
| 1457 | 1056 | CGGCGCCCUGCUGGACCGGAG | 2300 | CCGGUCCAGCAGGGCGCCGCC | 44.1 | 30.1 |
| 1458 | 1057 | GGCGCCCUGCUGGACCGGAGC | 2301 | UCCGGUCCAGCAGGGCGCCGC | 64.9 | 50.9 |
| 1459 | 1058 | GCGCCCUGCUGGACCGGAGCC | 2302 | CUCCGGUCCAGCAGGGCGCCG | 65.7 | 51.7 |
| 1460 | 1059 | CGCCCUGCUGGACCGGAGCCG | 2303 | GCUCCGGUCCAGCAGGGCGCC | 51.3 | 37.3 |
| 1461 | 1060 | GCCCUGCUGGAGCGGAGCCGG | 2304 | GGCUCCGGUCCAGCAGGGCGC | 48.3 | 34.3 |
| 1462 | 1061 | CCCUGCUGGACCGGAGCCGGG | 2305 | CGGCUCCGGUCCAGCAGGGCG | 51.1 | 37.1 |
| 1463 | 1062 | CCUGCUGGACCGGAGCCGGGC | 2306 | CCGGCUCCGGUCGAGCAGGGC | 53.1 | 39.1 |
| 1464 | 1063 | CUGCUGGACCGGAGCCGGGCG | 2307 | CCCGGCUCCGGUCCAGCAGGG | 55 | 41 |
| 1465 | 1064 | UGCUGGACCGGAGCCGGGCGG | 2308 | GCCCGGCUCCGGUCCAGCAGG | 44 | 30 |
| 1466 | 1065 | GCUGGACCGGAGCCGGGCGGG | 2309 | CGCCCGGCUCCGGUCCAGCAG | 57.1 | 43.1 |
| 1518 | 1066 | CCCGGCCGAGCAGGUCAACAA | 2310 | GUUGACCUGCUCGGCCGGGGA | 58.7 | 43.7 |
| 1519 | 1067 | CCGGCCGAGCAGGUCAACAAC | 2311 | UGUUGACCUGCUCGGCCGGGG | 75.2 | 60.2 |
| 1520 | 1068 | CGGCCGAGCAGGUCAACAACC | 2312 | UUGUUGACCUGCUCGGCCGGG | 95.7 | 80.7 |
| 1521 | 1069 | GGCCGAGCAGGUCAACAACCC | 2313 | GUUGUUGACCUGCUCGGCCGG | 78.4 | 63.4 |
| 1540 | 1070 | CCCGAGCUCAAGGUGGACGCC | 2314 | CGUCCACCUUGAGCUCGGGGU | 60.5 | 45.5 |
| 1541 | 1071 | CCGAGCUCAAGGUGGACGCCU | 2315 | GCGUCCACCUUGAGCUCGGGG | 65.5 | 50.5 |
| 1542 | 1072 | CGAGCUCAAGGUGGACGCCUC | 2316 | GGCGUCCACCUUGAGCUCGGG | 64.1 | 49.1 |
| 1543 | 1073 | GAGCUCAAGGUGGACGCCUCG | 2317 | AGGCGUCCACCUUGAGCUCGG | 68.9 | 53.9 |
| 1544 | 1074 | AGCUCAAGGUGGACGCCUCGG | 2318 | GAGGCGUCCACCUUGAGCUCG | 69.6 | 54.6 |
| 1545 | 1075 | GCUCAAGGUGGACGCCUCGGG | 2319 | CGAGGCGUCCACCUUGAGCUC | 62:3 | 47.3 |
| 1546 | 1076 | CUCAAGGUGGACGCGUCGGGC | 2320 | CCGAGGCGUCCACCUUGAGCU | 51.8 | 36.8 |
| 1547 | 1077 | UCAAGGUGGACGCCUCGGGCC | 2321 | CCCGAGGCGUCCACCUUGAGC | 42.4 | 27.4 |
| 1548 | 1078 | CAAGGUGGACGCCUCGGGCCC | 2322 | GCCCGAGGCGUCCACCUUGAG | 53.4 | 38.4 |
| 1567 | 1079 | CCCGAUGUCCCGACACGGCGG | 2323 | GCCGUGUCGGGACAUCGGGGC | 57.8 | 42.8 |
| 1568 | 1080 | CCGAUGUCCCGACACGGCGGC | 2324 | CGCCGUGUCGGGACAUCGGGG | 69.5 | 54.5 |
| 1569 | 1081 | CGAUGUCCCGACACGGCGGCG | 2325 | CCGCCGUGUCGGGACAUCGGG | 73.8 | 58.8 |
| 1570 | 1082 | GAUGUCCCGACACGGCGGCGU | 2326 | GCCGCCGUGUCGGGACAUCGG | 57 | 42 |
| 1571 | 1083 | AUGUCCCGACACGGCGGCGUC | 2327 | CGCCGCCGUGUCGGGACAUCG | 44.8 | 29.8 |
| 1572 | 1084 | UGUCCCGACACGGCGGCGUCG | 2328 | ACGCCGCCGUGUCGGGACAUC | 51.2 | 36.2 |
| 1573 | 1085 | GUCCCGACACGGCGGCGUCGG | 2329 | GACGCCGCCGUGUCGGGACAU | 60.6 | 45.6 |
| 1574 | 1086 | UCCCGACACGGCGGCGUCGGC | 2330 | CGACGCCGCCGUGUCGGGACA | 44.4 | 29.4 |
| 1575 | 1087 | CCCGACACGGCGGCGUCGGCU | 2331 | CCGACGCCGCCGUGUCGGGAC | 45.7 | 30.7 |
| 1576 | 1088 | CGGACACGGCGGCGUCGGCUA | 2332 | GCCGACGCCGCCGUGUCGGGA | 57 | 42 |
| 1577 | 1089 | CGACACGGCGGCGUCGGCUAC | 2333 | AGCGGACGCCGCCGUGUCGGG | 64.3 | 49.3 |
| 1578 | 1090 | GACACGGCGGCGUCGGCUACA | 2334 | UAGCCGACGCCGCCGUGUCGG | 84.8 | 69.8 |
| 1579 | 1091 | ACACGGCGGCGUCGGCUACAG | 2335 | GUAGCCGACGCCGCCGUGUCG | 60.7 | 45.7 |
| 1580 | 1092 | CACGGCGGCGUCGGCUACAGC | 2336 | UGUAGCCGACGCCGCCGUGUC | 64.8 | 49.8 |
| 1581 | 1093 | ACGGCGGCGUCGGCUACAGCU | 2337 | CUGUAGCCGACGCCGCCGUGU | 61.7 | 46.7 |
| 1582 | 1094 | CGGCGGCGUCGGCUACAGCUC | 2338 | GCUGUAGCCGACGCCGCCGUG | 64.6 | 49.6 |
| 1583 | 1095 | GGCGGCGUCGGCUACAGCUCC | 2339 | AGCUGUAGCCGACGCCGCCGU | 73.4 | 58.4 |
| 1584 | 1096 | GCGGCGUCGGCUACAGCUCCG | 2340 | GAGCUGUAGCCGACGCCGCCG | 69.6 | 54.6 |
| 1585 | 1097 | CGGCGUCGGCUACAGCUCCGG | 2341 | GGAGCUGUAGCCGACGCCGCC | 57.7 | 42.7 |
| 1586 | 1098 | GGCGUCGGCUACAGCUCCGGG | 2342 | CGGAGCUGUAGCCGACGCCGC | 61.1 | 46.1 |
| 1587 | 1099 | GCGUCGGCUACAGCUCCGGGC | 2343 | CCGGAGCUGUAGCCGACGCCG | 61.9 | 46.9 |
| 1588 | 1100 | CGUCGGCUACAGCUCCGGGCG | 2344 | CCCGGAGCUGUAGCCGACGCC | 53.9 | 38.9 |
| 1889 | 1101 | GUCGGCUACAGCUCCGGGCGG | 2345 | GCCCGGAGCUGUAGCCGACGC | 45.7 | 30.7 |
| 1590 | 1102 | UCGGCUACAGCUCCGGGCGGC | 2346 | CGCCCGGAGCUGUAGCCGACG | 46.9 | 31.9 |
| 1591 | 1103 | CGGCUACAGCUCCGGGCGGCC | 2347 | CCGCCCGGAGCUGUAGCCGAC | 54.9 | 39.9 |
| 1592 | 1104 | GGCUACAGCUCCGGGCGGCCA | 2348 | GCCGCCCGGAGCUGUAGCCGA | 54.2 | 39.2 |
| 1593 | 1105 | GCUACAGCUCCGGGCGGCCAC | 2349 | GGCCGCCCGGAGCUGUAGCCG | 51 | 36 |
| 1625 | 1106 | AAACGGCCGCACUGGGACACG | 2350 | UGUCCCAGUGCGGCCGUUUUC | 63 | 47 |
| 1626 | 1107 | AACGGCCGCACUGGGACACGA | 2351 | GUGUCCCAGUGCGGCCGUUUU | 46.3 | 30.3 |
| 1627 | 1108 | ACGGCCGCACUGGGACACGAC | 2352 | CGUGUCCCAGUGCGGCCGUUU | 46.2 | 30.2 |
| 1625 | 1109 | CGGCCGCACUGGGACACGACC | 2353 | UCGUGUCCCAGUGCGGCCGUU | 61.7 | 65.7 |
| 1629 | 1110 | GGCCGCACUGGGACACGACCU | 2354 | GUCGUGUCCCAGUGCGGCCGU | 66.1 | 50.1 |
| 1630 | 1111 | GCCGCACUGGGACACGACCUG | 2355 | GGUCGUGUCCCAGUGCGGCCG | 62.2 | 46.2 |
| 1631 | 1112 | CCGCACUGGGACACGACCUGG | 2356 | AGGUCGUGUCCCAGUGCGGCC | 63.3 | 47.3 |
| 1632 | 1113 | CGCACUGGGACACGACCUGGA | 2357 | CAGGUCGUGUCCCAGUGCGGC | 66.3 | 50.3 |
| 1633 | 1114 | GCACUGGGACACGACCUGGAC | 2358 | CCAGGUCGUGUCCCAGUGCGG | 67.9 | 51.9 |
| 1634 | 1115 | CACUGGGACACGACCUGGACG | 2359 | UCCAGGUCGUGUCCGAGUGCG | 81.8 | 65.8 |
| 1635 | 1116 | ACUGGGACACGACCUGGACGG | 2360 | GUCCAGGUCGUGUCCCAGUGC | 61.1 | 45.1 |
| 1636 | 1117 | CUGGGACACGACCUGGACGGG | 2361 | CGUCCAGGUCGUGUCCCAGUG | 63 | 47 |
| 1637 | 1118 | UGGGACACGACCUGGACGGGC | 2362 | CCGUCCAGGUCGUGUCCCAGU | 54.4 | 38.4 |
| 1638 | 1119 | GGGACACGACCUGGACGGGCA | 2363 | CCCGUCCAGGUCGUGUCCCAG | 63.3 | 47.3 |
| 1639 | 1120 | GGACACGACCUGGACGGGCAG | 2364 | GCCCGUCCAGGUCGUGUCCCA | 55.4 | 39.4 |
| 1640 | 1121 | GACACGACCUGGACGGGCAGG | 2365 | UGCCCGUCCAGGUCGUGUCCC | 74.5 | 58.5 |
| 1641 | 1122 | ACACGACCUGGACGGGCAGGA | 2366 | CUGCCCGUCCAGGUCGUGUCC | 57.4 | 41.4 |
| 1642 | 1123 | CACGACCUGGACGGGCAGGAC | 2367 | CCUGCCCGUCCAGGUCGUGUC | 47.7 | 31.7 |
| 1643 | 1124 | ACGACCUGGACGGGCAGGACG | 2368 | UCCUGCCCGUCCAGGUCGUGU | 66.3 | 50.3 |
| 1644 | 1125 | CGACCUGGACGGGCAGGACGC | 2369 | GUCCUGCCCGUCCAGGUCGUG | 69.4 | 53.4 |
| 1645 | 1126 | GACCUGGACGGGCAGGACGCG | 2370 | CGUCCUGCCCGUCCAGGUCGU | 62 | 46 |
| 1646 | 1127 | ACCUGGACGGGCAGGACGCGG | 2371 | GCGUCCUGCCCGUCCAGGUCG | 52.3 | 36.3 |
| 1647 | 1128 | CCUGGACGGGCAGGACGCGGA | 2372 | CGCGUCCUGCCCGUCCAGGUC | 49.1 | 33.1 |
| 1648 | 1129 | CUGGACGGGCAGGACGCGGAU | 2373 | CCGCGUCCUGCCCGUCCAGGU | 49.1 | 33.1 |
| 1649 | 1130 | UGGACGGGCAGGACGCGGAUG | 2374 | UCCGCGUCCUGCCCGUCCAGG | 68.8 | 52.8 |
| 1650 | 1131 | GGACGGGCAGGACGCGGAUGA | 2375 | AUCCGCGUCCUGCCCGUCCAG | 82.1 | 66.1 |
| 1651 | 1132 | GACGGGCAGGACGCGGAUGAG | 2376 | CAUCCGCGUCCUGCCCGUCCA | 60.5 | 44.5 |
| 1652 | 1133 | ACGGGCAGGACGCGGAUGAGG | 2377 | UCAUCCGCGUCCUGCCCGUCC | 61.8 | 45.8 |
| 1653 | 1134 | CGGGCAGGACGCGGAUGAGGA | 2378 | CUCAUCCGCGUCCUGCCCGUC | 62 | 46 |
| 1654 | 1135 | GGGCAGGACGCGGAUGAGGAU | 2379 | CCUCAUCCGCGUCCUGCCCGU | 61.4 | 45.4 |
| 1655 | 1136 | GGCAGGACGCGGAUGAGGAUG | 2380 | UCCUCAUCCGCGUCCUGCCCG | 81.5 | 65.5 |
| 1656 | 1137 | GCAGGACGCGGAUGAGGAUGC | 2381 | AUCCUCAUCCGCGUCCUGCCC | 76.3 | 60.3 |
| 1657 | 1138 | CAGGACGCGGAUGAGGAUGCC | 2382 | CAUCCUCAUCCGCGUCCUGCC | 54.5 | 38.5 |
| 1658 | 1139 | AGGACGCGGAUGAGGAUGCCA | 2383 | GCAUCCUCAUCCGCGUCCUGC | 54.7 | 38.7 |
| 1059 | 1140 | GGACGCGGAUGAGGAUGCCAG | 2384 | GGCAUCCUCAUCCGCGUCCUG | 64.7 | 48.7 |
| 1692 | 1141 | GGGACAGCAGUAUGCAGAUGA | 2385 | AUCUGCAUACUGCUGUCCCCC | 80.6 | 64.6 |
| 1693 | 1142 | GGACAGCAGUAUGCAGAUGAC | 2386 | CAUCUGCAUACUGCUGUCCCC | 72.5 | 56.5 |
| 1694 | 1143 | GACAGCAGUAUGCAGAUGACU | 2387 | UCAUCUGCAUACUGCUGUCCC | 76.4 | 62.4 |
| 1695 | 1144 | ACAGCAGUAUGCAGAUGACUG | 2388 | GUCAUCUGCAUACUGCUGUCC | 67.7 | 51.7 |
| 1696 | 1145 | CAGCAGUAUGCAGAUGACUGG | 2389 | AGUCAUCUGCAUACUGCUGUC | 74.6 | 58.6 |
| 1697 | 1146 | AGCAGUAUGCAGAUGACUGGA | 2390 | CAGUCAUCUGCAUACUGCUGU | 75.1 | 59.1 |
| 1698 | 1147 | GCAGUAUGCAGAUGACUGGAU | 2391 | CCAGUCAUCUGCAUACUGCUG | 77.4 | 61.4 |
| 1699 | 1148 | CAGUAUGCAGAUGACUGGAUG | 2392 | UCCAGUCAUCUGCAUACUGCU | 82.8 | 66.8 |
| 1700 | 1149 | AGUAUGCAGAUGACUGGAUGG | 2393 | AUCCAGUCAUCUGCAUACUGC | 75.8 | 58.8 |
| 1737 | 1150 | CCCAGCCCGGCCUCCUCGGCC | 2394 | CCGAGGAGGCCGGGCUGGGGG | 49.7 | 32.7 |
| 1738 | 1151 | CCAGCCCGGCCUCCUCGGCCU | 2395 | GCCGAGGAGGCCGGGCUGGGG | 45.9 | 28.9 |
| 1739 | 1152 | CAGCCCGGCCUCCUCGGCCUC | 2396 | GGCCGAGGAGGCCGGGCUGGG | 40.7 | 21.7 |
| 1740 | 1153 | AGCCCGGCCUCCUCGGCCUCC | 2397 | AGGCCGAGGAGGCCGGGCUGG | 61.3 | 44.3 |
| 1741 | 1154 | GCCCGGCCUCCUCGGCCUCCA | 2398 | GAGGCCGAGGAGGCCGGGCUG | 57.4 | 40.4 |
| 1742 | 1155 | CCCGGCCUCCUCGGCCUCCAU | 2399 | GGAGGCCGAGGAGGCCGGGCU | 38.8 | 21.8 |
| 1743 | 1156 | CCGGCCUCCUCGGCCUCCAUA | 2400 | UGGAGGCCGAGGAGGCCGGGC | 61.9 | 44.9 |
| 1744 | 1157 | CGGCCUCCUCGGCCUCCAUAC | 2401 | AUGGAGGCCGAGGAGGCCGGG | 76.2 | 59.2 |
| 1745 | 1158 | GGCCUCCUCGGCCUCCAUACC | 2402 | UAUGGAGGCCGAGGAGGCCGG | 76.6 | 59.6 |
| 1746 | 1159 | GCCUCCUCGGCCUCCAUACCC | 2403 | GUAUGGAGGCCGAGGAGGCCG | 60.5 | 43.5 |
| 1747 | 1160 | CCUCCUCGGCCUCCAUACCCU | 2404 | GGUAUGGAGGCCGAGGAGGCC | 49.3 | 32.3 |
| 1748 | 1161 | CUCCUCGGCCUCCAUACCCUC | 2405 | GGGUAUGGAGGCCGAGGAGGC | 40 | 23 |
| 1749 | 1162 | UCCUCGGCCUCCAUACCCUCC | 2406 | AGGGUAUGGAGGCCGAGGAGG | 59.5 | 42.5 |
| 1750 | 1163 | CCUCGGCCUCCAUACCCUCCU | 2407 | GAGGGUAUGGAGGCCGAGGAG | 63.5 | 46.5 |
| 1751 | 1164 | CUCGGCCUCCAUACCCUCCUA | 2408 | GGAGGGUAUGGAGGCCGAGGA | 48.1 | 31.1 |
| 1752 | 1165 | UCGGCCUCCAUACCCUCCUAG | 2409 | AGGAGGGUAUGGAGGCCGAGG | 55.6 | 38.6 |
| 1753 | 1166 | CGGCCUCCAUACCCUCCUAGA | 2410 | UAGGAGGGUAUGGAGGCCGAG | 88.9 | 71.9 |
| 1754 | 1167 | GGCCUCCAUACCCUCCUAGAA | 2411 | CUAGGAGGGUAUGGAGGCCGA | 63.7 | 46.7 |
| 1755 | 1168 | GCCUCCAUACCCUCCUAGAAG | 2412 | UCUAGGAGGGUAUGGAGGCCG | 81.7 | 64.7 |
| 1756 | 1169 | CCUCCAUACCCUCCUAGAAGG | 2413 | UUCUAGGAGGGUAUGGAGGCC | 81.5 | 64.5 |
| 1757 | 1170 | CUCCAUACCCUCCUAGAAGGG | 2414 | CUUCUAGGAGGGUAUGGAGGC | 60.2 | 43.2 |
| 1758 | 1171 | UCCAUACCCUCCUAGAAGGGA | 2415 | CCUUCUAGGAGGGUAUGGAGG | 58.9 | 41.9 |
| 1759 | 1172 | CCAUACCCUCCUAGAAGGGAU | 2416 | CCCUUCUAGGAGGGUAUGGAG | 59 | 42 |
| 1760 | 1173 | CAUACCCUCCUAGAAGGGAUG | 2417 | UCCCUUCUAGGAGGGUAUGGA | 71.2 | 54.2 |
| 1761 | 1174 | AUACCCUCCUAGAAGGGAUGG | 2418 | AUCCCUUCUAGGAGGGUAUGG | 73.4 | 56.4 |
| 1762 | 1175 | UACCCUCCUAGAAGGGAUGGU | 2419 | CAUCCCUUCUAGGAGGGUAUG | 61 | 44 |
| 1763 | 1176 | ACCCUCCUAGAAGGGAUGGUU | 2420 | CCAUCCCUUCUAGGAGGGUAU | 50.2 | 33.2 |
| 1764 | 1177 | CCCUCCUAGAAGGGAUGGUUC | 2421 | ACCAUCCCUUCUAGGAGGGUA | 68.6 | 51.6 |
| 1765 | 1178 | CCUCCUAGAAGGGAUGGUUCU | 2422 | AACCAUCCCUUCUAGGAGGGU | 80 | 63 |
| 1766 | 1179 | CUCCUAGAAGGGAUGGUUCUG | 2423 | GAACCAUCCCUUCUAGGAGGG | 68.3 | 51.3 |
| 1767 | 1180 | UCCUAGAAGGGAUGGUUCUGG | 2424 | AGAACCAUCCCUUCUAGGAGG | 73.5 | 56.5 |
| 1768 | 1181 | CCUAGAAGGGAUGGUUCUGGG | 2425 | CAGAACCAUCCCUUCUAGGAG | 72.6 | 55.6 |
| 1804 | 1182 | GGCAGUGCCCGCUACAACCAG | 2426 | GGUUGUAGCGGGCACUGCCAC | 60 | 42 |
| 1805 | 1183 | GCAGUGCCCGCUACAACCAGG | 2427 | UGGUUGUAGCGGGCACUGCCA | 79.5 | 61.5 |
| 1806 | 1184 | CAGUGCCCGCUACAACCAGGG | 2428 | CUGGUUGUAGCGGGCACUGCC | 59.4 | 41.4 |
| 1807 | 1185 | AGUGCCCGCUACAACCAGGGC | 2429 | CCUGGUUGUAGCGGGCACUGC | 56 | 38 |
| 1808 | 1186 | GUGCCCGCUACAACCAGGGCC | 2430 | CCCUGGUUGUAGCGGGCACUG | 53.7 | 35.7 |
| 1809 | 1187 | UGCCCGCUACAACCAGGGCCG | 2431 | GCCCUGGUUGUAGCGGGCACU | 44.1 | 26.1 |
| 1810 | 1188 | GCCCGCUACAACCAGGGCCGG | 2432 | GGCCCUGGUUGUAGCGGGCAC | 43 | 25 |
| 1811 | 1189 | CCCGCUACAACCAGGGCCGGA | 2433 | CGGCCCUGGUUGUAGCGGGCA | 52.2 | 34.2 |
| 1812 | 1190 | CCGCUACAACCAGGGCCGGAG | 2434 | CCGGCCCUGGUUGUAGCGGGC | 47.8 | 29.8 |
| 1813 | 1191 | CGCUACAACCAGGGCCGGAGC | 2435 | UCCGGCCCUGGUUGUAGCGGG | 71.9 | 53.9 |
| 1814 | 1192 | GCUACAACCAGGGCCGGAGCA | 2436 | CUCCGGCCCUGGUUGUAGCGG | 62.6 | 44.6 |
| 1815 | 1193 | CUACAACCAGGGCCGGAGCAG | 2437 | GCUCCGGCCCUGGUUGUAGCG | 50.9 | 32.9 |
| 1816 | 1194 | UACAACCAGGGCCGGAGCAGG | 2438 | UGCUCCGGCCCUGGUUGUAGC | 50.2 | 32.2 |
| 1817 | 1195 | ACAACCAGGGCCGGAGCAGGA | 2439 | CUGCUCCGGCCCUGGUUGUAG | 50.3 | 32.3 |
| 1818 | 1196 | CAACCAGGGCCGGAGCAGGAG | 2440 | CCUGCUCCGGCCCUGGUUGUA | 54 | 36 |
| 1819 | 1197 | AACCAGGGCCGGAGCAGGAGU | 2441 | UCCUGCUCCGGCCCUGGUUGU | 65.5 | 47.5 |
| 1820 | 1198 | ACCAGGGCCGGAGCAGGAGUG | 2442 | CUCCUGCUCCGGCCCUGGUUG | 52.7 | 34.7 |
| 1821 | 1199 | CCAGGGCCGGAGCAGGAGUGG | 2443 | ACUCCUGCUCCGGCCCUGGUU | 68.3 | 50.3 |
| 1822 | 1200 | CAGGGCCGGAGCAGGAGUGGG | 2444 | CACUCCUGCUCCGGCCCUGGU | 57.3 | 39.3 |
| 1858 | 1201 | UUUCACACCCAAACCAUCCUC | 2445 | GGAUGGUUUGGGUGUGAAAAC | 36.4 | 18.4 |
| 1859 | 1202 | UUCACACCCAAACCAUCCUCA | 2446 | AGGAUGGUUUGGGUGUGAAAA | 56.8 | 38.8 |
| 1860 | 1203 | UCACACCCAAACCAUCCUCAU | 2447 | GAGGAUGGUUUGGGUGUGAAA | 50.9 | 32.9 |
| 1861 | 1204 | CACACCCAAACCAUCCUCAUU | 2448 | UGAGGAUGGUUUGGGUGUGAA | 71.6 | 53.6 |
| 1862 | 1205 | ACACCCAAACCAUCCUCAUUC | 2449 | AUGAGGAUGGUUUGGGUGUGA | 74.3 | 56.3 |
| 1863 | 1206 | CACCCAAACCAUCCUCAUUCU | 2450 | AAUGAGGAUGGUUUGGGUGUG | 80.4 | 62.4 |
| 1864 | 1207 | ACCCAAACCAUCCUCAUUCUC | 2451 | GAAUGAGGAUGGUUUGGGUGU | 57.7 | 39.7 |
| 1865 | 1208 | CCCAAACCAUCCUCAUUCUCU | 2452 | AGAAUGAGGAUGGUUUGGGUG | 78 | 60 |
| 1866 | 1209 | CCAAACCAUCCUCAUUCUCUC | 2453 | GAGAAUGAGGAUGGUUUGGGU | 65.4 | 47.4 |
| 1867 | 1210 | CAAACCAUCCUCAUUCUCUCC | 2454 | AGAGAAUGAGGAUGGUUUGGG | 70.5 | 52.5 |
| 1868 | 1211 | AAACCAUCCUCAUUCUCUCCC | 2455 | GAGAGAAUGAGGAUGGUUUGG | 66.7 | 48.7 |
| 1869 | 1212 | AACCAUCCUCAUUCUCUCCCU | 2456 | GGAGAGAAUGAGGAUGGUUUG | 57.3 | 39.3 |
| 1870 | 1213 | ACCAUCCUCAUUCUCUCCCUC | 2457 | GGGAGAGAAUGAGGAUGGUUU | 46.4 | 28.4 |
| 1871 | 1214 | CCAUCCUCAUUCUCUCCCUCU | 2458 | AGGGAGAGAAUGAGGAUGGUU | 72.8 | 54.8 |
| 1872 | 1215 | CAUCCUCAUUCUCUCCCUCUC | 2459 | GAGGGAGAGAAUGAGGAUGGU | 66.2 | 48.2 |
| 1873 | 1216 | AUCCUCAUUCUCUCCCUCUCA | 2460 | AGAGGGAGAGAAUGAGGAUGG | 61 | 43 |
| 1874 | 1217 | UCCUCAUUCUCUCCCUCUCAG | 2461 | GAGAGGGAGAGAAUGAGGAUG | 64 | 46 |
| 1875 | 1218 | CCUCAUUCUCUCCCUCUCAGC | 2462 | UGAGAGGGAGAGAAUGAGGAU | 81.7 | 63.7 |
| 1876 | 1219 | CUCAUUCUCUCCCUCUCAGCC | 2463 | CUGAGAGGGAGAGAAUGAGGA | 66.7 | 48.7 |
| 1877 | 1220 | UCAUUCUCUCCCUCUCAGCCC | 2464 | GCUGAGAGGGAGAGAAUGAGG | 52.2 | 34.2 |
| 1878 | 1221 | CAUUCUCUCCCUCUCAGCCCU | 2465 | GGCUGAGAGGGAGAGAAUGAG | 56.3 | 38.3 |
| 1879 | 1222 | AUUCUCUCCCUCUCAGCCCUG | 2466 | GGGCUGAGAGGGAGAGAAUGA | 44 | 26 |
| 1880 | 1223 | UUCUCUCCCUCUCAGCCCUGG | 2467 | AGGGCUGAGAGGGAGAGAAUG | 60 | 42 |
| 1899 | 1224 | GGCCCUGCUUGGACCUCGAUA | 2468 | UCGAGGUCCAAGCAGGGCCAG | 89.2 | 71.2 |
| 1900 | 1225 | GCCCUGCUUGGACCUCGAUAA | 2469 | AUCGAGGUCCAAGCAGGGCCA | 78.2 | 59.2 |
| 1901 | 1226 | CCCUGCUUGGACCUCGAUAAC | 2470 | UAUCGAGGUCCAAGCAGGGCC | 77.8 | 58.8 |
| 1902 | 1227 | CCUGCUUGGACCUCGAUAACG | 2471 | UUAUCGAGGUCCAAGCAGGGC | 88.4 | 69.4 |
| 1903 | 1228 | CUGCUUGGACCUGGAUAACGG | 2472 | GUUAUCGAGGUCCAAGCAGGG | 72.3 | 53.3 |
| 1904 | 1229 | UGCUUGGACCUCGAUAACGGG | 2473 | CGUUAUCGAGGUCCAAGCAGG | 59.1 | 40.1 |
| 1929 | 1230 | GGGUGCCCUAGCAUCAGAAGG | 2474 | UUCUGAUGCUAGGGCACCCCU | 83.4 | 64.4 |
| 1930 | 1231 | GGUGCCCUAGCAUCAGAAGGG | 2475 | CUUCUGAUGCUAGGGCACCCC | 66.1 | 47.1 |
| 1931 | 1232 | GUGCCCUAGCAUCAGAAGGGU | 2476 | CCUUCUGAUGCUAGGGCACCC | 54.8 | 35.8 |
| 1932 | 1233 | UGCCCUAGCAUCAGAAGGGUU | 2477 | CCCUUCUGAUGCUAGGGCACC | 44.4 | 25.4 |
| 1933 | 1234 | GCCCUAGCAUCAGAAGGGUUC | 2478 | ACCCUUCUGAUGCUAGGGCAC | 65.7 | 46.7 |
| 1934 | 1235 | CCCUAGCAUCAGAAGGGUUCA | 2479 | AACCCUUCUGAUGCUAGGGCA | 79.8 | 60.8 |
| 1935 | 1236 | CCUAGCAUCAGAAGGGUUCAU | 2480 | GAACCCUUCUGAUGCUAGGGC | 63.1 | 44.1 |
| 1936 | 1237 | CUAGCAUCAGAAGGGUUCAUG | 2481 | UGAACCCUUCUGAUGCUAGGG | 84.6 | 65.6 |
| 1937 | 1238 | UAGCAUCAGAAGGGUUCAUGG | 2482 | AUGAACCCUUCUGAUGCUAGG | 72.2 | 53.2 |
| 2006 | 1239 | GGGCUGCAGAGAGGGUAGAGA | 2483 | UCUACCCUCUCUGCAGCCCCC | 73.3 | 53.3 |
| 2007 | 1240 | GGCUGCAGAGAGGGUAGAGAA | 2484 | CUCUACCCUCUCUGCAGCCCC | 60.5 | 40.5 |
| 2008 | 1241 | GCUGCAGAGAGGGUAGAGAAG | 2485 | UCUCUACCCUCUCUGCAGCCC | 75.2 | 55.2 |
| 2009 | 1242 | CUGCAGAGAGGGUAGAGAAGG | 2486 | UUCUCUACCCUCUCUGCAGCC | 80.6 | 60.6 |
| 2010 | 1243 | UGCAGAGAGGGUAGAGAAGGG | 2487 | CUUCUCUACCCUCUCUGCAGC | 62.7 | 42.7 |
| 2011 | 1244 | GCAGAGAGGGUAGAGAAGGGA | 2488 | CCUUCUCUACCCUCUCUGCAG | 67.6 | 47.6 |
| 2012 | 1245 | CAGAGAGGGUAGAGAAGGGAC | 2489 | CCCUUCUCUACCCUCUCUGCA | 64.4 | 44.4 |
| 2013 | 1246 | AGAGAGGGUAGAGAAGGGACU | 2490 | UCCCUUCUCUACCCUCUCUGC | 70.2 | 50.2 |
| 2014 | 1247 | GAGAGGGUAGAGAAGGGACUU | 2491 | GUCCCUUCUCUACCCUCUCUG | 71.7 | 51.7 |
| 2015 | 1248 | AGAGGGUAGAGAAGGGACUUU | 2492 | AGUCCCUUCUCUACCCUCUCU | 72.8 | 52.8 |
| 2016 | 1249 | GAGGGUAGAGAAGGGACUUUG | 2493 | AAGUCCCUUCUCUACCCUCUC | 73.6 | 53.6 |
| 2017 | 1250 | AGGGUAGAGAAGGGACUUUGC | 2494 | AAAGUCCCUUCUCUACCCUCU | 71.3 | 51.3 |
| 2018 | 1251 | GGGUAGAGAAGGGACUUUGCA | 2495 | CAAAGUCCCUUCUCUACCCUC | 69.2 | 49.2 |
| 2019 | 1252 | GGUAGAGAAGGGACUUUGCAG | 2496 | GCAAAGUCCCUUCUCUACCCU | 70.7 | 50.7 |
| 2020 | 1253 | GUAGAGAAGGGACUUUGCAGG | 2497 | UGCAAAGUCCCUUCUCUACCC | 72.2 | 52.2 |
| 2021 | 1254 | UAGAGAAGGGACUUUGCAGGU | 2498 | CUGCAAAGUCCCUUCUCUACC | 52.8 | 32.8 |
| 2022 | 1255 | AGAGAAGGGACUUUGCAGGUG | 2499 | CCUGCAAAGUCCCUUCUCUAC | 54.7 | 34.7 |
| 2023 | 1256 | GAGAAGGGACUUUGCAGGUGA | 2500 | ACCUGCAAAGUCCCUUCUCUA | 74.3 | 54.3 |
| 2024 | 1257 | AGAAGGGACUUUGCAGGUGAA | 2501 | CACCUGCAAAGUCCCUUCUCU | 62.1 | 42.1 |
| 2025 | 1258 | GAAGGGACUUUGCAGGUGAAU | 2502 | UCACCUGCAAAGUCCCUUCUC | 81 | 61 |
| 2026 | 1259 | AAGGGACUUUGCAGGUGAAUG | 2503 | UUCACCUGCAAAGUCCCUUCU | 82.9 | 62.9 |
| 2027 | 1260 | AGGGACUUUGCAGGUGAAUGG | 2504 | AUUCACCUGCAAAGUCCCUUC | 66.8 | 46.8 |
| 2071 | 1261 | UUUCAUCAGAGGUGGGUGGGU | 2505 | CCACCCACCUCUGAUGAAAAU | 47.7 | 27.7 |
| 2072 | 1262 | UUCAUCAGAGGUGGGUGGGUG | 2506 | CCCACCCACCUCUGAUGAAAA | 35.6 | 15.6 |
| 2073 | 1263 | UCAUCAGAGGUGGGUGGGUGU | 2507 | ACCCACCCACCUCUGAUGAAA | 55 | 35 |
| 2074 | 1264 | CAUCAGAGGUGGGUGGGUGUU | 2508 | CACCCACCCACCUCUGAUGAA | 59.8 | 39.8 |
| 2075 | 1265 | AUCAGAGGUGGGUGGGUGUUC | 2509 | ACACCCACCCACCUCUGAUGA | 64.5 | 44.5 |
| 2076 | 1266 | UCAGAGGUGGGUGGGUGUUCA | 2510 | AACACCCACCCACCUCUGAUG | 68.4 | 48.4 |
| 2077 | 1267 | CAGAGGUGGGUGGGUGUUCAC | 2511 | GAACACCCACCCACCUCUGAU | 63.2 | 43.2 |
| 2078 | 1268 | AGAGGUGGGUGGGUGUUCACA | 2512 | UGAACACCCACCCACCUCUGA | 81.5 | 61.5 |
| 2079 | 1289 | GAGGUGGGUGGGUGUUCACAA | 2513 | GUGAACACCCACCCACCUCUG | 73.4 | 53.4 |
| 2080 | 1270 | AGGUGGGUGGGUGUUCACAAU | 2514 | UGUGAACACCCACCCACCUCU | 74 | 54 |
| 2081 | 1271 | GGUGGGUGGGUGUUCACAAUA | 2515 | UUGUGAACACCCACCCACCUC | 89.9 | 69.9 |
| 2082 | 1272 | GUGGGUGGGUGUUCACAAUAU | 2516 | AUUGUGAACACCCACCCACCU | 83.7 | 63.7 |
| 2083 | 1273 | UGGGUGGGUGUUCACAAUAUU | 2517 | UAUUGUGAACACCCACCCACC | 74.1 | 54.1 |
| 2084 | 1274 | GGGUGGGUGUUCACAAUAUUU | 2518 | AUAUUGUGAACACCCACCCAC | 87.2 | 67.2 |
| 2085 | 1275 | GGUGGGUGUUCACAAUAUUUA | 2519 | AAUAUUGUGAACACCCACCCA | 96.5 | 76.5 |
| 2086 | 1276 | GUGGGUGUUCACAAUAUUUAU | 2520 | AAAUAUUGUGAACACCCACCC | 79.8 | 59.8 |
| 2087 | 1277 | UGGGUGUUCACAAUAUUUAUU | 2521 | UAAAUAUUGUGAACACCCACC | 78.4 | 58.4 |
| 2088 | 1278 | GGGUGUUCACAAUAUUUAUUU | 2522 | AUAAAUAUUGUGAACACCCAC | 93.3 | 73.3 |
| 2109 | 1279 | UUUCAUUUGGUAAUGGGAGGG | 2523 | CUCCCAUUACCAAAUGAAAAA | 48.7 | 27.7 |
| 2138 | 1280 | GGGUAUUUAUUUAGGAGGGAG | 2524 | CCCUCCUAAAUAAAUACCCCC | 57.6 | 36.6 |
| 2139 | 1281 | GGUAUUUAUUUAGGAGGGAGU | 2525 | UCCCUCCUAAAUAAAUACCCC | 71.9 | 50.9 |
| 2140 | 1282 | GUAUUUAUUUAGGAGGGAGUG | 2526 | CUCCCUCCUAAAUAAAUACCC | 54.9 | 33.9 |
| 2141 | 1283 | UAUUUAUUUAGGAGGGAGUGU | 2527 | ACUCCCUCCUAAAUAAAUACC | 53.4 | 32.4 |
| 2142 | 1284 | AUUUAUUUAGGAGGGAGUGUG | 2528 | CACUCCCUCCUAAAUAAAUAC | 47.6 | 26.6 |
| 2143 | 1285 | UUUAUUUAGGAGGGAGUGUGG | 2529 | ACACUCCCUCCUAAAUAAAUA | 55.8 | 34.8 |
| 2144 | 1286 | UUAUUUAGGAGGGAGUGUGGU | 2530 | CACACUCCCUCCUAAAUAAAU | 50.4 | 29.4 |
| 2145 | 1287 | UAUUUAGGAGGGAGUGUGGUU | 2531 | CCACACUCCCUCCUAAAUAAA | 53 | 32 |
| 2146 | 1288 | AUUUAGGAGGGAGUGUGGUUU | 2532 | ACCACACUCCCUCCUAAAUAA | 58.1 | 37.1 |
| 2147 | 1289 | UUUAGGAGGGAGUGUGGUUUC | 2533 | AACCACACUCCCUCCUAAAUA | 65.9 | 44.9 |
| 2148 | 1290 | UUAGGAGGGAGUGUGGUUUCC | 2534 | AAACCACACUCCCUCCUAAAU | 60.8 | 39.8 |
| 2149 | 1291 | UAGGAGGGAGUGUGGUUUCCU | 2535 | GAAACCACACUCCCUCCUAAA | 59 | 38 |
| 2150 | 1292 | AGGAGGGAGUGUGGUUUCCUU | 2536 | GGAAACCACACUCCCUCCUAA | 61.2 | 40.2 |
| 2151 | 1293 | GGAGGGAGUGUGGUUUCCUUA | 2537 | AGGAAACCACACUCCCUCCUA | 76.7 | 55.7 |
| 2152 | 1294 | GAGGGAGUGUGGUUUCCUUAG | 2538 | AAGGAAACCACACUCCCUCCU | 83 | 62 |
| 2153 | 1295 | AGGGAGUGUGGUUUCCUUAGA | 2539 | UAAGGAAACCACACUCCCUCC | 79.6 | 58.6 |
| 2154 | 1296 | GGGAGUGUGGUUUCCUUAGAA | 2540 | CUAAGGAAACCACACUCCCUC | 74.3 | 53.3 |
| 2155 | 1297 | GGAGUGUGGUUUCCUUAGAAG | 2541 | UCUAAGGAAACCACACUCCCU | 94.3 | 73.3 |
| 2156 | 1298 | GAGUGUGGUUUCCUUAGAAGG | 2542 | UUGUAAGGAAACCACACUCCC | 88.3 | 67.3 |
| 2157 | 1299 | AGUGUGGUUUCCUUAGAAGGU | 2543 | CUUCUAAGGAAACCACACUCC | 68.6 | 47.6 |
| 2158 | 1300 | GUGUGGUUUCCUUAGAAGGUA | 2544 | CCUUCUAAGGAAACCACACUC | 67.8 | 46.8 |
| 2159 | 1301 | UGUGGUUUCCUUAGAAGGUAU | 2545 | ACCUUCUAAGGAAACCACACU | 71.6 | 50.6 |
| 2160 | 1302 | GUGGUUUCCUUAGAAGGUAUA | 2546 | UACCUUCUAAGGAAACCACAC | 81.1 | 60.1 |
| 2161 | 1303 | UGGUUUCCUUAGAAGGUAUAG | 2547 | AUACCUUCUAAGGAAACCACA | 80.6 | 59.6 |
| 2162 | 1304 | GGUUUCCUUAGAAGGUAUAGU | 2548 | UAUACCUUCUAAGGAAACCAC | 88.3 | 67.3 |
| 2163 | 1305 | GUUUCCUUAGAAGGUAUAGUC | 2549 | CUAUACCUUCUAAGGAAACCA | 67.3 | 46.3 |
| 2164 | 1306 | UUUCCUUAGAAGGUAUAGUCU | 2550 | ACUAUACCUUCUAAGGAAACC | 53.6 | 32.6 |
| 2165 | 1307 | UUCCUUAGAAGGUAUAGUCUC | 2551 | GACUAUACCUUCUAAGGAAAC | 51.5 | 30.5 |
| 2166 | 1308 | UCCUUAGAAGGUAUAGUCUCU | 2552 | AGACUAUACCUUCUAAGGAAA | 68.5 | 47.5 |
| 2167 | 1309 | CCUUAGAAGGUAUAGUCUCUA | 2553 | GAGACUAUACCUUCUAAGGAA | 75.3 | 54.3 |
| 2168 | 1310 | CUUAGAAGGUAUAGUCUCUAG | 2554 | AGAGACUAUACCUUCUAAGGA | 81.1 | 60.1 |
| 2169 | 1311 | UUAGAAGGUAUAGUCUCUAGG | 2555 | UAGAGACUAUACCUUCUAAGG | 76.8 | 55.8 |
| 2170 | 1312 | UAGAAGGUAUAGUCUCUAGCC | 2556 | CUAGAGACUAUACCUUCUAAG | 72.1 | 51.1 |
| 2171 | 1313 | AGAAGGUAUAGUCUCUAGCCC | 2557 | GCUAGAGACUAUACCUUCUAA | 67.9 | 46.9 |
| 2172 | 1314 | GAAGGUAUAGUCUCUAGCCCU | 2558 | GGCUAGAGACUAUACCUUCUA | 66.9 | 45.9 |
| 2173 | 1315 | AAGGUAUAGUCUCUAGCCCUC | 2559 | GGGCUAGAGACUAUACCUUCU | 56.4 | 35.4 |
| 2174 | 1316 | AGGUAUAGUCUCUAGCCCUCU | 2560 | AGGGCUAGAGACUAUACCUUC | 70.8 | 49.8 |
| 2175 | 1317 | GGUAUAGUCUCUAGCCCUCUA | 2561 | GAGGGCUAGAGACUAUACCUU | 76.1 | 55.1 |
| 2176 | 1318 | GUAUAGUCUCUAGCCCUCUAA | 2562 | AGAGGGCUAGAGACUAUACCU | 69.1 | 48.1 |
| 2177 | 1319 | UAUAGUCUCUAGCCCUCUAAG | 2563 | UAGAGGGCUAGAGACUAUACC | 74.9 | 53.9 |
| 2178 | 1320 | AUAGUCUCUAGCCCUCUAAGG | 2564 | UUAGAGGGCUAGAGACUAUAC | 71.3 | 50.3 |
| 2179 | 1321 | UAGUCUCUAGCCCUCUAAGGC | 2565 | CUUAGAGGGCUAGAGACUAUA | 63.7 | 42.7 |
| 2180 | 1322 | AGUCUCUAGCCCUCUAAGGCU | 2566 | CCUUAGAGGGCUAGAGACUAU | 61.3 | 40.3 |
| 2181 | 1323 | GUCUCUAGCCCUCUAAGGCUG | 2567 | GCCUUAGAGGGCUAGAGACUA | 57.6 | 36.6 |
| 2182 | 1324 | UCUCUAGCCCUCUAAGGCUGG | 2568 | AGCCUUAGAGGGCUAGAGACU | 59.5 | 38.5 |
| 2183 | 1325 | CUCUAGCCCUCUAAGGCUGGG | 2569 | CAGCCUUAGAGGGCUAGAGAC | 60.5 | 39.5 |
| 2228 | 1326 | AAAUGAGGAGUUUAGAGUUGC | 2570 | AACUCUAAACUCCUCAUUUUC | 76.1 | 54.1 |
| 2229 | 1327 | AAUGAGGAGUUUAGAGUUGCA | 2571 | CAACUCUAAACUCCUCAUUUU | 73.9 | 51.9 |
| 2230 | 1328 | AUGAGGAGUUUAGAGUUGCAG | 2572 | GCAACUCUAAACUCCUCAUUU | 62.3 | 40.3 |
| 2231 | 1329 | UGAGGAGUUUAGAGUUGCAGC | 2573 | UGCAACUCUAAACUCCUCAUU | 86.6 | 64.6 |
| 2232 | 1330 | GAGGAGUUUAGAGUUGCAGCU | 2574 | CUGCAACUCUAAACUCCUCAU | 73.3 | 51.3 |
| 2233 | 1331 | AGGAGUUUAGAGUUGCAGCUG | 2575 | GCUGCAACUCUAAACUCCUCA | 66.2 | 44.2 |
| 2234 | 1332 | GGAGUUUAGAGUUGCAGCUGG | 2576 | AGCUGCAACUCUAAACUCCUC | 81.6 | 59.6 |
| 2235 | 1333 | GAGUUUAGAGUUGCAGCUGGG | 2577 | CAGCUGCAACUCUAAACUCCU | 72.1 | 50.1 |
| 2260 | 1334 | GGGUUUGAAGGAAGUUGGAAG | 2578 | UCCAACUUCCUUCAAACCCCU | 88.3 | 55.3 |
| 2261 | 1335 | GGUUUGAAGGAAGUUGGAAGU | 2579 | UUCCAACUUCCUUCAAACCCC | 83.2 | 61.2 |
| 2262 | 1335 | GGUUUGAAGGAAGUUGGAAGU | 2550 | CUUCCAACUUCCUUCAAACCC | 65.5 | 43.5 |
| 2263 | 1337 | UUUGAAGGAAGUUGGAAGUGG | 2581 | ACUUCCAACUUCCUUCAAACC | 56.7 | 34.7 |
| 2264 | 1338 | UUGAAGGAAGUUGGAAGUGGG | 2582 | CACUUCCAACUUCCUUCAAAC | 47.3 | 25.3 |
| 2294 | 1339 | GGGCAUCUGGUCUCAGAAAUG | 2583 | UUUCUGAGACCAGAUGCCCCC | 82.2 | 60.2 |
| 2295 | 1340 | GGCAUCUGGUCUCAGAAAUGG | 2584 | AUUUCUGAGACCAGAUGCCCC | 77.6 | 55.6 |
| 2296 | 1341 | GCAUCUGGUCUCAGAAAUGGA | 2585 | CAUUUCUGAGACCAGAUGCCC | 69.2 | 47.2 |
| 2297 | 1342 | CAUCUGGUCUCAGAAAUGGAC | 2586 | CCAUUUCUGAGACCAGAUGCC | 58.3 | 36.3 |
| 2298 | 1343 | AUCUGGUCUCAGAAAUGGACC | 2587 | UCCAUUUCUGAGACCAGAUGC | 74.3 | 52.3 |
| 2299 | 1344 | UCUGGUCUCAGAAAUGGACCA | 2588 | GUCCAUUUCUGAGACCAGAUG | 65.3 | 43.3 |
| 2300 | 1345 | CUGGUCUCAGAAAUGGACCAG | 2589 | GGUCCAUUUCUGAGACCAGAU | 56.3 | 33.3 |
| 2301 | 1346 | UGGUCUCAGAAAUGGACCAGC | 2590 | UGGUCCAUUUCUGAGACCAGA | 79.3 | 56.3 |
| 2302 | 1347 | GGUCUCAGAAAUGGACCAGCU | 2591 | CUGGUCCAUUUCUGAGACCAG | 71.2 | 48.2 |
| 2303 | 1348 | GUCUCAGAAAUGGACCAGCUG | 2592 | GCUGGUCCAUUUCUGAGACCA | 58 | 35 |
| 2304 | 1349 | UCUCAGAAAUGGACCAGCUGG | 2593 | AGCUGGUCCAUUUCUGAGACC | 62.6 | 39.6 |
| 2305 | 1350 | CUCAGAAAUGGACCAGCUGGA | 2594 | CAGCUGGUCCAUUUCUGAGAC | 63.7 | 40.7 |
| 2306 | 1351 | UCAGAAAUGGACCAGCUGGAU | 2595 | CCAGCUGGUCCAUUUCUGAGA | 59.7 | 36.7 |
| 2307 | 1352 | CAGAAAUGGACCAGCUGGAUG | 2596 | UCCAGCUGGUCCAUUUCUGAG | 84 | 61 |
| 2308 | 1353 | AGAAAUGGACCAGCUGGAUGC | 2597 | AUCCAGCUGGUCCAUUUCUGA | 77.5 | 54.5 |
| 2309 | 1354 | GAAAUGGACCAGCUGGAUGCA | 2598 | CAUCCAGCUGGUCCAUUUCUG | 72.6 | 49.6 |
| 2310 | 1355 | AAAUGGACCAGCUGGAUGCAG | 2599 | GCAUCCAGCUGGUCCAUUUCU | 58 | 35 |
| 2311 | 1356 | AAUGGACCAGCUGGAUGCAGG | 2600 | UGCAUCCAGCUGGUCCAUUUC | 69.3 | 46.3 |
| 2312 | 1357 | AUGGACCAGCUGGAUGCAGGG | 2601 | CUGCAUCCAGCUGGUCCAUUU | 57.4 | 34.4 |
| 2313 | 1358 | UGGACCAGCUGGAUGCAGGGC | 2602 | CCUGCAUCCAGCUGGUCCAUU | 57.9 | 34.9 |
| 2314 | 1359 | GGACCAGCUGGAUGCAGGGCA | 2603 | CCCUGCAUCCAGCUGGUCCAU | 67.8 | 44.8 |
| 2315 | 1360 | GACCAGCUGGAUGCAGGGCAG | 2604 | GCCCUGCAUCCAGCUGGUCCA | 51.3 | 28.3 |
| 2316 | 1361 | ACCAGCUGGAUGCAGGGCAGG | 2605 | UGCCCUGCAUCCAGCUGGUCC | 59.1 | 36.1 |
| 2317 | 1362 | CCAGCUGGAUGCAGGGCAGGG | 2606 | CUGCCCUGCAUCCAGCUGGUC | 66.2 | 43.2 |
| 2336 | 1363 | GGGACUGAGGGUGCUUGAGUA | 2607 | CUCAAGCACCCUCAGUCCCCU | 64.8 | 41.8 |
| 2337 | 1364 | GGACUGAGGGUGCUUGAGUAG | 2608 | ACUCAAGCACCCUCAGUCCCC | 67.6 | 44.6 |
| 2338 | 1365 | GACUGAGGGUGCUUGAGUAGG | 2609 | UACUCAAGCACCCUCAGUCCC | 81.7 | 58.7 |
| 2339 | 1366 | ACUGAGGGUGCUUGAGUAGGA | 2610 | CUACUCAAGCACCCUCAGUCC | 57.9 | 34.9 |
| 2340 | 1367 | CUGAGGGUGCUUGAGUAGGAU | 2611 | CCUACUCAAGCACCCUCAGUC | 52.4 | 29.4 |
| 2341 | 1365 | UGAGGGUGCUUGAGUAGGAUG | 2612 | UCCUACUCAAGCACCCUCAGU | 76.8 | 53.8 |
| 2342 | 1369 | GAGGGUGCUUGAGUAGGAUGU | 2613 | AUCCUACUCAAGCACCCUCAG | 80.6 | 57.6 |
| 2343 | 1370 | AGGGUGCUUGAGUAGGAUGUG | 2614 | CAUCCUACUCAAGCACCCUCA | 67.1 | 44.1 |
| 2344 | 1371 | GGGUGCUUGAGUAGGAUGUGA | 2615 | ACAUCCUACUCAAGCACCCUC | 76.7 | 53.7 |
| 2345 | 1372 | GGUGCUUGAGUAGGAUGUGAG | 2616 | CACAUCCUACUCAAGCACCCU | 75.4 | 52.4 |
| 2346 | 1373 | GUGCUUGAGUAGGAUGUGAGA | 2617 | UCACAUCCUACUCAAGCACCC | 77.5 | 54.5 |
| 2347 | 1374 | UGCUUGAGUAGGAUGUGAGAC | 2618 | CUCACAUCCUACUCAAGCACC | 57.7 | 34.7 |
| 2348 | 1375 | GCUUGAGUAGGAUGUGAGACU | 2619 | UCUCACAUCCUACUCAAGCAC | 86.9 | 63.9 |
| 2349 | 1376 | CUUGAGUAGGAUGUGAGACUU | 2620 | GUCUCACAUCCUACUCAAGCA | 62.2 | 39.2 |
| 2350 | 1377 | UUGAGUAGGAUGUGAGACUUC | 2621 | AGUCUCACAUCCUACUCAAGC | 60.7 | 37.7 |
| 2351 | 1378 | UGAGUAGGAUGUGAGACUUCA | 2622 | AAGUCUCACAUCCUACUCAAG | 80.4 | 57.4 |
| 2352 | 1379 | GAGUAGGAUGUGAGACUUCAU | 2623 | GAAGUCUCACAUCCUACUCAA | 76.7 | 53.7 |
| 2353 | 1380 | AGUAGGAUGUGAGACUUCAUG | 2624 | UGAAGUCUCACAUCCUACUCA | 88.9 | 65.9 |
| 2354 | 1381 | GUAGGAUGUGAGACUUCAUGG | 2625 | AUGAAGUCUCACAUCCUACUC | 85.4 | 62.4 |
| 2355 | 1382 | UAGGAUGUGAGACUUCAUGGG | 2626 | CAUGAAGUCUCACAUCCUACU | 62.3 | 39.3 |
| 2356 | 1383 | AGGAUGUGAGACUUCAUGGGC | 2627 | CCAUGAAGUCUCACAUCCUAC | 61.7 | 38.7 |
| 2357 | 1384 | GGAUGUGAGACUUCAUGGGCC | 2628 | CCCAUGAAGUCUCACAUCCUA | 74.5 | 51.5 |
| 2358 | 1385 | GAUGUGAGACUUCAUGGGCCU | 2629 | GCCCAUGAAGUCUCACAUCCU | 60.9 | 37.9 |
| 2359 | 1386 | AUGUGAGACUUCAUGGGCCUG | 2630 | GGCCCAUGAAGUCUCACAUCC | 42.4 | 19.4 |
| 2360 | 1387 | UGUGAGACUUCAUGGGCCUGG | 2631 | AGGCCCAUGAAGUCUCACAUC | 62.7 | 39.7 |
| 2361 | 1388 | GUGAGACUUCAUGGGCCUGGG | 2632 | CAGGCCCAUGAAGUCUCACAU | 59.6 | 36.6 |
| 2362 | 1389 | UGAGACUUCAUGGGCCUGGGU | 2633 | CCAGGCCCAUGAAGUCUCACA | 42.4 | 19.4 |
| 2363 | 1390 | GAGACUUCAUGGGCCUGGGUU | 2634 | CCCAGGCCCAUGAAGUCUCAC | 51.5 | 28.5 |
| 2364 | 1391 | AGACUUCAUGGGCCUGGGUUC | 2635 | ACCCAGGCCCAUGAAGUCUCA | 61.3 | 38.3 |
| 2365 | 1392 | GACUUCAUGGGCCUGGGUUCU | 2636 | AACCCAGGCCCAUGAAGUCUC | 61.7 | 38.7 |
| 2366 | 1393 | ACUUCAUGGGCCUGGGUUCUG | 2637 | GAACCCAGGCCCAUGAAGUCU | 56.1 | 33.1 |
| 2367 | 1394 | CUUCAUGGGCCUGGGUUCUGU | 2638 | AGAACCCAGGCCCAUGAAGUC | 56.2 | 33.2 |
| 2368 | 1395 | UUCAUGGGCCUGGGUUCUGUU | 2639 | CAGAACCCAGGCCCAUGAAGU | 43.1 | 20.1 |
| 2369 | 1396 | UCAUGGGCCUGGGUUCUGUUG | 2640 | ACAGAACCCAGGCCCAUGAAG | 59 | 36 |
| 2370 | 1397 | CAUGGGCCUGGGUUCUGUUGA | 2641 | AACAGAACCCAGGCCCAUGAA | 73.3 | 50.3 |
| 2371 | 1398 | AUGGGCCUGGGUUCUGUUGAG | 2642 | CAACAGAACCCAGGCCCAUGA | 54.5 | 31.5 |
| 2372 | 1399 | UGGGCCUGGGUUCUGUUGAGU | 2643 | UCAACAGAACCCAGGCCCAUG | 69.3 | 46.3 |
| 2373 | 1400 | GGGCCUGGGUUCUGUUGAGUU | 2644 | CUCAACAGAACCCAGGCCCAU | 72.9 | 49.9 |
| 2374 | 1401 | GGCCUGGGUUCUGUUGAGUUU | 2645 | ACUCAACAGAACCCAGGCCCA | 71.9 | 48.9 |
| 2395 | 1402 | UUUCAGUAUCAAUUUCUUAAA | 2646 | UAAGAAAUUGAUACUGAAAAA | 78.6 | 55.6 |
| 2396 | 1403 | UUCAGUAUCAAUUUCUUAAAC | 2647 | UUAAGAAAUUGAUACUGAAAA | 78.3 | 55.3 |
| 2397 | 1404 | UCAGUAUCAAUUUCUUAAACC | 2648 | UUUAAGAAAUUGAUACUGAAA | 83.6 | 60.6 |
| 2398 | 1405 | CAGUAUCAAUUUCUUAAACCA | 2649 | GUUUAAGAAAUUGAUACUGAA | 72.1 | 49.1 |
| 2399 | 1406 | AGUAUCAAUUUCUUAAACCAA | 2650 | GGUUUAAGAAAUUGAUACUGA | 59.3 | 36.3 |
| 2400 | 1407 | GUAUCAAUUUCUUAAACCAAA | 2651 | UGGUUUAAGAAAUUGAUACUG | 88.9 | 64.9 |
| 2401 | 1408 | UAUCAAUUUCUUAAACCAAAU | 2652 | UUGGUUUAAGAAAUUGAUACU | 79.7 | 55.7 |
| 2402 | 1409 | AUCAAUUUCUUAAACCAAAUU | 2653 | UUUGGUUUAAGAAAUUGAUAC | 77.6 | 53.6 |
| 2403 | 1410 | UCAAUUUCUUAAACCAAAUUU | 2654 | AUUUGGUUUAAGAAAUUGAUA | 78 | 54 |
| 2443 | 1411 | GGGUGCUCAUCUCGUGACCUC | 2655 | GGUCACGAGAUGAGCACCCCC | 57.6 | 33.6 |
| 2444 | 1412 | GGUGCUCAUCUCGUGACCUCU | 2656 | AGGUCACGAGAUGAGCACCCC | 66 | 42 |
| 2445 | 1413 | GUGCUCAUCUCGUGACCUCUG | 2657 | GAGGUCACGAGAUGAGCACCC | 57.1 | 33.1 |
| 2446 | 1414 | UGCUCAUCUCGUGACCUCUGC | 2658 | AGAGGUCACGAGAUGAGCACC | 59.8 | 35.8 |
| 2447 | 1415 | GCUCAUCUCGUGACCUCUGCC | 2659 | CAGAGGUCACGAGAUGAGCAC | 65.9 | 41.9 |
| 2448 | 1416 | CUCAUCUCGUGACCUCUGCCA | 2660 | GCAGAGGUCACGAGAUGAGCA | 54.4 | 30.4 |
| 2449 | 1417 | UCAUCUCGUGACCUCUGCCAC | 2661 | GGCAGAGGUCACGAGAUGAGC | 42 | 18 |
| 2468 | 1418 | ACCCACAUCCUUCACAAACUC | 2662 | GUUUGUGAAGGAUGUGGGUGG | 62.2 | 38.2 |
| 2469 | 1419 | CCCACAUCCUUCACAAACUCC | 2663 | AGUUUGUGAAGGAUGUGGGUG | 79.7 | 55.7 |
| 2470 | 1420 | CCACAUCCUUCACAAACUCCA | 2664 | GAGUUUGUGAAGGAUGUGGGU | 74.1 | 50.1 |
| 2471 | 1421 | CACAUCCUUCACAAACUCCAU | 2665 | GGAGUUUGUGAAGGAUGUGGG | 62 | 38 |
| 2472 | 1422 | ACAUCCUUCACAAACUCCAUG | 2666 | UGGAGUUUGUGAAGGAUGUGG | 80.5 | 56.5 |
| 2473 | 1423 | CAUCCUUCACAAACUCCAUGU | 2667 | AUGGAGUUUGUGAAGGAUGUG | 86.6 | 62.6 |
| 2474 | 1424 | AUCCUUCACAAACUCCAUGUU | 2668 | CAUGGAGUUUGUGAAGGAUGU | 58.6 | 34.6 |
| 2475 | 1425 | UCCUUCACAAACUCCAUGUUU | 2669 | ACAUGGAGUUUGUGAAGGAUG | 66.5 | 42.5 |
| 2476 | 1426 | CCUUCACAAACUCCAUGUUUC | 2670 | AACAUGGAGUUUGUGAAGGAU | 84.7 | 60.7 |
| 2477 | 1427 | CUUCACAAACUCCAUGUUUCA | 2671 | AAACAUGGAGUUUGUGAAGGA | 79.4 | 55.4 |
| 2478 | 1428 | UUCACAAACUCCAUGUUUCAG | 2672 | GAAACAUGGAGUUUGUGAAGG | 62.9 | 38.9 |
| 2479 | 1429 | UCACAAACUCCAUGUUUCAGU | 2673 | UGAAACAUGGAGUUUGUGAAG | 82.2 | 58.2 |
| 2480 | 1430 | CACAAACUCCAUGUUUCAGUG | 2674 | CUGAAACAUGGAGUUUGUGAA | 69.2 | 45.2 |
| 2481 | 1431 | ACAAACUCCAUGUUUCAGUGU | 2675 | ACUGAAACAUGGAGUUUGUGA | 69.6 | 45.6 |
| 2482 | 1432 | CAAACUCCAUGUUUCAGUGUU | 2676 | CACUGAAACAUGGAGUUUGUG | 76.8 | 52.8 |
| 2483 | 1433 | AAACUCCAUGUUUCAGUGUUU | 2677 | ACACUGAAACAUGGAGUUUGU | 70 | 46 |
| 2484 | 1434 | AACUCCAUGUUUCAGUGUUUG | 2678 | AACACUGAAACAUGGAGUUUG | 83.6 | 59.6 |
| 2485 | 1435 | ACUCCAUGUUUCAGUGUUUGA | 2679 | AAACACUGAAACAUGGAGUUU | 89.1 | 65.1 |
| 2486 | 1436 | CUCCAUGUUUCAGUGUUUGAG | 2680 | CAAACACUGAAACAUGGAGUU | 73.6 | 49.6 |
| 2487 | 1437 | UCCAUGUUUCAGUGUUUGAGU | 2681 | UCAAACACUGAAACAUGGAGU | 82.9 | 58.9 |
| 2488 | 1438 | CCAUGUUUCAGUGUUUGAGUC | 2682 | CUCAAACACUGAAACAUGGAG | 75.3 | 51.3 |
| 2489 | 1439 | CAUGUUUCAGUGUUUGAGUCC | 2683 | ACUCAAACACUGAAACAUGGA | 81.8 | 57.8 |
| 2490 | 1440 | AUGUUUCAGUGUUUGAGUCCA | 2684 | GACUCAAACACUGAAACAUGG | 70 | 46 |
| 2491 | 1441 | UGUUUCAGUGUUUGAGUCCAU | 2685 | GGACUCAAACACUGAAACAUG | 62.2 | 38.2 |
| 2492 | 1442 | GUUUCAGUGUUUGAGUCCAUG | 2686 | UGGACUCAAACACUGAAACAU | 86.5 | 62.5 |
| 2493 | 1443 | UUUCAGUGUUUGAGUCCAUGU | 2687 | AUGGACUCAAACACUGAAACA | 72.2 | 48.2 |
| 2494 | 1444 | UUCAGUGUUUGAGUCCAUGUU | 2688 | CAUGGACUCAAACACUGAAAC | 52.2 | 28.2 |
| 2495 | 1445 | UCAGUGUUUGAGUCCAUGUUU | 2689 | ACAUGGACUCAAACACUGAAA | 73.1 | 49.1 |
| 2496 | 1446 | CAGUGUUUGAGUCCAUGUUUA | 2690 | AACAUGGACUCAAACACUGAA | 89.3 | 65.3 |
| 2497 | 1447 | AGUGUUUGAGUCCAUGUUUAU | 2691 | AAACAUGGACUCAAACACUGA | 87.4 | 63.4 |
| 2498 | 1448 | GUGUUUGAGUCCAUGUUUAUU | 2692 | UAAACAUGGACUCAAACACUG | 97.8 | 73.8 |
| 2499 | 1449 | UGUUUGAGUCCAUGUUUAUUC | 2693 | AUAAACAUGGACUCAAACACU | 82.2 | 58.2 |
| 2500 | 1450 | GUUUGAGUCCAUGUUUAUUCU | 2694 | AAUAAACAUGGACUCAAACAC | 75.1 | 50.1 |
| 2501 | 1451 | UUUGAGUCCAUGUUUAUUCUG | 2695 | GAAUAAACAUGGACUCAAACA | 53.2 | 28.2 |
| 2502 | 1452 | UUGAGUCCAUGUUUAUUCUGC | 2696 | AGAAUAAACAUGGACUCAAAC | 61.9 | 36.9 |
| 2503 | 1453 | UGAGUCCAUGUUUAUUCUGCA | 2697 | CAGAAUAAACAUGGACUCAAA | 63.3 | 38.3 |
| 2504 | 1454 | GAGUCCAUGUUUAUUCUGCAA | 2698 | GCAGAAUAAACAUGGACUCAA | 66 | 41 |
| 2505 | 1455 | AGUCCAUGUUUAUUCUGCAAA | 2699 | UGCAGAAUAAACAUGGACUCA | 84.9 | 59.9 |
| 2506 | 1456 | GUCCAUGUUUAUUCUGCAAAU | 2700 | UUGCAGAAUAAACAUGGACUC | 88.8 | 63.8 |
| 2507 | 1457 | UCCAUGUUUAUUCUGCAAAUA | 2701 | UUUGCAGAAUAAACAUGGACU | 75.8 | 50.8 |
| 2508 | 1458 | CCAUGUUUAUUCUGCAAAUAA | 2702 | AUUUGCAGAAUAAACAUGGAC | 85.3 | 60.3 |
| 2509 | 1459 | CAUGUUUAUUCUGCAAAUAAA | 2703 | UAUUUGCAGAAUAAACAUGGA | 93.1 | 68.1 |
| 2510 | 1460 | AUGUUUAUUCUGCAAAUAAAU | 2704 | UUAUUUGCAGAAUAAACAUGG | 89.9 | 64.9 |
| 2511 | 1461 | UGUUUAUUCUGCAAAUAAAUG | 2705 | UUUAUUUGCAGAAUAAACAUG | 97 | 72 |
| 2512 | 1462 | GUUUAUUCUGCAAAUAAAUGG | 2706 | AUUUAUUUGCAGAAUAAACAU | 86.3 | 61.3 |
| 2513 | 1463 | UUUAUUCUGCAAAUAAAUGGU | 2707 | CAUUUAUUUGCAGAAUAAACA | 54.4 | 29.4 |
| 2514 | 1464 | UUAUUCUGCAAAUAAAUGGUA | 2708 | CCAUUUAUUUGCAGAAUAAAC | 45.6 | 20.6 |
| 2515 | 1465 | UAUUCUGCAAAUAAAUGGUAA | 2709 | ACCAUUUAUUUGCAGAAUAAA | 67.8 | 42.8 |
| 2516 | 1466 | AUUCUGCAAAUAAAUGGUAAU | 2710 | UACCAUUUAUUUGCAGAAUAA | 77.9 | 52.9 |
| 2517 | 1467 | UUCUGCAAAUAAAUGGUAAUG | 2711 | UUACCAUUUAUUUGCAGAAUA | 77.6 | 52.6 |
| 2518 | 1468 | UCUGCAAAUAAAUGGUAAUGU | 2712 | AUUACCAUUUAUUUGCAGAAU | 76.4 | 51.4 |
| 2519 | 1469 | CUGCAAAUAAAUGGUAAUGUA | 2713 | CAUUACCAUUUAUUUGCAGAA | 65.2 | 40.2 |
| 2520 | 1470 | UGCAAAUAAAUGGUAAUGUAU | 2714 | ACAUUACCAUUUAUUUGCAGA | 67.1 | 42.1 |
| 2521 | 1471 | GCAAAUAAAUGGUAAUGUAUU | 2715 | UACAUUACCAUUUAUUUGCAG | 100.6 | 75.6 |
| 2522 | 1472 | CAAAUAAAUGGUAAUGUAUUG | 2716 | AUACAUUACCAUUUAUUUGCA | 84.8 | 59.8 |
| 2523 | 1473 | AAAUAAAUGGUAAUGUAUUGG | 2717 | AAUACAUUACCAUUUAUUUGC | 73.1 | 48.1 |
| 2524 | 1474 | AAUAAAUGGUAAUGUAUUGGA | 2718 | CAAUACAUUACCAUUUAUUUG | 75.5 | 50.5 |
| 2525 | 1475 | AUAAAUGGUAAUGUAUUGGA | 2719 | CCAAUACAUUACCAUUUAUUU | 54.7 | 29.7 |
| 2526 | 1476 | UAAAUGGUAAUGUAUUGGA | 2720 | UCCAAUACAUUACCAUUUAUU | 73.6 | 48.6 |

In Table 4, “Pos.” refers to the position of the siRNA in the GPC2 input sequence. In this case, position 3 of SEQ ID NO: 1 corresponds to position 1 of siRNA positions listed in Table 4. “Score” refers to the predicted efficacy calculated from the SDIR 21 bp model, and “Corr. Score”, or Corrected Score refers to the previous efficacy score minored by the penalties from some intrinsic target features that can influence siRNA efficacy. Each row in Table 4 includes a sense region and complementary antisense region of a duplex of a representative siRNA of the disclosure.

In some embodiments, the sense region comprises a sequence selected from the group listed in Table 4. In some embodiments, the anti-sense region comprises a sequence selected from the group listed in Table 4. In some embodiments, the sense and anti-sense regions comprise complementary sequences selected from the group listed in Table 4.

In some embodiments, the siRNA comprises an RNA duplex that is 19 nucleotides in length. In some embodiments, the RNA duplex comprises a sense region and an antisense region that are selected from the group of sequences in Table 5.

TABLE 5
siRNA sense regions and anti-sense regions, 19 bp in length

| | SEQ | | SEQ | | | |
|---|---|---|---|---|---|---|
| | ID | | ID | | | Corr. |
| Pos. | NO: | Sense Region | NO: | Antisense Region | Score | Score |
| 1 | 2721 | GCUCCCAUUGUCUCGGCAG | 4057 | CUGCCGAGACAAUGGGAGC | 66.7 | 66.7 |
| 2 | 2722 | CUCCCAUUGUCUCGGCAGA | 4058 | UCUGCCGAGACAAUGGGAG | 77.3 | 77.3 |
| 3 | 2723 | UCCCAUUGUCUCGGCAGAU | 4059 | AUCUGCCGAGACAAUGGGA | 65 | 65 |
| 4 | 2724 | CCCAUUGUCUCGGCAGAUG | 4060 | CAUCUGCCGAGACAAUGGG | 66.2 | 66.2 |
| 5 | 2725 | CCAUUGUCUCGGCAGAUGC | 4061 | GCAUCUGCCGAGACAAUGG | 65.2 | 65.2 |
| 6 | 2726 | CAUUGUCUCGGCAGAUGCC | 4062 | GGCAUCUGCCGAGACAAUG | 61.9 | 61.9 |
| 7 | 2727 | AUUGUCUCGGCAGAUGCCG | 4063 | CGGCAUCUGCCGAGACAAU | 46.6 | 46.6 |
| 8 | 2728 | UUGUCUCGGCAGAUGCCGC | 4064 | GCGGCAUCUGCCGAGACAA | 43.7 | 43.7 |
| 9 | 2729 | UGUCUCGGCAGAUGCCGCC | 4065 | GGCGGCAUCUGCCGAGACA | 42.8 | 42.8 |
| 10 | 2730 | GUCUCGGCAGAUGCCGCCU | 4066 | AGGCGGCAUCUGCCGAGAC | 61.5 | 61.5 |
| 11 | 2731 | UCUCGGCAGAUGCCGCCUG | 4067 | CAGGCGGCAUCUGCCGAGA | 49.7 | 49.7 |
| 12 | 2732 | CUCGGCAGAUGCCGCCUGG | 4068 | CCAGGCGGCAUCUGCCGAG | 52.4 | 52.4 |
| 13 | 2733 | UCGGCAGAUGCCGCCUGGU | 4069 | ACCAGGCGGCAUCUGCCGA | 51 | 51 |
| 14 | 2734 | CGGCAGAUGCCGCCUGGUC | 4070 | GACCAGGCGGCAUCUGCCG | 60.6 | 60.6 |
| 15 | 2735 | GGCAGAUGCCGCCUGGUCC | 4071 | GGACCAGGCGGCAUCUGCC | 60 | 60 |
| 16 | 2736 | GCAGAUGCCGCCUGGUCCA | 4072 | UGGACCAGGCGGCAUCUGC | 79.3 | 79.3 |
| 17 | 2737 | CAGAUGCCGCCUGGUCCAG | 4073 | CUGGACCAGGCGGCAUCUG | 58.4 | 58.4 |
| 18 | 2738 | AGAUGCCGCCUGGUCCAGC | 4074 | GCUGGACCAGGCGGCAUCU | 45 | 45 |
| 19 | 2739 | GAUGCCGCCUGGUCCAGCU | 4075 | AGCUGGACCAGGCGGCAUC | 66.8 | 66.8 |
| 20 | 2740 | AUGCCGCCUGGUCCAGCUA | 4076 | UAGCUGGACCAGGCGGCAU | 70.7 | 70.7 |
| 21 | 2741 | UGCCGCCUGGUCCAGCUAU | 4077 | AUAGCUGGACCAGGCGGCA | 64.7 | 64.7 |
| 22 | 2742 | GCCGCCUGGUCCAGCUAUC | 4078 | GAUAGCUGGACCAGGCGGC | 63.7 | 63.7 |
| 23 | 2743 | CCGCCUGGUCCAGCUAUCG | 4079 | CGAUAGCUGGACCAGGCGG | 57.4 | 57.4 |
| 24 | 2744 | CGCCUGGUCCAGCUAUCGU | 4080 | ACGAUAGCUGGACCAGGCG | 69.8 | 69.8 |
| 25 | 2745 | GCCUGGUCCAGCUAUCGUG | 4081 | CACGAUAGCUGGACCAGGC | 68.9 | 68.9 |
| 26 | 2746 | CCUGGUCCAGCUAUCGUGC | 4082 | GCACGAUAGCUGGACCAGG | 60.3 | 60.3 |
| 27 | 2747 | CUGGUCCAGCUAUCGUGCU | 4083 | AGCACGAUAGCUGGACCAG | 65.2 | 65.2 |
| 28 | 2748 | UGGUCCAGCUAUCGUGCUC | 4084 | GAGCACGAUAGCUGGACCA | 59.9 | 59.9 |
| 29 | 2749 | GGUCCAGCUAUCGUGCUCG | 4085 | CGAGCACGAUAGCUGGACC | 63.3 | 63.3 |
| 30 | 2750 | GUCCAGCUAUCGUGCUCGG | 4086 | CCGAGCACGAUAGCUGGAC | 64.1 | 64.1 |
| 31 | 2751 | UCCAGCUAUCGUGCUCGGU | 4087 | ACCGAGCACGAUAGCUGGA | 55.9 | 55.9 |
| 32 | 2752 | CCAGCUAUCGUGCUCGGUA | 4088 | UACCGAGCACGAUAGCUGG | 82.1 | 82.1 |
| 33 | 2753 | CAGCUAUCGUGCUCGGUAU | 4089 | AUACCGAGCACGAUAGCUG | 84.6 | 84.6 |
| 34 | 2754 | AGCUAUCGUGCUCGGUAUU | 4090 | AAUACCGAGCACGAUAGCU | 77.8 | 77.8 |
| 35 | 2755 | GCUAUCGUGCUCGGUAUUC | 4091 | GAAUACCGAGCACGAUAGC | 63 | 63 |
| 36 | 2756 | CUAUCGUGCUCGGUAUUCA | 4092 | UGAAUACCGAGCACGAUAG | 78.5 | 78.5 |
| 37 | 2757 | UAUCGUGCUCGGUAUUCAG | 4093 | CUGAAUACCGAGCACGAUA | 63 | 63 |
| 38 | 2758 | AUCGUGCUCGGUAUUCAGU | 4094 | ACUGAAUACCGAGCACGAU | 61.9 | 61.9 |
| 39 | 2759 | UCGUGCUCGGUAUUCAGUU | 4095 | AACUGAAUACCGAGCACGA | 66.8 | 66.8 |
| 40 | 2760 | CGUGCUCGGUAUUCAGUUU | 4096 | AAACUGAAUACCGAGCACG | 87.3 | 87.3 |
| 57 | 2761 | UUUCCGGAGCAGCGCUCUU | 4097 | AAGAGCGCUGCUCCGGAAA | 63.6 | 63.6 |
| 58 | 2762 | UUCCGGAGCAGCGCUCUUU | 4098 | AAAGAGCGCUGCUCCGGAA | 60.2 | 60.2 |
| 59 | 2763 | UCCGGAGCAGCGCUCUUUC | 4099 | GAAAGAGCGCUGCUCCGGA | 55.6 | 55.6 |
| 60 | 2764 | CCGGAGCAGCGCUCUUUCU | 4100 | AGAAAGAGCGCUGCUCCGG | 74.7 | 74.7 |
| 61 | 2765 | CGGAGCAGCGCUCUUUCUC | 4101 | GAGAAAGAGCGCUGCUCCG | 64.3 | 64.3 |
| 62 | 2766 | GGAGCAGCGCUCUUUCUCU | 4102 | AGAGAAAGAGCGCUGCUCC | 78.6 | 78.6 |
| 63 | 2767 | GAGCAGCGCUCUUUCUCUG | 4103 | CAGAGAAAGAGCGCUGCUC | 71.7 | 71.7 |
| 64 | 2768 | AGCAGCGCUCUUUCUCUGG | 4104 | CCAGAGAAAGAGCGCUGCU | 58.5 | 58.5 |
| 81 | 2769 | GGCCCGCGGAGCGGUCCCG | 4105 | CGGGACCGCUCCGCGGGCC | 51.7 | 51.7 |
| 82 | 2770 | GCCCGCGGAGCGGUCCCGC | 4106 | GCGGGACCGCUCCGCGGGC | 43.5 | 43.5 |
| 83 | 2771 | CCCGCGGAGCGGUCCCGCG | 4107 | CGCGGGACCGCUCCGCGGG | 53.5 | 53.5 |
| 84 | 2772 | CCGCGGAGCGGUCCCGCGG | 4108 | CCGCGGGACCGCUCCGCGG | 55.6 | 55.6 |
| 85 | 2773 | CGCGGAGCGGUCCCGCGGC | 4109 | GCCGCGGGACCGCUCCGCG | 52.1 | 52.1 |
| 86 | 2774 | GCGGAGCGGUCCCGCGGCC | 4110 | GGCCGCGGGACCGCUCCGC | 53.1 | 53.1 |
| 87 | 2775 | CGGAGCGGUCCCGCGGCCG | 4111 | CGGCCGCGGGACCGCUCCG | 49.3 | 49.3 |
| 88 | 2776 | GGAGCGGUCCCGCGGCCGA | 4112 | UCGGCCGCGGGACCGCUCC | 77.5 | 77.5 |
| 89 | 2777 | GAGCGGUCCCGCGGCCGAG | 4113 | CUCGGCCGCGGGACCGCUC | 60.3 | 60.3 |
| 90 | 2778 | AGCGGUCCCGCGGCCGAGU | 4114 | ACUCGGCCGCGGGACCGCU | 59.7 | 59.7 |
| 91 | 2779 | GCGGUCCCGCGGCCGAGUA | 4115 | UACUCGGCCGCGGGACCGC | 77 | 77 |
| 92 | 2780 | CGGUCCCGCGGCCGAGUAC | 4116 | GUACUCGGCCGCGGGACCG | 62.2 | 62.2 |
| 93 | 2781 | GGUCCCGCGGCCGAGUACC | 4117 | GGUACUCGGCCGCGGGACC | 53.9 | 53.9 |
| 94 | 2782 | GUCCCGCGGCCGAGUACCG | 4118 | CGGUACUCGGCCGCGGGAC | 55.3 | 55.3 |
| 95 | 2783 | UCCCGCGGCCGAGUACCGG | 4119 | CCGGUACUCGGCCGCGGGA | 39.4 | 39.4 |
| 96 | 2784 | CCCGCGGCCGAGUACCGGA | 4120 | UCCGGUACUCGGCCGCGGG | 71.2 | 71.2 |
| 97 | 2785 | CCGCGGCCGAGUACCGGAU | 4121 | AUCCGGUACUCGGCCGCGG | 72.5 | 72.5 |
| 98 | 2786 | CGCGGCCGAGUACCGGAUU | 4122 | AAUCCGGUACUCGGCCGCG | 73.3 | 73.3 |
| 99 | 2787 | GCGGCCGAGUACCGGAUUC | 4123 | GAAUCCGGUACUCGGCCGC | 66.5 | 66.5 |
| 100 | 2788 | CGGCCGAGUACCGGAUUCC | 4124 | GGAAUCCGGUACUCGGCCG | 58.4 | 57.4 |
| 101 | 2789 | GGCCGAGUACCGGAUUCCC | 4125 | GGGAAUCCGGUACUCGGCC | 61.5 | 60.5 |
| 102 | 2790 | GCCGAGUACCGGAUUCCCG | 4126 | CGGGAAUCCGGUACUCGGC | 64.4 | 63.4 |
| 103 | 2791 | CCGAGUACCGGAUUCCCGA | 4127 | UCGGGAAUCCGGUACUCGG | 82.3 | 81.3 |
| 104 | 2792 | CGAGUACCGGAUUCCCGAG | 4128 | CUCGGGAAUCCGGUACUCG | 72.3 | 71.3 |
| 105 | 2793 | GAGUACCGGAUUCCCGAGU | 4129 | ACUCGGGAAUCCGGUACUC | 69.7 | 68.7 |
| 106 | 2794 | AGUACCGGAUUCCCGAGUU | 4130 | AACUCGGGAAUCCGGUACU | 76.4 | 75.4 |
| 107 | 2795 | GUACCGGAUUCCCGAGUUU | 4131 | AAACUCGGGAAUCCGGUAC | 82.4 | 81.4 |
| 108 | 2796 | UACCGGAUUCCCGAGUUUG | 4132 | CAAACUCGGGAAUCCGGUA | 60.4 | 59.4 |
| 109 | 2797 | ACCGGAUUCCCGAGUUUGG | 4133 | CCAAACUCGGGAAUCCGGU | 63.2 | 62.2 |
| 110 | 2798 | CCGGAUUCCCGAGUUUGGG | 4134 | CCCAAACUCGGGAAUCCGG | 57.2 | 56.2 |
| 111 | 2799 | CGGAUUCCCGAGUUUGGGA | 4135 | UCCCAAACUCGGGAAUCCG | 77.8 | 76.8 |
| 112 | 2800 | GGAUUCCCGAGUUUGGGAG | 4136 | CUCCCAAACUCGGGAAUCC | 66.1 | 65.1 |
| 113 | 2801 | GAUUCCCGAGUUUGGGAGG | 4137 | CCUCCCAAACUCGGGAAUC | 59.9 | 58.9 |
| 114 | 2802 | AUUCCCGAGUUUGGGAGGC | 4138 | GCCUCCCAAACUCGGGAAU | 39.9 | 38.9 |
| 115 | 2803 | UUCCCGAGUUUGGGAGGCU | 4139 | AGCCUCCCAAACUCGGGAA | 52.9 | 51.9 |
| 116 | 2804 | UCCCGAGUUUGGGAGGCUC | 4140 | GAGCCUCCCAAACUCGGGA | 53.8 | 52.8 |
| 117 | 2805 | CCCGAGUUUGGGAGGCUCU | 4141 | AGAGCCUCCCAAACUCGGG | 73 | 72 |
| 118 | 2806 | CCGAGUUUGGGAGGCUCUG | 4142 | CAGAGCCUCCCAAACUCGG | 65.8 | 64.8 |
| 119 | 2807 | CGAGUUUGGGAGGCUCUGC | 4143 | GCAGAGCCUCCCAAACUCG | 60 | 59 |
| 120 | 2808 | GAGUUUGGGAGGCUCUGCU | 4144 | AGCAGAGCCUCCCAAACUC | 69.7 | 68.7 |
| 121 | 2809 | AGUUUGGGAGGCUCUGCUU | 4145 | AAGCAGAGCCUCCCAAACU | 76.8 | 75.8 |
| 122 | 2810 | GUUUGGGAGGCUCUGCUUU | 4146 | AAAGCAGAGCCUCCCAAAC | 74.8 | 73.8 |
| 123 | 2811 | UUUGGGAGGCUCUGCUUUC | 4147 | GAAAGCAGAGCCUCCCAAA | 50.8 | 49.8 |
| 124 | 2812 | UUGGGAGGCUCUGCUUUCC | 4148 | GGAAAGCAGAGCCUCCCAA | 44.3 | 43.3 |
| 125 | 2813 | UGGGAGGCUCUGCUUUCCU | 4149 | AGGAAAGCAGAGCCUCCCA | 61.3 | 60.3 |
| 126 | 2814 | GGGAGGCUCUGCUUUCCUC | 4150 | GAGGAAAGCAGAGCCUCCC | 68.6 | 67.6 |
| 127 | 2815 | GGAGGCUCUGCUUUCCUCC | 4151 | GGAGGAAAGCAGAGCCUCC | 61.6 | 60.6 |
| 128 | 2816 | GAGGCUCUGCUUUCCUCCU | 4152 | AGGAGGAAAGCAGAGCCUC | 73.5 | 72.5 |
| 129 | 2817 | AGGCUCUGCUUUCCUCCUU | 4153 | AAGGAGGAAAGCAGAGCCU | 74 | 73 |
| 130 | 2818 | GGCUCUGCUUUCCUCCUUA | 4154 | UAAGGAGGAAAGCAGAGCC | 92.8 | 91.8 |
| 131 | 2819 | GCUCUGCUUUCCUCCUUAG | 4155 | CUAAGGAGGAAAGCAGAGC | 78.7 | 77.7 |
| 132 | 2820 | CUCUGCUUUCCUCCUUAGG | 4156 | CCUAAGGAGGAAAGCAGAG | 59.6 | 58.6 |
| 133 | 2821 | UCUGCUUUCCUCCUUAGGA | 4157 | UCCUAAGGAGGAAAGCAGA | 69.4 | 68.4 |
| 134 | 2822 | CUGCUUUCCUCCUUAGGAC | 4158 | GUCCUAAGGAGGAAAGCAG | 64.4 | 63.4 |
| 135 | 2823 | UGCUUUCCUCCUUAGGACC | 4159 | GGUCCUAAGGAGGAAAGCA | 55.9 | 54.9 |
| 136 | 2824 | GCUUUCCUCCUUAGGACCC | 4160 | GGGUCCUAAGGAGGAAAGC | 60 | 59 |
| 137 | 2825 | CUUUCCUCCUUAGGACCCA | 4161 | UGGGUCCUAAGGAGGAAAG | 72.1 | 71.1 |
| 138 | 2826 | UUUCCUCCUUAGGACCCAC | 4162 | GUGGGUCCUAAGGAGGAAA | 50.3 | 49.3 |
| 139 | 2827 | UUCCUCCUUAGGACCCACU | 4163 | AGUGGGUCCUAAGGAGGAA | 54 | 53 |
| 140 | 2828 | UCCUCCUUAGGACCCACUU | 4164 | AAGUGGGUCCUAAGGAGGA | 67.1 | 66.1 |
| 141 | 2829 | CCUCCUUAGGACCCACUUU | 4165 | AAAGUGGGUCCUAAGGAGG | 79.3 | 78.3 |
| 142 | 2830 | CUCCUUAGGACCCACUUUG | 4166 | CAAAGUGGGUCCUAAGGAG | 64.6 | 63.6 |
| 143 | 2831 | UCCUUAGGACCCACUUUGC | 4167 | GCAAAGUGGGUCCUAAGGA | 54.2 | 53.2 |
| 144 | 2832 | CCUUAGGACCCACUUUGCC | 4168 | GGCAAAGUGGGUCCUAAGG | 56.6 | 55.6 |
| 145 | 2833 | CUUAGGACCCACUUUGCCG | 4169 | CGGCAAAGUGGGUCCUAAG | 56.1 | 55.1 |
| 146 | 2834 | UUAGGACCCACUUUGCCGU | 4170 | ACGGCAAAGUGGGUCCUAA | 51.4 | 50.4 |
| 147 | 2835 | UAGGACCCACUUUGCCGUC | 4171 | GACGGCAAAGUGGGUCCUA | 45.6 | 44.6 |
| 148 | 2836 | AGGACCCACUUUGCCGUCC | 4172 | GGACGGCAAAGUGGGUCCU | 46.8 | 45.8 |
| 149 | 2837 | GGACCCACUUUGCCGUCCU | 4173 | AGGACGGCAAAGUGGGUCC | 71.8 | 70.8 |
| 150 | 2838 | GACCCACUUUGCCGUCCUG | 4174 | CAGGACGGCAAAGUGGGUC | 66.3 | 65.3 |
| 151 | 2839 | ACCCACUUUGCCGUCCUGG | 4175 | CCAGGACGGCAAAGUGGGU | 43.4 | 42.4 |
| 152 | 2840 | CCCACUUUGCCGUCCUGGG | 4176 | CCCAGGACGGCAAAGUGGG | 52.2 | 51.2 |
| 173 | 2841 | GGCUGCAGUUAUGUCCGCG | 4177 | CGCGGACAUAACUGCAGCC | 64.5 | 63.5 |
| 174 | 2842 | GCUGCAGUUAUGUCCGCGC | 4178 | GCGCGGACAUAACUGCAGC | 63.3 | 62.3 |
| 175 | 2843 | CUGCAGUUAUGUCCGCGCU | 4179 | AGCGCGGACAUAACUGCAG | 73.6 | 72.6 |
| 176 | 2844 | UGCAGUUAUGUCCGCGCUG | 4180 | CAGCGCGGACAUAACUGCA | 63.9 | 62.9 |
| 177 | 2845 | GCAGUUAUGUCCGCGCUGC | 4181 | GCAGCGCGGACAUAACUGC | 64.7 | 63.7 |
| 178 | 2846 | CAGUUAUGUCCGCGCUGCG | 4182 | CGCAGCGCGGACAUAACUG | 65.1 | 64.1 |
| 179 | 2847 | AGUUAUGUCCGCGCUGCGA | 4183 | UCGCAGCGCGGACAUAACU | 80.4 | 79.4 |
| 180 | 2848 | GUUAUGUCCGCGCUGCGAC | 4184 | GUCGCAGCGCGGACAUAAC | 64.7 | 63.7 |
| 181 | 2849 | UUAUGUCCGCGCUGCGACC | 4185 | GGUCGCAGCGCGGACAUAA | 50 | 49 |
| 182 | 2850 | UAUGUCCGCGCUGCGACCU | 4186 | AGGUCGCAGCGCGGACAUA | 55.1 | 54.1 |
| 183 | 2851 | AUGUCCGCGCUGCGACCUC | 4187 | GAGGUCGCAGCGCGGACAU | 53.9 | 52.9 |
| 184 | 2852 | UGUCCGCGCUGCGACCUCU | 4188 | AGAGGUCGCAGCGCGGACA | 67.7 | 66.7 |
| 185 | 2853 | GUCCGCGCUGCGACCUCUC | 4189 | GAGAGGUCGCAGCGCGGAC | 62.1 | 61.1 |
| 186 | 2854 | UCCGCGCUGCGACCUCUCC | 4190 | GGAGAGGUCGCAGCGCGGA | 50.6 | 49.6 |
| 187 | 2855 | CCGCGCUGCGACCUCUCCU | 4191 | AGGAGAGGUCGCAGCGCGG | 68.7 | 67.7 |
| 188 | 2856 | CGCGCUGCGACCUCUCCUG | 4192 | CAGGAGAGGUCGCAGCGCG | 68.7 | 67.7 |
| 189 | 2857 | GCGCUGCGACCUCUCCUGC | 4193 | GCAGGAGAGGUCGCAGCGC | 62.6 | 61.6 |
| 190 | 2858 | CGCUGCGACCUCUCCUGCU | 4194 | AGCAGGAGAGGUCGCAGCG | 74.8 | 73.8 |
| 191 | 2859 | GCUGCGACCUCUCCUGCUU | 4195 | AAGCAGGAGAGGUCGCAGC | 86.7 | 85.7 |
| 192 | 2860 | CUGCGACCUCUCCUGCUUC | 4196 | GAAGCAGGAGAGGUCGCAG | 65.2 | 64.2 |
| 193 | 2861 | UGCGACCUCUCCUGCUUCU | 4197 | AGAAGCAGGAGAGGUCGCA | 71 | 70 |
| 194 | 2862 | GCGACCUCUCCUGCUUCUG | 4198 | CAGAAGCAGGAGAGGUCGC | 68.9 | 67.9 |
| 195 | 2863 | CGACCUCUCCUGCUUCUGC | 4199 | GCAGAAGCAGGAGAGGUCG | 65.4 | 64.4 |
| 196 | 2864 | GACCUCUCCUGCUUCUGCU | 4200 | AGCAGAAGCAGGAGAGGUC | 74.3 | 73.3 |
| 197 | 2865 | ACCUCUCCUGCUUCUGCUG | 4201 | CAGCAGAAGCAGGAGAGGU | 64.2 | 63.2 |
| 198 | 2866 | CCUCUCCUGCUUCUGCUGC | 4202 | GCAGCAGAAGCAGGAGAGG | 56 | 55 |
| 199 | 2867 | CUCUCCUGCUUCUGCUGCU | 4203 | AGCAGCAGAAGCAGGAGAG | 70.4 | 69.4 |
| 200 | 2868 | UCUCCUGCUUCUGCUGCUG | 4204 | CAGCAGCAGAAGCAGGAGA | 59.6 | 57.6 |
| 201 | 2869 | CUCCUGCUUCUGCUGCUGC | 4205 | GCAGCAGCAGAAGCAGGAG | 55.8 | 53.8 |
| 202 | 2870 | UCCUGCUUCUGCUGCUGCC | 4206 | GGCAGCAGCAGAAGCAGGA | 51.3 | 49.3 |
| 203 | 2871 | CCUGCUUCUGCUGCUGCCU | 4207 | AGGCAGCAGCAGAAGCAGG | 68.8 | 66.8 |
| 204 | 2872 | CUGCUUCUGCUGCUGCCUC | 4208 | GAGGCAGCAGCAGAAGCAG | 58.8 | 56.8 |
| 205 | 2873 | UGCUUCUGCUGCUGCCUCU | 4209 | AGAGGCAGCAGCAGAAGCA | 68.6 | 66.6 |
| 206 | 2874 | GCUUCUGCUGCUGCCUCUG | 4210 | CAGAGGCAGCAGCAGAAGC | 68.9 | 66.9 |
| 207 | 2875 | CUUCUGCUGCUGCCUCUGU | 4211 | ACAGAGGCAGCAGCAGAAG | 68.9 | 66.9 |
| 208 | 2876 | UUCUGCUGCUGCCUCUGUG | 4212 | CACAGAGGCAGCAGCAGAA | 53.8 | 51.8 |
| 209 | 2877 | UCUGCUGCUGCCUCUGUGU | 4213 | ACACAGAGGCAGCAGCAGA | 65.6 | 63.6 |
| 210 | 2878 | CUGCUGCUGCCUCUGUGUC | 4214 | GACACAGAGGCAGCAGCAG | 59.7 | 57.7 |
| 211 | 2879 | UGCUGCUGCCUCUGUGUCC | 4215 | GGACACAGAGGCAGCAGCA | 54.8 | 52.8 |
| 212 | 2880 | GCUGCUGCCUCUGUGUCCC | 4216 | GGGACACAGAGGCAGCAGC | 60.7 | 58.7 |
| 213 | 2881 | CUGCUGCCUCUGUGUCCCG | 4217 | CGGGACACAGAGGCAGCAG | 57.1 | 55.1 |
| 214 | 2882 | UGCUGCCUCUGUGUCCCGG | 4218 | CCGGGACACAGAGGCAGCA | 49.3 | 47.3 |
| 215 | 2883 | GCUGCCUCUGUGUCCCGGU | 4219 | ACCGGGACACAGAGGCAGC | 65.6 | 66.6 |
| 216 | 2884 | CUGCCUCUGUGUCCCGGUC | 4220 | GACCGGGACACAGAGGCAG | 60 | 58 |
| 217 | 2885 | UGCCUCUGUGUCCCGGUCC | 4221 | GGACCGGGACACAGAGGCA | 48.2 | 46.2 |
| 218 | 2886 | GCCUCUGUGUCCCGGUCCU | 4222 | AGGACCGGGACACAGAGGC | 76.7 | 74.7 |
| 219 | 2887 | CCUCUGUGUCCCGGUCCUG | 4223 | CAGGACCGGGACACAGAGG | 64.8 | 62.8 |
| 220 | 2888 | CUCUGUGUCCCGGUCCUGG | 4224 | CCAGGACCGGGACACAGAG | 55.8 | 53.8 |
| 221 | 2889 | UCUGUGUCCCGGUCCUGGU | 4225 | ACCAGGACCGGGACACAGA | 63.4 | 61.4 |
| 222 | 2890 | CUGUGUCCCGGUCCUGGUC | 4226 | GACCAGGACCGGGACACAG | 61 | 59 |
| 223 | 2891 | UGUGUCCCGGUCCUGGUCC | 4227 | GGACCAGGACCGGGACACA | 47.5 | 45.5 |
| 224 | 2892 | GUGUCCCGGUCCUGGUCCC | 4228 | GGGACCAGGACCGGGACAC | 56 | 54 |
| 225 | 2893 | UGUCCCGGUCCUGGUCCCG | 4229 | CGGGACCAGGACCGGGACA | 46 | 44 |
| 226 | 2894 | GUCCCGGUCCUGGUCCCGG | 4230 | CCGGGACCAGGACCGGGAC | 49.5 | 47.5 |
| 227 | 2895 | UCCCGGUCCUGGUCCCGGA | 4231 | UCCGGGACCAGGACCGGGA | 67.1 | 65.1 |
| 228 | 2896 | CCCGGUCCUGGUCCCGGAC | 4232 | GUCCGGGACCAGGACCGGG | 59.4 | 57.4 |
| 229 | 2897 | CCGGUCCUGGUCCCGGACC | 4233 | GGUCCGGGACCAGGACCGG | 50.3 | 48.3 |
| 230 | 2898 | CGGUCCUGGUCCCGGACCC | 4234 | GGGUCCGGGACCAGGACCG | 58 | 56 |
| 231 | 2899 | GGUCCUGGUCCCGGACCCG | 4235 | CGGGUCCGGGACCAGGACC | 60.4 | 58.4 |
| 232 | 2900 | GUCCUGGUCCCGGACCCGG | 4236 | CCGGGUCCGGGACCAGGAC | 51.5 | 49.5 |
| 233 | 2901 | UCCUGGUCCCGGACCCGGG | 4237 | CCCGGGUCCGGGACCAGGA | 45.8 | 43.8 |
| 234 | 2902 | CCUGGUCCCGGACCCGGGA | 4238 | UCCCGGGUCCGGGACCAGG | 69.8 | 67.8 |
| 235 | 2903 | CUGGUCCCGGACCCGGGAG | 4239 | CUCCCGGGUCCGGGACCAG | 49.4 | 47.4 |
| 236 | 2904 | UGGUCCCGGACCCGGGAGC | 4240 | GCUCCCGGGUCCGGGACCA | 39.2 | 37.2 |
| 237 | 2905 | GGUCCCGGACCCGGGAGCG | 4241 | CGCUCCCGGGUCCGGGACC | 52 | 50 |
| 238 | 2906 | GUCCCGGACCCGGGAGCGA | 4242 | UCGCUCCCGGGUCCGGGAC | 68.7 | 66.7 |
| 239 | 2907 | UCCCGGACCCGGGAGCGAG | 4243 | CUCGCUCCCGGGUCCGGGA | 49.9 | 47.9 |
| 240 | 2908 | CCCGGACCCGGGAGCGAGG | 4244 | CCUCGCUCCCGGGUCCGGG | 56.2 | 54.2 |
| 241 | 2909 | CCGGACCCGGGAGCGAGGC | 4245 | GCCUCGCUCCCGGGUCCGG | 43.7 | 41.7 |
| 242 | 2910 | CGGACCCGGGAGCGAGGCA | 4246 | UGCCUCGCUCCCGGGUCCG | 70.6 | 68.6 |
| 243 | 2911 | GGACCCGGGAGCGAGGCAA | 4247 | UUGCCUCGCUCCCGGGUCC | 80.4 | 78.4 |
| 244 | 2912 | GACCCGGGAGCGAGGCAAA | 4248 | UUUGCCUCGCUCCCGGGUC | 85.2 | 83.2 |
| 245 | 2913 | ACCCGGGAGCGAGGCAAAG | 4249 | CUUUGCCUCGCUCCCGGGU | 58.6 | 56.6 |
| 246 | 2914 | CCCGGGAGCGAGGCAAAGG | 4250 | CCUUUGCCUCGCUCCCGGG | 55.6 | 53.6 |
| 247 | 2915 | CCGGGAGCGAGGCAAAGGU | 4251 | ACCUUUGCCUCGCUCCCGG | 68.9 | 66.9 |
| 248 | 2916 | CGGGAGCGAGGCAAAGGUC | 4252 | GACCUUUGCCUCGCUCCCG | 67.7 | 65.7 |
| 249 | 2917 | GGGAGCGAGGCAAAGGUCA | 4253 | UGACCUUUGCCUCGCUCCC | 84.3 | 82.3 |
| 250 | 2918 | GGAGCGAGGCAAAGGUCAC | 4254 | GUGACCUUUGCCUCGCUCC | 76.6 | 74.6 |
| 251 | 2919 | GAGCGAGGCAAAGGUCACC | 4255 | GGUGACCUUUGCCUCGCUC | 60.4 | 58.4 |
| 252 | 2920 | AGCGAGGCAAAGGUCACCC | 4256 | GGGUGACCUUUGCCUCGCU | 52.5 | 50.5 |
| 253 | 2921 | GCGAGGCAAAGGUCACCCG | 4257 | CGGGUGACCUUUGCCUCGC | 66.5 | 64.5 |
| 254 | 2922 | CGAGGCAAAGGUCACCCGG | 4258 | CCGGGUGACCUUUGCCUCG | 64.1 | 62.1 |
| 255 | 2923 | GAGGCAAAGGUCACCCGGA | 4259 | UCCGGGUGACCUUUGCCUC | 78.1 | 76.1 |
| 256 | 2924 | AGGCAAAGGUCACCCGGAG | 4260 | CUCCGGGUGACCUUUGCCU | 65.4 | 63.4 |
| 257 | 2925 | GGCAAAGGUCACCCGGAGU | 4261 | ACUCCGGGUGACCUUUGCC | 75.8 | 73.8 |
| 258 | 2926 | GCAAAGGUCACCCGGAGUU | 4262 | AACUCCGGGUGACCUUUGC | 79.5 | 77.5 |
| 259 | 2927 | CAAAGGUCACCCGGAGUUG | 4263 | CAACUCCGGGUGACCUUUG | 69.5 | 67.5 |
| 260 | 2928 | AAAGGUCACCCGGAGUUGU | 4264 | ACAACUCCGGGUGACCUUU | 68.3 | 66.3 |
| 261 | 2929 | AAGGUCACCCGGAGUUGUG | 4265 | CACAACUCCGGGUGACCUU | 62.2 | 60.2 |
| 262 | 2930 | AGGUCACCCGGAGUUGUGC | 4266 | GCACAACUCCGGGUGACCU | 56.9 | 54.9 |
| 263 | 2931 | GGUCACCCGGAGUUGUGCA | 4267 | UGCACAACUCCGGGUGACC | 82.6 | 80.6 |
| 264 | 2932 | GUCACCCGGAGUUGUGCAG | 4268 | CUGCACAACUCCGGGUGAC | 65.2 | 63.2 |
| 265 | 2933 | UCACCCGGAGUUGUGCAGA | 4269 | UCUGCACAACUCCGGGUGA | 66.2 | 64.2 |
| 266 | 2934 | CACCCGGAGUUGUGCAGAG | 4270 | CUCUGCACAACUCCGGGUG | 72.2 | 70.2 |
| 267 | 2935 | ACCCGGAGUUGUGCAGAGA | 4271 | UCUCUGCACAACUCCGGGU | 80.3 | 78.3 |
| 268 | 2936 | CCCGGAGUUGUGCAGAGAC | 4272 | GUCUCUGCACAACUCCGGG | 71 | 69 |
| 269 | 2937 | CCGGAGUUGUGCAGAGACC | 4273 | GGUCUCUGCACAACUCCGG | 71.6 | 69.6 |
| 270 | 2938 | CGGAGUUGUGCAGAGACCC | 4274 | GGGUCUCUGCACAACUCCG | 67.4 | 65.4 |
| 271 | 2939 | GGAGUUGUGCAGAGACCCG | 4275 | CGGGUCUCUGCACAACUCC | 75.5 | 73.5 |
| 272 | 2940 | GAGUUGUGCAGAGACCCGG | 4276 | CCGGGUCUCUGCACAACUC | 66.7 | 64.7 |
| 273 | 2941 | AGUUGUGCAGAGACCCGGC | 4277 | GCCGGGUCUCUGCACAACU | 63 | 61 |
| 274 | 2942 | GUUGUGCAGAGACCCGGCA | 4278 | UGCCGGGUCUCUGCACAAC | 78.6 | 76.6 |
| 275 | 2943 | UUGUGCAGAGACCCGGCAG | 4279 | CUGCCGGGUCUCUGCACAA | 55.4 | 53.4 |
| 276 | 2944 | UGUGCAGAGACCCGGCAGG | 4280 | CCUGCCGGGUCUCUGCACA | 55 | 53 |
| 277 | 2945 | GUGCAGAGACCCGGCAGGU | 4281 | ACCUGCCGGGUCUCUGCAC | 66.3 | 64.3 |
| 278 | 2946 | UGCAGAGACCCGGCAGGUG | 4282 | CACCUGCCGGGUCUCUGCA | 56.7 | 54.7 |
| 279 | 2947 | GCAGAGACCCGGCAGGUGC | 4283 | GCACCUGCCGGGUCUCUGC | 64.8 | 62.8 |
| 280 | 2948 | CAGAGACCCGGCAGGUGCU | 4284 | AGCACCUGCCGGGUCUCUG | 72.8 | 70.8 |
| 281 | 2949 | AGAGACCCGGCAGGUGCUG | 4285 | CAGCACCUGCCGGGUCUCU | 57.8 | 55.8 |
| 282 | 2950 | GAGACCCGGCAGGUGCUGG | 4286 | CCAGCACCUGCCGGGUCUC | 53.6 | 51.6 |
| 283 | 2951 | AGACCCGGCAGGUGCUGGG | 4287 | CCCAGCACCUGCCGGGUCU | 50.9 | 48.9 |
| 308 | 2952 | GGGAUAUAGCUUAAACCUA | 4288 | UAGGUUUAAGCUAUAUCCC | 94.8 | 91.8 |
| 309 | 2953 | GGAUAUAGCUUAAACCUAA | 4289 | UUAGGUUUAAGCUAUAUCC | 103 | 100.3 |
| 310 | 2954 | GAUAUAGCUUAAACCUAAU | 4290 | AUUAGGUUUAAGCUAUAUC | 90.2 | 87.2 |
| 311 | 2955 | AUAUAGCUUAAACCUAAUC | 4291 | GAUUAGGUUUAAGCUAUAU | 59.9 | 56.9 |
| 312 | 2956 | UAUAGCUUAAACCUAAUCC | 4292 | GGAUUAGGUUUAAGCUAUA | 50.2 | 47.2 |
| 313 | 2957 | AUAGCUUAAACCUAAUCCC | 4293 | GGGAUUAGGUUUAAGCUAU | 54.7 | 51.7 |
| 314 | 2958 | UAGCUUAAACCUAAUCCCU | 4294 | AGGGAUUAGGUUUAAGCUA | 66.5 | 63.5 |
| 315 | 2959 | AGCUUAAACCUAAUCCCUC | 4295 | GAGGGAUUAGGUUUAAGCU | 63.9 | 60.9 |
| 316 | 2960 | GCUUAAACCUAAUCCCUCC | 4296 | GGAGGGAUUAGGUUUAAGC | 68.5 | 65.5 |
| 317 | 2961 | CUUAAACCUAAUCCCUCCC | 4297 | GGGAGGGAUUAGGUUUAAG | 52.5 | 49.5 |
| 318 | 2962 | UUAAACCUAAUCCCUCCCG | 4298 | CGGGAGGGAUUAGGUUUAA | 39.9 | 36.9 |
| 319 | 2963 | UAAACCUAAUCCCUCCCGC | 4299 | GCGGGAGGGAUUAGGUUUA | 41.8 | 38.8 |
| 320 | 2964 | AAACCUAAUCCCUCCCGCC | 4300 | GGCGGGAGGGAUUAGGUUU | 45.5 | 42.5 |
| 321 | 2965 | AACCUAAUCCCUCCCGCCC | 4301 | GGGCGGGAGGGAUUAGGUU | 43.6 | 40.6 |
| 322 | 2966 | ACCUAAUCCCUCCCGCCCU | 4302 | AGGGCGGGAGGGAUUAGGU | 59.8 | 56.8 |
| 323 | 2967 | CCUAAUCCCUCCCGCCCUG | 4303 | CAGGGCGGGAGGGAUUAGG | 60.2 | 57.2 |
| 324 | 2968 | CUAAUCCCUCCCGCCCUGA | 4304 | UCAGGGCGGGAGGGAUUAG | 60.3 | 57.3 |
| 325 | 2969 | UAAUCCCUCCCGCCCUGAU | 4305 | AUCAGGGCGGGAGGGAUUA | 55.6 | 52.6 |
| 326 | 2970 | AAUCCCUCCCGCCCUGAUC | 4306 | GAUCAGGGCGGGAGGGAUU | 48 | 45 |
| 327 | 2971 | AUCCCUCCCGCCCUGAUCU | 4307 | AGAUCAGGGCGGGAGGGAU | 52.6 | 49.6 |
| 328 | 2972 | UCCCUCCCGCCCUGAUCUC | 4308 | GAGAUCAGGGCGGGAGGGA | 44 | 41 |
| 329 | 2973 | CCCUCCCGCCCUGAUCUCA | 4309 | UGAGAUCAGGGCGGGAGGG | 66.3 | 63.3 |
| 330 | 2974 | CCUCCCGCCCUGAUCUCAG | 4310 | CUGAGAUCAGGGCGGGAGG | 58.8 | 55.8 |
| 331 | 2975 | CUCCCGCCCUGAUCUCAGG | 4311 | CCUGAGAUCAGGGCGGGAG | 54.1 | 51.1 |
| 332 | 2976 | UCCCGCCCUGAUCUCAGGU | 4312 | ACCUGAGAUCAGGGCGGGA | 48.9 | 45.9 |
| 333 | 2977 | CCCGCCCUGAUCUCAGGUG | 4313 | CACCUGAGAUCAGGGCGGG | 54 | 51 |
| 334 | 2978 | CCGCCCUGAUCUCAGGUGA | 4314 | UCACCUGAGAUCAGGGCGG | 75.9 | 72.9 |
| 335 | 2979 | CGCCCUGAUCUCAGGUGAG | 4315 | CUCACCUGAGAUCAGGGCG | 67.4 | 64.4 |
| 336 | 2980 | GCCCUGAUCUCAGGUGAGC | 4316 | GCUCACCUGAGAUCAGGGC | 57.9 | 54.9 |
| 337 | 2981 | CCCUGAUCUCAGGUGAGCA | 4317 | UGCUCACCUGAGAUCAGGG | 75.6 | 72.6 |
| 338 | 2982 | CCUGAUCUCAGGUGAGCAC | 4318 | GUGCUCACCUGAGAUCAGG | 67.2 | 64.2 |
| 339 | 2983 | CUGAUCUCAGGUGAGCACC | 4319 | GGUGCUCACCUGAGAUCAG | 51.7 | 48.7 |
| 340 | 2984 | UGAUCUCAGGUGAGCACCU | 4320 | AGGUGCUCACCUGAGAUCA | 68.5 | 65.5 |
| 341 | 2985 | GAUCUCAGGUGAGCACCUC | 4321 | GAGGUGCUCACCUGAGAUC | 65.4 | 62.4 |
| 342 | 2986 | AUCUCAGGUGAGCACCUCC | 4322 | GGAGGUGCUCACCUGAGAU | 53 | 50 |
| 343 | 2987 | UCUCAGGUGAGCACCUCCG | 4323 | CGGAGGUGCUCACCUGAGA | 54.3 | 51.3 |
| 344 | 2988 | CUCAGGUGAGCACCUCCGG | 4324 | CCGGAGGUGCUCACCUGAG | 57.8 | 54.8 |
| 345 | 2989 | UCAGGUGAGCACCUCCGGG | 4325 | CCCGGAGGUGCUCACCUGA | 46.8 | 43.8 |
| 346 | 2990 | CAGGUGAGCACCUCCGGGU | 4326 | ACCCGGAGGUGCUCACCUG | 63.8 | 60.8 |
| 347 | 2991 | AGGUGAGCACCUCCGGGUC | 4327 | GACCCGGAGGUGCUCACCU | 59.3 | 56.3 |
| 348 | 2992 | GGUGAGCACCUCCGGGUCU | 4328 | AGACCCGGAGGUGCUCACC | 74.6 | 71.6 |
| 349 | 2993 | GUGAGCACCUCCGGGUCUG | 4329 | CAGACCCGGAGGUGCUCAC | 59.4 | 56.4 |
| 350 | 2994 | UGAGCACCUCCGGGUCUGU | 4330 | ACAGACCCGGAGGUGCUCA | 60.1 | 57.1 |
| 351 | 2995 | GAGCACCUCCGGGUCUGUC | 4331 | GACAGACCCGGAGGUGCUC | 55.9 | 52.9 |
| 352 | 2996 | AGCACCUCCGGGUCUGUCC | 4332 | GGACAGACCCGGAGGUGCU | 53.5 | 50.5 |
| 353 | 2997 | GCACCUCCGGGUCUGUCCC | 4333 | GGGACAGACCCGGAGGUGC | 57.9 | 54.9 |
| 370 | 2998 | CCCAGGAGUACACCUGCUG | 4334 | CAGCAGGUGUACUCCUGGG | 65.9 | 62.9 |
| 371 | 2999 | CCAGGAGUACACCUGCUGU | 4335 | ACAGCAGGUGUACUCCUGG | 75.8 | 72.8 |
| 372 | 3000 | CAGGAGUACACCUGCUGUU | 4336 | AACAGCAGGUGUACUCCUG | 81.1 | 78.1 |
| 373 | 3001 | AGGAGUACACCUGCUGUUC | 4337 | GAACAGCAGGUGUACUCCU | 67.2 | 64.2 |
| 374 | 3002 | GGAGUACACCUGCUGUUCC | 4338 | GGAACAGCAGGUGUACUCC | 70 | 67 |
| 375 | 3003 | GAGUACACCUGCUGUUCCA | 4339 | UGGAACAGCAGGUGUACUC | 89 | 86 |
| 376 | 3004 | AGUACACCUGCUGUUCCAG | 4340 | CUGGAACAGCAGGUGUACU | 66.4 | 63.4 |
| 377 | 3005 | GUACACCUGCUGUUCCAGU | 4341 | ACUGGAACAGCAGGUGUAC | 71.4 | 68.4 |
| 378 | 3006 | UACACCUGCUGUUCCAGUG | 4342 | CACUGGAACAGCAGGUGUA | 61.4 | 58.4 |
| 379 | 3007 | ACACCUGCUGUUCCAGUGA | 4343 | UCACUGGAACAGCAGGUGU | 80.6 | 77.6 |
| 380 | 3008 | CACCUGCUGUUCCAGUGAG | 4344 | CUCACUGGAACAGCAGGUG | 72.5 | 69.5 |
| 381 | 3009 | ACCUGCUGUUCCAGUGAGA | 4345 | UCUCACUGGAACAGCAGGU | 81.7 | 78.7 |
| 382 | 3010 | CCUGCUGUUCCAGUGAGAC | 4346 | GUCUCACUGGAACAGCAGG | 66.3 | 63.3 |
| 383 | 3011 | CUGCUGUUCCAGUGAGACA | 4347 | UGUCUCACUGGAACAGCAG | 84.8 | 81.8 |
| 384 | 3012 | UGCUGUUCCAGUGAGACAG | 4348 | CUGUCUCACUGGAACAGCA | 68.3 | 65.3 |
| 385 | 3013 | GCUGUUCCAGUGAGACAGA | 4349 | UCUGUCUCACUGGAACAGC | 91.8 | 88.8 |
| 386 | 3014 | CUGUUCCAGUGAGACAGAG | 4350 | CUCUGUCUCACUGGAACAG | 68.1 | 65.1 |
| 387 | 3015 | UGUUCCAGUGAGACAGAGC | 4351 | GCUCUGUCUCACUGGAACA | 60 | 57 |
| 388 | 3016 | GUUCCAGUGAGACAGAGCA | 4352 | UGCUCUGUCUCACUGGAAC | 84.3 | 81.3 |
| 389 | 3017 | UUCCAGUGAGACAGAGCAG | 4353 | CUGCUCUGUCUCACUGGAA | 62.3 | 59.3 |
| 390 | 3018 | UCCAGUGAGACAGAGCAGA | 4354 | UCUGCUCUGUCUCACUGGA | 72.7 | 69.7 |
| 391 | 3019 | CCAGUGAGACAGAGCAGAG | 4355 | CUCUGCUCUGUCUCACUGG | 74.9 | 71.9 |
| 392 | 3020 | CAGUGAGACAGAGCAGAGG | 4356 | CCUCUGCUCUGUCUCACUG | 65.9 | 62.9 |
| 393 | 3021 | AGUGAGACAGAGCAGAGGC | 4357 | GCCUCUGCUCUGUCUCACU | 61.3 | 58.3 |
| 394 | 3022 | GUGAGACAGAGCAGAGGCU | 4358 | AGCCUCUGCUCUGUCUCAC | 74 | 71 |
| 395 | 3023 | UGAGACAGAGCAGAGGCUG | 4359 | CAGCCUCUGCUCUGUCUCA | 59.2 | 56.2 |
| 396 | 3024 | GAGACAGAGCAGAGGCUGA | 4360 | UCAGCCUCUGCUCUGUCUC | 87 | 84 |
| 397 | 3025 | AGACAGAGCAGAGGCUGAU | 4361 | AUCAGCCUCUGCUCUGUCU | 77.2 | 74.2 |
| 398 | 3026 | GACAGAGCAGAGGCUGAUC | 4362 | GAUCAGCCUCUGCUCUGUC | 67.5 | 64.5 |
| 399 | 3027 | ACAGAGCAGAGGCUGAUCA | 4363 | UGAUCAGCCUCUGCUCUGU | 76.7 | 73.7 |
| 400 | 3028 | CAGAGCAGAGGCUGAUCAG | 4364 | CUGAUCAGCCUCUGCUCUG | 71.3 | 67.3 |
| 401 | 3029 | AGAGCAGAGGCUGAUCAGG | 4365 | CCUGAUCAGCCUCUGCUCU | 59.5 | 55.5 |
| 402 | 3030 | GAGCAGAGGCUGAUCAGGG | 4366 | CCCUGAUCAGCCUCUGCUC | 59.8 | 55.8 |
| 403 | 3031 | AGCAGAGGCUGAUCAGGGA | 4367 | UCCCUGAUCAGCCUCUGCU | 78.3 | 74.3 |
| 404 | 3032 | GCAGAGGCUGAUCAGGGAG | 4368 | CUCCCUGAUCAGCCUCUGC | 68.1 | 64.1 |
| 405 | 3033 | CAGAGGCUGAUCAGGGAGA | 4369 | UCUCCCUGAUCAGCCUCUG | 76.5 | 72.5 |
| 406 | 3034 | AGAGGCUGAUCAGGGAGAC | 4370 | GUCUCCCUGAUCAGCCUCU | 59.4 | 55.4 |
| 407 | 3035 | GAGGCUGAUCAGGGAGACU | 4371 | AGUCUCCCUGAUCAGCCUC | 70 | 66 |
| 408 | 3036 | AGGCUGAUCAGGGAGACUG | 4372 | CAGUCUCCCUGAUCAGCCU | 60.4 | 56.4 |
| 409 | 3037 | GGCUGAUCAGGGAGACUGA | 4373 | UCAGUCUCCCUGAUCAGCC | 88.9 | 84.9 |
| 410 | 3038 | GCUGAUCAGGGAGACUGAG | 4374 | CUCAGUCUCCCUGAUCAGC | 68.4 | 64.4 |
| 411 | 3039 | CUGAUCAGGGAGACUGAGG | 4375 | CCUCAGUCUCCCUGAUCAG | 53.5 | 49.5 |
| 412 | 3040 | UGAUCAGGGAGACUGAGGC | 4376 | GCCUCAGUCUCCCUGAUCA | 47.3 | 43.3 |
| 413 | 3041 | GAUCAGGGAGACUGAGGCC | 4377 | GGCCUCAGUCUCCCUGAUC | 56 | 52 |
| 430 | 3042 | CCACCUUCCGAGGCCUGGU | 4378 | ACCAGGCCUCGGAAGGUGG | 63.7 | 59.7 |
| 431 | 3043 | CACCUUCCGAGGCCUGGUG | 4379 | CACCAGGCCUCGGAAGGUG | 59.6 | 55.6 |
| 432 | 3044 | ACCUUCCGAGGCCUGGUGG | 4380 | CCACCAGGCCUCGGAAGGU | 49.4 | 45.4 |
| 433 | 3045 | CCUUCCGAGGCCUGGUGGA | 4381 | UCCACCAGGCCUCGGAAGG | 72.5 | 68.5 |
| 434 | 3046 | CUUCCGAGGCCUGGUGGAG | 4382 | CUCCACCAGGCCUCGGAAG | 56.1 | 52.1 |
| 435 | 3047 | UUCCGAGGCCUGGUGGAGG | 4383 | CCUCCACCAGGCCUCGGAA | 38.8 | 34.8 |
| 436 | 3048 | UCCGAGGCCUGGUGGAGGA | 4384 | UCCUCCACCAGGCCUCGGA | 66.5 | 62.5 |
| 437 | 3049 | CCGAGGCCUGGUGGAGGAC | 4385 | GUCCUCCACCAGGCCUCGG | 56.9 | 52.9 |
| 438 | 3050 | CGAGGCCUGGUGGAGGACA | 4386 | UGUCCUCCACCAGGCCUCG | 73.5 | 69.5 |
| 439 | 3051 | GAGGCCUGGUGGAGGACAG | 4387 | CUGUCCUCCACCAGGCCUC | 67 | 63 |
| 440 | 3052 | AGGCCUGGUGGAGGACAGC | 4388 | GCUGUCCUCCACCAGGCCU | 51.2 | 47.2 |
| 441 | 3053 | GGCCUGGUGGAGGACAGCG | 4389 | CGCUGUCCUCCACCAGGCC | 58.8 | 54.8 |
| 442 | 3054 | GCCUGGUGGAGGACAGCGG | 4390 | CCGCUGUCCUCCACCAGGC | 64.2 | 60.2 |
| 443 | 3055 | CCUGGUGGAGGACAGCGGC | 4391 | GCCGCUGUCCUCCACCAGG | 58.4 | 54.4 |
| 444 | 3056 | CUGGUGGAGGACAGCGGCU | 4392 | AGCCGCUGUCCUCCACCAG | 66.2 | 62.2 |
| 445 | 3057 | UGGUGGAGGACAGCGGCUC | 4393 | GAGCCGCUGUCCUCCACCA | 54.8 | 50.8 |
| 446 | 3058 | GGUGGAGGACAGCGGCUCC | 4394 | GGAGCCGCUGUCCUCCACC | 67.4 | 63.4 |
| 447 | 3059 | GUGGAGGACAGCGGCUCCU | 4395 | AGGAGCCGCUGUCCUCCAC | 67.1 | 63.1 |
| 448 | 3060 | UGGAGGACAGCGGCUCCUU | 4396 | AAGGAGCCGCUGUCCUCCA | 68 | 64 |
| 449 | 3061 | GGAGGACAGCGGCUCCUUU | 4397 | AAAGGAGCCGCUGUCCUCC | 83.2 | 79.2 |
| 450 | 3062 | GAGGACAGCGGCUCCUUUC | 4398 | GAAAGGAGCCGCUGUCCUC | 66.6 | 62.6 |
| 451 | 3063 | AGGACAGCGGCUCCUUUCU | 4399 | AGAAAGGAGCCGCUGUCCU | 71.8 | 67.8 |
| 452 | 3064 | GGACAGCGGCUCCUUUCUG | 4400 | CAGAAAGGAGCCGCUGUCC | 72.2 | 68.2 |
| 453 | 3065 | GACAGCGGCUCCUUUCUGG | 4401 | CCAGAAAGGAGCCGCUGUC | 62.8 | 58.8 |
| 454 | 3066 | ACAGCGGCUCCUUUCUGGU | 4402 | ACCAGAAAGGAGCCGCUGU | 64.8 | 60.8 |
| 455 | 3067 | CAGCGGCUCCUUUCUGGUU | 4403 | AACCAGAAAGGAGCCGCUG | 76.9 | 72.9 |
| 456 | 3068 | AGCGGCUCCUUUCUGGUUC | 4404 | GAACCAGAAAGGAGCCGCU | 62.8 | 58.8 |
| 457 | 3069 | GCGGCUCCUUUCUGGUUCA | 4405 | UGAACCAGAAAGGAGCCGC | 88.2 | 84.2 |
| 458 | 3070 | CGGCUCCUUUCUGGUUCAC | 4406 | GUGAACCAGAAAGGAGCCG | 65.9 | 61.9 |
| 459 | 3071 | GGCUCCUUUCUGGUUCACA | 4407 | UGUGAACCAGAAAGGAGCC | 80.4 | 76.4 |
| 460 | 3072 | GCUCCUUUCUGGUUCACAC | 4408 | GUGUGAACCAGAAAGGAGC | 76.2 | 72.2 |
| 513 | 3073 | UUUCUGGAGAUGCUCUCAG | 4409 | CUGAGAGCAUCUCCAGAAA | 55.3 | 50.3 |
| 514 | 3074 | UUCUGGAGAUGCUCUCAGU | 4410 | ACUGAGAGCAUCUCCAGAA | 69 | 64 |
| 515 | 3075 | UCUGGAGAUGCUCUCAGUA | 4411 | UACUGAGAGCAUCUCCAGA | 78.9 | 73.9 |
| 516 | 3076 | CUGGAGAUGCUCUCAGUAG | 4412 | CUACUGAGAGCAUCUCCAG | 73 | 68 |
| 517 | 3077 | UGGAGAUGCUCUCAGUAGC | 4413 | GCUACUGAGAGCAUCUCCA | 64.1 | 59.1 |
| 518 | 3078 | GGAGAUGCUCUCAGUAGCC | 4414 | GGCUACUGAGAGCAUCUCC | 68.6 | 63.6 |
| 519 | 3079 | GAGAUGCUCUCAGUAGCCC | 4415 | GGGCUACUGAGAGCAUCUC | 60.2 | 55.2 |
| 520 | 3080 | AGAUGCUCUCAGUAGCCCA | 4416 | UGGGCUACUGAGAGCAUCU | 81 | 76 |
| 521 | 3081 | GAUGCUCUCAGUAGCCCAG | 4417 | CUGGGCUACUGAGAGCAUC | 71.5 | 66.5 |
| 522 | 3082 | AUGCUCUCAGUAGCCCAGC | 4418 | GCUGGGCUACUGAGAGCAU | 43.7 | 38.7 |
| 523 | 3083 | UGCUCUCAGUAGCCCAGCA | 4419 | UGCUGGGCUACUGAGAGCA | 74.4 | 69.4 |
| 524 | 3084 | GCUCUCAGUAGCCCAGCAC | 4420 | GUGCUGGGCUACUGAGAGC | 65.2 | 60.2 |
| 525 | 3085 | CUCUCAGUAGCCCAGCACU | 4421 | AGUGCUGGGCUACUGAGAG | 69.9 | 64.9 |
| 526 | 3086 | UCUCAGUAGCCCAGCACUC | 4422 | GAGUGCUGGGCUACUGAGA | 57.8 | 52.8 |
| 527 | 3087 | CUCAGUAGCCCAGCACUCU | 4423 | AGAGUGCUGGGCUACUGAG | 67.5 | 62.5 |
| 528 | 3088 | UCAGUAGCCCAGCACUCUC | 4424 | GAGAGUGCUGGGCUACUGA | 57.4 | 52.4 |
| 529 | 3089 | CAGUAGCCCAGCACUCUCU | 4425 | AGAGAGUGCUGGGCUACUG | 73.5 | 68.5 |
| 530 | 3090 | AGUAGCCCAGCACUCUCUG | 4426 | CAGAGAGUGCUGGGCUACU | 61.1 | 56.1 |
| 531 | 3091 | GUAGCCCAGCACUCUCUGA | 4427 | UCAGAGAGUGCUGGGCUAC | 74.8 | 69.8 |
| 532 | 3092 | UAGCCCAGCACUCUCUGAC | 4428 | GUCAGAGAGUGCUGGGCUA | 47.3 | 42.3 |
| 533 | 3093 | AGCCCAGCACUCUCUGACC | 4429 | GGUCAGAGAGUGCUGGGCU | 55.5 | 50.5 |
| 534 | 3094 | GCCCAGCACUCUCUGACCC | 4430 | GGGUCAGAGAGUGCUGGGC | 58.1 | 53.1 |
| 535 | 3095 | CCCAGCACUCUCUGACCCA | 4431 | UGGGUCAGAGAGUGCUGGG | 75.1 | 70.1 |
| 536 | 3096 | CCAGCACUCUCUGACCCAG | 4432 | CUGGGUCAGAGAGUGCUGG | 67 | 62 |
| 537 | 3097 | CAGCACUCUCUGACCCAGC | 4433 | GCUGGGUCAGAGAGUGCUG | 57.1 | 52.1 |
| 538 | 3098 | AGCACUCUCUGACCCAGCU | 4434 | AGCUGGGUCAGAGAGUGCU | 70.9 | 65.9 |
| 539 | 3099 | GCACUCUCUGACCCAGCUC | 4435 | GAGCUGGGUCAGAGAGUGC | 65.2 | 60.2 |
| 540 | 3100 | CACUCUCUGACCCAGCUCU | 4436 | AGAGCUGGGUCAGAGAGUG | 72.1 | 67.1 |
| 541 | 3101 | ACUCUCUGACCCAGCUCUU | 4437 | AAGAGCUGGGUCAGAGAGU | 73 | 68 |
| 542 | 3102 | CUCUCUGACCCAGCUCUUC | 4438 | GAAGAGCUGGGUCAGAGAG | 61 | 56 |
| 543 | 3103 | UCUCUGACCCAGCUCUUCU | 4439 | AGAAGAGCUGGGUCAGAGA | 65 | 60 |
| 544 | 3104 | CUCUGACCCAGCUCUUCUC | 4440 | GAGAAGAGCUGGGUCAGAG | 63.7 | 58.7 |
| 545 | 3105 | UCUGACCCAGCUCUUCUCC | 4441 | GGAGAAGAGCUGGGUCAGA | 46.8 | 41.8 |
| 546 | 3106 | CUGACCCAGCUCUUCUCCC | 4442 | GGGAGAAGAGCUGGGUCAG | 50.1 | 45.1 |
| 547 | 3107 | UGACCCAGCUCUUCUCCCA | 4443 | UGGGAGAAGAGCUGGGUCA | 72 | 67 |
| 548 | 3108 | GACCCAGCUCUUCUCCCAC | 4444 | GUGGGAGAAGAGCUGGGUC | 64.9 | 59.9 |
| 549 | 3109 | ACCCAGCUCUUCUCCCACU | 4445 | AGUGGGAGAAGAGCUGGGU | 67.9 | 62.9 |
| 550 | 3110 | CCCAGCUCUUCUCCCACUC | 4446 | GAGUGGGAGAAGAGCUGGG | 63.1 | 58.1 |
| 551 | 3111 | CCAGCUCUUCUCCCACUCC | 4447 | GGAGUGGGAGAAGAGCUGG | 60.7 | 55.7 |
| 552 | 3112 | CAGCUCUUCUCCCACUCCU | 4448 | AGGAGUGGGAGAAGAGCUG | 72.9 | 67.9 |
| 553 | 3113 | AGCUCUUCUCCCACUCCUA | 4449 | UAGGAGUGGGAGAAGAGCU | 87.4 | 82.4 |
| 554 | 3114 | GCUCUUCUCCCACUCCUAC | 4450 | GUAGGAGUGGGAGAAGAGC | 70.5 | 65.5 |
| 555 | 3115 | CUCUUCUCCCACUCCUACG | 4451 | CGUAGGAGUGGGAGAAGAG | 59.4 | 54.4 |
| 556 | 3116 | UCUUCUCCCACUCCUACGG | 4452 | CCGUAGGAGUGGGAGAAGA | 52.1 | 47.1 |
| 557 | 3117 | CUUCUCCCACUCCUACGGC | 4453 | GCCGUAGGAGUGGGAGAAG | 47.5 | 42.5 |
| 558 | 3118 | UUCUCCCACUCCUACGGCC | 4454 | GGCCGUAGGAGUGGGAGAA | 39.7 | 34.7 |
| 559 | 3119 | UCUCCCACUCCUACGGCCG | 4455 | CGGCCGUAGGAGUGGGAGA | 46 | 41 |
| 560 | 3120 | CUCCCACUCCUACGGCCGC | 4456 | GCGGCCGUAGGAGUGGGAG | 47.5 | 42.5 |
| 561 | 3121 | UCCCACUCCUACGGCCGCC | 4457 | GGCGGCCGUAGGAGUGGGA | 34.9 | 29.9 |
| 562 | 3122 | CCCACUCCUACGGCCGCCU | 4458 | AGGCGGCCGUAGGAGUGGG | 55.9 | 50.9 |
| 563 | 3123 | CCACUCCUACGGCCGCCUG | 4459 | CAGGCGGCCGUAGGAGUGG | 56.4 | 51.4 |
| 564 | 3124 | CACUCCUACGGCCGCCUGU | 4460 | ACAGGCGGCCGUAGGAGUG | 63.9 | 58.9 |
| 565 | 3125 | ACUCCUACGGCCGCCUGUA | 4461 | UACAGGCGGCCGUAGGAGU | 69.9 | 64.9 |
| 566 | 3126 | CUCCUACGGCCGCCUGUAU | 4462 | AUACAGGCGGCCGUAGGAG | 68.7 | 63.7 |
| 567 | 3127 | UCCUACGGCCGCCUGUAUG | 4463 | CAUACAGGCGGCCGUAGGA | 50.2 | 45.2 |
| 568 | 3128 | CCUACGGCCGCCUGUAUGC | 4464 | GCAUACAGGCGGCCGUAGG | 54.5 | 49.5 |
| 569 | 3129 | CUACGGCCGCCUGUAUGCC | 4465 | GGCAUACAGGCGGCCGUAG | 43.2 | 38.2 |
| 570 | 3130 | UACGGCCGCCUGUAUGCCC | 4466 | GGGCAUACAGGCGGCCGUA | 36.1 | 31.1 |
| 571 | 3131 | ACGGCCGCCUGUAUGCCCA | 4467 | UGGGCAUACAGGCGGCCGU | 62.2 | 57.2 |
| 572 | 3132 | CGGCCGCCUGUAUGCCCAG | 4468 | CUGGGCAUACAGGCGGCCG | 59.8 | 54.8 |
| 573 | 3133 | GGCCGCCUGUAUGCCCAGC | 4469 | GCUGGGCAUACAGGCGGCC | 49 | 44 |
| 574 | 3134 | GCCGCCUGUAUGCCCAGCA | 4470 | UGCUGGGCAUACAGGCGGC | 66.7 | 61.7 |
| 575 | 3135 | CCGCCUGUAUGCCCAGCAC | 4471 | GUGCUGGGCAUACAGGCGG | 63.2 | 58.2 |
| 576 | 3136 | CGCCUGUAUGCCCAGCACG | 4472 | CGUGCUGGGCAUACAGGCG | 60.2 | 55.2 |
| 577 | 3137 | GCCUGUAUGCCCAGCACGC | 4473 | GCGUGCUGGGCAUACAGGC | 59.2 | 54.2 |
| 578 | 3138 | CCUGUAUGCCCAGCACGCC | 4474 | GGCGUGCUGGGCAUACAGG | 49.6 | 44.6 |
| 579 | 3139 | CUGUAUGCCCAGCACGCCC | 4475 | GGGCGUGCUGGGCAUACAG | 50.3 | 45.3 |
| 580 | 3140 | UGUAUGCCCAGCACGCCCU | 4476 | AGGGCGUGCUGGGCAUACA | 57.5 | 52.5 |
| 581 | 3141 | GUAUGCCCAGCACGCCCUC | 4477 | GAGGGCGUGCUGGGCAUAC | 53.3 | 48.3 |
| 582 | 3142 | UAUGCCCAGCACGCCCUCA | 4478 | UGAGGGCGUGCUGGGCAUA | 55.6 | 50.6 |
| 583 | 3143 | AUGCCCAGCACGCCCUCAU | 4479 | AUGAGGGCGUGCUGGGCAU | 56.7 | 51.7 |
| 584 | 3144 | UGCCCAGCACGCCCUCAUA | 4480 | UAUGAGGGCGUGCUGGGCA | 71.7 | 66.7 |
| 585 | 3145 | GCCCAGCACGCCCUCAUAU | 4481 | AUAUGAGGGCGUGCUGGGC | 71.9 | 66.9 |
| 586 | 3146 | CCCAGCACGCCCUCAUAUU | 4482 | AAUAUGAGGGCGUGCUGGG | 70.6 | 65.6 |
| 587 | 3147 | CCAGCACGCCCUCAUAUUC | 4483 | GAAUAUGAGGGCGUGCUGG | 62.6 | 57.6 |
| 588 | 3148 | CAGCACGCCCUCAUAUUCA | 4484 | UGAAUAUGAGGGCGUGCUG | 74.8 | 69.8 |
| 589 | 3149 | AGCACGCCCUCAUAUUCAA | 4485 | UUGAAUAUGAGGGCGUGCU | 85.4 | 80.4 |
| 590 | 3150 | GCACGCCCUCAUAUUCAAU | 4486 | AUUGAAUAUGAGGGCGUGC | 77.1 | 72.1 |
| 591 | 3151 | CACGCCCUCAUAUUCAAUG | 4487 | CAUUGAAUAUGAGGGCGUG | 58.6 | 53.6 |
| 592 | 3152 | ACGCCCUCAUAUUCAAUGG | 4488 | CCAUUGAAUAUGAGGGCGU | 56.9 | 51.9 |
| 609 | 3153 | GGCCUGUUCUCUCGGCUGC | 4489 | GCAGCCGAGAGAACAGGCC | 64.9 | 58.9 |
| 610 | 3154 | GCCUGUUCUCUCGGCUGCG | 4490 | CGCAGCCGAGAGAACAGGC | 62.3 | 56.3 |
| 611 | 3155 | CCUGUUCUCUCGGCUGCGA | 4491 | UCGCAGCCGAGAGAACAGG | 80.9 | 74.9 |
| 612 | 3156 | CUGUUCUCUCGGCUGCGAG | 4492 | CUCGCAGCCGAGAGAACAG | 63.8 | 57.8 |
| 613 | 3157 | UGUUCUCUCGGCUGCGAGA | 4493 | UCUCGCAGCCGAGAGAACA | 79.7 | 73.7 |
| 614 | 3158 | GUUCUCUCGGCUGCGAGAC | 4494 | GUCUCGCAGCCGAGAGAAC | 60.6 | 54.6 |
| 615 | 3159 | UUCUCUCGGCUGCGAGACU | 4495 | AGUCUCGCAGCCGAGAGAA | 61.3 | 55.3 |
| 616 | 3160 | UCUCUCGGCUGCGAGACUU | 4496 | AAGUCUCGCAGCCGAGAGA | 68.1 | 62.1 |
| 617 | 3161 | CUCUCGGCUGCGAGACUUC | 4497 | GAAGUCUCGCAGCCGAGAG | 67 | 61 |
| 618 | 3162 | UCUCGGCUGCGAGACUUCU | 4498 | AGAAGUCUCGCAGCCGAGA | 67.5 | 61.5 |
| 619 | 3163 | CUCGGCUGCGAGACUUCUA | 4499 | UAGAAGUCUCGCAGCCGAG | 85.8 | 79.8 |
| 620 | 3164 | UCGGCUGCGAGACUUCUAU | 4500 | AUAGAAGUCUCGCAGCCGA | 73.5 | 67.5 |
| 621 | 3165 | CGGCUGCGAGACUUCUAUG | 4501 | CAUAGAAGUCUCGCAGCCG | 76.6 | 70.6 |
| 622 | 3166 | GGCUGCGAGACUUCUAUGG | 4502 | CCAUAGAAGUCUCGCAGCC | 71.5 | 65.5 |
| 623 | 3167 | GCUGCGAGACUUCUAUGGG | 4503 | CCCAUAGAAGUCUCGCAGC | 68.4 | 62.4 |
| 654 | 3168 | GGGUUGGAUGACACCCUGG | 4504 | CCAGGGUGUCAUCCAACCC | 69.4 | 63.4 |
| 671 | 3169 | GGCGGAUUUCUGGGCACAG | 4505 | CUGUGCCCAGAAAUCCGCC | 67.2 | 61.2 |
| 672 | 3170 | GCGGAUUUCUGGGCACAGC | 4506 | GCUGUGCCCAGAAAUCCGC | 60.3 | 54.3 |
| 673 | 3171 | CGGAUUUCUGGGCACAGCU | 4507 | AGCUGUGCCCAGAAAUCCG | 72.2 | 66.2 |
| 674 | 3172 | GGAUUUCUGGGCACAGCUC | 4508 | GAGCUGUGCCCAGAAAUCC | 70 | 64 |
| 675 | 3173 | GAUUUCUGGGCACAGCUCC | 4509 | GGAGCUGUGCCCAGAAAUC | 58.3 | 52.3 |
| 676 | 3174 | AUUUCUGGGCACAGCUCCU | 4510 | AGGAGCUGUGCCCAGAAAU | 62.8 | 56.8 |
| 677 | 3175 | UUUCUGGGCACAGCUCCUG | 4511 | CAGGAGCUGUGCCCAGAAA | 45 | 39 |
| 678 | 3176 | UUCUGGGCACAGCUCCUGG | 4512 | CCAGGAGCUGUGCCCAGAA | 46.3 | 40.3 |
| 679 | 3177 | UCUGGGCACAGCUCCUGGA | 4513 | UCCAGGAGCUGUGCCCAGA | 65.8 | 59.8 |
| 680 | 3178 | CUGGGCACAGCUCCUGGAG | 4514 | CUCCAGGAGCUGUGCCCAG | 56.3 | 50.3 |
| 681 | 3179 | UGGGCACAGCUCCUGGAGA | 4515 | UCUCCAGGAGCUGUGCCCA | 68.2 | 62.2 |
| 682 | 3180 | GGGCACAGCUCCUGGAGAG | 4516 | CUCUCCAGGAGCUGUGCCC | 69.4 | 63.4 |
| 683 | 3181 | GGCACAGCUCCUGGAGAGA | 4517 | UCUCUCCAGGAGCUGUGCC | 82.4 | 76.4 |
| 684 | 3182 | GCACAGCUCCUGGAGAGAG | 4518 | CUCUCUCCAGGAGCUGUGC | 69.3 | 63.3 |
| 685 | 3183 | CACAGCUCCUGGAGAGAGU | 4519 | ACUCUCUCCAGGAGCUGUG | 75.9 | 69.9 |
| 686 | 3184 | ACAGCUCCUGGAGAGAGUG | 4520 | CACUCUCUCCAGGAGCUGU | 62.3 | 56.3 |
| 687 | 3185 | CAGCUCCUGGAGAGAGUGU | 4521 | ACACUCUCUCCAGGAGCUG | 73.3 | 67.3 |
| 688 | 3186 | AGCUCCUGGAGAGAGUGUU | 4522 | AACACUCUCUCCAGGAGCU | 74.9 | 68.9 |
| 689 | 3187 | GCUCCUGGAGAGAGUGUUC | 4523 | GAACACUCUCUCCAGGAGC | 76.9 | 70.9 |
| 690 | 3188 | CUCCUGGAGAGAGUGUUCC | 4524 | GGAACACUCUCUCCAGGAG | 56.9 | 50.9 |
| 691 | 3189 | UCCUGGAGAGAGUGUUCCC | 4525 | GGGAACACUCUCUCCAGGA | 57.6 | 51.6 |
| 692 | 3190 | CCUGGAGAGAGUGUUCCCG | 4526 | CGGGAACACUCUCUCCAGG | 61.3 | 55.3 |
| 693 | 3191 | CUGGAGAGAGUGUUCCCGC | 4527 | GCGGGAACACUCUCUCCAG | 56.6 | 50.6 |
| 694 | 3192 | UGGAGAGAGUGUUCCCGCU | 4528 | AGCGGGAACACUCUCUCCA | 71.5 | 65.5 |
| 695 | 3193 | GGAGAGAGUGUUCCCGCUG | 4529 | CAGCGGGAACACUCUCUCC | 75.5 | 69.5 |
| 696 | 3194 | GAGAGAGUGUUCCCGCUGC | 4530 | GCAGCGGGAACACUCUCUC | 67.8 | 61.8 |
| 697 | 3195 | AGAGAGUGUUCCCGCUGCU | 4531 | AGCAGCGGGAACACUCUCU | 76.9 | 70.9 |
| 698 | 3196 | GAGAGUGUUCCCGCUGCUG | 4532 | CAGCAGCGGGAACACUCUC | 69.4 | 63.4 |
| 699 | 3197 | AGAGUGUUCCCGCUGCUGC | 4533 | GCAGCAGCGGGAACACUCU | 60.3 | 54.3 |
| 700 | 3198 | GAGUGUUCCCGCUGCUGCA | 4534 | UGCAGCAGCGGGAACACUC | 86.8 | 79.8 |
| 701 | 3199 | AGUGUUCCCGCUGCUGCAC | 4535 | GUGCAGCAGCGGGAACACU | 61.8 | 54.8 |
| 702 | 3200 | GUGUUCCCGCUGCUGCACC | 4536 | GGUGCAGCAGCGGGAACAC | 54.1 | 47.1 |
| 703 | 3201 | UGUUCCCGCUGCUGCACCC | 4537 | GGGUGCAGCAGCGGGAACA | 52.9 | 45.9 |
| 704 | 3202 | GUUCCCGCUGCUGCACCCA | 4538 | UGGGUGCAGCAGCGGGAAC | 69.3 | 62.3 |
| 705 | 3203 | UUCCCGCUGCUGCACCCAC | 4539 | GUGGGUGCAGCAGCGGGAA | 47.3 | 40.3 |
| 706 | 3204 | UCCCGCUGCUGCACCCACA | 4540 | UGUGGGUGCAGCAGCGGGA | 68.5 | 61.5 |
| 707 | 3205 | CCCGCUGCUGCACCCACAG | 4541 | CUGUGGGUGCAGCAGCGGG | 64.6 | 57.6 |
| 708 | 3206 | CCGCUGCUGCACCCACAGU | 4542 | ACUGUGGGUGCAGCAGCGG | 70.4 | 63.4 |
| 709 | 3207 | CGCUGCUGCACCCACAGUA | 4543 | UACUGUGGGUGCAGCAGCG | 83.9 | 76.9 |
| 710 | 3208 | GCUGCUGCACCCACAGUAC | 4544 | GUACUGUGGGUGCAGCAGC | 74.2 | 67.2 |
| 711 | 3209 | CUGCUGCACCCACAGUACA | 4545 | UGUACUGUGGGUGCAGCAG | 79.4 | 72.4 |
| 712 | 3210 | UGCUGCACCCACAGUACAG | 4546 | CUGUACUGUGGGUGCAGCA | 64.7 | 57.7 |
| 713 | 3211 | GCUGCACCCACAGUACAGC | 4547 | GCUGUACUGUGGGUGCAGC | 58.4 | 51.4 |
| 714 | 3212 | CUGCACCCACAGUACAGCU | 4548 | AGCUGUACUGUGGGUGCAG | 67.7 | 60.7 |
| 715 | 3213 | UGCACCCACAGUACAGCUU | 4549 | AAGCUGUACUGUGGGUGCA | 70.2 | 63.2 |
| 716 | 3214 | GCACCCACAGUACAGCUUC | 4550 | GAAGCUGUACUGUGGGUGC | 70.3 | 63.3 |
| 717 | 3215 | CACCCACAGUACAGCUUCC | 4551 | GGAAGCUGUACUGUGGGUG | 67 | 60 |
| 718 | 3216 | ACCCACAGUACAGCUUCCC | 4552 | GGGAAGCUGUACUGUGGGU | 49.1 | 42.1 |
| 737 | 3217 | CCCUGACUACCUGCUCUGC | 4553 | GCAGAGCAGGUAGUCAGGG | 59.5 | 52.5 |
| 738 | 3218 | CCUGACUACCUGCUCUGCC | 4554 | GGCAGAGCAGGUAGUCAGG | 54.5 | 47.5 |
| 739 | 3219 | CUGACUACCUGCUCUGCCU | 4555 | AGGCAGAGCAGGUAGUCAG | 72 | 65 |
| 740 | 3220 | UGACUACCUGCUCUGCCUC | 4556 | GAGGCAGAGCAGGUAGUCA | 56.6 | 49.6 |
| 741 | 3221 | GACUACCUGCUCUGCCUCU | 4557 | AGAGGCAGAGCAGGUAGUC | 73.1 | 66.1 |
| 742 | 3222 | ACUACCUGCUCUGCCUCUC | 4558 | GAGAGGCAGAGCAGGUAGU | 56.7 | 49.7 |
| 743 | 3223 | CUACCUGCUCUGCCUCUCA | 4559 | UGAGAGGCAGAGCAGGUAG | 77.8 | 70.8 |
| 744 | 3224 | UACCUGCUCUGCCUCUCAC | 4560 | GUGAGAGGCAGAGCAGGUA | 56 | 49 |
| 745 | 3225 | ACCUGCUCUGCCUCUCACG | 4561 | CGUGAGAGGCAGAGCAGGU | 55.2 | 48.2 |
| 746 | 3226 | CCUGCUCUGCCUCUCACGC | 4562 | GCGUGAGAGGCAGAGCAGG | 54.3 | 47.3 |
| 747 | 3227 | CUGCUCUGCCUCUCACGCU | 4563 | AGCGUGAGAGGCAGAGCAG | 62.6 | 55.6 |
| 748 | 3228 | UGCUCUGCCUCUCACGCUU | 4564 | AAGCGUGAGAGGCAGAGCA | 73 | 66 |
| 749 | 3229 | GCUCUGCCUCUCACGCUUG | 4565 | CAAGCGUGAGAGGCAGAGC | 70.9 | 63.9 |
| 750 | 3230 | CUCUGCCUCUCACGCUUGG | 4566 | CCAAGCGUGAGAGGCAGAG | 57.8 | 50.8 |
| 783 | 3231 | GGCUCUCUGCAGCCCUUUG | 4567 | CAAAGGGCUGCAGAGAGCC | 71.6 | 64.6 |
| 784 | 3232 | GCUCUCUGCAGCCCUUUGG | 4568 | CCAAAGGGCUGCAGAGAGC | 60.5 | 53.5 |
| 785 | 3233 | CUCUCUGCAGCCCUUUGGG | 4569 | CCCAAAGGGCUGCAGAGAG | 50.4 | 43.4 |
| 811 | 3234 | CCCGCCGCCUCCGCCUGCA | 4570 | UGCAGGCGGAGGCGGCGGG | 57.3 | 49.3 |
| 812 | 3235 | CCGCCGCCUCCGCCUGCAG | 4571 | CUGCAGGCGGAGGCGGCGG | 53.3 | 45.3 |
| 813 | 3236 | CGCCGCCUCCGCCUGCAGA | 4572 | UCUGCAGGCGGAGGCGGCG | 66.1 | 58.1 |
| 814 | 3237 | GCCGCCUCCGCCUGCAGAU | 4573 | AUCUGCAGGCGGAGGCGGC | 66.9 | 58.9 |
| 815 | 3238 | CCGCCUCCGCCUGCAGAUA | 4574 | UAUCUGCAGGCGGAGGCGG | 74.5 | 66.5 |
| 816 | 3239 | CGCCUCCGCCUGCAGAUAA | 4575 | UUAUCUGCAGGCGGAGGCG | 80.1 | 72.1 |
| 817 | 3240 | GCCUCCGCCUGCAGAUAAC | 4576 | GUUAUCUGCAGGCGGAGGC | 66.1 | 58.1 |
| 818 | 3241 | CCUCCGCCUGCAGAUAACC | 4577 | GGUUAUCUGCAGGCGGAGG | 53.1 | 45.1 |
| 819 | 3242 | CUCCGCCUGCAGAUAACCC | 4578 | GGGUUAUCUGCAGGCGGAG | 48.2 | 40.2 |
| 820 | 3243 | UCCGCCUGCAGAUAACCCG | 4579 | CGGGUUAUCUGCAGGCGGA | 45.8 | 37.8 |
| 821 | 3244 | CCGCCUGCAGAUAACCCGG | 4580 | CCGGGUUAUCUGCAGGCGG | 58.2 | 50.2 |
| 822 | 3245 | CGCCUGCAGAUAACCCGGA | 4581 | UCCGGGUUAUCUGCAGGCG | 73.7 | 65.7 |
| 823 | 3246 | GCCUGCAGAUAACCCGGAC | 4582 | GUCCGGGUUAUCUGCAGGC | 65.5 | 57.5 |
| 824 | 3247 | CCUGCAGAUAACCCGGACC | 4583 | GGUCCGGGUUAUCUGCAGG | 57.6 | 49.6 |
| 825 | 3248 | CUGCAGAUAACCCGGACCC | 4584 | GGGUCCGGGUUAUCUGCAG | 53.2 | 45.2 |
| 826 | 3249 | UGCAGAUAACCCGGACCCU | 4585 | AGGGUCCGGGUUAUCUGCA | 62.9 | 54.9 |
| 827 | 3250 | GCAGAUAACCCGGACCCUG | 4586 | CAGGGUCCGGGUUAUCUGC | 66.7 | 58.7 |
| 828 | 3251 | CAGAUAACCCGGACCCUGG | 4587 | CCAGGGUCCGGGUUAUCUG | 61.3 | 53.3 |
| 829 | 3252 | AGAUAACCCGGACCCUGGU | 4588 | ACCAGGGUCCGGGUUAUCU | 67.4 | 59.4 |
| 830 | 3253 | GAUAACCCGGACCCUGGUG | 4589 | CACCAGGGUCCGGGUUAUC | 59.2 | 51.2 |
| 831 | 3254 | AUAACCCGGACCCUGGUGG | 4590 | CCACCAGGGUCCGGGUUAU | 40.4 | 32.4 |
| 848 | 3255 | GGCUGCCCGAGCCUUUGUG | 4591 | CACAAAGGCUCGGGCAGCC | 62.5 | 54.5 |
| 849 | 3256 | GCUGCCCGAGCCUUUGUGC | 4592 | GCACAAAGGCUCGGGCAGC | 55.7 | 47.7 |
| 850 | 3257 | CUGCCCGAGCCUUUGUGCA | 4593 | UGCACAAAGGCUCGGGCAG | 68 | 60 |
| 851 | 3258 | UGCCCGAGCCUUUGUGCAG | 4594 | CUGCACAAAGGCUCGGGCA | 59.8 | 51.8 |
| 852 | 3259 | GCCCGAGCCUUUGUGCAGG | 4595 | CCUGCACAAAGGCUCGGGC | 59.3 | 51.3 |
| 853 | 3260 | CCCGAGCCUUUGUGCAGGG | 4596 | CCCUGCACAAAGGCUCGGG | 58 | 50 |
| 854 | 3261 | CCGAGCCUUUGUGCAGGGC | 4597 | GCCCUGCACAAAGGCUCGG | 50 | 42 |
| 855 | 3262 | CGAGCCUUUGUGCAGGGCC | 4598 | GGCCCUGCACAAAGGCUCG | 52.5 | 44.5 |
| 856 | 3263 | GAGCCUUUGUGCAGGGCCU | 4599 | AGGCCCUGCACAAAGGCUC | 71 | 63 |
| 857 | 3264 | AGCCUUUGUGCAGGGCCUG | 4600 | CAGGCCCUGCACAAAGGCU | 54.6 | 46.6 |
| 858 | 3265 | GCCUUUGUGCAGGGCCUGG | 4601 | CCAGGCCCUGCACAAAGGC | 54.8 | 46.8 |
| 859 | 3266 | CCUUUGUGCAGGGCCUGGA | 4602 | UCCAGGCCCUGCACAAAGG | 69.4 | 61.4 |
| 860 | 3267 | CUUUGUGCAGGGCCUGGAG | 4603 | CUCCAGGCCCUGCACAAAG | 59.8 | 51.8 |
| 861 | 3268 | UUUGUGCAGGGCCUGGAGA | 4604 | UCUCCAGGCCCUGCACAAA | 58.5 | 50.5 |
| 862 | 3269 | UUGUGCAGGGCCUGGAGAC | 4605 | GUCUCCAGGCCCUGCACAA | 45.8 | 37.8 |
| 863 | 3270 | UGUGCAGGGCCUGGAGACU | 4606 | AGUCUCCAGGCCCUGCACA | 59.3 | 51.3 |
| 864 | 3271 | GUGCAGGGCCUGGAGACUG | 4607 | CAGUCUCCAGGCCCUGCAC | 58.3 | 50.3 |
| 865 | 3272 | UGCAGGGCCUGGAGACUGG | 4608 | CCAGUCUCCAGGCCCUGCA | 54.8 | 46.8 |
| 866 | 3273 | GCAGGGCCUGGAGACUGGA | 4609 | UCCAGUCUCCAGGCCCUGC | 73.5 | 65.5 |
| 867 | 3274 | CAGGGCCUGGAGACUGGAA | 4610 | UUCCAGUCUCCAGGCCCUG | 75.3 | 67.3 |
| 868 | 3275 | AGGGCCUGGAGACUGGAAG | 4611 | CUUCCAGUCUCCAGGCCCU | 55.4 | 47.4 |
| 869 | 3276 | GGGCCUGGAGACUGGAAGA | 4612 | UCUUCCAGUCUCCAGGCCC | 81.8 | 73.8 |
| 870 | 3277 | GGCCUGGAGACUGGAAGAA | 4613 | UUCUUCCAGUCUCCAGGCC | 83.1 | 75.1 |
| 871 | 3278 | GCCUGGAGACUGGAAGAAA | 4614 | UUUCUUCCAGUCUCCAGGC | 89 | 81 |
| 872 | 3279 | CCUGGAGACUGGAAGAAAU | 4615 | AUUUCUUCCAGUCUCCAGG | 87.9 | 79.9 |
| 873 | 3280 | CUGGAGACUGGAAGAAAUG | 4616 | CAUUUCUUCCAGUCUCCAG | 70.4 | 62.4 |
| 874 | 3281 | UGGAGACUGGAAGAAAUGU | 4617 | ACAUUUCUUCCAGUCUCCA | 69.7 | 61.7 |
| 875 | 3282 | GGAGACUGGAAGAAAUGUG | 4618 | CACAUUUCUUCCAGUCUCC | 76.2 | 68.2 |
| 876 | 3283 | GAGACUGGAAGAAAUGUGG | 4619 | CCACAUUUCUUCCAGUCUC | 71.5 | 63.5 |
| 877 | 3284 | AGACUGGAAGAAAUGUGGU | 4620 | ACCACAUUUCUUCCAGUCU | 75.1 | 67.1 |
| 878 | 3285 | GACUGGAAGAAAUGUGGUC | 4621 | GACCACAUUUCUUCCAGUC | 70.9 | 62.9 |
| 879 | 3286 | ACUGGAAGAAAUGUGGUCA | 4622 | UGACCACAUUUCUUCCAGU | 76.1 | 68.1 |
| 880 | 3287 | CUGGAAGAAAUGUGGUCAG | 4623 | CUGACCACAUUUCUUCCAG | 70.2 | 62.2 |
| 881 | 3288 | UGGAAGAAAUGUGGUCAGC | 4624 | GCUGACCACAUUUCUUCCA | 58.3 | 50.3 |
| 882 | 3289 | GGAAGAAAUGUGGUCAGCG | 4625 | CGCUGACCACAUUUCUUCC | 67.6 | 59.6 |
| 883 | 3290 | GAAGAAAUGUGGUCAGCGA | 4626 | UCGCUGACCACAUUUCUUC | 93.7 | 85.7 |
| 884 | 3291 | AAGAAAUGUGGUCAGCGAA | 4627 | UUCGCUGACCACAUUUCUU | 88.1 | 80.1 |
| 885 | 3292 | AGAAAUGUGGUCAGCGAAG | 4628 | CUUCGCUGACCACAUUUCU | 74.9 | 66.9 |
| 886 | 3293 | GAAAUGUGGUCAGCGAAGC | 4629 | GCUUCGCUGACCACAUUUC | 66.5 | 58.5 |
| 887 | 3294 | AAAUGUGGUCAGCGAAGCG | 4630 | CGCUUCGCUGACCACAUUU | 64.1 | 56.1 |
| 888 | 3295 | AAUGUGGUCAGCGAAGCGC | 4631 | GCGCUUCGCUGACCACAUU | 54 | 46 |
| 889 | 3296 | AUGUGGUCAGCGAAGCGCU | 4632 | AGCGCUUCGCUGACCACAU | 70.5 | 62.5 |
| 890 | 3297 | UGUGGUCAGCGAAGCGCUU | 4633 | AAGCGCUUCGCUGACCACA | 79.3 | 71.3 |
| 891 | 3298 | GUGGUCAGCGAAGCGCUUA | 4634 | UAAGCGCUUCGCUGACCAC | 82.8 | 74.8 |
| 892 | 3299 | UGGUCAGCGAAGCGCUUAA | 4635 | UUAAGCGCUUCGCUGACCA | 85.5 | 77.5 |
| 893 | 3300 | GGUCAGCGAAGCGCUUAAG | 4636 | CUUAAGCGCUUCGCUGACC | 76.2 | 68.2 |
| 894 | 3301 | GUCAGCGAAGCGCUUAAGG | 4637 | CCUUAAGCGCUUCGCUGAC | 60.7 | 52.7 |
| 895 | 3302 | UCAGCGAAGCGCUUAAGGU | 4638 | ACCUUAAGCGCUUCGCUGA | 66.1 | 58.1 |
| 896 | 3303 | CAGCGAAGCGCUUAAGGUG | 4639 | CACCUUAAGCGCUUCGCUG | 72.5 | 64.5 |
| 897 | 3304 | AGCGAAGCGCUUAAGGUGC | 4640 | GCACCUUAAGCGCUUCGCU | 63.8 | 55.8 |
| 898 | 3305 | GCGAAGCGCUUAAGGUGCC | 4641 | GGCACCUUAAGCGCUUCGC | 65.7 | 57.7 |
| 899 | 3306 | CGAAGCGCUUAAGGUGCCG | 4642 | CGGCACCUUAAGCGCUUCG | 62.7 | 54.7 |
| 900 | 3307 | GAAGCGCUUAAGGUGCCGG | 4643 | CCGGCACCUUAAGCGCUUC | 55 | 46 |
| 901 | 3308 | AAGCGCUUAAGGUGCCGGU | 4644 | ACCGGCACCUUAAGCGCUU | 60.4 | 51.4 |
| 902 | 3309 | AGCGCUUAAGGUGCCGGUG | 4645 | CACCGGCACCUUAAGCGCU | 59.7 | 50.7 |
| 903 | 3310 | GCGCUUAAGGUGCCGGUGU | 4646 | ACACCGGCACCUUAAGCGC | 74.3 | 65.3 |
| 904 | 3311 | CGCUUAAGGUGCCGGUGUC | 4647 | GACACCGGCACCUUAAGCG | 70.1 | 61.1 |
| 905 | 3312 | GCUUAAGGUGCCGGUGUCU | 4648 | AGACACCGGCACCUUAAGC | 75.4 | 66.4 |
| 906 | 3313 | CUUAAGGUGCCGGUGUCUG | 4649 | CAGACACCGGCACCUUAAG | 60.2 | 51.2 |
| 907 | 3314 | UUAAGGUGCCGGUGUCUGA | 4650 | UCAGACACCGGCACCUUAA | 69.5 | 60.5 |
| 908 | 3315 | UAAGGUGCCGGUGUCUGAA | 4651 | UUCAGACACCGGCACCUUA | 71.8 | 62.8 |
| 909 | 3316 | AAGGUGCCGGUGUCUGAAG | 4652 | CUUCAGACACCGGCACCUU | 61.7 | 52.7 |
| 910 | 3317 | AGGUGCCGGUGUCUGAAGG | 4653 | CCUUCAGACACCGGCACCU | 58.5 | 49.5 |
| 911 | 3318 | GGUGCCGGUGUCUGAAGGC | 4654 | GCCUUCAGACACCGGCACC | 58 | 49 |
| 912 | 3319 | GUGCCGGUGUCUGAAGGCU | 4655 | AGCCUUCAGACACCGGCAC | 67.5 | 58.5 |
| 913 | 3320 | UGCCGGUGUCUGAAGGCUG | 4656 | CAGCCUUCAGACACCGGCA | 61.7 | 52.7 |
| 914 | 3321 | GCCGGUGUCUGAAGGCUGC | 4657 | GCAGCCUUCAGACACCGGC | 66.1 | 57.1 |
| 915 | 3322 | CCGGUGUCUGAAGGCUGCA | 4658 | UGCAGCCUUCAGACACCGG | 76.3 | 67.3 |
| 916 | 3323 | CGGUGUCUGAAGGCUGCAG | 4659 | CUGCAGCCUUCAGACACCG | 69.4 | 60.4 |
| 917 | 3324 | GGUGUCUGAAGGCUGCAGC | 4660 | GCUGCAGCCUUCAGACACC | 61.4 | 52.4 |
| 918 | 3325 | GUGUCUGAAGGCUGCAGCC | 4661 | GGCUGCAGCCUUCAGACAC | 58.6 | 49.6 |
| 935 | 3326 | CCAGGCUCUGAUGCGUCUC | 4662 | GAGACGCAUCAGAGCCUGG | 59.8 | 50.8 |
| 936 | 3327 | CAGGCUCUGAUGCGUCUCA | 4663 | UGAGACGCAUCAGAGCCUG | 78.3 | 69.3 |
| 937 | 3328 | AGGCUCUGAUGCGUCUCAU | 4664 | AUGAGACGCAUCAGAGCCU | 73.1 | 64.1 |
| 938 | 3329 | GGCUCUGAUGCGUCUCAUC | 4665 | GAUGAGACGCAUCAGAGCC | 72.1 | 63.1 |
| 939 | 3330 | GCUCUGAUGCGUCUCAUCG | 4666 | CGAUGAGACGCAUCAGAGC | 67.3 | 58.3 |
| 940 | 3331 | CUCUGAUGCGUCUCAUCGG | 4667 | CCGAUGAGACGCAUCAGAG | 61.4 | 52.4 |
| 941 | 3332 | UCUGAUGCGUCUCAUCGGC | 4668 | GCCGAUGAGACGCAUCAGA | 49.6 | 40.6 |
| 942 | 3333 | CUGAUGCGUCUCAUCGGCU | 4669 | AGCCGAUGAGACGCAUCAG | 63.4 | 54.4 |
| 943 | 3334 | UGAUGCGUCUCAUCGGCUG | 4670 | CAGCCGAUGAGACGCAUCA | 61.2 | 52.2 |
| 944 | 3335 | GAUGCGUCUCAUCGGCUGU | 4671 | ACAGCCGAUGAGACGCAUC | 75.6 | 66.6 |
| 945 | 3336 | AUGCGUCUCAUCGGCUGUC | 4672 | GACAGCCGAUGAGACGCAU | 49.5 | 40.5 |
| 946 | 3337 | UGCGUCUCAUCGGCUGUCC | 4673 | GGACAGCCGAUGAGACGCA | 48.5 | 39.5 |
| 947 | 3338 | GCGUCUCAUCGGCUGUCCC | 4674 | GGGACAGCCGAUGAGACGC | 60.6 | 51.6 |
| 981 | 3339 | CCCUCACUUAUGCCCUGCC | 4675 | GGCAGGGCAUAAGUGAGGG | 50.7 | 41.7 |
| 998 | 3340 | CCAGGGCUUCUGCCUCAAC | 4676 | GUUGAGGCAGAAGCCCUGG | 61.8 | 52.8 |
| 999 | 3341 | CAGGGCUUCUGCCUCAACG | 4677 | CGUUGAGGCAGAAGCCCUG | 57.8 | 48.8 |
| 1000 | 3342 | AGGGCUUCUGCCUCAACGU | 4678 | ACGUUGAGGCAGAAGCCCU | 65.7 | 55.7 |
| 1001 | 3343 | GGGCUUCUGCCUCAACGUG | 4679 | CACGUUGAGGCAGAAGCCC | 66.8 | 56.8 |
| 1002 | 3344 | GGCUUCUGCCUCAACGUGG | 4680 | CCACGUUGAGGCAGAAGCC | 64.3 | 54.3 |
| 1003 | 3345 | GCUUCUGCCUCAACGUGGU | 4681 | ACCACGUUGAGGCAGAAGC | 77.4 | 67.4 |
| 1004 | 3346 | CUUCUGCCUCAACGUGGUU | 4682 | AACCACGUUGAGGCAGAAG | 72.3 | 62.3 |
| 1005 | 3347 | UUCUGCCUCAACGUGGUUC | 4683 | GAACCACGUUGAGGCAGAA | 43.5 | 33.5 |
| 1006 | 3348 | UCUGCCUCAACGUGGUUCG | 4684 | CGAACCACGUUGAGGCAGA | 50.9 | 40.9 |
| 1007 | 3349 | CUGCCUCAACGUGGUUCGU | 4685 | ACGAACCACGUUGAGGCAG | 64.3 | 54.3 |
| 1008 | 3350 | UGCCUCAACGUGGUUCGUG | 4686 | CACGAACCACGUUGAGGCA | 48.7 | 38.7 |
| 1009 | 3351 | GCCUCAACGUGGUUCGUGG | 4687 | CCACGAACCACGUUGAGGC | 66.3 | 56.3 |
| 1026 | 3352 | GGCUGUCUCAGCAGCAGGG | 4688 | CCCUGCUGCUGAGACAGCC | 65.7 | 55.7 |
| 1043 | 3353 | GGGACUGGAGCCUGACUGG | 4689 | CCAGUCAGGCUCCAGUCCC | 63.3 | 53.3 |
| 1044 | 3354 | GGACUGGAGCCUGACUGGG | 4690 | CCCAGUCAGGCUCCAGUCC | 57.5 | 47.5 |
| 1061 | 3355 | GGGCAACUAUCUGGAUGGU | 4691 | ACCAUCCAGAUAGUUGCCC | 75.4 | 65.4 |
| 1062 | 3356 | GGCAACUAUCUGGAUGGUC | 4692 | GACCAUCCAGAUAGUUGCC | 65 | 55 |
| 1063 | 3357 | GCAACUAUCUGGAUGGUCU | 4693 | AGACCAUCCAGAUAGUUGC | 83.5 | 73.5 |
| 1064 | 3358 | CAACUAUCUGGAUGGUCUC | 4694 | GAGACCAUCCAGAUAGUUG | 71.7 | 61.7 |
| 1065 | 3359 | AACUAUCUGGAUGGUCUCC | 4695 | GGAGACCAUCCAGAUAGUU | 53.7 | 43.7 |
| 1066 | 3360 | ACUAUCUGGAUGGUCUCCU | 4696 | AGGAGACCAUCCAGAUAGU | 60.9 | 50.9 |
| 1067 | 3361 | CUAUCUGGAUGGUCUCCUG | 4697 | CAGGAGACCAUCCAGAUAG | 71.8 | 61.8 |
| 1068 | 3362 | UAUCUGGAUGGUCUCCUGA | 4698 | UCAGGAGACCAUCCAGAUA | 70.3 | 60.3 |
| 1069 | 3363 | AUCUGGAUGGUCUCCUGAU | 4699 | AUCAGGAGACCAUCCAGAU | 71.8 | 61.8 |
| 1070 | 3364 | UCUGGAUGGUCUCCUGAUC | 4700 | GAUCAGGAGACCAUCCAGA | 61.4 | 51.4 |
| 1071 | 3365 | CUGGAUGGUCUCCUGAUCC | 4701 | GGAUCAGGAGACCAUCCAG | 57.1 | 47.1 |
| 1072 | 3366 | UGGAUGGUCUCCUGAUCCU | 4702 | AGGAUCAGGAGACCAUCCA | 70.7 | 60.7 |
| 1073 | 3367 | GGAUGGUCUCCUGAUCCUG | 4703 | CAGGAUCAGGAGACCAUCC | 74.2 | 64.2 |
| 1074 | 3368 | GAUGGUCUCCUGAUCCUGG | 4704 | CCAGGAUCAGGAGACCAUC | 65.9 | 55.9 |
| 1091 | 3369 | GGCUGAUAAGCUCCAGGGC | 4705 | GCCCUGGAGCUUAUCAGCC | 61.2 | 51.2 |
| 1092 | 3370 | GCUGAUAAGCUCCAGGGCC | 4706 | GGCCCUGGAGCUUAUCAGC | 56.9 | 46.9 |
| 1093 | 3371 | CUGAUAAGCUCCAGGGCCC | 4707 | GGGCCCUGGAGCUUAUCAG | 51.4 | 41.4 |
| 1114 | 3372 | UUUCCUUUGAGCUGACGGC | 4708 | GCCGUCAGCUCAAAGGAAA | 42.5 | 31.5 |
| 1115 | 3373 | UUCCUUUGAGCUGACGGCC | 4709 | GGCCGUCAGCUCAAAGGAA | 37.3 | 26.3 |
| 1116 | 3374 | UCCUUUGAGCUGACGGCCG | 4710 | CGGCCGUCAGCUCAAAGGA | 49.1 | 38.1 |
| 1117 | 3375 | CCUUUGAGCUGACGGCCGA | 4711 | UCGGCCGUCAGCUCAAAGG | 80.5 | 69.5 |
| 1118 | 3376 | CUUUGAGCUGACGGCCGAG | 4712 | CUCGGCCGUCAGCUCAAAG | 58.9 | 47.9 |
| 1119 | 3377 | UUUGAGCUGACGGCCGAGU | 4713 | ACUCGGCCGUCAGCUCAAA | 49.9 | 38.9 |
| 1120 | 3378 | UUGAGCUGACGGCCGAGUC | 4714 | GACUCGGCCGUCAGCUCAA | 47.5 | 36.5 |
| 1121 | 3379 | UGAGCUGACGGCCGAGUCC | 4715 | GGACUCGGCCGUCAGCUCA | 53.5 | 42.5 |
| 1122 | 3380 | GAGCUGACGGCCGAGUCCA | 4716 | UGGACUCGGCCGUCAGCUC | 77 | 66 |
| 1123 | 3381 | AGCUGACGGCCGAGUCCAU | 4717 | AUGGACUCGGCCGUCAGCU | 76.9 | 65.9 |
| 1124 | 3382 | GCUGACGGCCGAGUCCAUU | 4718 | AAUGGACUCGGCCGUCAGC | 71.9 | 60.9 |
| 1125 | 3383 | CUGACGGCCGAGUCCAUUG | 4719 | CAAUGGACUCGGCCGUCAG | 62.8 | 51.8 |
| 1126 | 3384 | UGACGGCCGAGUCCAUUGG | 4720 | CCAAUGGACUCGGCCGUCA | 55.1 | 44.1 |
| 1127 | 3385 | GACGGCCGAGUCCAUUGGG | 4721 | CCCAAUGGACUCGGCCGUC | 54.9 | 43.9 |
| 1144 | 3386 | GGGUGAAGAUCUCGGAGGG | 4722 | CCCUCCGAGAUCUUCACCC | 66.3 | 55.3 |
| 1145 | 3387 | GGUGAAGAUCUCGGAGGGU | 4723 | ACCCUCCGAGAUCUUCACC | 71.7 | 60.7 |
| 1146 | 3388 | GUGAAGAUCUCGGAGGGUU | 4724 | AACCCUCCGAGAUCUUCAC | 74.9 | 63.9 |
| 1147 | 3389 | UGAAGAUCUCGGAGGGUUU | 4725 | AAACCCUCCGAGAUCUUCA | 75.5 | 64.5 |
| 1148 | 3390 | GAAGAUCUCGGAGGGUUUG | 4726 | CAAACCCUCCGAGAUCUUC | 69.2 | 58.2 |
| 1149 | 3391 | AAGAUCUCGGAGGGUUUGA | 4727 | UCAAACCCUCCGAGAUCUU | 68.2 | 57.2 |
| 1150 | 3392 | AGAUCUCGGAGGGUUUGAU | 4728 | AUCAAACCCUCCGAGAUCU | 71.3 | 60.3 |
| 1151 | 3393 | GAUCUCGGAGGGUUUGAUG | 4729 | CAUCAAACCCUCCGAGAUC | 65.5 | 54.5 |
| 1152 | 3394 | AUCUCGGAGGGUUUGAUGU | 4730 | ACAUCAAACCCUCCGAGAU | 63.4 | 52.4 |
| 1153 | 3395 | UCUCGGAGGGUUUGAUGUA | 4731 | UACAUCAAACCCUCCGAGA | 76.8 | 65.8 |
| 1154 | 3396 | CUCGGAGGGUUUGAUGUAC | 4732 | GUACAUCAAACCCUCCGAG | 63.5 | 52.5 |
| 1155 | 3397 | UCGGAGGGUUUGAUGUACC | 4733 | GGUACAUCAAACCCUCCGA | 53.1 | 42.1 |
| 1156 | 3398 | CGGAGGGUUUGAUGUACCU | 4734 | AGGUACAUCAAACCCUCCG | 80.7 | 69.7 |
| 1157 | 3399 | GGAGGGUUUGAUGUACCUG | 4735 | CAGGUACAUCAAACCCUCC | 70.9 | 59.9 |
| 1158 | 3400 | GAGGGUUUGAUGUACCUGC | 4736 | GCAGGUACAUCAAACCCUC | 64.1 | 53.1 |
| 1159 | 3401 | AGGGUUUGAUGUACCUGCA | 4737 | UGCAGGUACAUCAAACCCU | 83.8 | 72.8 |
| 1160 | 3402 | GGGUUUGAUGUACCUGCAG | 4738 | CUGCAGGUACAUCAAACCC | 76.6 | 65.6 |
| 1161 | 3403 | GGUUUGAUGUACCUGCAGG | 4739 | CCUGCAGGUACAUCAAACC | 73.4 | 62.4 |
| 1162 | 3404 | GUUUGAUGUACCUGCAGGA | 4740 | UCCUGCAGGUACAUCAAAC | 82.5 | 71.5 |
| 1163 | 3405 | UUUGAUGUACCUGCAGGAA | 4741 | UUCCUGCAGGUACAUCAAA | 71.2 | 60.2 |
| 1164 | 3406 | UUGAUGUACCUGCAGGAAA | 4742 | UUUCCUGCAGGUACAUCAA | 76.2 | 65.2 |
| 1181 | 3407 | AAACAGUGCGAAGGUGUCC | 4743 | GGACACCUUCGCACUGUUU | 58.4 | 47.4 |
| 1182 | 3408 | AACAGUGCGAAGGUGUCCG | 4744 | CGGACACCUUCGCACUGUU | 56.5 | 45.5 |
| 1183 | 3409 | ACAGUGCGAAGGUGUCCGC | 4745 | GCGGACACCUUCGCACUGU | 58.4 | 47.4 |
| 1184 | 3410 | CAGUGCGAAGGUGUCCGCC | 4746 | GGCGGACACCUUCGCACUG | 53.4 | 42.4 |
| 1185 | 3411 | AGUGCGAAGGUGUCCGCCC | 4747 | GGGCGGACACCUUCGCACU | 57.5 | 46.5 |
| 1186 | 3412 | GUGCGAAGGUGUCCGCCCA | 4748 | UGGGCGGACACCUUCGCAC | 81.4 | 70.4 |
| 1187 | 3413 | UGCGAAGGUGUCCGCCCAG | 4749 | CUGGGCGGACACCUUCGCA | 60.1 | 49.1 |
| 1188 | 3414 | GCGAAGGUGUCCGCCCAGG | 4750 | CCUGGGCGGACACCUUCGC | 61 | 50 |
| 1189 | 3415 | CGAAGGUGUCCGCCCAGGU | 4751 | ACCUGGGCGGACACCUUCG | 72.1 | 61.1 |
| 1190 | 3416 | GAAGGUGUCCGCCCAGGUG | 4752 | CACCUGGGCGGACACCUUC | 66.1 | 55.1 |
| 1191 | 3417 | AAGGUGUCCGCCCAGGUGU | 4753 | ACACCUGGGCGGACACCUU | 63.9 | 52.9 |
| 1192 | 3418 | AGGUGUCCGCCCAGGUGUU | 4754 | AACACCUGGGCGGACACCU | 75.8 | 64.8 |
| 1193 | 3419 | GGUGUCCGCCCAGGUGUUU | 4755 | AAACACCUGGGCGGACACC | 74.8 | 63.8 |
| 1194 | 3420 | GUGUCCGCCCAGGUGUUUC | 4756 | GAAACACCUGGGCGGACAC | 56.9 | 45.9 |
| 1195 | 3421 | UGUCCGCCCAGGUGUUUCA | 4757 | UGAAACACCUGGGCGGACA | 73.1 | 62.1 |
| 1196 | 3422 | GUCCGCCCAGGUGUUUCAG | 4758 | CUGAAACACCUGGGCGGAC | 58.2 | 47.2 |
| 1197 | 3423 | UCCGCCCAGGUGUUUCAGG | 4759 | CCUGAAACACCUGGGCGGA | 43.4 | 32.4 |
| 1198 | 3424 | CCGCCCAGGUGUUUCAGGA | 4760 | UCCUGAAACACCUGGGCGG | 73.3 | 62.3 |
| 1199 | 3425 | CGCCCAGGUGUUUCAGGAG | 4761 | CUCCUGAAACACCUGGGCG | 67.1 | 56.1 |
| 1200 | 3426 | GCCCAGGUGUUUCAGGAGU | 4762 | ACUCCUGAAACACCUGGGC | 76.4 | 64.4 |
| 1201 | 3427 | CCCAGGUGUUUCAGGAGUG | 4763 | CACUCCUGAAACACCUGGG | 72 | 60 |
| 1202 | 3428 | CCAGGUGUUUCAGGAGUGC | 4764 | GCACUCCUGAAACACCUGG | 64.8 | 52.8 |
| 1203 | 3429 | CAGGUGUUUCAGGAGUGCG | 4765 | CGCACUCCUGAAACACCUG | 64 | 52 |
| 1204 | 3430 | AGGUGUUUCAGGAGUGCGG | 4766 | CCGCACUCCUGAAACACCU | 66.4 | 54.4 |
| 1205 | 3431 | GGUGUUUCAGGAGUGCGGC | 4767 | GCCGCACUCCUGAAACACC | 62.1 | 50.1 |
| 1206 | 3432 | GUGUUUCAGGAGUGCGGCC | 4768 | GGCCGCACUCCUGAAACAC | 56.9 | 44.9 |
| 1207 | 3433 | UGUUUCAGGAGUGCGGCCC | 4769 | GGGCCGCACUCCUGAAACA | 44 | 32 |
| 1227 | 3434 | CCCGACCCGGUGCCUGCCC | 4770 | GGGCAGGCACCGGGUCGGG | 45.8 | 33.8 |
| 1228 | 3435 | CCGACCCGGUGCCUGCCCG | 4771 | CGGGCAGGCACCGGGUCGG | 51.8 | 39.8 |
| 1229 | 3436 | CGACCCGGUGCCUGCCCGC | 4772 | GCGGGCAGGCACCGGGUCG | 50.8 | 38.8 |
| 1230 | 3437 | GACCCGGUGCCUGCCCGCA | 4773 | UGCGGGCAGGCACCGGGUC | 65.8 | 53.8 |
| 1231 | 3438 | ACCCGGUGCCUGCCCGCAA | 4774 | UUGCGGGCAGGCACCGGGU | 72.6 | 60.6 |
| 1232 | 3439 | CCCGGUGCCUGCCCGCAAC | 4775 | GUUGCGGGCAGGCACCGGG | 59.7 | 47.7 |
| 1233 | 3440 | CCGGUGCCUGCCCGCAACC | 4776 | GGUUGCGGGCAGGCACCGG | 49.5 | 37.5 |
| 1234 | 3441 | CGGUGCCUGCCCGCAACCG | 4777 | CGGUUGCGGGCAGGCACCG | 51.8 | 39.8 |
| 1235 | 3442 | GGUGCCUGCCCGCAACCGU | 4778 | ACGGUUGCGGGCAGGCACC | 65.8 | 53.8 |
| 1236 | 3443 | GUGCCUGCCCGCAACCGUC | 4779 | GACGGUUGCGGGCAGGCAC | 55.5 | 43.5 |
| 1237 | 3444 | UGCCUGCCCGCAACCGUCG | 4780 | CGACGGUUGCGGGCAGGCA | 45.8 | 33.8 |
| 1238 | 3445 | GCCUGCCCGCAACCGUCGA | 4781 | UCGACGGUUGCGGGCAGGC | 72.1 | 60.1 |
| 1239 | 3446 | CCUGCCCGCAACCGUCGAG | 4782 | CUCGACGGUUGCGGGCAGG | 54.6 | 42.6 |
| 1240 | 3447 | CUGCCCGCAACCGUCGAGC | 4783 | GCUCGACGGUUGCGGGCAG | 39.2 | 27.2 |
| 1241 | 3448 | UGCCCGCAACCGUCGAGCC | 4784 | GGCUCGACGGUUGCGGGCA | 43.2 | 31.2 |
| 1242 | 3449 | GCCCGCAACCGUCGAGCCC | 4785 | GGGCUCGACGGUUGCGGGC | 50.8 | 38.8 |
| 1267 | 3450 | CCCGGGAAGAGGCGGGCCG | 4786 | CGGCCCGCCUCUUCCCGGG | 48.2 | 36.2 |
| 1268 | 3451 | CCGGGAAGAGGCGGGCCGG | 4787 | CCGGCCCGCCUCUUCCCGG | 47.9 | 35.9 |
| 1269 | 3452 | CGGGAAGAGGCGGGCCGGC | 4788 | GCCGGCCCGCCUCUUCCCG | 43.6 | 31.6 |
| 1270 | 3453 | GGGAAGAGGCGGGCCGGCU | 4789 | AGCCGGCCCGCCUCUUCCC | 60.7 | 48.7 |
| 1271 | 3454 | GGAAGAGGCGGGCCGGCUG | 4790 | CAGCCGGCCCGCCUCUUCC | 63.9 | 51.9 |
| 1272 | 3455 | GAAGAGGCGGGCCGGCUGU | 4791 | ACAGCCGGCCCGCCUCUUC | 66.7 | 54.7 |
| 1273 | 3456 | AAGAGGCGGGCCGGCUGUG | 4792 | CACAGCCGGCCCGCCUCUU | 48.5 | 36.5 |
| 1274 | 3457 | AGAGGCGGGCCGGCUGUGG | 4793 | CCACAGCCGGCCCGCCUCU | 46 | 34 |
| 1275 | 3458 | GAGGCGGGCCGGCUGUGGU | 4794 | ACCACAGCCGGCCCGCCUC | 61.8 | 49.8 |
| 1276 | 3459 | AGGCGGGCCGGCUGUGGUC | 4795 | GACCACAGCCGGCCCGCCU | 53.6 | 41.6 |
| 1277 | 3460 | GGCGGGCCGGCUGUGGUCG | 4796 | CGACCACAGCCGGCCCGCC | 53.1 | 41.1 |
| 1278 | 3461 | GCGGGCCGGCUGUGGUCGA | 4797 | UCGACCACAGCCGGCCCGC | 74.2 | 62.2 |
| 1279 | 3462 | CGGGCCGGCUGUGGUCGAU | 4798 | AUCGACCACAGCCGGCCCG | 70.7 | 58.7 |
| 1280 | 3463 | GGGCCGGCUGUGGUCGAUG | 4799 | CAUCGACCACAGCCGGCCC | 52.7 | 50.7 |
| 1281 | 3464 | GGCCGGCUGUGGUCGAUGG | 4800 | CCAUCGACCACAGCCGGCC | 69.7 | 57.7 |
| 1282 | 3465 | GCCGGCUGUGGUCGAUGGU | 4801 | ACCAUCGACCACAGCCGGC | 72 | 60 |
| 1283 | 3466 | CCGGCUGUGGUCGAUGGUG | 4802 | CACCAUCGACCACAGCCGG | 64.9 | 52.9 |
| 1284 | 3467 | CGGCUGUGGUCGAUGGUGA | 4803 | UCACCAUCGACCACAGCCG | 88.4 | 76.4 |
| 1285 | 3468 | GGCUGUGGUCGAUGGUGAC | 4804 | GUCACCAUCGACCACAGCC | 78.1 | 66.1 |
| 1286 | 3469 | GCUGUGGUCGAUGGUGACC | 4805 | GGUCACCAUCGACCACAGC | 63.9 | 51.9 |
| 1287 | 3470 | CUGUGGUCGAUGGUGACCG | 4806 | CGGUCACCAUCGACCACAG | 58 | 46 |
| 1288 | 3471 | UGUGGUCGAUGGUGACCGA | 4807 | UCGGUCACCAUCGACCACA | 84.6 | 72.6 |
| 1289 | 3472 | GUGGUCGAUGGUGACCGAG | 4808 | CUCGGUCACCAUCGACCAC | 64.7 | 52.7 |
| 1290 | 3473 | UGGUCGAUGGUGACCGAGG | 4809 | CCUCGGUCACCAUCGACCA | 60.6 | 48.6 |
| 1291 | 3474 | GGUCGAUGGUGACCGAGGA | 4810 | UCCUCGGUCACCAUCGACC | 92.2 | 80.2 |
| 1292 | 3475 | GUCGAUGGUGACCGAGGAG | 4811 | CUCCUCGGUCACCAUCGAC | 69.3 | 57.3 |
| 1293 | 3476 | UCGAUGGUGACCGAGGAGG | 4812 | CCUCCUCGGUCACCAUCGA | 49.8 | 37.8 |
| 1294 | 3477 | CGAUGGUGACCGAGGAGGA | 4813 | UCCUCCUCGGUCACCAUCG | 88.4 | 76.4 |
| 1295 | 3478 | GAUGGUGACCGAGGAGGAG | 4814 | CUCCUCCUCGGUCACCAUC | 69 | 57 |
| 1296 | 3479 | AUGGUGACCGAGGAGGAGC | 4815 | GCUCCUCCUCGGUCACCAU | 46.7 | 34.7 |
| 1297 | 3480 | UGGUGACCGAGGAGGAGCG | 4816 | CGCUCCUCCUCGGUCACCA | 55.8 | 43.8 |
| 1298 | 3481 | GGUGACCGAGGAGGAGCGG | 4817 | CCGCUCCUCCUCGGUCACC | 60.9 | 48.9 |
| 1299 | 3482 | GUGACCGAGGAGGAGCGGC | 4818 | GCCGCUCCUCCUCGGUCAC | 47.3 | 35.3 |
| 1300 | 3483 | UGACCGAGGAGGAGCGGCC | 4819 | GGCCGCUCCUCCUCGGUCA | 46.7 | 33.7 |
| 1301 | 3484 | GACCGAGGAGGAGCGGCCC | 4820 | GGGCCGCUCCUCCUCGGUC | 53.4 | 40.4 |
| 1302 | 3485 | ACCGAGGAGGAGCGGCCCA | 4821 | UGGGCCGCUCCUCCUCGGU | 67.1 | 54.1 |
| 1303 | 3486 | CCGAGGAGGAGCGGCCCAC | 4822 | GUGGGCCGCUCCUCCUCGG | 55.3 | 42.3 |
| 1304 | 3487 | CGAGGAGGAGCGGCCCACG | 4823 | CGUGGGCCGCUCCUCCUCG | 57.6 | 44.6 |
| 1305 | 3488 | GAGGAGGAGCGGCCCACGA | 4824 | UCGUGGGCCGCUCCUCCUC | 76.3 | 63.3 |
| 1306 | 3489 | AGGAGGAGCGGCCCACGAC | 4825 | GUCGUGGGCCGCUCCUCCU | 59 | 46 |
| 1307 | 3490 | GGAGGAGCGGCCCACGACG | 4826 | CGUCGUGGGCCGCUCCUCC | 64.5 | 51.5 |
| 1308 | 3491 | GAGGAGCGGCCCACGACGG | 4827 | CCGUCGUGGGCCGCUCCUC | 59.4 | 46.4 |
| 1309 | 3492 | AGGAGCGGCCCACGACGGC | 4828 | GCCGUCGUGGGCCGCUCCU | 47.1 | 34.1 |
| 1310 | 3493 | GGAGCGGCCCACGACGGCC | 4829 | GGCCGUCGUGGGCCGCUCC | 52.1 | 39.1 |
| 1311 | 3494 | GAGCGGCCCACGACGGCCG | 4830 | CGGCCGUCGUGGGCCGCUC | 54.8 | 41.8 |
| 1312 | 3495 | AGCGGCCCACGACGGCCGC | 4831 | GCGGCCGUCGUGGGCCGCU | 45 | 32 |
| 1313 | 3496 | GCGGCCCACGACGGCCGCA | 4832 | UGCGGCCGUCGUGGGCCGC | 63.7 | 50.7 |
| 1314 | 3497 | CGGCCCACGACGGCCGCAG | 4833 | CUGCGGCCGUCGUGGGCCG | 53.2 | 40.2 |
| 1315 | 3498 | GGCCCACGACGGCCGCAGG | 4834 | CCUGCGGCCGUCGUGGGCC | 58 | 45 |
| 1316 | 3499 | GCCCACGACGGCCGCAGGC | 4835 | GCCUGCGGCCGUCGUGGGC | 47.6 | 34.6 |
| 1317 | 3500 | CCCACGACGGCCGCAGGCA | 4836 | UGCCUGCGGCCGUCGUGGG | 66.5 | 53.5 |
| 1318 | 3501 | CCACGACGGCCGCAGGCAC | 4837 | GUGCCUGCGGCCGUCGUGG | 61.6 | 48.6 |
| 1319 | 3502 | CACGACGGCCGCAGGCACC | 4838 | GGUGCCUGCGGCCGUCGUG | 52.3 | 39.3 |
| 1320 | 3503 | ACGACGGCCGCAGGCACCA | 4839 | UGGUGCCUGCGGCCGUCGU | 65 | 52 |
| 1321 | 3504 | CGACGGCCGCAGGCACCAA | 4840 | UUGGUGCCUGCGGCCGUCG | 82.7 | 69.7 |
| 1322 | 3505 | GACGGCCGCAGGCACCAAC | 4841 | GUUGGUGCCUGCGGCCGUC | 57 | 44 |
| 1323 | 3506 | ACGGCCGCAGGCACCAACC | 4842 | GGUUGGUGCCUGCGGCCGU | 45.2 | 32.2 |
| 1324 | 3507 | CGGCCGCAGGCACCAACCU | 4843 | AGGUUGGUGCCUGCGGCCG | 66.8 | 53.8 |
| 1325 | 3508 | GGCCGCAGGCACCAACCUG | 4844 | CAGGUUGGUGCCUGCGGCC | 63.5 | 50.5 |
| 1326 | 3509 | GCCGCAGGCACCAACCUGC | 4845 | GCAGGUUGGUGCCUGCGGC | 56.5 | 43.5 |
| 1327 | 3510 | CCGCAGGCACCAACCUGCA | 4846 | UGCAGGUUGGUGCCUGCGG | 77.1 | 64.1 |
| 1328 | 3511 | CGCAGGCACCAACCUGCAC | 4847 | GUGCAGGUUGGUGCCUGCG | 65.9 | 52.9 |
| 1329 | 3512 | GCAGGCACCAACCUGCACC | 4848 | GGUGCAGGUUGGUGCCUGC | 55.6 | 42.6 |
| 1330 | 3513 | CAGGCACCAACCUGCACCG | 4849 | CGGUGCAGGUUGGUGCCUG | 58.3 | 45.3 |
| 1331 | 3514 | AGGCACCAACCUGCACCGG | 4850 | CCGGUGCAGGUUGGUGCCU | 50.4 | 37.4 |
| 1332 | 3515 | GGCACCAACCUGCACCGGC | 4851 | GCCGGUGCAGGUUGGUGCC | 54.5 | 41.5 |
| 1333 | 3516 | GCACCAACCUGCACCGGCU | 4852 | AGCCGGUGCAGGUUGGUGC | 75.5 | 62.5 |
| 1334 | 3517 | CACCAACCUGCACCGGCUG | 4853 | CAGCCGGUGCAGGUUGGUG | 63.2 | 50.2 |
| 1335 | 3518 | ACCAACCUGCACCGGCUGG | 4854 | CCAGCCGGUGCAGGUUGGU | 50.1 | 37.1 |
| 1336 | 3519 | CCAACCUGCACCGGCUGGU | 4855 | ACCAGCCGGUGCAGGUUGG | 59.4 | 46.4 |
| 1337 | 3520 | CAACCUGCACCGGCUGGUG | 4856 | CACCAGCCGGUGCAGGUUG | 60.6 | 47.6 |
| 1338 | 3521 | AACCUGCACCGGCUGGUGU | 4857 | ACACCAGCCGGUGCAGGUU | 62.4 | 49.4 |
| 1339 | 3522 | ACCUGCACCGGCUGGUGUG | 4858 | CACACCAGCCGGUGCAGGU | 60.1 | 47.1 |
| 1340 | 3523 | CCUGCACCGGCUGGUGUGG | 4859 | CCACACCAGCCGGUGCAGG | 57.3 | 44.3 |
| 1341 | 3524 | CUGCACCGGCUGGUGUGGG | 4860 | CCCACACCAGCCGGUGCAG | 44.7 | 31.7 |
| 1342 | 3525 | UGCACCGGCUGGUGUGGGA | 4861 | UCCCACACCAGCCGGUGCA | 67 | 54 |
| 1343 | 3526 | GCACCGGCUGGUGUGGGAG | 4862 | CUCCCACACCAGCCGGUGC | 61.3 | 48.3 |
| 1344 | 3527 | CACCGGCUGGUGUGGGAGC | 4863 | GCUCCCACACCAGCCGGUG | 54.1 | 41.1 |
| 1345 | 3528 | ACCGGCUGGUGUGGGAGCU | 4864 | AGCUCCCACACCAGCCGGU | 59.3 | 46.3 |
| 1346 | 3529 | CCGGCUGGUGUGGGAGCUC | 4865 | GAGCUCCCACACCAGCCGG | 58 | 45 |
| 1347 | 3530 | CGGCUGGUGUGGGAGCUCC | 4866 | GGAGCUCCCACACCAGCCG | 60.9 | 47.9 |
| 1348 | 3531 | GGCUGGUGUGGGAGCUCCG | 4867 | CGGAGCUCCCACACCAGCC | 69.2 | 56.2 |
| 1349 | 3532 | GCUGGUGUGGGAGCUCCGC | 4868 | GOGGAGCUCCCACACCAGC | 57.5 | 44.5 |
| 1350 | 3533 | CUGGUGUGGGAGCUCCGCG | 4869 | CGCGGAGCUCCCACACCAG | 51.8 | 38.8 |
| 1351 | 3534 | UGGUGUGGGAGCUCCGCGA | 4870 | UCGCGGAGCUCCCACACCA | 73.2 | 60.2 |
| 1352 | 3535 | GGUGUGGGAGCUCCGCGAG | 4871 | CUCGCGGAGCUCCCACACC | 70.5 | 57.5 |
| 1353 | 3536 | GUGUGGGAGCUCCGCGAGC | 4872 | GCUCGCGGAGCUCCCACAC | 57.9 | 44.9 |
| 1354 | 3537 | UGUGGGAGCUCCGCGAGCG | 4873 | CGCUCGCGGAGCUCCCACA | 52.9 | 39.9 |
| 1355 | 3538 | GUGGGAGCUCCGCGAGCGU | 4874 | ACGCUCGCGGAGCUCCCAC | 67.6 | 54.6 |
| 1356 | 3539 | UGGGAGCUCCGCGAGCGUC | 4875 | GACGCUCGCGGAGCUCCCA | 50.5 | 37.5 |
| 1357 | 3540 | GGGAGCUCCGCGAGCGUCU | 4876 | AGACGCUCGCGGAGCUCCC | 73 | 60 |
| 1358 | 3541 | GGAGCUCCGCGAGCGUCUG | 4877 | CAGACGCUCGCGGAGCUCC | 69.9 | 56.9 |
| 1359 | 3542 | GAGCUCCGCGAGCGUCUGG | 4878 | CCAGACGCUCGCGGAGCUC | 58.9 | 45.9 |
| 1388 | 3543 | GGGCUUCUGGGCCCGGCUG | 4879 | CAGCCGGGCCCAGAAGCCC | 58.5 | 45.5 |
| 1389 | 3544 | GGCUUCUGGGCCCGGCUGU | 4880 | ACAGCCGGGCCCAGAAGCC | 65.1 | 52.1 |
| 1390 | 3545 | GCUUCUGGGCCCGGCUGUC | 4881 | GACAGCCGGGCCCAGAAGC | 57 | 44 |
| 1391 | 3546 | CUUCUGGGCCCGGCUGUCC | 4882 | GGACAGCCGGGCCCAGAAG | 44.4 | 31.4 |
| 1392 | 3547 | UUCUGGGCCCGGCUGUCCC | 4883 | GGGACAGCCGGGCCCAGAA | 36.5 | 23.5 |
| 1393 | 3548 | UCUGGGCCCGGCUGUCCCU | 4884 | AGGGACAGCCGGGCCCAGA | 53.2 | 40.2 |
| 1394 | 3549 | CUGGGCCCGGCUGUCCCUG | 4885 | CAGGGACAGCCGGGCCCAG | 43.5 | 30.5 |
| 1395 | 3550 | UGGGCCCGGCUGUCCCUGA | 4886 | UCAGGGACAGCCGGGCCCA | 59 | 46 |
| 1396 | 3551 | GGGCCCGGCUGUCCCUGAC | 4887 | GUCAGGGACAGCCGGGCCC | 59.4 | 46.4 |
| 1397 | 3552 | GGCCCGGCUGUCCCUGACG | 4888 | CGUCAGGGACAGCCGGGCC | 58.9 | 45.9 |
| 1398 | 3553 | GCCCGGCUGUCCCUGACGG | 4889 | CCGUCAGGGACAGCCGGGC | 58.6 | 45.6 |
| 1399 | 3554 | CCCGGCUGUCCCUGACGGU | 4890 | ACCGUCAGGGACAGCCGGG | 64.7 | 51.7 |
| 1400 | 3555 | CCGGCUGUCCCUGACGGUG | 4891 | CACCGUCAGGGACAGCCGG | 60.9 | 46.9 |
| 1401 | 3556 | CGGCUGUCCCUGACGGUGU | 4892 | ACACCGUCAGGGACAGCCG | 76.4 | 62.4 |
| 1402 | 3557 | GGCUGUCCCUGACGGUGUG | 4893 | CACACCGUCAGGGACAGCC | 76.7 | 62.7 |
| 1403 | 3558 | GCUGUCCCUGACGGUGUGC | 4894 | GCACACCGUCAGGGACAGC | 57.6 | 43.6 |
| 1404 | 3559 | CUGUCCCUGACGGUGUGCG | 4895 | CGCACACCGUCAGGGACAG | 49.9 | 35.9 |
| 1405 | 3560 | UGUCCCUGACGGUGUGCGG | 4896 | CCGCACACCGUCAGGGACA | 56.2 | 42.2 |
| 1406 | 3561 | GUCCCUGACGGUGUGCGGA | 4897 | UCCGCACACCGUCAGGGAC | 72.1 | 58.1 |
| 1407 | 3562 | UCCCUGACGGUGUGCGGAG | 4898 | CUCCGCACACCGUCAGGGA | 56.2 | 42.2 |
| 1408 | 3563 | CCCUGACGGUGUGCGGAGA | 4899 | UCUCCGCACACCGUCAGGG | 80.8 | 66.8 |
| 1409 | 3564 | CCUGACGGUGUGCGGAGAC | 4900 | GUCUCCGCACACCGUCAGG | 63.8 | 49.8 |
| 1410 | 3565 | CUGACGGUGUGCGGAGACU | 4901 | AGUCUCCGCACACCGUCAG | 71.5 | 57.8 |
| 1411 | 3566 | UGACGGUGUGCGGAGACUC | 4902 | GAGUCUCCGCACACCGUCA | 61.6 | 47.6 |
| 1412 | 3567 | GACGGUGUGCGGAGACUCU | 4903 | AGAGUCUCCGCACACCGUC | 83.1 | 69.1 |
| 1413 | 3568 | ACGGUGUGCGGAGACUCUC | 4904 | GAGAGUCUCCGCACACCGU | 63.3 | 49.3 |
| 1414 | 3569 | CGGUGUGCGGAGACUCUCG | 4905 | CGAGAGUCUCCGCACACCG | 72.6 | 58.6 |
| 1415 | 3570 | GGUGUGCGGAGACUCUCGC | 4906 | GCGAGAGUCUCCGCACACC | 63.9 | 49.9 |
| 1416 | 3571 | GUGUGCGGAGACUCUCGCA | 4907 | UGCGAGAGUCUCCGCACAC | 78.1 | 64.1 |
| 1417 | 3572 | UGUGCGGAGACUCUCGCAU | 4908 | AUGCGAGAGUCUCCGCACA | 70.1 | 56.1 |
| 1418 | 3573 | GUGCGGAGACUCUCGCAUG | 4909 | CAUGCGAGAGUCUCCGCAC | 69.2 | 55.2 |
| 1419 | 3574 | UGCGGAGACUCUCGCAUGG | 4910 | CCAUGCGAGAGUCUCCGCA | 59.3 | 45.3 |
| 1436 | 3575 | GGCAGCGGACGCCUCGCUG | 4911 | CAGCGAGGCGUCCGCUGCC | 62.1 | 48.1 |
| 1437 | 3576 | GCAGCGGACGCCUCGCUGG | 4912 | CCAGCGAGGCGUCCGCUGC | 60.3 | 46.3 |
| 1438 | 3577 | CAGCGGACGCCUCGCUGGA | 4913 | UCCAGCGAGGCGUCCGCUG | 72.9 | 58.9 |
| 1439 | 3578 | AGCGGACGCCUCGCUGGAG | 4914 | CUCCAGCGAGGCGUCCGCU | 54.6 | 40.6 |
| 1440 | 3579 | GCGGACGCCUCGCUGGAGG | 4915 | CCUCCAGCGAGGCGUCCGC | 54.5 | 40.5 |
| 1441 | 3580 | CGGACGCCUCGCUGGAGGC | 4916 | GCCUCCAGCGAGGCGUCCG | 51.8 | 37.8 |
| 1442 | 3581 | GGACGCCUCGCUGGAGGCG | 4917 | CGCCUCCAGCGAGGCGUCC | 51 | 37 |
| 1443 | 3582 | GACGCCUCGCUGGAGGCGG | 4918 | CCGCCUCCAGCGAGGCGUC | 48 | 34 |
| 1444 | 3583 | ACGCCUCGCUGGAGGCGGC | 4919 | GCCGCCUCCAGCGAGGCGU | 45 | 31 |
| 1445 | 3584 | CGCCUCGCUGGAGGCGGCG | 4920 | CGCCGCCUCCAGCGAGGCG | 45.6 | 31.6 |
| 1446 | 3585 | GCCUCGCUGGAGGCGGCGC | 4921 | GCGCCGCCUCCAGCGAGGC | 46.8 | 32.8 |
| 1447 | 3586 | CCUCGCUGGAGGCGGCGCC | 4922 | GGCGCCGCCUCCAGCGAGG | 43.7 | 29.7 |
| 1448 | 3587 | CUCGCUGGAGGCGGCGCCC | 4923 | GGGCGCCGCCUCCAGCGAG | 40.3 | 26.3 |
| 1449 | 3588 | UCGCUGGAGGCGGCGCCCU | 4924 | AGGGCGCCGCCUCCAGCGA | 46.4 | 32.4 |
| 1450 | 3589 | CGCUGGAGGCGGCGCCCUG | 4925 | CAGGGCGCCGCCUCCAGCG | 61.1 | 47.1 |
| 1451 | 3590 | GCUGGAGGCGGCGCCCUGC | 4926 | GCAGGGCGCCGCCUCCAGC | 51.4 | 37.4 |
| 1452 | 3591 | CUGGAGGCGGCGCCCUGCU | 4927 | AGCAGGGCGCCGCCUCCAG | 57.2 | 43.2 |
| 1453 | 3592 | UGGAGGCGGCGCCCUGCUG | 4928 | CAGCAGGGCGCCGCCUCCA | 51.8 | 37.8 |
| 1454 | 3593 | GGAGGCGGCGCCCUGCUGG | 4929 | CCAGCAGGGCGCCGCCUCC | 53.9 | 39.9 |
| 1455 | 3594 | GAGGCGGCGCCCUGCUGGA | 4930 | UCCAGCAGGGCGCCGCCUC | 70.9 | 56.9 |
| 1456 | 3595 | AGGCGGCGCCCUGCUGGAC | 4931 | GUCCAGCAGGGCGCCGCCU | 50.3 | 36.3 |
| 1457 | 3596 | GGCGGCGCCCUGCUGGACC | 4932 | GGUCCAGCAGGGCGCCGCC | 51 | 37 |
| 1458 | 3597 | GCGGCGCCCUGCUGGACCG | 4933 | CGGUCCAGCAGGGCGCCGC | 60.4 | 46.4 |
| 1459 | 3598 | CGGCGCCCUGCUGGACCGG | 4934 | CCGGUCCAGCAGGGCGCCG | 49.7 | 35.7 |
| 1460 | 3599 | GGCGCCCUGCUGGACCGGA | 4935 | UCCGGUCCAGCAGGGCGCC | 67.2 | 53.2 |
| 1461 | 3600 | GCGCCCUGCUGGACCGGAG | 4936 | CUCCGGUCCAGCAGGGCGC | 63.8 | 49.8 |
| 1462 | 3601 | CGCCCUGCUGGACCGGAGC | 4937 | GCUCCGGUCCAGCAGGGCG | 55.2 | 41.2 |
| 1463 | 3602 | GCCCUGCUGGACCGGAGCC | 4938 | GGCUCCGGUCCAGCAGGGC | 51.1 | 37.1 |
| 1464 | 3603 | CCCUGCUGGACCGGAGCCG | 4939 | CGGCUCCGGUCCAGCAGGG | 49.5 | 35.5 |
| 1465 | 3604 | CCUGCUGGACCGGAGCCGG | 4940 | CCGGCUCCGGUCCAGCAGG | 54.3 | 40.3 |
| 1466 | 3605 | CUGCUGGACCGGAGCCGGG | 4941 | CCCGGCUCCGGUCCAGCAG | 50 | 36 |
| 1467 | 3606 | UGCUGGACCGGAGCCGGGC | 4942 | GCCCGGCUCCGGUCCAGCA | 40.2 | 26.2 |
| 1468 | 3607 | GCUGGACCGGAGCCGGGCG | 4943 | CGCCCGGCUCCGGUCCAGC | 54.4 | 40.4 |
| 1469 | 3608 | CUGGACCGGAGCCGGGCGG | 4944 | CCGCCCGGCUCCGGUCCAG | 42.5 | 28.5 |
| 1470 | 3609 | UGGACCGGAGCCGGGCGGG | 4945 | CCCGCCCGGCUCCGGUCCA | 33.5 | 19.5 |
| 1520 | 3610 | CCCGGCCGAGCAGGUCAAC | 4946 | GUUGACCUGCUCGGCCGGG | 55.8 | 40.8 |
| 1521 | 3611 | CCGGCCGAGCAGGUCAACA | 4947 | UGUUGACCUGCUCGGCCGG | 70.4 | 55.4 |
| 1522 | 3612 | CGGCCGAGCAGGUCAACAA | 4948 | UUGUUGACCUGCUCGGCCG | 90.5 | 75.5 |
| 1523 | 3613 | GGCCGAGCAGGUCAACAAC | 4949 | GUUGUUGACCUGCUCGGCC | 74.6 | 59.6 |
| 1524 | 3614 | GCCGAGCAGGUCAACAACC | 4950 | GGUUGUUGACCUGCUCGGC | 64.6 | 49.6 |
| 1525 | 3615 | CCGAGCAGGUCAACAACCC | 4951 | GGGUUGUUGACCUGCUCGG | 63.2 | 48.2 |
| 1542 | 3616 | CCCGAGCUCAAGGUGGACG | 4952 | CGUCCACCUUGAGCUCGGG | 58.3 | 43.3 |
| 1543 | 3617 | CCGAGCUCAAGGUGGACGC | 4953 | GCGUCCACCUUGAGCUCGG | 59.7 | 44.7 |
| 1544 | 3618 | CGAGCUCAAGGUGGACGCC | 4954 | GGCGUCCACCUUGAGCUCG | 59.8 | 44.8 |
| 1545 | 3619 | GAGCUCAAGGUGGACGCCU | 4955 | AGGCGUCCACCUUGAGCUC | 66 | 51 |
| 1546 | 3620 | AGCUCAAGGUGGACGCCUC | 4956 | GAGGCGUCCACCUUGAGCU | 67.3 | 52.3 |
| 1547 | 3621 | GCUCAAGGUGGACGCCUCG | 4957 | CGAGGCGUCCACCUUGAGC | 66.4 | 51.4 |
| 1548 | 3622 | CUCAAGGUGGACGCCUCGG | 4958 | CCGAGGCGUCCACCUUGAG | 51.9 | 36.9 |
| 1549 | 3623 | UCAAGGUGGACGCCUCGGG | 4959 | CCCGAGGCGUCCACCUUGA | 44.5 | 29.5 |
| 1550 | 3624 | CAAGGUGGACGCCUCGGGC | 4960 | GCCCGAGGCGUCCACCUUG | 50.3 | 35.3 |
| 1551 | 3625 | AAGGUGGACGCCUCGGGCC | 4961 | GGCCCGAGGCGUCCACCUU | 39.8 | 24.8 |
| 1552 | 3626 | AGGUGGACGCCUCGGGCCC | 4962 | GGGCCCGAGGCGUCCACCU | 45.5 | 30.5 |
| 1569 | 3627 | CCCGAUGUCCCGACACGGC | 4963 | GCCGUGUCGGGACAUCGGG | 61.1 | 46.1 |
| 1570 | 3628 | CCGAUGUCCCGACACGGCG | 4964 | CGCCGUGUCGGGACAUCGG | 65.6 | 50.6 |
| 1571 | 3629 | CGAUGUCCCGACACGGCGG | 4965 | CCGCCGUGUCGGGACAUCG | 70.7 | 55.7 |
| 1572 | 3630 | GAUGUCCCGACACGGCGGC | 4966 | GCCGCCGUGUCGGGACAUC | 53.3 | 38.3 |
| 1573 | 3831 | AUGUCCCGACACGGCGGCG | 4987 | CGCCGCCGUGUCGGGACAU | 44.7 | 29.7 |
| 1574 | 3632 | UGUCCCGACACGGCGGCGU | 4968 | ACGCCGCCGUGUCGGGACA | 54.4 | 39.4 |
| 1575 | 3833 | GUCCCGACACGGCGGCGUC | 4969 | GACGCCGCCGUGUCGGGAC | 59.9 | 44.9 |
| 1576 | 3634 | UCCCGACACGGCGGCGUCG | 4970 | CGACGCCGCCGUGUCGGGA | 47.2 | 32.2 |
| 1577 | 3635 | CCCGACACGGCGGCGUCGG | 4971 | CCGACGCCGCCGUGUCGGG | 50.6 | 35.6 |
| 1578 | 3636 | CCGACACGGCGGCGUCGGC | 4972 | GCCGACGCCGCCGUGUCGG | 52.6 | 37.6 |
| 1579 | 3637 | CGACACGGCGGCGUCGGCU | 4973 | AGCCGACGCCGCCGUGUCG | 62.6 | 47.6 |
| 1580 | 3638 | GACACGGCGGCGUCGGCUA | 4974 | UAGCCGACGCCGCCGUGUC | 81.9 | 66.9 |
| 1581 | 3639 | ACACGGCGGCGUCGGCUAC | 4975 | GUAGCCGACGCCGCCGUGU | 59.6 | 44.6 |
| 1582 | 3640 | CACGGCGGCGUCGGCUACA | 4976 | UGUAGCCGACGCCGCCGUG | 69.5 | 54.5 |
| 1583 | 3641 | ACGGCGGCGUCGGCUACAG | 4977 | CUGUAGCCGACGCCGCCGU | 58.7 | 43.7 |
| 1584 | 3642 | CGGCGGCGUCGGCUACAGC | 4978 | GCUGUAGCCGACGCCGCCG | 59 | 44 |
| 1585 | 3643 | GGCGGCGUCGGCUACAGCU | 4979 | AGCUGUAGCCGACGCCGCC | 72.2 | 57.2 |
| 1586 | 3644 | GCGGCGUCGGCUACAGCUC | 4980 | GAGCUGUAGCCGACGCCGC | 68.1 | 53.1 |
| 1587 | 3645 | CGGCGUCGGCUACAGCUCC | 4981 | GGAGCUGUAGCCGACGCCG | 63.5 | 48.5 |
| 1588 | 3646 | GGCGUCGGCUACAGCUCCG | 4982 | CGGAGCUGUAGCCGACGCC | 66.2 | 51.2 |
| 1589 | 3647 | GCGUCGGCUACAGCUCCGG | 4983 | CCGGAGCUGUAGCCGACGC | 59.9 | 44.9 |
| 1590 | 3648 | CGUCGGCUACAGCUCCGGG | 4984 | CCCGGAGCUGUAGCCGACG | 59.1 | 44.1 |
| 1591 | 3649 | GUCGGCUACAGCUCCGGGC | 4985 | GCCCGGAGCUGUAGCCGAC | 49.4 | 34.4 |
| 1592 | 3650 | UCGGCUACAGCUCCGGGCG | 4986 | CGCCCGGAGCUGUAGCCGA | 47.4 | 32.4 |
| 1593 | 3651 | CGGCUACAGCUCCGGGCGG | 4987 | CCGCCCGGAGCUGUAGCCG | 59.9 | 44.9 |
| 1594 | 3652 | GGCUACAGCUCCGGGCGGC | 4988 | GCCGCCCGGAGCUGUAGCC | 53.9 | 38.9 |
| 1595 | 3653 | GCUACAGCUCCGGGCGGCC | 4989 | GGCCGCCCGGAGCUGUAGC | 51.1 | 36.1 |
| 1627 | 3654 | AAACGGCCGCACUGGGACA | 4990 | UGUCCCAGUGCGGCCGUUU | 65.7 | 49.7 |
| 1628 | 3655 | AACGGCCGCACUGGGACAC | 4991 | GUGUCCCAGUGCGGCCGUU | 42.2 | 26.2 |
| 1629 | 3656 | ACGGCCGCACUGGGACACG | 4992 | CGUGUCCCAGUGCGGCCGU | 43.4 | 27.4 |
| 1630 | 3657 | CGGCCGCACUGGGACACGA | 4993 | UCGUGUCCCAGUGCGGCCG | 76.3 | 60.3 |
| 1631 | 3658 | GGCCGCACUGGGACACGAC | 4994 | GUCGUGUCCCAGUGCGGCC | 64.2 | 48.2 |
| 1632 | 3659 | GCCGCACUGGGACACGACC | 4995 | GGUCGUGUCCCAGUGCGGC | 59.2 | 43.2 |
| 1633 | 3660 | CCGCACUGGGACACGACCU | 4996 | AGGUCGUGUCCCAGUGCGG | 69.2 | 53.2 |
| 1634 | 3661 | CGCACUGGGACACGACCUG | 4997 | CAGGUCGUGUCCCAGUGCG | 67.7 | 51.7 |
| 1635 | 3662 | GCACUGGGACACGACCUGG | 4998 | CCAGGUCGUGUCCCAGUGC | 64 | 48 |
| 1636 | 3663 | CACUGGGACACGACCUGGA | 4999 | UCCAGGUCGUGUCCCAGUG | 78.7 | 62.7 |
| 1637 | 3664 | ACUGGGACACGACCUGGAC | 5000 | GUCCAGGUCGUGUCCCAGU | 62.5 | 46.5 |
| 1638 | 3665 | CUGGGACACGACCUGGACG | 5001 | CGUCCAGGUCGUGUCCCAG | 57.9 | 41.9 |
| 1639 | 3666 | UGGGACACGACCUGGACGG | 5002 | CCGUCCAGGUCGUGUCCCA | 51.3 | 35.3 |
| 1640 | 3667 | GGGACACGACCUGGACGGG | 5003 | CCCGUCCAGGUCGUGUCCC | 59.6 | 43.6 |
| 1641 | 3668 | GGACACGACCUGGACGGGC | 5004 | GCCCGUCCAGGUCGUGUCC | 55.3 | 39.3 |
| 1642 | 3669 | GACACGACCUGGACGGGCA | 5005 | UGCCCGUCCAGGUCGUGUC | 82 | 66 |
| 1643 | 3670 | ACACGACCUGGACGGGCAG | 5006 | CUGCCCGUCCAGGUCGUGU | 63.6 | 47.6 |
| 1644 | 3671 | CACGACCUGGACGGGCAGG | 5007 | CCUGCCCGUCCAGGUCGUG | 51.2 | 35.2 |
| 1645 | 3672 | ACGACCUGGACGGGCAGGA | 5008 | UCCUGCCCGUCCAGGUCGU | 61.7 | 45.7 |
| 1646 | 3673 | CGACCUGGACGGGCAGGAC | 5009 | GUCCUGCCCGUCCAGGUCG | 63.9 | 47.9 |
| 1647 | 3674 | GACCUGGACGGGCAGGACG | 5010 | CGUCCUGCCCGUCCAGGUC | 60.6 | 44.6 |
| 1648 | 3675 | ACCUGGACGGGCAGGACGC | 5011 | GCGUCCUGCCCGUCCAGGU | 50.8 | 34.8 |
| 1649 | 3676 | CCUGGACGGGCAGGACGCG | 5012 | CGCGUCCUGCCCGUCCAGG | 52.2 | 36.2 |
| 1650 | 3677 | CUGGACGGGCAGGACGCGG | 5013 | CCGCGUCCUGCCCGUCCAG | 45.9 | 29.9 |
| 1651 | 3678 | UGGACGGGCAGGACGCGGA | 5014 | UCCGCGUCCUGCCCGUCCA | 65 | 49 |
| 1652 | 3679 | GGACGGGCAGGACGCGGAU | 5015 | AUCCGCGUCCUGCCCGUCC | 79.4 | 63.4 |
| 1653 | 3680 | GACGGGCAGGACGCGGAUG | 5016 | CAUCCGCGUCCUGCCCGUC | 61.6 | 45.6 |
| 1654 | 3681 | ACGGGCAGGACGCGGAUGA | 5017 | UCAUCCGCGUCCUGCCCGU | 66.8 | 50.8 |
| 1655 | 3682 | CGGGCAGGACGCGGAUGAG | 5018 | CUCAUCCGCGUCCUGCCCG | 64.4 | 48.4 |
| 1656 | 3683 | GGGCAGGACGCGGAUGAGG | 5019 | CCUCAUCCGCGUCCUGCCC | 59 | 43 |
| 1657 | 3684 | GGCAGGACGCGGAUGAGGA | 5020 | UCCUCAUCCGCGUCCUGCC | 81.1 | 65.1 |
| 1658 | 3685 | GCAGGACGCGGAUGAGGAU | 5021 | AUCCUCAUCCGCGUCCUGC | 82.1 | 66.1 |
| 1659 | 3686 | CAGGACGCGGAUGAGGAUG | 5022 | CAUCCUCAUCCGCGUCCUG | 60.9 | 44.9 |
| 1660 | 3687 | AGGACGCGGAUGAGGAUGC | 5023 | GCAUCCUCAUCCGCGUCCU | 56.2 | 40.2 |
| 1661 | 3688 | GGACGCGGAUGAGGAUGCC | 5024 | GGCAUCCUCAUCCGCGUCC | 60.4 | 44.4 |
| 1694 | 3689 | GGGACAGCAGUAUGCAGAU | 5025 | AUCUGCAUACUGCUGUCCC | 86.9 | 70.9 |
| 1695 | 3690 | GGACAGCAGUAUGCAGAUG | 5026 | CAUCUGCAUACUGCUGUCC | 79.5 | 63.5 |
| 1696 | 3691 | GACAGCAGUAUGCAGAUGA | 5027 | UCAUCUGCAUACUGCUGUC | 85.6 | 69.6 |
| 1697 | 3692 | ACAGCAGUAUGCAGAUGAC | 5028 | GUCAUCUGCAUACUGCUGU | 73.3 | 57.3 |
| 1698 | 3693 | CAGCAGUAUGCAGAUGACU | 5029 | AGUCAUCUGCAUACUGCUG | 77.2 | 61.2 |
| 1699 | 3694 | AGCAGUAUGCAGAUGACUG | 5030 | CAGUCAUCUGCAUACUGCU | 72.7 | 56.7 |
| 1700 | 3695 | GCAGUAUGCAGAUGACUGG | 5031 | CCAGUCAUCUGCAUACUGC | 72.9 | 55.9 |
| 1701 | 3696 | CAGUAUGCAGAUGACUGGA | 5032 | UCCAGUCAUCUGCAUACUG | 82.4 | 65.4 |
| 1702 | 3697 | AGUAUGCAGAUGACUGGAU | 5033 | AUCCAGUCAUCUGCAUACU | 78.9 | 61.9 |
| 1703 | 3698 | GUAUGCAGAUGACUGGAUG | 5034 | CAUCCAGUCAUCUGCAUAC | 72.9 | 55.9 |
| 1704 | 3699 | UAUGCAGAUGACUGGAUGG | 5035 | CCAUCCAGUCAUCUGCAUA | 59 | 42 |
| 1739 | 3700 | CCCAGCCCGGCCUCCUCGG | 5036 | CCGAGGAGGCCGGGCUGGG | 46 | 29 |
| 1740 | 3701 | CCAGCCCGGCCUCCUCGGC | 5037 | GCCGAGGAGGCCGGGCUGG | 40.6 | 23.6 |
| 1741 | 3702 | CAGCCCGGCCUCCUCGGCC | 5038 | GGCCGAGGAGGCCGGGCUG | 37.2 | 20.2 |
| 1742 | 3703 | AGCCCGGCCUCCUCGGCCU | 5039 | AGGCCGAGGAGGCCGGGCU | 57.2 | 40.2 |
| 1743 | 3704 | GCCCGGCCUCCUCGGCCUC | 5040 | GAGGCCGAGGAGGCCGGGC | 52.7 | 35.7 |
| 1744 | 3705 | CCCGGCCUCCUCGGCCUCC | 5041 | GGAGGCCGAGGAGGCCGGG | 40.5 | 23.5 |
| 1745 | 3706 | CCGGCCUCCUCGGCCUCCA | 5042 | UGGAGGCCGAGGAGGCCGG | 64.4 | 47.4 |
| 1746 | 3707 | CGGCCUCCUCGGCCUCCAU | 5043 | AUGGAGGCCGAGGAGGCCG | 71 | 54 |
| 1747 | 3708 | GGCCUCCUCGGCCUCCAUA | 5044 | UAUGGAGGCCGAGGAGGCC | 74.4 | 57.4 |
| 1748 | 3709 | GCCUCCUCGGCCUCCAUAC | 5045 | GUAUGGAGGCCGAGGAGGC | 59.4 | 42.4 |
| 1749 | 3710 | CCUCCUCGGCCUCCAUACC | 5046 | GGUAUGGAGGCCGAGGAGG | 54.2 | 37.2 |
| 1750 | 3711 | CUCCUCGGCCUCCAUACCC | 5047 | GGGUAUGGAGGCCGAGGAG | 44.6 | 27.6 |
| 1751 | 3712 | UCCUCGGCCUCCAUACCCU | 5048 | AGGGUAUGGAGGCCGAGGA | 57.6 | 40.6 |
| 1752 | 3713 | CCUCGGCCUCCAUACCCUC | 5049 | GAGGGUAUGGAGGCCGAGG | 59.4 | 42.4 |
| 1753 | 3714 | CUCGGCCUCCAUACCCUCC | 5050 | GGAGGGUAUGGAGGCCGAG | 47.2 | 30.2 |
| 1754 | 3715 | UCGGCCUCCAUACCCUCCU | 5051 | AGGAGGGUAUGGAGGCCGA | 51.3 | 34.3 |
| 1755 | 3716 | CGGCCUCCAUACCCUCCUA | 5052 | UAGGAGGGUAUGGAGGCCG | 85.3 | 68.3 |
| 1756 | 3717 | GGCCUCCAUACCCUCCUAG | 5053 | CUAGGAGGGUAUGGAGGCC | 63.5 | 46.5 |
| 1757 | 3718 | GCCUCCAUACCCUCCUAGA | 5054 | UCUAGGAGGGUAUGGAGGC | 80.9 | 63.9 |
| 1758 | 3719 | CCUCCAUACCCUCCUAGAA | 5055 | UUCUAGGAGGGUAUGGAGG | 87.4 | 70.4 |
| 1759 | 3720 | CUCCAUACCCUCCUAGAAG | 5056 | CUUCUAGGAGGGUAUGGAG | 66.2 | 49.2 |
| 1760 | 3721 | UCCAUACCCUCCUAGAAGG | 5057 | CCUUCUAGGAGGGUAUGGA | 56.5 | 39.5 |
| 1761 | 3722 | CCAUACCCUCCUAGAAGGG | 5058 | CCCUUCUAGGAGGGUAUGG | 58 | 41 |
| 1762 | 3723 | CAUACCCUCCUAGAAGGGA | 5059 | UCCCUUCUAGGAGGGUAUG | 70 | 53 |
| 1763 | 3724 | AUACCCUCCUAGAAGGGAU | 5060 | AUCCCUUCUAGGAGGGUAU | 69.6 | 52.6 |
| 1764 | 3725 | UACCCUCCUAGAAGGGAUG | 5061 | CAUCCCUUCUAGGAGGGUA | 57.9 | 40.9 |
| 1765 | 3726 | ACCCUCCUAGAAGGGAUGG | 5062 | CCAUCCCUUCUAGGAGGGU | 50.6 | 33.6 |
| 1766 | 3727 | CCCUCCUAGAAGGGAUGGU | 5063 | ACCAUCCCUUCUAGGAGGG | 64.3 | 47.3 |
| 1767 | 3728 | CCUCCUAGAAGGGAUGGUU | 5064 | AACCAUCCCUUCUAGGAGG | 77.1 | 60.1 |
| 1768 | 3729 | CUCCUAGAAGGGAUGGUUC | 5065 | GAACCAUCCCUUCUAGGAG | 66.9 | 49.9 |
| 1769 | 3730 | UCCUAGAAGGGAUGGUUCU | 5066 | AGAACCAUCCCUUCUAGGA | 72.2 | 55.2 |
| 1770 | 3731 | CCUAGAAGGGAUGGUUCUG | 5067 | CAGAACCAUCCCUUCUAGG | 70.8 | 53.8 |
| 1771 | 3732 | CUAGAAGGGAUGGUUCUGG | 5068 | CCAGAACCAUCCCUUCUAG | 57.6 | 40.6 |
| 1772 | 3733 | UAGAAGGGAUGGUUCUGGG | 5069 | CCCAGAACCAUCCCUUCUA | 53.5 | 36.5 |
| 1806 | 3734 | GGCAGUGCCCGCUACAACC | 5070 | GGUUGUAGCGGGCACUGCC | 65.6 | 47.6 |
| 1807 | 3735 | GCAGUGCCCGCUACAACCA | 5071 | UGGUUGUAGCGGGCACUGC | 82.2 | 64.2 |
| 1808 | 3736 | CAGUGCCCGCUACAACCAG | 5072 | CUGGUUGUAGCGGGCACUG | 66.1 | 48.1 |
| 1809 | 3737 | AGUGCCCGCUACAACCAGG | 5073 | CCUGGUUGUAGCGGGCACU | 58.7 | 40.7 |
| 1810 | 3738 | GUGCCCGCUACAACCAGGG | 5074 | CCCUGGUUGUAGCGGGCAC | 49.7 | 31.7 |
| 1811 | 3739 | UGCCCGCUACAACCAGGGC | 5075 | GCCCUGGUUGUAGCGGGCA | 43.9 | 25.9 |
| 1812 | 3740 | GCCCGCUACAACCAGGGCC | 5076 | GGCCCUGGUUGUAGCGGGC | 47.4 | 29.4 |
| 1813 | 3741 | CCCGCUACAACCAGGGCCG | 5077 | CGGCCCUGGUUGUAGCGGG | 53.5 | 35.5 |
| 1814 | 3742 | CCGCUACAACCAGGGCCGG | 5078 | CCGGCCCUGGUUGUAGCGG | 51.1 | 33.1 |
| 1815 | 3743 | CGCUACAACCAGGGCCGGA | 5079 | UCCGGCCCUGGUUGUAGCG | 68.4 | 50.4 |
| 1816 | 3744 | GCUACAACCAGGGCCGGAG | 5080 | CUCCGGCCCUGGUUGUAGC | 59.8 | 41.8 |
| 1817 | 3745 | CUACAACCAGGGCCGGAGC | 5081 | GCUCCGGCCCUGGUUGUAG | 50 | 32 |
| 1818 | 3746 | UACAACCAGGGCCGGAGCA | 5082 | UGCUCCGGCCCUGGUUGUA | 56.5 | 38.5 |
| 1819 | 3747 | ACAACCAGGGCCGGAGCAG | 5083 | CUGCUCCGGCCCUGGUUGU | 49.1 | 31.1 |
| 1820 | 3748 | CAACCAGGGCCGGAGCAGG | 5084 | CCUGCUCCGGCCCUGGUUG | 50.5 | 32.5 |
| 1821 | 3749 | AACCAGGGCCGGAGCAGGA | 5085 | UCCUGCUCCGGCCCUGGUU | 63.2 | 45.2 |
| 1822 | 3750 | ACCAGGGCCGGAGCAGGAG | 5086 | CUCCUGCUCCGGCCCUGGU | 49.6 | 31.6 |
| 1823 | 3751 | CCAGGGCCGGAGCAGGAGU | 5087 | ACUCCUGCUCCGGCCCUGG | 63.6 | 45.6 |
| 1824 | 3752 | CAGGGCCGGAGCAGGAGUG | 5088 | CACUCCUGCUCCGGCCCUG | 55 | 37 |
| 1825 | 3753 | AGGGCCGGAGCAGGAGUGG | 5089 | CCACUCCUGCUCCGGCCCU | 46.5 | 28.5 |
| 1826 | 3754 | GGGCCGGAGCAGGAGUGGG | 5090 | CCCACUCCUGCUCCGGCCC | 53.3 | 35.3 |
| 1860 | 3755 | UUUCACACCCAAACCAUCC | 5091 | GGAUGGUUUGGGUGUGAAA | 44 | 26 |
| 1861 | 3756 | UUCACACCCAAACCAUCCU | 5092 | AGGAUGGUUUGGGUGUGAA | 56.1 | 38.1 |
| 1862 | 3757 | UCACACCCAAACCAUCCUC | 5093 | GAGGAUGGUUUGGGUGUGA | 50.3 | 32.3 |
| 1863 | 3758 | CACACCCAAACCAUCCUCA | 5094 | UGAGGAUGGUUUGGGUGUG | 72.6 | 54.6 |
| 1864 | 3759 | ACACCCAAACCAUCCUCAU | 5095 | AUGAGGAUGGUUUGGGUGU | 71.6 | 53.6 |
| 1865 | 3760 | CACCCAAACCAUCCUCAUU | 5096 | AAUGAGGAUGGUUUGGGUG | 75.6 | 57.6 |
| 1866 | 3761 | ACCCAAACCAUCCUCAUUC | 5097 | GAAUGAGGAUGGUUUGGGU | 56 | 38 |
| 1867 | 3762 | CCCAAACCAUCCUCAUUCU | 5098 | AGAAUGAGGAUGGUUUGGG | 75.4 | 57.4 |
| 1868 | 3763 | CCAAACCAUCCUCAUUCUC | 5099 | GAGAAUGAGGAUGGUUUGG | 62.7 | 44.7 |
| 1869 | 3764 | CAAACCAUCCUCAUUCUCU | 5100 | AGAGAAUGAGGAUGGUUUG | 69.9 | 51.9 |
| 1870 | 3765 | AAACCAUCCUCAUUCUCUC | 5101 | GAGAGAAUGAGGAUGGUUU | 61.5 | 43.5 |
| 1871 | 3766 | AACCAUCCUCAUUCUCUCC | 5102 | GGAGAGAAUGAGGAUGGUU | 54 | 36 |
| 1872 | 3767 | ACCAUCCUCAUUCUCUCCC | 5103 | GGGAGAGAAUGAGGAUGGU | 45 | 27 |
| 1873 | 3768 | CCAUCCUCAUUCUCUCCCU | 5104 | AGGGAGAGAAUGAGGAUGG | 71.6 | 53.6 |
| 1874 | 3769 | CAUCCUCAUUCUCUCCCUC | 5105 | GAGGGAGAGAAUGAGGAUG | 64 | 46 |
| 1875 | 3770 | AUCCUCAUUCUCUCCCUCU | 5106 | AGAGGGAGAGAAUGAGGAU | 60.3 | 42.3 |
| 1876 | 3771 | UCCUCAUUCUCUCCCUCUC | 5107 | GAGAGGGAGAGAAUGAGGA | 59.3 | 41.3 |
| 1877 | 3772 | CCUCAUUCUCUCCCUCUCA | 5108 | UGAGAGGGAGAGAAUGAGG | 81.8 | 63.8 |
| 1878 | 3773 | CUCAUUCUCUCCCUCUCAG | 5109 | CUGAGAGGGAGAGAAUGAG | 65.2 | 47.2 |
| 1879 | 3774 | UCAUUCUCUCCCUCUCAGC | 5110 | GCUGAGAGGGAGAGAAUGA | 49 | 31 |
| 1880 | 3775 | CAUUCUCUCCCUCUCAGCC | 5111 | GGCUGAGAGGGAGAGAAUG | 53.9 | 35.9 |
| 1881 | 3776 | AUUCUCUCCCUCUCAGCCC | 5112 | GGGCUGAGAGGGAGAGAAU | 43.8 | 25.8 |
| 1882 | 3777 | UUCUCUCCCUCUCAGCCCU | 5113 | AGGGCUGAGAGGGAGAGAA | 57.1 | 39.1 |
| 1883 | 3778 | UCUCUCCCUCUCAGCCCUG | 5114 | CAGGGCUGAGAGGGAGAGA | 50.4 | 32.4 |
| 1884 | 3779 | CUCUCCCUCUCAGCCCUGG | 5115 | CCAGGGCUGAGAGGGAGAG | 49.2 | 31.2 |
| 1901 | 3780 | GGCCCUGCUUGGACCUCGA | 5116 | UCGAGGUCCAAGCAGGGCC | 86.5 | 67.5 |
| 1902 | 3781 | GCCCUGCUUGGACCUCGAU | 5117 | AUCGAGGUCCAAGCAGGGC | 79 | 60 |
| 1903 | 3782 | CCCUGCUUGGACCUCGAUA | 5118 | UAUCGAGGUCCAAGCAGGG | 84.1 | 65.1 |
| 1904 | 3783 | CCUGCUUGGACCUCGAUAA | 5119 | UUAUCGAGGUCCAAGCAGG | 90.9 | 71.9 |
| 1905 | 3784 | CUGCUUGGACCUCGAUAAC | 5120 | GUUAUCGAGGUCCAAGCAG | 67.5 | 48.5 |
| 1906 | 3785 | UGCUUGGACCUCGAUAACG | 5121 | CGUUAUCGAGGUCCAAGCA | 57.4 | 38.4 |
| 1907 | 3786 | GCUUGGACCUCGAUAACGG | 5122 | CCGUUAUCGAGGUCCAAGC | 71.9 | 52.9 |
| 1908 | 3787 | CUUGGACCUCGAUAACGGG | 5123 | CCCGUUAUCGAGGUCCAAG | 59.3 | 40.3 |
| 1931 | 3788 | GGGUGCCCUAGCAUCAGAA | 5124 | UUCUGAUGCUAGGGCACCC | 85.5 | 66.5 |
| 1932 | 3789 | GGUGCCCUAGCAUCAGAAG | 5125 | CUUCUGAUGCUAGGGCACC | 73.8 | 54.8 |
| 1933 | 3790 | GUGCCCUAGCAUCAGAAGG | 5126 | CCUUCUGAUGCUAGGGCAC | 60.7 | 41.7 |
| 1934 | 3791 | UGCCCUAGCAUCAGAAGGG | 5127 | CCCUUCUGAUGCUAGGGCA | 51.8 | 32.8 |
| 1935 | 3792 | GCCCUAGCAUCAGAAGGGU | 5128 | ACCCUUCUGAUGCUAGGGC | 71.1 | 52.1 |
| 1936 | 3793 | CCCUAGCAUCAGAAGGGUU | 5129 | AACCCUUCUGAUGCUAGGG | 80.8 | 61.8 |
| 1937 | 3794 | CCUAGCAUCAGAAGGGUUC | 5130 | GAACCCUUCUGAUGCUAGG | 68.3 | 49.3 |
| 1938 | 3795 | CUAGCAUCAGAAGGGUUCA | 5131 | UGAACCCUUCUGAUGCUAG | 81.4 | 62.4 |
| 1939 | 3796 | UAGCAUCAGAAGGGUUCAU | 5132 | AUGAACCCUUCUGAUGCUA | 68.2 | 49.2 |
| 1940 | 3797 | AGCAUCAGAAGGGUUCAUG | 5133 | CAUGAACCCUUCUGAUGCU | 59.3 | 40.3 |
| 1941 | 3798 | GCAUCAGAAGGGUUCAUGG | 5134 | CCAUGAACCCUUCUGAUGC | 69.9 | 50.9 |
| 2008 | 3799 | GGGCUGCAGAGAGGGUAGA | 5135 | UCUACCCUCUCUGCAGCCC | 79.1 | 59.1 |
| 2009 | 3800 | GGCUGCAGAGAGGGUAGAG | 5136 | CUCUACCCUCUCUGCAGCC | 68.1 | 48.1 |
| 2010 | 3801 | GCUGCAGAGAGGGUAGAGA | 5137 | UCUCUACCCUCUCUGCAGC | 80.9 | 60.9 |
| 2011 | 3802 | CUGCAGAGAGGGUAGAGAA | 5138 | UUCUCUACCCUCUCUGCAG | 86.8 | 66.8 |
| 2012 | 3803 | UGCAGAGAGGGUAGAGAAG | 5139 | CUUCUCUACCCUCUCUGCA | 68.6 | 48.6 |
| 2013 | 3804 | GCAGAGAGGGUAGAGAAGG | 5140 | CCUUCUCUACCCUCUCUGC | 65.8 | 45.8 |
| 2014 | 3805 | CAGAGAGGGUAGAGAAGGG | 5141 | CCCUUCUCUACCCUCUCUG | 64.6 | 44.6 |
| 2015 | 3806 | AGAGAGGGUAGAGAAGGGA | 5142 | UCCCUUCUCUACCCUCUCU | 73 | 53 |
| 2016 | 3807 | GAGAGGGUAGAGAAGGGAC | 5143 | GUCCCUUCUCUACCCUCUC | 67.7 | 47.7 |
| 2017 | 3808 | AGAGGGUAGAGAAGGGACU | 5144 | AGUCCCUUCUCUACCCUCU | 72.3 | 52.3 |
| 2018 | 3809 | GAGGGUAGAGAAGGGACUU | 5145 | AAGUCCCUUCUCUACCCUC | 77.2 | 57.2 |
| 2019 | 3810 | AGGGUAGAGAAGGGACUUU | 5146 | AAAGUCCCUUCUCUACCCU | 71.1 | 51.1 |
| 2020 | 3811 | GGGUAGAGAAGGGACUUUG | 5147 | CAAAGUCCCUUCUCUACCC | 72.9 | 52.9 |
| 2021 | 3812 | GGUAGAGAAGGGACUUUGC | 5148 | GCAAAGUCCCUUCUCUACC | 72.2 | 52.2 |
| 2022 | 3813 | GUAGAGAAGGGACUUUGCA | 5149 | UGCAAAGUCCCUUCUCUAC | 80.5 | 60.5 |
| 2023 | 3814 | UAGAGAAGGGACUUUGCAG | 5150 | CUGCAAAGUCCCUUCUCUA | 61.1 | 41.1 |
| 2024 | 3815 | AGAGAAGGGACUUUGCAGG | 5151 | CCUGCAAAGUCCCUUCUCU | 59.1 | 39.1 |
| 2025 | 3816 | GAGAAGGGACUUUGCAGGU | 5152 | ACCUGCAAAGUCCCUUCUC | 72.5 | 52.5 |
| 2026 | 3817 | AGAAGGGACUUUGCAGGUG | 5153 | CACCUGCAAAGUCCCUUCU | 64.1 | 44.1 |
| 2027 | 3818 | GAAGGGACUUUGCAGGUGA | 5154 | UCACCUGCAAAGUCCCUUC | 85.3 | 65.3 |
| 2028 | 3819 | AAGGGACUUUGCAGGUGAA | 5155 | UUCACCUGCAAAGUCCCUU | 83.6 | 63.6 |
| 2029 | 3820 | AGGGACUUUGCAGGUGAAU | 5156 | AUUCACCUGCAAAGUCCCU | 69.9 | 49.9 |
| 2030 | 3821 | GGGACUUUGCAGGUGAAUG | 5157 | CAUUCACCUGCAAAGUCCC | 68.8 | 48.8 |
| 2031 | 3822 | GGACUUUGCAGGUGAAUGG | 5158 | CCAUUCACCUGCAAAGUCC | 71.3 | 51.3 |
| 2073 | 3823 | UUUCAUCAGAGGUGGGUGG | 5159 | CCACCCACCUCUGAUGAAA | 47.2 | 27.2 |
| 2074 | 3824 | UUCAUCAGAGGUGGGUGGG | 5160 | CCCACCCACCUCUGAUGAA | 36.8 | 16.8 |
| 2075 | 3825 | UCAUCAGAGGUGGGUGGGU | 5161 | ACCCACCCACCUCUGAUGA | 54 | 34 |
| 2076 | 3826 | CAUCAGAGGUGGGUGGGUG | 5162 | CACCCACCCACCUCUGAUG | 60.3 | 40.3 |
| 2077 | 3827 | AUCAGAGGUGGGUGGGUGU | 5163 | ACACCCACCCACCUCUGAU | 62.7 | 42.7 |
| 2078 | 3825 | UCAGAGGUGGGUGGGUGUU | 5164 | AACACCCACCCACCUCUGA | 64.8 | 44.8 |
| 2079 | 3829 | CAGAGGUGGGUGGGUGUUC | 5165 | GAACACCCACCCACCUCUG | 62.5 | 42.5 |
| 2080 | 3830 | AGAGGUGGGUGGGUGUUCA | 5166 | UGAACACCCACCCACCUCU | 79.5 | 59.5 |
| 2081 | 3831 | GAGGUGGGUGGGUGUUCAC | 5167 | GUGAACACCCACCCACCUC | 68.7 | 48.7 |
| 2082 | 3832 | AGGUGGGUGGGUGUUCACA | 5168 | UGUGAACACCCACCCACCU | 75.5 | 55.5 |
| 2083 | 3833 | GGUGGGUGGGUGUUCACAA | 5169 | UUGUGAACACCCACCCACC | 93.7 | 73.7 |
| 2084 | 3834 | GUGGGUGGGUGUUCACAAU | 5170 | AUUGUGAACACCCACCCAC | 84.9 | 64.9 |
| 2085 | 3835 | UGGGUGGGUGUUCACAAUA | 5171 | UAUUGUGAACACCCACCCA | 80.9 | 60.9 |
| 2086 | 3836 | GGGUGGGUGUUCACAAUAU | 5172 | AUAUUGUGAACACCCACCC | 93.1 | 73.1 |
| 2087 | 3837 | GGUGGGUGUUCACAAUAUU | 5173 | AAUAUUGUGAACACCCACC | 94.8 | 74.8 |
| 2088 | 3838 | GUGGGUGUUCACAAUAUUU | 5174 | AAAUAUUGUGAACACCCAC | 85.8 | 65.8 |
| 2089 | 3839 | UGGGUGUUCACAAUAUUUA | 5175 | UAAAUAUUGUGAACACCCA | 84.3 | 64.3 |
| 2090 | 3840 | GGGUGUUCACAAUAUUUAU | 5176 | AUAAAUAUUGUGAACACCC | 96 | 76 |
| 2091 | 3841 | GGUGUUCACAAUAUUUAUU | 5177 | AAUAAAUAUUGUGAACACC | 89.4 | 69.4 |
| 2092 | 3842 | GUGUUCACAAUAUUUAUUU | 5178 | AAAUAAAUAUUGUGAACAC | 79.7 | 59.7 |
| 2111 | 3843 | UUUCAUUUGGUAAUGGGAG | 5179 | CUCCCAUUACCAAAUGAAA | 49.8 | 28.8 |
| 2112 | 3844 | UUCAUUUGGUAAUGGGAGG | 5180 | CCUCCCAUUACCAAAUGAA | 47.5 | 26.5 |
| 2113 | 3845 | UCAUUUGGUAAUGGGAGGG | 5181 | CCCUCCCAUUACCAAAUGA | 40.3 | 19.3 |
| 2140 | 3846 | GGGUAUUUAUUUAGGAGGG | 5182 | CCCUCCUAAAUAAAUACCC | 64.6 | 43.6 |
| 2141 | 3847 | GGUAUUUAUUUAGGAGGGA | 5183 | UCCCUCCUAAAUAAAUACC | 78.8 | 57.8 |
| 2142 | 3848 | GUAUUUAUUUAGGAGGGAG | 5184 | CUCCCUCCUAAAUAAAUAC | 62.4 | 41.4 |
| 2143 | 3849 | UAUUUAUUUAGGAGGGAGU | 5185 | ACUCCCUCCUAAAUAAAUA | 59.9 | 38.9 |
| 2144 | 3850 | AUUUAUUUAGGAGGGAGUG | 5186 | CACUCCCUCCUAAAUAAAU | 53.3 | 32.3 |
| 2145 | 3851 | UUUAUUUAGGAGGGAGUGU | 5187 | ACACUCCCUCCUAAAUAAA | 54.1 | 33.1 |
| 2146 | 3852 | UUAUUUAGGAGGGAGUGUG | 5188 | CACACUCCCUCCUAAAUAA | 50 | 29 |
| 2147 | 3853 | UAUUUAGGAGGGAGUGUGG | 5189 | CCACACUCCCUCCUAAAUA | 54.3 | 33.3 |
| 2148 | 3854 | AUUUAGGAGGGAGUGUGGU | 5190 | ACCACACUCCCUCCUAAAU | 57.2 | 36.2 |
| 2149 | 3855 | UUUAGGAGGGAGUGUGGUU | 5191 | AACCACACUCCCUCCUAAA | 64.1 | 43.1 |
| 2150 | 3856 | UUAGGAGGGAGUGUGGUUU | 5192 | AAACCACACUCCCUCCUAA | 61.2 | 40.2 |
| 2151 | 3857 | UAGGAGGGAGUGUGGUUUC | 5193 | GAAACCACACUCCCUCCUA | 58.6 | 37.6 |
| 2152 | 3858 | AGGAGGGAGUGUGGUUUCC | 5194 | GGAAACCACACUCCCUCCU | 59.7 | 38.7 |
| 2153 | 3859 | GGAGGGAGUGUGGUUUCCU | 5195 | AGGAAACCACACUCCCUCC | 74.6 | 53.6 |
| 2154 | 3860 | GAGGGAGUGUGGUUUCCUU | 5196 | AAGGAAACCACACUCCCUC | 83.7 | 62.7 |
| 2155 | 3861 | AGGGAGUGUGGUUUCCUUA | 5197 | UAAGGAAACCACACUCCCU | 86.5 | 65.5 |
| 2156 | 3862 | GGGAGUGUGGUUUCCUUAG | 5198 | CUAAGGAAACCACACUCCC | 79.6 | 58.6 |
| 2157 | 3863 | GGAGUGUGGUUUCCUUAGA | 5199 | UCUAAGGAAACCACACUCC | 95.1 | 74.1 |
| 2158 | 3864 | GAGUGUGGUUUCCUUAGAA | 5200 | UUCUAAGGAAACCACACUC | 96.5 | 75.5 |
| 2159 | 3865 | AGUGUGGUUUCCUUAGAAG | 5201 | CUUCUAAGGAAACCACACU | 76.7 | 55.7 |
| 2160 | 3866 | GUGUGGUUUCCUUAGAAGG | 5202 | CCUUCUAAGGAAACCACAC | 71.6 | 50.6 |
| 2161 | 3867 | UGUGGUUUCCUUAGAAGGU | 5203 | ACCUUCUAAGGAAACCACA | 73.5 | 52.5 |
| 2162 | 3868 | GUGGUUUCCUUAGAAGGUA | 5204 | UACCUUCUAAGGAAACCAC | 87.8 | 66.8 |
| 2163 | 3869 | UGGUUUCCUUAGAAGGUAU | 5205 | AUACCUUCUAAGGAAACCA | 83 | 62 |
| 2164 | 3870 | GGUUUCCUUAGAAGGUAUA | 5206 | UAUACCUUCUAAGGAAACC | 94.8 | 73.8 |
| 2165 | 3871 | GUUUCCUUAGAAGGUAUAG | 5207 | CUAUACCUUCUAAGGAAAC | 71 | 50 |
| 2166 | 3872 | UUUCCUUAGAAGGUAUAGU | 5208 | ACUAUACCUUCUAAGGAAA | 60 | 39 |
| 2167 | 3873 | UUCCUUAGAAGGUAUAGUC | 5209 | GACUAUACCUUCUAAGGAA | 57.5 | 36.5 |
| 2168 | 3874 | UCCUUAGAAGGUAUAGUCU | 5210 | AGACUAUACCUUCUAAGGA | 72.3 | 51.3 |
| 2169 | 3875 | CCUUAGAAGGUAUAGUCUC | 5211 | GAGACUAUACCUUCUAAGG | 75.3 | 54.3 |
| 2170 | 3876 | CUUAGAAGGUAUAGUCUCU | 5212 | AGAGACUAUACCUUCUAAG | 82 | 61 |
| 2171 | 3877 | UUAGAAGGUAUAGUCUCUA | 5213 | UAGAGACUAUACCUUCUAA | 75.2 | 54.2 |
| 2172 | 3878 | UAGAAGGUAUAGUCUCUAG | 5214 | CUAGAGACUAUACCUUCUA | 72.6 | 51.6 |
| 2173 | 3879 | AGAAGGUAUAGUCUCUAGC | 5215 | GCUAGAGACUAUACCUUCU | 65.7 | 44.7 |
| 2174 | 3880 | GAAGGUAUAGUCUCUAGCC | 5216 | GGCUAGAGACUAUACCUUC | 66.5 | 45.5 |
| 2175 | 3881 | AAGGUAUAGUCUCUAGCCC | 5217 | GGGCUAGAGACUAUACCUU | 57.7 | 36.7 |
| 2176 | 3882 | AGGUAUAGUCUCUAGCCCU | 5218 | AGGGCUAGAGACUAUACCU | 75.3 | 54.3 |
| 2177 | 3883 | GGUAUAGUCUCUAGCCCUC | 5219 | GAGGGCUAGAGACUAUACC | 75.5 | 54.5 |
| 2178 | 3884 | GUAUAGUCUCUAGCCCUCU | 5220 | AGAGGGCUAGAGACUAUAC | 73.3 | 52.3 |
| 2179 | 3885 | UAUAGUCUCUAGCCCUCUA | 5221 | UAGAGGGCUAGAGACUAUA | 82.5 | 61.5 |
| 2180 | 3886 | AUAGUCUCUAGCCCUCUAA | 5222 | UUAGAGGGCUAGAGACUAU | 78.2 | 57.2 |
| 2181 | 3887 | UAGUCUCUAGCCCUCUAAG | 5223 | CUUAGAGGGCUAGAGACUA | 62.8 | 41.8 |
| 2182 | 3888 | AGUCUCUAGCCCUCUAAGG | 5224 | CCUUAGAGGGCUAGAGACU | 60.6 | 39.6 |
| 2183 | 3889 | GUCUCUAGCCCUCUAAGGC | 5225 | GCCUUAGAGGGCUAGAGAC | 56.1 | 35.1 |
| 2184 | 3890 | UCUCUAGCCCUCUAAGGCU | 5226 | AGCCUUAGAGGGCUAGAGA | 62.9 | 41.9 |
| 2185 | 3891 | CUCUAGCCCUCUAAGGCUG | 5227 | CAGCCUUAGAGGGCUAGAG | 67.1 | 46.1 |
| 2186 | 3892 | UCUAGCCCUCUAAGGCUGG | 5228 | CCAGCCUUAGAGGGCUAGA | 50.6 | 29.6 |
| 2187 | 3893 | CUAGCCCUCUAAGGCUGGG | 5229 | CCCAGCCUUAGAGGGCUAG | 48.7 | 27.7 |
| 2230 | 3894 | AAAUGAGGAGUUUAGAGUU | 5230 | AACUCUAAACUCCUCAUUU | 79.8 | 57.8 |
| 2231 | 3895 | AAUGAGGAGUUUAGAGUUG | 5231 | CAACUCUAAACUCCUCAUU | 73.3 | 51.3 |
| 2232 | 3896 | AUGAGGAGUUUAGAGUUGC | 5232 | GCAACUCUAAACUCCUCAU | 59.4 | 37.4 |
| 2233 | 3897 | UGAGGAGUUUAGAGUUGCA | 5233 | UGCAACUCUAAACUCCUCA | 84.9 | 62.9 |
| 2234 | 3898 | GAGGAGUUUAGAGUUGCAG | 5234 | CUGCAACUCUAAACUCCUC | 73.3 | 51.3 |
| 2235 | 3899 | AGGAGUUUAGAGUUGCAGC | 5235 | GCUGCAACUCUAAACUCCU | 66.1 | 44.1 |
| 2236 | 3900 | GGAGUUUAGAGUUGCAGCU | 5236 | AGCUGCAACUCUAAACUCC | 85.2 | 63.2 |
| 2237 | 3901 | GAGUUUAGAGUUGCAGCUG | 5237 | CAGCUGCAACUCUAAACUC | 75.7 | 53.7 |
| 2238 | 3902 | AGUUUAGAGUUGCAGCUGG | 5238 | CCAGCUGCAACUCUAAACU | 73.1 | 51.1 |
| 2239 | 3903 | GUUUAGAGUUGCAGCUGGG | 5239 | CCCAGCUGCAACUCUAAAC | 70.2 | 48.2 |
| 2262 | 3904 | GGGUUUGAAGGAAGUUGGA | 5240 | UCCAACUUCCUUCAAACCC | 90.2 | 68.2 |
| 2263 | 3905 | GGUUUGAAGGAAGUUGGAA | 5241 | UUCCAACUUCCUUCAAACC | 91.9 | 69.9 |
| 2264 | 3906 | GUUUGAAGGAAGUUGGAAG | 5242 | CUUCCAACUUCCUUCAAAC | 73.8 | 51.8 |
| 2265 | 3907 | UUUGAAGGAAGUUGGAAGU | 5243 | ACUUCCAACUUCCUUCAAA | 64.3 | 42.3 |
| 2266 | 3908 | UUGAAGGAAGUUGGAAGUG | 5244 | CACUUCCAACUUCCUUCAA | 55.7 | 33.7 |
| 2267 | 3909 | UGAAGGAAGUUGGAAGUGG | 5245 | CCACUUCCAACUUCCUUCA | 62.8 | 40.8 |
| 2268 | 3910 | GAAGGAAGUUGGAAGUGGG | 5246 | CCCACUUCCAACUUCCUUC | 72.5 | 50.5 |
| 2296 | 3911 | GGGCAUCUGGUCUCAGAAA | 5247 | UUUCUGAGACCAGAUGCCC | 90.5 | 68.5 |
| 2297 | 3912 | GGCAUCUGGUCUCAGAAAU | 5248 | AUUUCUGAGACCAGAUGCC | 84.6 | 62.6 |
| 2298 | 3913 | GCAUCUGGUCUCAGAAAUG | 5249 | CAUUUCUGAGACCAGAUGC | 77.1 | 55.1 |
| 2299 | 3914 | CAUCUGGUCUCAGAAAUGG | 5250 | CCAUUUCUGAGACCAGAUG | 65.4 | 43.4 |
| 2300 | 3915 | AUCUGGUCUCAGAAAUGGA | 5251 | UCCAUUUCUGAGACCAGAU | 76.8 | 53.8 |
| 2301 | 3916 | UCUGGUCUCAGAAAUGGAC | 5252 | GUCCAUUUCUGAGACCAGA | 60.2 | 37.2 |
| 2302 | 3917 | CUGGUCUCAGAAAUGGACC | 5253 | GGUCCAUUUCUGAGACCAG | 55.4 | 32.4 |
| 2303 | 3918 | UGGUCUCAGAAAUGGACCA | 5254 | UGGUCCAUUUCUGAGACCA | 76 | 53 |
| 2304 | 3919 | GGUCUCAGAAAUGGACCAG | 5255 | CUGGUCCAUUUCUGAGACC | 69.7 | 46.7 |
| 2305 | 3920 | GUCUCAGAAAUGGACCAGC | 5256 | GCUGGUCCAUUUCUGAGAC | 57.5 | 34.5 |
| 2306 | 3921 | UCUCAGAAAUGGACCAGCU | 5257 | AGCUGGUCCAUUUCUGAGA | 70.1 | 47.1 |
| 2307 | 3922 | CUCAGAAAUGGACCAGCUG | 5258 | CAGCUGGUCCAUUUCUGAG | 68.4 | 45.4 |
| 2308 | 3923 | UCAGAAAUGGACCAGCUGG | 5259 | CCAGCUGGUCCAUUUCUGA | 57.5 | 34.5 |
| 2309 | 3924 | CAGAAAUGGACCAGCUGGA | 5260 | UCCAGCUGGUCCAUUUCUG | 80.1 | 57.1 |
| 2310 | 3925 | AGAAAUGGACCAGCUGGAU | 5261 | AUCCAGCUGGUCCAUUUCU | 75.1 | 52.1 |
| 2311 | 3926 | GAAAUGGACCAGCUGGAUG | 5262 | CAUCCAGCUGGUCCAUUUC | 69.3 | 46.3 |
| 2312 | 3927 | AAAUGGACCAGCUGGAUGC | 5263 | GCAUCCAGCUGGUCCAUUU | 57.6 | 34.6 |
| 2313 | 3928 | AAUGGACCAGCUGGAUGCA | 5264 | UGCAUCCAGCUGGUCCAUU | 72.5 | 49.5 |
| 2314 | 3929 | AUGGACCAGCUGGAUGCAG | 5265 | CUGCAUCCAGCUGGUCCAU | 54.7 | 31.7 |
| 2315 | 3930 | UGGACCAGCUGGAUGCAGG | 5266 | CCUGCAUCCAGCUGGUCCA | 55.8 | 32.8 |
| 2316 | 3931 | GGACCAGCUGGAUGCAGGG | 5267 | CCCUGCAUCCAGCUGGUCC | 65.9 | 42.9 |
| 2317 | 3932 | GACCAGCUGGAUGCAGGGC | 5268 | GCCCUGCAUCCAGCUGGUC | 52.3 | 29.3 |
| 2318 | 3933 | ACCAGCUGGAUGCAGGGCA | 5269 | UGCCCUGCAUCCAGCUGGU | 65.5 | 42.5 |
| 2319 | 3934 | CCAGCUGGAUGCAGGGCAG | 5270 | CUGCCCUGCAUCCAGCUGG | 70.5 | 47.5 |
| 2320 | 3935 | CAGCUGGAUGCAGGGCAGG | 5271 | CCUGCCCUGCAUCCAGCUG | 55.3 | 32.3 |
| 2321 | 3936 | AGCUGGAUGCAGGGCAGGG | 5272 | CCCUGCCCUGCAUCCAGCU | 51 | 28 |
| 2338 | 3937 | GGGACUGAGGGUGCUUGAG | 5273 | CUCAAGCACCCUCAGUCCC | 66 | 43 |
| 2339 | 3938 | GGACUGAGGGUGCUUGAGU | 5274 | ACUCAAGCACCCUCAGUCC | 74.5 | 51.5 |
| 2340 | 3939 | GACUGAGGGUGCUUGAGUA | 5275 | UACUCAAGCACCCUCAGUC | 89.2 | 66.2 |
| 2341 | 3940 | ACUGAGGGUGCUUGAGUAG | 5276 | CUACUCAAGCACCCUCAGU | 66.2 | 43.2 |
| 2342 | 3941 | CUGAGGGUGCUUGAGUAGG | 5277 | CCUACUCAAGCACCCUCAG | 56.4 | 33.4 |
| 2343 | 3942 | UGAGGGUGCUUGAGUAGGA | 5278 | UCCUACUCAAGCACCCUCA | 76.1 | 53.1 |
| 2344 | 3943 | GAGGGUGCUUGAGUAGGAU | 5279 | AUCCUACUCAAGCACCCUC | 79.7 | 56.7 |
| 2345 | 3944 | AGGGUGCUUGAGUAGGAUG | 5280 | CAUCCUACUCAAGCACCCU | 67.9 | 44.9 |
| 2346 | 3945 | GGGUGCUUGAGUAGGAUGU | 5281 | ACAUCCUACUCAAGCACCC | 80.9 | 57.9 |
| 2347 | 3946 | GGUGCUUGAGUAGGAUGUG | 5282 | CACAUCCUACUCAAGCACC | 76.9 | 53.9 |
| 2348 | 3947 | GUGCUUGAGUAGGAUGUGA | 5283 | UCACAUCCUACUCAAGCAC | 85.4 | 62.4 |
| 2349 | 3948 | UGCUUGAGUAGGAUGUGAG | 5284 | CUCACAUCCUACUCAAGCA | 66.3 | 43.3 |
| 2350 | 3949 | GCUUGAGUAGGAUGUGAGA | 5285 | UCUCACAUCCUACUCAAGC | 93.2 | 70.2 |
| 2351 | 3950 | CUUGAGUAGGAUGUGAGAC | 5286 | GUCUCACAUCCUACUCAAG | 63.4 | 40.4 |
| 2352 | 3951 | UUGAGUAGGAUGUGAGACU | 5287 | AGUCUCACAUCCUACUCAA | 65 | 42 |
| 2353 | 3952 | UGAGUAGGAUGUGAGACUU | 5288 | AAGUCUCACAUCCUACUCA | 78.8 | 55.8 |
| 2354 | 3953 | GAGUAGGAUGUGAGACUUC | 5289 | GAAGUCUCACAUCCUACUC | 77.3 | 54.3 |
| 2355 | 3954 | AGUAGGAUGUGAGACUUCA | 5290 | UGAAGUCUCACAUCCUACU | 89.3 | 66.3 |
| 2356 | 3955 | GUAGGAUGUGAGACUUCAU | 5291 | AUGAAGUCUCACAUCCUAC | 85.3 | 65.3 |
| 2357 | 3956 | UAGGAUGUGAGACUUCAUG | 5292 | CAUGAAGUCUCACAUCCUA | 63 | 40 |
| 2358 | 3957 | AGGAUGUGAGACUUCAUGG | 5293 | CCAUGAAGUCUCACAUCCU | 65.9 | 42.9 |
| 2359 | 3958 | GGAUGUGAGACUUCAUGGG | 5294 | CCCAUGAAGUCUCACAUCC | 70.7 | 47.7 |
| 2360 | 3959 | GAUGUGAGACUUCAUGGGC | 5295 | GCCCAUGAAGUCUCACAUC | 61.5 | 38.5 |
| 2361 | 3960 | AUGUGAGACUUCAUGGGCC | 5296 | GGCCCAUGAAGUCUCACAU | 49.9 | 26.9 |
| 2362 | 3961 | UGUGAGACUUCAUGGGCCU | 5297 | AGGCCCAUGAAGUCUCACA | 66.8 | 43.8 |
| 2363 | 3962 | GUGAGACUUCAUGGGCCUG | 5298 | CAGGCCCAUGAAGUCUCAC | 59.5 | 36.5 |
| 2364 | 3963 | UGAGACUUCAUGGGCCUGG | 5299 | CCAGGCCCAUGAAGUCUCA | 43.8 | 20.8 |
| 2365 | 3964 | GAGACUUCAUGGGCCUGGG | 5300 | CCCAGGCCCAUGAAGUCUC | 55.5 | 32.5 |
| 2366 | 3965 | AGACUUCAUGGGCCUGGGU | 5301 | ACCCAGGCCCAUGAAGUCU | 61 | 38 |
| 2367 | 3966 | GACUUCAUGGGCCUGGGUU | 5302 | AACCCAGGCCCAUGAAGUC | 67 | 44 |
| 2368 | 3967 | ACUUCAUGGGCCUGGGUUC | 5303 | GAACCCAGGCCCAUGAAGU | 56.4 | 33.4 |
| 2369 | 3968 | CUUCAUGGGCCUGGGUUCU | 5304 | AGAACCCAGGCCCAUGAAG | 61.3 | 38.3 |
| 2370 | 3969 | UUCAUGGGCCUGGGUUCUG | 5305 | CAGAACCCAGGCCCAUGAA | 42.7 | 19.7 |
| 2371 | 3970 | UCAUGGGCCUGGGUUCUGU | 5306 | ACAGAACCCAGGCCCAUGA | 57.2 | 34.2 |
| 2372 | 3971 | CAUGGGCCUGGGUUCUGUU | 5307 | AACAGAACCCAGGCCCAUG | 71.8 | 48.8 |
| 2373 | 3972 | AUGGGCCUGGGUUCUGUUG | 5308 | CAACAGAACCCAGGCCCAU | 53.4 | 30.4 |
| 2374 | 3973 | UGGGCCUGGGUUCUGUUGA | 5309 | UCAACAGAACCCAGGCCCA | 65.7 | 42.7 |
| 2375 | 3974 | GGGCCUGGGUUCUGUUGAG | 5310 | CUCAACAGAACCCAGGCCC | 71.5 | 48.5 |
| 2376 | 3975 | GGCCUGGGUUCUGUUGAGU | 5311 | ACUCAACAGAACCCAGGCC | 72.9 | 49.9 |
| 2377 | 3976 | GCCUGGGUUCUGUUGAGUU | 5312 | AACUCAACAGAACCCAGGC | 82.3 | 59.3 |
| 2378 | 3977 | CCUGGGUUCUGUUGAGUUU | 5313 | AAACUCAACAGAACCCAGG | 88.6 | 65.6 |
| 2397 | 3978 | UUUCAGUAUCAAUUUCUUA | 5314 | UAAGAAAUUGAUACUGAAA | 78.5 | 55.5 |
| 2398 | 3979 | UUCAGUAUCAAUUUCUUAA | 5315 | UUAAGAAAUUGAUACUGAA | 78.5 | 55.5 |
| 2399 | 3980 | UCAGUAUCAAUUUCUUAAA | 5316 | UUUAAGAAAUUGAUACUGA | 83.1 | 60.1 |
| 2400 | 3981 | CAGUAUCAAUUUCUUAAAC | 5317 | GUUUAAGAAAUUGAUACUG | 71.7 | 47.7 |
| 2401 | 3982 | AGUAUCAAUUUCUUAAACC | 5318 | GGUUUAAGAAAUUGAUACU | 59.3 | 35.3 |
| 2402 | 3983 | GUAUCAAUUUCUUAAACCA | 5319 | UGGUUUAAGAAAUUGAUAC | 86.3 | 62.3 |
| 2403 | 3984 | UAUCAAUUUCUUAAACCAA | 5320 | UUGGUUUAAGAAAUUGAUA | 82.9 | 58.9 |
| 2404 | 3985 | AUCAAUUUCUUAAACCAAA | 5321 | UUUGGUUUAAGAAAUUGAU | 84.3 | 60.3 |
| 2405 | 3986 | UCAAUUUCUUAAACCAAAU | 5322 | AUUUGGUUUAAGAAAUUGA | 76.7 | 52.7 |
| 2406 | 3987 | CAAUUUCUUAAACCAAAUU | 5323 | AAUUUGGUUUAAGAAAUUG | 79.7 | 55.7 |
| 2407 | 3988 | AAUUUCUUAAACCAAAUUU | 5324 | AAAUUUGGUUUAAGAAAUU | 71.1 | 47.1 |
| 2445 | 3989 | GGGUGCUCAUCUCGUGACC | 5325 | GGUCACGAGAUGAGCACCC | 64 | 40 |
| 2446 | 3990 | GGUGCUCAUCUCGUGACCU | 5326 | AGGUCACGAGAUGAGCACC | 72.9 | 48.9 |
| 2447 | 3991 | GUGCUCAUCUCGUGACCUC | 5327 | GAGGUCACGAGAUGAGCAC | 63.8 | 39.8 |
| 2448 | 3992 | UGCUCAUCUCGUGACCUCU | 5328 | AGAGGUCACGAGAUGAGCA | 66.7 | 42.7 |
| 2449 | 3993 | GCUCAUCUCGUGACCUCUG | 5329 | CAGAGGUCACGAGAUGAGC | 71.5 | 47.5 |
| 2450 | 3994 | CUCAUCUCGUGACCUCUGC | 5330 | GCAGAGGUCACGAGAUGAG | 54.8 | 30.8 |
| 2451 | 3995 | UCAUCUCGUGACCUCUGCC | 5331 | GGCAGAGGUCACGAGAUGA | 45.1 | 21.1 |
| 2468 | 3996 | CCACCCACAUCCUUCACAA | 5332 | UUGUGAAGGAUGUGGGUGG | 86.4 | 62.4 |
| 2469 | 3997 | CACCCACAUCCUUCACAAA | 5333 | UUUGUGAAGGAUGUGGGUG | 88.7 | 64.7 |
| 2470 | 3998 | ACCCACAUCCUUCACAAAC | 5334 | GUUUGUGAAGGAUGUGGGU | 59.7 | 35.7 |
| 2471 | 3999 | CCCACAUCCUUCACAAACU | 5335 | AGUUUGUGAAGGAUGUGGG | 77.3 | 53.3 |
| 2472 | 4000 | CCACAUCCUUCACAAACUC | 5336 | GAGUUUGUGAAGGAUGUGG | 71.6 | 47.6 |
| 2473 | 4001 | CACAUCCUUCACAAACUCC | 5337 | GGAGUUUGUGAAGGAUGUG | 61.1 | 37.1 |
| 2474 | 4002 | ACAUCCUUCACAAACUCCA | 5338 | UGGAGUUUGUGAAGGAUGU | 76.9 | 52.9 |
| 2475 | 4003 | CAUCCUUCACAAACUCCAU | 5339 | AUGGAGUUUGUGAAGGAUG | 83 | 59 |
| 2476 | 4004 | AUCCUUCACAAACUCCAUG | 5340 | CAUGGAGUUUGUGAAGGAU | 57 | 33 |
| 2477 | 4005 | UCCUUCACAAACUCCAUGU | 5341 | ACAUGGAGUUUGUGAAGGA | 63.5 | 39.5 |
| 2478 | 4006 | CCUUCACAAACUCCAUGUU | 5342 | AACAUGGAGUUUGUGAAGG | 82.2 | 58.2 |
| 2479 | 4007 | CUUCACAAACUCCAUGUUU | 5343 | AAACAUGGAGUUUGUGAAG | 78.4 | 54.4 |
| 2480 | 4008 | UUCACAAACUCCAUGUUUC | 5344 | GAAACAUGGAGUUUGUGAA | 59.9 | 35.9 |
| 2481 | 4009 | UCACAAACUCCAUGUUUCA | 5345 | UGAAACAUGGAGUUUGUGA | 79.7 | 55.7 |
| 2482 | 4010 | CACAAACUCCAUGUUUCAG | 5346 | CUGAAACAUGGAGUUUGUG | 68.6 | 44.6 |
| 2483 | 4011 | ACAAACUCCAUGUUUCAGU | 5347 | ACUGAAACAUGGAGUUUGU | 66.8 | 42.8 |
| 2484 | 4012 | CAAACUCCAUGUUUCAGUG | 5348 | CACUGAAACAUGGAGUUUG | 72.7 | 48.7 |
| 2485 | 4013 | AAACUCCAUGUUUCAGUGU | 5349 | ACACUGAAACAUGGAGUUU | 68.1 | 44.1 |
| 2486 | 4014 | AACUCCAUGUUUCAGUGUU | 5350 | AACACUGAAACAUGGAGUU | 77.6 | 53.6 |
| 2487 | 4015 | ACUCCAUGUUUCAGUGUUU | 5351 | AAACACUGAAACAUGGAGU | 85.1 | 61.1 |
| 2488 | 4016 | CUCCAUGUUUCAGUGUUUG | 5352 | CAAACACUGAAACAUGGAG | 71.2 | 47.2 |
| 2489 | 4017 | UCCAUGUUUCAGUGUUUGA | 5353 | UCAAACACUGAAACAUGGA | 80.2 | 56.2 |
| 2490 | 4018 | CCAUGUUUCAGUGUUUGAG | 5354 | CUCAAACACUGAAACAUGG | 71.9 | 47.9 |
| 2491 | 4019 | CAUGUUUCAGUGUUUGAGU | 5355 | ACUCAAACACUGAAACAUG | 79.2 | 55.2 |
| 2492 | 4020 | AUGUUUCAGUGUUUGAGUC | 5356 | GACUCAAACACUGAAACAU | 65.4 | 41.4 |
| 2493 | 4021 | UGUUUCAGUGUUUGAGUCC | 5357 | GGACUCAAACACUGAAACA | 60.4 | 36.4 |
| 2494 | 4022 | GUUUCAGUGUUUGAGUCCA | 5358 | UGGACUCAAACACUGAAAC | 86.3 | 62.3 |
| 2495 | 4023 | UUUCAGUGUUUGAGUCCAU | 5359 | AUGGACUCAAACACUGAAA | 73.5 | 49.5 |
| 2496 | 4024 | UUCAGUGUUUGAGUCCAUG | 5360 | CAUGGACUCAAACACUGAA | 58.6 | 34.6 |
| 2497 | 4025 | UCAGUGUUUGAGUCCAUGU | 5361 | ACAUGGACUCAAACACUGA | 71.3 | 47.3 |
| 2498 | 4026 | CAGUGUUUGAGUCCAUGUU | 5362 | AACAUGGACUCAAACACUG | 86.7 | 62.7 |
| 2499 | 4027 | AGUGUUUGAGUCCAUGUUU | 5363 | AAACAUGGACUCAAACACU | 85.1 | 61.1 |
| 2500 | 4028 | GUGUUUGAGUCCAUGUUUA | 5364 | UAAACAUGGACUCAAACAC | 95.3 | 70.3 |
| 2501 | 4029 | UGUUUGAGUCCAUGUUUAU | 5365 | AUAAACAUGGACUCAAACA | 83 | 58 |
| 2502 | 4030 | GUUUGAGUCCAUGUUUAUU | 5366 | AAUAAACAUGGACUCAAAC | 80.4 | 55.4 |
| 2503 | 4031 | UUUGAGUCCAUGUUUAUUC | 5367 | GAAUAAACAUGGACUCAAA | 54.1 | 29.1 |
| 2504 | 4032 | UUGAGUCCAUGUUUAUUCU | 5368 | AGAAUAAACAUGGACUCAA | 68.4 | 43.4 |
| 2505 | 4033 | UGAGUCCAUGUUUAUUCUG | 5369 | CAGAAUAAACAUGGACUCA | 62.9 | 37.9 |
| 2506 | 4034 | GAGUCCAUGUUUAUUCUGC | 5370 | GCAGAAUAAACAUGGACUC | 65.5 | 40.5 |
| 2507 | 4035 | AGUCCAUGUUUAUUCUGCA | 5371 | UGCAGAAUAAACAUGGACU | 84.3 | 59.3 |
| 2508 | 4036 | GUCCAUGUUUAUUCUGCAA | 5372 | UUGCAGAAUAAACAUGGAC | 93.4 | 68.4 |
| 2509 | 4037 | UCCAUGUUUAUUCUGCAAA | 5373 | UUUGCAGAAUAAACAUGGA | 78.1 | 53.1 |
| 2510 | 4038 | CCAUGUUUAUUCUGCAAAU | 5374 | AUUUGCAGAAUAAACAUGG | 90 | 65 |
| 2511 | 4039 | CAUGUUUAUUCUGCAAAUA | 5375 | UAUUUGCAGAAUAAACAUG | 91.3 | 66.3 |
| 2512 | 4040 | AUGUUUAUUCUGCAAAUAA | 5376 | UUAUUUGCAGAAUAAACAU | 87 | 62 |
| 2513 | 4041 | UGUUUAUUCUGCAAAUAAA | 5377 | UUUAUUUGCAGAAUAAACA | 94.1 | 69.1 |
| 2514 | 4042 | GUUUAUUCUGCAAAUAAAU | 5378 | AUUUAUUUGCAGAAUAAAC | 86.6 | 61.6 |
| 2515 | 4043 | UUUAUUCUGCAAAUAAAUG | 5379 | CAUUUAUUUGCAGAAUAAA | 57.2 | 32.2 |
| 2516 | 4044 | UUAUUCUGCAAAUAAAUGG | 5380 | CCAUUUAUUUGCAGAAUAA | 51.5 | 26.5 |
| 2517 | 4045 | UAUUCUGCAAAUAAAUGGU | 5381 | ACCAUUUAUUUGCAGAAUA | 66.6 | 41.6 |
| 2518 | 4046 | AUUCUGCAAAUAAAUGGUA | 5382 | UACCAUUUAUUUGCAGAAU | 77.5 | 52.5 |
| 2519 | 4047 | UUCUGCAAAUAAAUGGUAA | 5383 | UUACCAUUUAUUUGCAGAA | 78.4 | 53.4 |
| 2520 | 4048 | UCUGCAAAUAAAUGGUAAU | 5384 | AUUACCAUUUAUUUGCAGA | 74.4 | 49.4 |
| 2521 | 4049 | CUGCAAAUAAAUGGUAAUG | 5385 | CAUUACCAUUUAUUUGCAG | 64.5 | 39.5 |
| 2522 | 4050 | UGCAAAUAAAUGGUAAUGU | 5386 | ACAUUACCAUUUAUUUGCA | 66.3 | 41.3 |
| 2523 | 4051 | GCAAAUAAAUGGUAAUGUA | 5387 | UACAUUACCAUUUAUUUGC | 98.6 | 73.6 |
| 2524 | 4052 | CAAAUAAAUGGUAAUGUAU | 5388 | AUACAUUACCAUUUAUUUG | 87.1 | 62.1 |
| 2525 | 4053 | AAAUAAAUGGUAAUGUAUU | 5389 | AAUACAUUACCAUUUAUUU | 77.4 | 52.4 |
| 2526 | 4054 | AAUAAAUGGUAAUGUAUUG | 5390 | CAAUACAUUACCAUUUAUU | 71.2 | 46.2 |
| 2527 | 4055 | AUAAAUGGUAAUGUAUUGG | 5391 | CCAAUACAUUACCAUUUAU | 52.6 | 27.6 |
| 2528 | 4056 | UAAAUGGUAAUGUAUUGGA | 5392 | UCCAAUACAUUACCAUUUA | 71 | 46 |

In Table 5, “Pos.” refers to the position of the siRNA in the GPC2 input sequence. In this case, position 1 of SEQ ID NO: 1 corresponds to position 1 of siRNA positions listed in Table 5. “Score” refers to the predicted efficacy calculated from the SDIR 19 bp model, and “Corr. Score”, or Corrected Score refers to the previous efficacy score minored by the penalties from some intrinsic target features that can influence siRNA efficacy. Each row in Table 5 includes a sense region and complementary antisense region of a duplex of a representative siRNA of the disclosure.

In some embodiments, the sense region comprises a sequence selected from the group listed in Table 5. In some embodiments, the anti-sense region comprises a sequence selected from the group listed in Table 5. In some embodiments, the sense and anti-sense regions comprise complementary sequences selected from the group listed in Table 45

In some embodiments, the siRNA comprises a linker, sometimes referred to as a loop. siRNAs comprising a linker or loop are sometimes referred to as short hairpin RNAs (shRNAs). In some embodiments, both the sense and the anti-sense regions of the siRNA are encoded by one single-stranded RNA. In these embodiments, and the anti-sense region and the sense region hybridize to form a duplex region. The sense and anti-sense regions are joined by a linker sequence, forming a “hairpin” or “stem-loop” structure. The siRNA can have complementary sense and anti-sense regions at opposing ends of a single stranded molecule, so that the molecule can form a duplex region with the complementary sequence portions, and the strands are linked at one end of the duplex region by a linker. The linker can be either a nucleotide or non-nucleotide linker. The linker can interact with the first, and optionally, second strands through covalent bonds or non-covalent interactions.

Any suitable nucleotide linker sequence is envisaged as within the scope of the disclosure. An siRNA of this disclosure may include a nucleotide, non-nucleotide, or mixed nucleotide/non-nucleotide linker that joins the sense region of the nucleic acid to the anti-sense region of the nucleic acid. A nucleotide linker can be a linker of >2 nucleotides in length, for example about 3, 4, 5, 6, 7, 8, 9, or 10 nucleotides in length.

Examples of a non-nucleotide linker include an abasic nucleotide, polyether, polyamine, polyamide, peptide, carbohydrate, lipid, polyhydrocarbon, or other polymeric agents, for example polyethylene glycols such as those having from 2 to 100 ethylene glycol units. Some examples are described in Seela et al., Nucleic Acids Research, 1987, Vol. 15, pp. 3113-3129; Cload et al., J. Am. Chem. Soc, 1991, Vol. 113, pp. 6324-6326; Jaeschke et al., Tetrahedron Lett., 1993, Vol. 34, pp. 301; Arnold et al., WO 1989/002439; Usman et al., WO 1995/006731; Dudycz et al., WO 1995/011910, and Ferentz et al., J. Am. Chem. Soc, 1991, Vol. 113, pp. 4000-4002.

Examples of nucleotide linker sequences include, but are not limited to, AUG, CCC, UUCG, CCACC, AAGCAA, CCACACC and UUCAAGAGA.

In some embodiments, the siRNA encoded by a single RNA further comprises an overhang region, as described herein.

In some embodiments, an siRNA can be a dsRNA of a length suitable as a Dicer substrate, which can be processed to produce a RISC active siRNA molecule. See, e.g., Rossi et al., US2005/0244858.

A Dicer substrate double stranded RNA (dsRNA) can be of a length sufficient that it is processed by Dicer to produce an active siRNA, and may further include one or more of the following properties: (i) the Dicer substrate dsRNA can be asymmetric, for example, having a 3′ overhang on the anti-sense strand, (ii) the Dicer substrate dsRNA can have a modified 3′ end on the sense strand to direct orientation of Dicer binding and processing of the dsRNA to an active siRNA, for example the incorporation of one or more DNA nucleotides, and (iii) the first and second strands of the Dicer substrate ds RNA can from 21-30 bp in length.

In some embodiments, the siRNA comprises at least one modified nucleotide. In some embodiments, the at least one modified nucleotide increases the stability of the RNA duplex, and siRNA.

Modifications that increase RNA stability include, but are not limited to locked nucleic acids. As used herein, the term “locked nucleic acid” or “LNA” includes, but is not limited to, a modified RNA nucleotide in which the ribose moiety comprises a methylene bridge connecting the 2′ oxygen and the 4′ carbon. This methylene bridge locks the ribose in the 3′-endo confirmation, also known as the north confirmation, that is found in A-form RNA duplexes. The term inaccessible RNA can be used interchangeably with LNA. LNAs having a 2′-4′ cyclic linkage, as described in the International Patent Application WO 99/14226, WO 00/56746, WO 00/56748, and WO 00/66604, the contents of which are incorporated herein by reference.

In some embodiments, the at least one modified nucleotide comprises a phosphorothioate derivative or an acridinine substituted nucleotide.

In some embodiments, the modified nucleotide comprises 5-fluorouracil, 5-bromouracil, 5-chlorouracil, 5-iodouracil, hypoxanthine, xanthine, 4-acetylcytosine, 5-(carboxyhydroxylmethyl) uracil, 5-carboxymethylaminomethyl-2-thiouridine, 5-carboxymethylaminomet-hyluracil, dihydrouracil, beta-D-galactosylqueosine, inosine, N6-isopentenyladenine, 1-methylguanine, 1-methylinosine, 2,2-dimethylguanine, 2-methyladenine, 2-methylguanine, 3-methylcytosine, 5-methylcytosine, N6-adenine, 7-methylguanine, 5-methyl-aminomethyluracil, 5-methoxyaminomethyl-2-thiouracil, beta-D-mannosylqueosine, 5′-methoxycarboxymethyluracil, 5-methoxyuracil, 2-methylthio-N-isopenten-yladenine, uracil-5-oxyacetic acid (v), wybutoxosine, pseudouracil, queosine, 2-thiocytosine, 5-methyl-2-thiouracil, 2-thiouracil, 4-thiouracil, 5-methyluracil, uracil-5-oxyacetic acid methylester, uracil-5-oxyacetic acid (v), 5-methyl-2-thiouracil, 3-(3-amino-3-N-2-carboxypropyl) uracil, (acp3)w, or 2,6-diaminopurine.

### Nanoparticles

The disclosure provides nanoparticles comprising dsRNAs targeting a GPC2 mRNA for degradation. In some embodiments, the dsRNA is an siRNA, as described herein.

In some embodiments, the nanoparticles comprise siRNAs targeting a GPC2 mRNA sequence, and the siRNAs comprise a sense region and anti-sense region complementary to the sense region that together form an RNA duplex, and the sense region comprises a sequence at least 70% identical to a glypican-2 (GPC2) mRNA sequence.

In some embodiments, the nanoparticle comprises a liposome, a micelle, a polymer-based nanoparticle, a lipid-polymer based nanoparticle, a metal based nanoparticle, a carbon nanotube based nanoparticle, a nanocrystal or a polymeric micelle. In some embodiments, the polymer-based nanoparticle comprises a multiblock copolymer, a diblock copolymer, a polymeric micelle or a hyperbranched macromolecule. In some embodiments, the polymer-based nanoparticle comprises a multiblock copolymer a diblock copolymer. In some embodiments, the polymer-based nanoparticle is pH responsive. In some embodiments, the polymer-based nanoparticle further comprises a buffering component.

In some embodiments, the nanoparticle comprises a liposome. Liposomes are spherical vesicles having at least one lipid bilayer, and in some embodiments, an aqueous core. In some embodiments, the lipid bilayer of the liposome may comprise phospholipids. An exemplary but non-limiting example of a phospholipid is phosphatidylcholine, but the lipid bilayer may comprise additional lipids, such as phosphatidylethanolamine. Liposomes may be multilamellar, i.e. consisting of several lamellar phase lipid bilayers, or unilamellar liposomes with a single lipid bilayer. Liposomes can be made in a particular size range that makes them viable targets for phagocytosis. Liposomes can range in size from 20 nm to 100 nm, 100 nm to 400 nm, 1 μM and larger, or 200 nm to 3 μM. Examples of lipidoids and lipid-based formulations are provided in U.S. Published Application 20090023673. In other embodiments, the one or more lipids are one or more cationic lipids. One skilled in the art will recognize which liposomes are appropriate for siRNA encapsulation.

In some embodiments, the nanoparticle comprises a micelle. A micelle is an aggregate of surfactant molecules. An exemplary micelle comprises an aggregate of amphiphilic macromolecules, polymers or copolymers in aqueous solution, wherein the hydrophilic head portions contact the surrounding solvent, while the hydrophobic tail regions are sequestered in the center of the micelle.

In some embodiments, the nanoparticle comprises a nanocrystal. Exemplary nanocrystals are crystalline particles with at least one dimension of less than 1000 nanometers, preferably of less than 100 nanometers.

In some embodiments, the nanoparticle comprises a polymer based nanoparticle. In some embodiments, the polymer comprises a multiblock copolymer, a diblock copolymer, a polymeric micelle or a hyperbranched macromolecule. In some embodiments, the particle comprises one or more cationic polymers. In some embodiments, the cationic polymer is chitosan, protamine, polylysine, polyhistidine, polyarginine or poly(ethylene)imine. In other embodiments, the one or more polymers contain the buffering component, degradable component, hydrophilic component, cleavable bond component or some combination thereof.

In some embodiments, the nanoparticles or some portion thereof are degradable. In other embodiments, the lipids and/or polymers of the nanoparticles are degradable.

In some embodiments, any of these nanoparticles can comprise a buffering component. In other embodiments, any of the nanoparticles can comprise a buffering component and a degradable component. In still other embodiments, any of the nanoparticles can comprise a buffering component and a hydrophilic component. In yet other embodiments, any of the nanoparticles can comprise a buffering component and a cleavable bond component. In yet other embodiments, any of the nanoparticles can comprise a buffering component, a degradable component and a hydrophilic component. In still other embodiments, any of the nanoparticles can comprise a buffering component, a degradable component and a cleavable bond component. In further embodiments, any of the nanoparticles can comprise a buffering component, a hydrophilic component and a cleavable bond component. In yet another embodiment, any of the nanoparticles can comprise a buffering component, a degradable component, a hydrophilic component and a cleavable bond component. In some embodiments, the particle is composed of one or more polymers that contain any of the aforementioned combinations of components.

In further embodiments, the GPC2 targeting siRNA or dsRNA is conjugated to, complexed to, or encapsulated by the one or more lipids or polymers of the nanoparticle. GPC2 targeting dsRNAs or siRNAs can be encapsulated in the hollow core of a nanoparticle. Alternatively, or in addition, GPC2 targeting dsRNAs or siRNAs can be incorporated into the lipid or polymer based shell of the nanoparticle, for example via intercalation. Alternatively, or in addition, GPC2 targeting dsRNAs or siRNAs can be attached to the surface of the nanoparticle. In some embodiments, the GPC2 targeting siRNA or dsRNA is conjugated to one or more lipids or polymers of the nanoparticle, e.g. via covalent attachment.

In some embodiments, the nanoparticle further comprises a targeting agent. In some embodiments, the targeting agent comprises a peptide ligand, a nucleotide ligand, a polysaccharide ligand, a fatty acid ligand, a lipid ligand, a small molecule ligand, an antibody, an antibody fragment, an antibody mimetic or an antibody mimetic fragment. In some embodiments, the polysaccharide ligand is hyaluronic acid (HA). In some embodiments, the targeting agent binds to the surface of a cell of the cancer of the subject. In some embodiments, the targeting agent is on the surface and/or within the nanoparticle.

In some embodiments, the targeting agent comprises hyaluronic acid (HA). HA binds to CD44, a transmembrane peptidoglycan expressed on the surface of many types of cancer cells. CD44 integrates cellular environmental cues with growth factors and cytokine signals, and plays a role in the progression of many cancers. Targeting of CD44+ cells by HA nanoparticles thus provides superior delivery and specificity of the compositions of the disclosure to cancer cells.

In some embodiments, the nanoparticle further comprises a blending polymer. In some embodiments, the blending polymer is a copolymer comprising a degradable component and hydrophilic component. In some embodiments, the degradable component of the blending polymer is a polyester, poly(ortho ester), poly(ethylene imine), poly(caprolactone), polyanhydride, poly(acrylic acid), polyglycolide or poly(urethane). In some embodiments, the degradable component of the blending polymer is poly(lactic acid) (PLA) or poly(lactic-co-glycolic acid) (PLGA). In some embodiments, the hydrophilic component of the blending polymer is a polyalkylene glycol or a polyalkylene oxide. In some embodiments, the polyalkylene glycol is polyethylene glycol (PEG). In other embodiments, the polyalkylene oxide is polyethylene oxide (PEO).

In some embodiments, the nanoparticle is a polymer based nanoparticle. Polymer based nanoparticles comprise one or more polymers. In some embodiments, the one or more polymers comprise a polyester, poly(ortho ester), poly(ethylene imine), poly(caprolactone), polyanhydride, poly(acrylic acid), polyglycolide or poly(urethane). In still other embodiments, the one or more polymers comprise poly(lactic acid) (PLA) or poly(lactic-co-glycolic acid) (PLGA). In some embodiments, the one or more polymers comprise poly(lactic-co-glycolic acid) (PLGA). In some embodiments, the one or more polymers comprise poly(lactic acid) (PLA). In some embodiments, the one or more polymers comprise polyalkylene glycol or a polyalkylene oxide. In some embodiments, the polyalkylene glycol is polyethylene glycol (PEG) or the polyalkylene oxide is polyethylene oxide (PEO).

In some embodiments, the nanoparticle comprising the GPC2 siRNA is a polymer based nanoparticle. In some embodiments, the polymer-based nanoparticle comprises poly(lactic-co-glycolic acid) PLGA polymers. In some embodiments, the PLGA nanoparticle further comprises a targeting agent, such as HA.

In some embodiments, the nanoparticle has an average characteristic dimension of less than about 500 nm, 400 nm, 300 nm, 250 nm, 200 nm, 180 nm, 150 nm, 120 nm, 100 nm, 90 nm, 80 nm, 70 nm, 60 nm, 50 nm, 40 nm, 30 nm or 20 nm. In other embodiments, the nanoparticle has an average characteristic dimension of 10 nm, 20 nm, 30 nm, 40 nm, 50 nm, 60 nm, 70 nm, 80 nm, 90 nm, 100 nm, 120 nm, 150 nm, 180 nm, 200 nm, 250 nm or 300 nm. In further embodiments, the nanoparticle has an average characteristic dimension of 10-500 nm, 10-400 nm, 10-300 nm, 10-250 nm, 10-200 nm, 10-150 nm, 10-100 nm, 10-75 nm, 10-50 nm, 50-500 nm, 50-400 nm, 50-300 nm, 50-200 nm, 50-150 nm, 50-100 nm, 50-75 nm, 100-500 nm, 100-400 nm, 100-300 nm, 100-250 nm, 100-200 nm, 100-150 nm, 150-500 nm, 150-400 nm, 150-300 nm, 150-250 nm, 150-200 nm, 200-500 nm, 200-400 nm, 200-300 nm, 200-250 nm, 200-500 nm, 200-400 nm or 200-300 nm.

In some embodiments, for example those embodiments where nanoparticles comprising GPC2-targeting siRNAs are administered with one or more additional cancer therapies, the nanoparticle can comprise at least one siRNA targeting GPC2 and one or more therapeutic or chemotherapeutic agents. For example, the nanoparticle comprises at least one siRNA targeting GPC2 and a platinum based antineoplastic agent, or a DNA alkylating agent, or a DNA intercalating agent, or a topoisomerase inhibitor.

Chemotherapeutic agents that can be incorporated into the nanoparticles described herein include, but are not limited to, Cisplatin, Carboplatin, Doxorubicin, Cyclophosphamide, Etoposide, and Topotecan.

### Therapeutic Agents

Provided herein are therapeutic agents, such as chemotherapeutic agents, which can be administered with the nanoparticles comprising siRNAs targeting GPC2 described herein.

In some embodiments, the additional therapeutic agent is incorporated into a nanoparticle comprising at least one siRNA targeting GPC2. In some embodiments, the additional therapeutic agent is conjugated to, complexed to, or encapsulated by the one or more lipids or polymers of the nanoparticle. Additional therapeutic agents can be encapsulated in the hollow core of a nanoparticle. Alternatively, or in addition, Additional therapeutic agents can be incorporated into the lipid or polymer based shell of the nanoparticle, for example via intercalation. Alternatively, or in addition, additional therapeutic agents can be attached to the surface of the nanoparticle. In some embodiments, the additional therapeutic agents are conjugated to one or more lipids or polymers of the nanoparticle, e.g. via covalent attachment.

In some embodiments, the additional therapeutic agent and the nanoparticles comprising siRNAs targeting GPC2 are formulated in the same composition. For example the nanoparticles comprising siRNAs targeting GPC2 and the additional therapeutic agent can be formulated in the same pharmaceutical composition.

In some embodiments, the additional therapeutic agent and the nanoparticles comprising siRNAs targeting GPC2 are formulated as separate compositions, e.g. for separate administration to a subject.

In some embodiments, the additional therapeutic agent is a chemotherapeutic agent. In some embodiments, the chemotherapeutic agent comprises a platinum based antineoplastic agent, a DNA alkylating agent, a DNA intercalating agent, or a topoisomerase inhibitor.

In some embodiments, the chemotherapeutic agent may comprise platinum based antineoplastic drug (platins). One mechanism of action by which platins work is through the crosslinking of DNA, which inhibits DNA repair, DNA synthesis, or both in cancer cells. Exemplary, but not limiting platins comprise Cisplatin or Carboplatin, and analogs or derivatives thereof.

In some embodiments, the chemotherapeutic agent may comprise a Topoisomerase I inhibitor or Topoisomerase II. Topoisomerase I and II are enzymes which regulates DNA structure by breaking and rejoining the phosphodiester backbone of the DNA during the cell cycle (e.g., during DNA synthesis). Without functional Topoisomerase I or II, single and double strand breaks accumulate, leading to cell death. Exemplary but not limiting Topoisomerase I inhibitors comprise Irinotecan, Topotecan, and analogs or derivatives thereof. Exemplary but not limiting Topoisomerase II inhibitors comprise Etoposide and analogs or derivatives thereof.

In some embodiments, the chemotherapeutic agent may comprise a DNA alkylating agent. DNA alkylating agents attach an alkyl group to DNA, typically to the guanine base of the DNA. This causes DNA damage, and may kill the cancer cells or stop them from dividing. In some embodiments, the DNA alkylating agent comprises Dacarbazine, Temozolomide, Cyclophosphamide or Ifosfamide and analogs or derivatives thereof.

In some embodiments, the chemotherapeutic agent may comprise a DNA intercalating agent. DNA intercalating agents insert themselves into the structure of the DNA within a cell and bind to the DNA, causing DNA damage. This may kill cancer cells, or stop them from dividing. Exemplary but not limiting DNA intercalating agents comprise Doxorubicin and analogs or derivatives thereof.

In some embodiments, the chemotherapeutic agent may comprise a taxane. Taxanes are a class of diterpenes that have long been used in cancer treatment. Taxanes act by disrupting microtubule function, which in turn disrupts cell division. Typically, taxanes act by stabilizing GDP bound tubulin in the microtubule, disrupting microtubule depolymerization and dynamic instability of microtubules. Exemplary taxanes comprise Paclitaxel or Docetaxel, and analogs or derivatives thereof.

In some embodiments, the chemotherapeutic agent may comprise a Vinca alkaloid. Like taxanes, Vinca alkaloids also act upon tubulin. Vinca alkaloids prevent microtubule polymerization, thus preventing cell division. Exemplary but not limiting Vinca alkaloids comprise Vinblastine, Vincristine, Vinorelbine, Vincaminol, Vineridine, Vinburnine, Vindesine, Vincamine and analogs or derivatives thereof.

In some embodiments, the chemotherapeutic agent may comprise a thymidylate synthase inhibitor. Thymidylate synthase is a key enzyme involved in DNA synthesis. Exemplary but not limiting thymidylate synthase inhibitors comprise 5-Fluorouracil and analogs or derivatives thereof.

Additional chemotherapeutic agents that cause DNA damage, such as through the binding of DNA or interfering with DNA synthesis, are also considered as within the scope of the invention.

### Pharmaceutical Compositions

Provided herein are pharmaceutical compositions comprising nanoparticles, the nanoparticles comprising dsRNAs targeting GPC2 as described herein.

Provided herein are pharmaceutical compositions comprising the nanoparticles comprising siRNAs targeting GPC2 as described herein.

The pharmaceutical compositions of the disclosure can optionally comprise therapeutic agents, pharmaceutical agents, carriers, adjuvants, dispersing agents, diluents, and the like.

In some embodiments, the pharmaceutical composition comprises a therapeutic agent, such as a chemotherapeutic agent. In some embodiments, the therapeutic agent is formulated in the same nanoparticle as the dsRNA or siRNA targeting GPC2.

In some embodiments, the therapeutic agent is not formulated in the nanoparticle comprising the dsRNA or siRNA targeting GPC2, but both the nanoparticle and the therapeutic agent are formulated in the same pharmaceutical composition. In some embodiments, the therapeutic agent is not formulated in the nanoparticle comprising the dsRNA or siRNA targeting GPC2, and the nanoparticle and therapeutic agent are formulated in separate pharmaceutical compositions.

Pharmaceutical compositions can contain any of the reagents discussed above, and one or more of a pharmaceutically acceptable carrier, a diluent or an excipient.

A “pharmaceutical composition” is a formulation comprising the nanoparticles described herein, in a form suitable for administration to a subject. In one embodiment, the pharmaceutical composition is in bulk or in unit dosage form. The unit dosage form is any of a variety of forms, including, for example, a capsule, an IV bag, a tablet, a single pump on an aerosol inhaler or a vial. The quantity of active ingredient (e.g., a formulation of the disclosed agent) in a unit dose of composition is an effective amount and is varied according to the particular treatment involved. One skilled in the art will appreciate that it is sometimes necessary to make routine variations to the dosage depending on the age and condition of the patient. The dosage will also depend on the route of administration. A variety of routes are contemplated, including oral, pulmonary, rectal, parenteral, transdermal, subcutaneous, intravenous, intramuscular, intraperitoneal, inhalational, buccal, sublingual, intrapleural, intrathecal, intranasal, and the like. Dosage forms for the topical or transdermal administration of a of this invention include powders, sprays, ointments, pastes, creams, lotions, gels, solutions, patches and inhalants. In one embodiment, the active agent is mixed under sterile conditions with a pharmaceutically acceptable carrier, and with any preservatives, buffers, or propellants that are required.

As used herein, the phrase “pharmaceutically acceptable” refers to those compounds, anions, cations, materials, compositions, carriers, and/or dosage forms which are, within the scope of sound medical judgment, suitable for use in contact with the tissues of human beings and animals without excessive toxicity, irritation, allergic response, or other problem or complication, commensurate with a reasonable benefit/risk ratio.

“Pharmaceutically acceptable excipient” means an excipient that is useful in preparing a pharmaceutical composition that is generally safe, non-toxic and neither biologically nor otherwise undesirable, and includes excipient that is acceptable for veterinary use as well as human pharmaceutical use. A “pharmaceutically acceptable excipient” as used in the specification and claims includes both one and more than one such excipient.

A pharmaceutical composition of the invention is formulated to be compatible with its intended route of administration. Examples of routes of administration include parenteral, e.g., intravenous, intradermal, subcutaneous, oral (e.g., inhalation), transdermal (topical), intraperitoneal (into the body cavity) and transmucosal administration. Solutions or suspensions used for parenteral, intradermal, intraperitoneal or subcutaneous application can include the following components: a sterile diluent such as water for injection, saline solution, fixed oils, polyethylene glycols, glycerine, propylene glycol or other synthetic solvents; antibacterial agents such as benzyl alcohol or methyl parabens; antioxidants such as ascorbic acid or sodium bisulfite; chelating agents such as ethylenediaminetetraacetic acid; buffers such as acetates, citrates or phosphates, and agents for the adjustment of tonicity such as sodium chloride or dextrose. The pH can be adjusted with acids or bases, such as hydrochloric acid or sodium hydroxide. The parenteral preparation can be enclosed in ampoules, disposable syringes or multiple dose vials made of glass or plastic. These preparations can contain antioxidants, buffers, bacteriostats and solutes which render the formulation isotonic with the blood of the intended recipient. Aqueous and non-aqueous sterile suspensions can include suspending agents and thickening agents. The formulations can be presented in unit/dose or multi-dose containers, for example sealed ampoules and vials, and can be stored in a freeze-dried (lyophilized) condition requiring only the addition of the sterile liquid carrier, for example, saline or water-for-injection immediately prior to use.

The pharmaceutical compositions containing the nanoparticles described herein may be manufactured in a manner that is generally known, e.g., by means of conventional mixing, dissolving, granulating, dragee-making, levigating, emulsifying, encapsulating, entrapping, or lyophilizing processes. Pharmaceutical compositions may be formulated in a conventional manner using one or more pharmaceutically acceptable carriers comprising excipients and/or auxiliaries that facilitate processing of the active agents into preparations that can be used pharmaceutically. Of course, the appropriate formulation is dependent upon the route of administration chosen.

Pharmaceutical compositions suitable for injectable use include sterile aqueous solutions (where water soluble) or dispersions and sterile powders for the extemporaneous preparation of sterile injectable solutions or dispersion. For intravenous administration, suitable carriers include physiological saline, bacteriostatic water, Cremophor EL™ (BASF, Parsippany, N.J.) or phosphate buffered saline (PBS). In all cases, the composition must be sterile and should be fluid to the extent that easy syringeability exists. It must be stable under the conditions of manufacture and storage and must be preserved against the contaminating action of microorganisms such as bacteria and fungi. The carrier can be a solvent or dispersion medium containing, for example, water, ethanol, polyol (for example, glycerol, propylene glycol, and liquid polyethylene glycol, and the like), and suitable mixtures thereof. The proper fluidity can be maintained, for example, by the use of a coating such as lecithin, by the maintenance of the required nanoparticle size in the case of dispersion and by the use of surfactants. Prevention of the action of microorganisms can be achieved by various antibacterial and antifungal agents, for example, parabens, chlorobutanol, phenol, ascorbic acid, thimerosal, and the like. In many cases, it will be preferable to include isotonic agents, for example, sugars, polyalcohols such as manitol and sorbitol, and sodium chloride in the composition. Prolonged absorption of the injectable compositions can be brought about by including in the composition an agent which delays absorption, for example, aluminum monostearate and gelatin.

Oral compositions generally include an inert diluent or an edible pharmaceutically acceptable carrier. They can be enclosed in gelatin capsules or compressed into tablets. For the purpose of oral therapeutic administration, the active age can be incorporated with excipients and used in the form of tablets, troches, or capsules. Oral compositions can also be prepared using a fluid carrier for use as a mouthwash, wherein the agents in the fluid carrier is applied orally and swished and expectorated or swallowed. Pharmaceutically compatible binding agents, and/or adjuvant materials can be included as part of the composition. The tablets, pills, capsules, troches and the like can contain any of the following ingredients, or agents of a similar nature: a binder such as microcrystalline cellulose, gum tragacanth or gelatin; an excipient such as starch or lactose, a disintegrating agent such as alginic acid, Primogel, or corn starch; a lubricant such as magnesium stearate or Sterotes; a glidant such as colloidal silicon dioxide; a sweetening agent such as sucrose or saccharin; or a flavoring agent such as peppermint, methyl salicylate, or orange flavoring.

For administration by inhalation, the agents are delivered in the form of an aerosol spray from pressured container or dispenser, which contains a suitable propellant, e.g., a gas such as carbon dioxide, or a nebulizer.

The nanoparticles comprising dsRNAs or siRNAs can be prepared with pharmaceutically acceptable carriers that will protect the dsRNAs or siRNAs against rapid elimination from the body, such as a controlled release formulation, including implants and microencapsulated delivery systems. Biodegradable, biocompatible polymers can be used, such as ethylene vinyl acetate, polyanhydrides, polyglycolic acid, collagen, polyorthoesters, and polylactic acid. Methods for preparation of such formulations will be apparent to those skilled in the art, and the materials can be obtained commercially. Liposomal suspensions (including liposomes targeted to infected cells with monoclonal antibodies to viral antigens) can also be used as pharmaceutically acceptable carriers. These can be prepared according to methods known to those skilled in the art, for example, as described in U.S. Pat. No. 4,522,811.

It is especially advantageous to formulate oral or parenteral compositions in dosage unit form for ease of administration and uniformity of dosage. Dosage unit form as used herein refers to physically discrete units suited as unitary dosages for the subject to be treated; each unit containing a predetermined quantity of active agent calculated to produce the desired therapeutic effect in association with the required pharmaceutical carrier. The specification for the dosage unit forms of the invention are dictated by and directly dependent on the unique characteristics of the active agent and the particular therapeutic effect to be achieved.

The pharmaceutical compositions can be included in a container, pack, or dispenser together with instructions for administration.

As used herein, “pharmaceutically acceptable salts” refer to derivatives of the compounds of the present invention wherein the parent compound is modified by making acid or base salts thereof. Examples of pharmaceutically acceptable salts include, but are not limited to, mineral or organic acid salts of basic residues such as amines, alkali or organic salts of acidic residues such as carboxylic acids, and the like. The pharmaceutically acceptable salts include the conventional non-toxic salts or the quaternary ammonium salts of the parent compound formed, for example, from non-toxic inorganic or organic acids.

Techniques for formulation and administration of the disclosed compositions of the invention can be found in Remington: the Science and Practice of Pharmacy, 19th edition, Mack Publishing Co., Easton, Pa. (1995).

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### Nucleic Acids and Vectors

The disclosure provides nucleic acids comprising the sequences encoding the dsRNAs or siRNAs targeting GPC2 described herein.

In some embodiments, the nucleic acids are ribonucleic acids (RNAs).

In some embodiments, the nucleic acids are deoxyribonucleic acids (DNAs). The DNAs may be a vector or a plasmid, e.g., an expression vector.

A “vector” is any nucleic acid molecule for the cloning of and/or transfer of a nucleic acid into a cell. A vector may be a replicon to which another nucleotide sequence may be attached to allow for replication of the attached nucleotide sequence. A “replicon” can be any genetic element (e.g., plasmid, phage, cosmid, chromosome, viral genome) that functions as an autonomous unit of nucleic acid replication in vivo, i.e., capable of replication under its own control. The term “vector” includes both viral and nonviral (e.g., plasmid) nucleic acid molecules for introducing a nucleic acid into a cell in vitro, ex vivo, and/or in vivo. A large number of vectors known in the art may be used to manipulate nucleic acids, incorporate response elements and promoters into genes, etc. For example, the insertion of the nucleic acid fragments corresponding to response elements and promoters into a suitable vector can be accomplished by ligating the appropriate nucleic acid fragments into a chosen vector that has complementary cohesive termini. Alternatively, the ends of the nucleic acid molecules may be enzymatically modified or any site may be produced by ligating nucleotide sequences (linkers) to the nucleic acid termini Such vectors may be engineered to contain sequences encoding selectable markers that provide for the selection of cells that contain the vector and/or have incorporated the nucleic acid of the vector into the cellular genome. Such markers allow identification and/or selection of host cells that incorporate and express the proteins encoded by the marker. A “recombinant” vector refers to a viral or non-viral vector that comprises one or more heterologous nucleotide sequences (i.e., transgenes), e.g., two, three, four, five or more heterologous nucleotide sequences.

By the term “express” or “expression” of a polynucleotide coding sequence, it is meant that the sequence is transcribed, and optionally, translated. Typically, according to the present invention, expression of a coding sequence of the invention will result in production of the polypeptide of the invention. The entire expressed polypeptide or fragment can also function in intact cells without purification.

In some embodiments, the vector is an expression vector for manufacturing siRNAs of the disclosure. Exemplary expression vectors may comprise a sequence encoding the sense and/or anti-sense strand of the siRNA under the control of a suitable promoter for transcription. Interfering RNAs may be expressed from a variety of eukaryotic promoters known to those of ordinary skill in the art, including pol III promoters, such as the U6 or H1 promoters, or pol II promoters, such as the cytomegalovirus promoter. Those of skill in the art will recognize that these promoters can also be adapted to allow inducible expression of the interfering RNA.

dsRNAs and siRNAs can be expressed endogenously from plasmid or viral expression vectors, or from minimal expression cassettes, for example, PCR generated fragments comprising one or more promoters and an appropriate template or templates for transcribing the siRNA. Examples of commercially available plasmid-based expression vectors for shRNA include members of the pSilencer series (Ambion, Austin. Tex.) and pCpG-siRNA (InvivoGen. San Diego, Calif.). Examples of kits for production of PCR-generated shRNA expression cassettes include Silencer Express (Ambion, Austin, Tex.) and siXpress (Minis, Madison. Wis.).

Viral vectors for the in vivo expression of siRNAs and dsRNAs in eukaryotic cells are also contemplated as within the scope of the instant disclosure. Viral vectors may be derived from a variety of viruses including adenovirus, adeno-associated virus, lentivirus (e.g., HIV, FIV, and EIAV), and herpes virus. Examples of commercially available viral vectors for shRNA expression include pSilencer adeno (Ambion, Austin, Tex.) and pLenti6/BLOCK-iT™-DEST (Invitrogen, Carlsbad, Calif.). Selection of viral vectors, methods for expressing the siRNA from the vector and methods of delivering the viral vector, for example incorporated within a nanoparticle, are within the ordinary skill of one in the art.

It will be apparent to those skilled in the art that any suitable vector, optionally incorporated into a nanoparticle, can be used to deliver the dsRNAs or siRNAs described herein to a cell or subject. The vector can be delivered to cells in vivo. In other embodiments, the vector can be delivered to cells ex vivo, and then cells containing the vector are delivered to the subject. The choice of delivery vector can be made based on a number of factors known in the art, including age and species of the target host, in vitro versus in vivo delivery, level and persistence of expression desired, intended purpose (e.g., for therapy or screening), the target cell or organ, route of delivery, size of the isolated polynucleotide, safety concerns, and the like.

Methods of Making dsRNAs

Provided herein are methods of making dsRNAs or siRNAs targeting GPC2, and nanoparticles comprising same.

siRNAs may be generated exogenously by chemical synthesis, by in vitro transcription, or by cleavage of longer double-stranded RNA with Dicer or another appropriate nuclease with similar activity. Chemically synthesized siRNAs, produced from protected ribonucleoside phosphoramidites using a conventional DNA/RNA synthesizer, may be obtained from commercial suppliers such as Millipore Sigma (Houston, Tex.), Ambion Inc. (Austin, Tex.). Invitrogen (Carlsbad, Calif.), or Dharmacon (Lafayette, Colo.). siRNAs can be purified by extraction with a solvent or resin, precipitation, electrophoresis, chromatography, or a combination thereof, for example. Alternatively, siRNAs may be used with little if any purification to avoid losses due to sample processing.

In alternative embodiments, dsRNAs and siRNAs can be produced using an expression vector into which a nucleic acid encoding the double stranded RNA has been cloned, for example under control of a suitable promoter.

In some embodiments, dsRNAs or siRNAs can be incorporated in a nanoparticle.

Nanoparticles comprising dsRNAs or siRNAs of the disclosure can be prepared by any suitable means known in the art. For example, polymeric nanoparticles can be prepared using various methods including, but not limited to, solvent evaporation, spontaneous emulsification, solvent diffusion, desolvation, dialysis, ionic gelation, nanoprecipitation, salting out, spray drying and supercritical fluid methods. The dispersion of preformed polymers and the polymerization of monomers are two additional strategies for preparation of polymeric nanoparticles. However, the choice of an appropriate method depends upon various factors, which will be known to the person of ordinary skill in the art.

Sterile injectable solutions comprising a nanoparticle of the disclosure can be prepared by incorporating the GPC2-targeting siRNA in the nanoparticles in the required amount in an appropriate solvent with one or a combination of ingredients enumerated herein, as required, followed by filtered sterilization. Alternatively, or in addition, sterilization can be achieved through other means such as radiation or gas. Generally, dispersions are prepared by incorporating the nanoparticles into a sterile vehicle that contains a basic dispersion medium and the required other ingredients from those enumerated above. In the case of sterile powders for the preparation of sterile injectable solutions, methods of preparation are vacuum drying and freeze drying that yields a powder of GPC2 siRNA nanoparticles plus any additional desired ingredient from a previously sterile filtered solution thereof.

### Inhibition of GPC2 Expression

Provided herein are methods of reducing or inhibiting GPC2 expression or activity in a cell, comprising contacting the cell with an siRNA targeting GPC2 as described herein. siRNAs of the disclosure that target GPC2 can reduce or inhibit GPC2 activity through the RNAi pathway. The cell can be in vitro, in vivo or ex vivo. For example, the cell can be from a cell line, or in vivo in a cancer patient.

In some embodiments, siRNAs of the disclosure are capable of inducing RNAi-mediated degradation of a GPC2 mRNA in a cancer cell of a subject.

In some embodiments, administration of nanoparticles comprising siRNAs targeting GPC2 or pharmaceutical compositions comprising same decreases viability of a cell of the cancer.

In some embodiments, administration of nanoparticles comprising siRNAs targeting GPC2 or pharmaceutical compositions comprising same increases apoptosis of cells.

As used herein, the terms “contacting,” “introducing” and “administering” are used interchangeably, and refer to a process by which dsRNA or siRNA of the present disclosure or a nucleic acid molecule encoding a dsRNA or siRNA of this disclosure is delivered to a cell, in order to inhibit or alter or modify expression of a target gene. The dsRNA may be administered in a number of ways, including, but not limited to, direct introduction into a cell (i.e., intracellularly) and/or extracellular introduction into a cavity, interstitial space, or into the circulation of the organism.

“Introducing” in the context of a cell or organism means presenting the nucleic acid molecule to the organism and/or cell in such a manner that the nucleic acid molecule gains access to the interior of a cell. Where more than one nucleic acid molecule is to be introduced these nucleic acid molecules can be assembled as part of a single polynucleotide or nucleic acid construct, or as separate polynucleotide or nucleic acid constructs, and can be located on the same or different nucleic acid constructs. Accordingly, these polynucleotides can be introduced into cells in a single transformation event or in separate transformation events. Thus, the term “transformation” as used herein refers to the introduction of a heterologous nucleic acid into a cell. Transformation of a cell may be stable or transient.

The term “inhibit” or “reduce” or grammatical variations thereof, as used herein, refer to a decrease or diminishment in the specified level or activity of at least about 5%, about 10%, about 15%, about 25%, about 35%, about 40%, about 50%, about 60%, about 75%, about 80%, about 90%, about 95% or more. In some embodiments, the inhibition or reduction results in little or essentially no detectible activity (at most, an insignificant amount, e.g., less than about 10% or even 5%).

In contrast, the term “increase” or grammatical variations thereof as used herein refers to an increase or elevation in the specified level or activity of at least about 5%, about 10%, about 15%, about 25%, about 35%, about 40%, about 50%, about 60%, about 75%, about 80%, about 90%, about 95% or more. Increases in activity can be described in terms of fold change. For example, activity can be increased 1.2×, 1.5×, 2×, 3×, 5×, 6×, 7×, 8×, 9×, 10× or more compared to a baseline level of activity.

As used herein, the term “IC50” or “IC₅₀ value” refers to the concentration of an agent where cell viability is reduced by half. The IC₅₀ is thus a measure of the effectiveness of an agent in inhibiting a biological process. In an exemplary model, cancerous cell lines are cultured using standard techniques, treated with a GPC2 siRNA and the IC₅₀ value of the GPC2 siRNA is calculated after 24, 48 and/or 72 hours to determine its effectiveness in killing the cancer cells or inhibiting cell growth.

Methods of monitoring of GPC2 mRNA and/or protein expression can be used to characterize gene silencing, and to determine the effectiveness of the compositions described herein. Expression of GPC2 may be evaluated by any known technique. Examples thereof include immunoprecipitations methods, utilizing GPC2 antibodies in assays such as ELISAs, Western Blot, or immunohistochemistry to visualize GPC2 protein expression in cells, or flow cytometry. Additional methods include various hybridization methods utilizing a nucleic acid that specifically hybridizes with a nucleic acid encoding GPC2 or a unique fragment thereof, or a transcription product (e.g., mRNA) or splicing product of said nucleic acid, Northern Blot methods, Southern blot methods, and various PCR-based methods such as RT-PCR, qPCR or digital droplet PCR. GPC2 mRNA expression may additionally be assessed using high throughput sequencing techniques. Using high throughput sequencing of RNA libraries, the level of GPC2 mRNA from a sample of cells can be calculated from the number of reads that map to a GPC2 reference sequence using techniques known in the art. GPC2 expression can be assayed in cultured cells, spheroid bodies, patient derived xenograft cancer models, or patient samples.

Methods of assaying the effect of individual dsRNAs or siRNAs on tumor cells include transfecting representative cell lines with dsRNAs or siRNAs targeting GPC2, and measuring viability. For example, cells from representative cell lines can be transfected using methods known in the art, such as the RNAiMAX Lipofectamine kit (Invitrogen, Carlsbad, Calif.), and cultured using any suitable technique known in the art. Optionally additional therapeutic agents as described herein can be added at variable concentrations to cell culture media following transfection. Following a suitable incubation period, such as 24-96 hours, cell viability can be measured using methods such as Cell Titer Glo 2.0 (Promega, CA) to determine cell viability, and/or GPC2 mRNA and protein levels can be assessed using the methods described herein.

### Treatment of Cancer

Provided herein are methods of treating a cancer in a subject, comprising administering to the subject a therapeutically effective amount of a nanoparticle comprising a dsRNA or siRNA targeting GPC2. In some embodiments, the methods comprise administering a chemotherapeutic agent to the subject. The chemotherapeutic agent can be formulated in the nanoparticles, in the same pharmaceutical composition as the nanoparticles, or in a separate composition. In some embodiments, the chemotherapeutic agent comprises a platinum based antineoplastic agent, a DNA alkylating agent, a DNA intercalating agent, or a topoisomerase inhibitor as described herein.

In some embodiments, the methods comprise administering to the subject a therapeutically effective amount of a composition comprising a nanoparticle, the nanoparticle comprising a small interfering RNA (siRNA), wherein the siRNA comprises a sense region and anti-sense region complementary to the sense region that together form an RNA duplex, wherein the sense region comprises a sequence at least 70% identical to a glypican-2 (GPC2) mRNA sequence of SEQ ID NO: 1 or SEQ ID NO: 2.

Suitable subjects include mammals. The term “mammal” as used herein includes, but is not limited to, humans, bovines, ovines, caprines, equines, felines, canines, lagomorphs, etc. Human subjects include neonates, infants, juveniles, and adults. In some embodiments, the subject is an animal model of cancer, for example a mouse or a rat model of cancer. In certain embodiments, the subject has or is at risk for cancer.

In some embodiments, the cancer is a cancer that expresses GPC2 on the cells of the cancer.

In some embodiments, the cancer is selected from the group consisting of astrocytoma, breast cancer, colorectal cancer, Ewing's sarcoma, gastric cancer, leiomyosarcoma, liver cancer, lung cancer, mesothelioma, ovarian cancer, pancreatic cancer, renal cancer, rhabdomyosarcoma and neuroblastoma.

In some embodiments, the cancer is astrocytoma. Astrocytomas are a type of cancer of the brain. Astrocytomas originate from a type of glial cells, star-shaped brain cells in the cerebrum called astrocytes. This type of tumor does not usually spread outside the brain and spinal cord.

In some embodiments, the cancer is breast cancer. Breast cancers are cancers that arise from cells in the breast. Breast cancers can occur in both men and women. Exemplary types of breast cancer include ductal carcinoma in situ, invasive breast cancers such as invasive ductal carcinoma and invasive lobular carcinoma, and inflammatory breast cancer.

In some embodiments, the cancer is colorectal cancer. Colorectal cancers comprise cancers of the colon or rectum. The rectum is the passageway that connects the colon to the anus. In certain embodiments, the colorectal cancer comprises a colorectal carcinoma.

In some embodiments, the cancer is Ewing's sarcoma. Ewing's sarcoma comprises tumors of the bones, the soft tissue surrounding bones such as cartilage and nerves, or a combination thereof. Ewing's sarcoma typically affects children and young adults, although it can occur at any age. Ewing's sarcoma can occur in any bone. In some more frequent embodiments, Ewing's sarcoma begins in the leg bones, hipbones, arm bones, and bones in the chest, skull or spine. In some less common embodiments, Ewing's sarcoma occurs in the soft tissues of the arms, legs, abdomen, chest, neck, head or a combination thereof. In some embodiments of Ewing's sarcoma, there is no bone involvement. In some embodiments, treatments for Ewing's sarcoma comprise chemotherapy, surgery, or a combination thereof. In some embodiments, Ewing's sarcoma is associated with a chromosomal translocations affecting the EWSR1 (EWS RNA binding protein 1), FLI1 (Fli-1 proto-oncogene), ERG (ERG, ETS transcription factor) and ETV1 (ETS variant 1) genes.

In some embodiments, the cancer is gastric cancer. Gastric cancers comprise cancers which form from the cells of the lining of the stomach. In some embodiments, the gastric cancer comprises a gastrointestinal stromal cell tumor (GIST), a lymphoma, a carcinoid tumor, a squamous cell carcinoma, a small cell carcinoma or a leiomyosarcoma. GISTs may be malignant or benign. GISTs are most commonly found in the stomach and small intestine, but may be found anywhere in in or near the gastrointestinal tract. In some embodiments, a GIST may arise from the interstitial cells of Cajal. A lymphoma is a cancer of the lymph nodes and lymphatic system. A gastric lymphoma may be a primary lymphoma (i.e., a lymphoma that originates in the stomach itself), or a secondary lymphoma that originated elsewhere and metastasized to the stomach. Gastric squamous cell carcinomas are extremely rare. Squamous cell carcinomas arise from abnormal squamous cells, which are cells in the upper layer of the skin. Small cell carcinomas are a highly malignant type of cancer that most frequently occur in the lungs, but can arise in the cervix, prostate, liver pancreas, gastrointestinal tract or bladder.

In some embodiments, the cancer is Leiomyosarcoma. Leiomyosarcomas are a type of soft tissue sarcoma. In some embodiments, the leiomyosarcoma comprises a malignant tumor that arises from smooth muscle cells. Smooth muscles cells are the cells of involuntary muscles, i.e. muscles over which the brain has no voluntary control. Exemplary involuntary muscles comprise the walls of the digestive tract and muscles controlling salivary gland secretions. In some embodiments, the leiomyosarcoma grows and spreads into surrounding tissues. In some embodiments, the leiomyosarcoma spreads to distant sites of the body via the bloodstream or lymphatic system, or both. In some embodiments, a leiomyosarcoma can form almost anywhere where there are blood vessels, such as the heart, liver, pancreas, genitourinary and gastrointestinal tract, the space behind the abdominal cavity (retroperitoneum), the uterus or skin. In some of the more common embodiments, the leiomyosarcoma forms in the uterus. Symptoms, diagnosis and treatment of leiomyosarcomas varies depending on the location and stage of the cancer.

In some embodiments, the cancer is liver cancer. Liver cancers comprise cancers that form from cells of the liver. Exemplary but non-limiting liver cancers include hepatocellular carcinoma, cholangiocarcinoma and hepatoblastoma. In some embodiments, the liver cancer comprises a hepatocellular carcinoma. In some embodiments, the hepatocellular carcinoma occurs in a patient with chronic liver disease and cirrhosis. In some embodiments, the hepatocellular carcinoma forms from hepatic stem cells. In some embodiments, the liver cancer comprises a cholangiocarcinoma. In some embodiments, the cholangiocarcinoma forms in the bile ducts just outside the liver. In some embodiments, the cholangiocarcinoma is intrahepatic, extrahepatic (i.e., perihilar) or a distal extrahepatic cholangiocarcinoma. In some embodiments, the liver cancer comprises a hepatoblastoma. In some embodiments, the hepatoblastoma occurs in a child or an infant. In some embodiments, the hepatoblastoma originates from immature liver precursor cells. In some embodiments, the hepatoblastoma originates from pluripotent stem cells. In some embodiments, risk factors for liver cancer include obesity, diet, smoking, and genetic factors.

In some embodiments, the cancer is lung cancer. In some embodiments, the lung cancer is a small cell lung cancer. In some embodiments, the small cell lung cancer is a small cell carcinoma (oat cell cancer) or a combined small cell carcinoma. In some embodiments, the small cell carcinoma comprises a neuroendocrine subtype of lung cancer that likely arises from neuroendocrine cells in the lung. Risk factors include asbestos exposure and smoking. In some embodiments, the lung cancer is a non-small cell lung cancer. In some embodiments, the non-small cell lung cancer is a non-small cell lung carcinoma. In some embodiments, the non-small cell lung carcinoma is an epithelial lung cancer other than small cell lung carcinoma. In some embodiments the non-small cell lung cancer is an adenocarcinoma, a squamous cell (epidermoid) carcinoma, an adenosquamous carcinoma or a sarcomatoid carcinoma. Squamous cells are flat cells that line the insides of the airways in the lungs.

In some embodiments, the cancer is mesothelioma. Mesotheliomas comprise cancers that develop from the mesothelial, a thin layer of tissue lining lungs, abdomen or heart. In some embodiments, mesotheliomas affect the pleura that surrounds the lungs (pleural mesothelioma). In some embodiments, mesotheliomas affect the tissue of the abdomen (peritoneal mesothelioma). Risk factors for mesothelioma comprise asbestos exposure.

In some embodiments, the cancer is ovarian cancer. Ovarian cancers are cancers arising from cells of the ovaries. Ovarian cancers include, but are not limited to, ovarian epithelial cancers, germ cell tumors, ovarian carcinomas, ovarian stromal tumors and ovarian sarcoma.

In some embodiments, the cancer is pancreatic cancer. Pancreatic cancers typically arise from the cells in the pancreas, a glandular organ behind the stomach. In some, more frequent, embodiments, the pancreatic cancer is a pancreatic adenocarcinoma. Typically, pancreatic adenocarcinomas arise from the part of the pancreas which makes digestive enzymes. In some embodiments, the pancreatic cancer is a neuroendocrine tumor, which arises from the hormone producing cells of the pancreas.

In some embodiments, the cancer is renal cancer. Renal cancers are cancers that arise from cells of the kidney. In some embodiments, the renal cancer first appears in the tubules of the kidney. In some embodiments, the renal cancer is an adult cancer. In some embodiments, the renal cancer is a pediatric cancer. In some embodiments, the renal cancer is a renal cell carcinoma, an inherited papillary renal cell carcinoma, a urothelial cell carcinoma of the renal pelvis, a squamous cell carcinoma, a juxtaglomerular cell tumor (reninoma), an angiomyolipoma, a renal oncocytoma, a Bellini duct carcinoma, a clear-cell sarcoma of the kidney, a mesoblastic nephroma, a Wilms' tumor (usually diagnosed in children under 5 years of age) or a mixed epithelial stromal tumor.

In some embodiments, the cancer is rhabdomyosarcoma. In some embodiments, the rhabdomyosarcoma comprises an embryonal rhabdomyosarcoma. Embryonal rhabdomyosarcomas typically affect children in their first five years of life. The cells of an embryonal rhabdomyosarcoma comprise cells that resemble the developing muscle cells of a six to eight week embryo. In some embodiments, embryonal rhabdomyosarcomas comprise rhabdomyosarcomas of the head and neck area, bladder vagina, or in or around the prostate and testicles. In some embodiments, embryonal rhabdomyosarcomas comprise botryoid and spindle rhabdomyosarcomas. In some embodiments, the rhabdomyosarcoma comprises an alveolar rhabdomyosarcoma. Alveolar rhabdomyosarcomas typically affect all age groups equally. Alveolar rhabdomyosarcomas typically occur in the large muscles of the trunk, arms and legs. The cells of an alveolar rhabdomyosarcoma comprise cells that resemble those of normal muscle cells seen in a ten week old fetus. In some embodiments, the rhabdomyosarcoma comprises an anaplastic rhabdomyosarcoma.

In some embodiments, the cancer is a neuroblastoma. In some embodiments, neuroblastomas are cancers that begin in certain forms of nerve cells typically found in an embryo or fetus. In some embodiments, the nerve cells that give rise to the neuroblastoma are neuroblasts. Neuroblastomas occur most frequently in infants and young children, and are found only rarely in subjects older than ten years of age. In some embodiments, the neuroblastoma starts in the adrenal gland. In some embodiments, the neuroblastoma starts in the sympathetic nerve ganglia in the abdomen. In some embodiments, the neuroblastoma starts in the sympathetic nerve ganglia near the spine in the chest, neck or pelvis. In some embodiments, the neuroblastoma is a ganglioneuroblastoma. In some embodiments, the ganglioneuroblastoma comprises both malignant and benign components.

In some embodiments of the methods of treating cancer of the disclosure, the compositions of the disclosure, e.g. comprising nanoparticles comprising dsRNAs or siRNAs targeting GPC2, can be administered as a monotherapy.

In other embodiments, the nanoparticles comprising dsRNAs or siRNAs targeting GPC2 are administered in conjunction with agents useful for treating cancer, such as chemotherapeutic agents, or standards of care for the cancer. In some embodiments, the nanoparticle compositions of the disclosure can be administered as a combination therapy, i.e. in conjunction with one or more additional therapeutic agents. For example, a composition comprising nanoparticles comprising siRNAs targeting GPC2 can be administered with a chemotherapeutic agent. The chemotherapeutic agent can be platinum based antineoplastic agent, a DNA alkylating agent, a DNA intercalating agent, or a topoisomerase inhibitor. In some embodiments, the chemotherapeutic agent is selected from the group consisting of Cisplatin, Carboplatin, Cyclophosphamide, Doxorubicin, Topotecan or Etoposide.

In some embodiments, administration of nanoparticles comprising siRNAs targeting GPC2 or pharmaceutical compositions comprising same increases sensitivity of the cancer to an additional chemotherapeutic agent. For example, administering nanoparticle compositions can increase sensitivity to the additional chemotherapeutic agent such as Cisplatin, Carboplatin, Cyclophosphamide, Doxorubicin, Topotecan or Etoposide, thereby lowering the therapeutically effective amount of the chemotherapeutic agent. Administering nanoparticles comprising dsRNAs or siRNAs targeting GPC2 can, in some embodiments, decrease the IC50 of chemotherapeutic agents (the half maximal inhibitory concentration). In some embodiments, administering nanoparticles comprising dsRNAs or siRNAs targeting GPC2 can decrease the IC50 of a chemotherapeutic agent by at least 1 fold, at least 2 fold, at least 3 fold, at least 4 fold, at least 5 fold, at least 6 fold, at least 7 fold, at least 8 fold, at least 9 fold, at least 10 fold, at least 15 fold, at least 20 fold, at least 25 fold, at least 30 fold, at least 35 fold, at least 40 fold, at least 45 fold, at least 50 fold, at least 55 fold, at least 60 fold, at least 65 fold, at least 70 fold, at least 75 fold, or at least 80 fold. In some embodiments, administering nanoparticles comprising dsRNAs or siRNAs targeting GPC2 can decrease the IC50 of a chemotherapeutic agent by between about 1 and 80 fold, 1 and 70 fold, 1 and 60 fold, 1 and 50 fold, 1 and 40 fold, 1 and 30 fold, 1 and 20 fold, 1 and 10 fold, 3 and 80 fold, 3 and 70 fold, 3 and 60 fold, 3 and 50 fold, 3 and 40 fold, 3 and 30 fold, 3 and 20 fold, 3 and 10 fold, 5 and 70 fold, 5 and 60 fold, 5 and 55 fold, 5 and 50 fold, 5 and 45 fold, 5 and 40 fold, 5 and 35 fold, 5 and 30 fold, 5 and 25 fold, 5 and 20 fold, 5 and 15 fold, 5 and 10 fold, 10 and 70 fold, 10 and 50 fold, 20 and 50 fold, 20 and 40 fold, or 20 and 30 fold. In some embodiments, administering nanoparticles comprising dsRNAs or siRNAs targeting GPC2 can decrease the IC50 of a chemotherapeutic agent by between about 3 and 70 fold. In some embodiments, administering nanoparticles comprising dsRNAs or siRNAs targeting GPC2 can decrease the IC50 of a chemotherapeutic agent by between about 5 and 50 fold. In some embodiments, administering nanoparticles comprising dsRNAs or siRNAs targeting GPC2 can decrease the IC50 of a chemotherapeutic agent by between about 10 and 30 fold. In some embodiments, administration of nanoparticle compositions increases the effectiveness of the chemotherapeutic agent in the treatment of cancer. In some embodiments, administration of nanoparticles comprising dsRNAs or siRNAs targeting GPC2 can reduce a side effect of the chemotherapeutic agent. For example, if the chemotherapeutic agent is more effective when GPC2 expression is reduced, a lower amount of the chemotherapeutic agent can be administered to the subject, thereby reducing side effects. In some embodiments, administration of nanoparticles comprising dsRNAs or siRNAs targeting GPC2 reduces a sign or a symptom of the cancer.

The nanoparticle compositions of the disclosure, and the one or more additional therapeutic agent(s) can be administered simultaneously. For example, the nanoparticles comprising dsRNAs or siRNAs targeting GPC2 and the additional therapeutic agents(s) can be formulated in the same pharmaceutical composition, and are administered simultaneously to a subject. As a further example, the nanoparticles comprising dsRNAs or siRNAs targeting GPC2 may further comprise one or more additional chemotherapeutic agents, and are administered simultaneously to a subject.

Alternatively, or in addition, the additional therapeutic agent(s) can formulated in separate pharmaceutical composition from the nanoparticles comprising siRNAs of the disclosure, and can be administered separately to a subject. For example, the nanoparticles of the disclosure and the one or more additional therapeutic agent(s) may be delivered via different routes of administration, or on different delivery schedules.

In some embodiments, the one or more additional therapeutic agents are delivered in temporal proximity with the nanoparticles comprising siRNAs of the disclosure. As used herein, “temporal proximity” means sufficiently close in time to produce a combined effect (that is, temporal proximity can be simultaneously, or it can be two or more events occurring within a short time period before or after each other).

The composition comprising nanoparticles comprising dsRNAs or siRNAs targeting GPC2 can be administered to a subject with a cancer. In some embodiments, the administration occurs once a month. In some embodiments, the administration occurs every two weeks. In some embodiments, the administration occurs once a week. In some embodiments, the administration occurs once a day. In some embodiments, the administration occurs twice a day. In some embodiments, the administration occurs three times a day. In some embodiments, the administration occurs four or more times a day. In some embodiments, the subject is administered a composition comprising a therapeutically effective amount of the composition for at least a week, at least a month, at least 2 months, at least 3 months, at least 4 months, at least 5 months, at least 6 months, at least 1 year, at least 2 years, at least 3 years or until the cancer is alleviated.

In some embodiments, the composition comprising nanoparticles comprising dsRNAs or siRNAs targeting GPC2 is administered daily, every day, without a holiday. In some embodiments, the composition comprising nanoparticles comprising dsRNAs or siRNAs targeting GPC2 is administered with a holiday. In some embodiments, this holiday is once a week. In some embodiments, this holiday is twice a week. In some embodiments, this holiday is once every other week. In some embodiments, this holiday is once a month. In some embodiments, this holiday is determined by the effectiveness of the composition comprising nanoparticles comprising dsRNAs or siRNAs targeting GPC2 in alleviating a sign or a symptom of the cancer, and/or how well the subject with the cancer tolerates the administration of the composition.

In some embodiments, the composition comprising nanoparticles comprising dsRNAs or siRNAs targeting GPC2, and optionally, a chemotherapeutic agent, is administered simultaneously with one or more additional cancer therapies. In some embodiments, the nanoparticle composition is administered before an additional cancer therapy. In some embodiments, the nanoparticle composition is administered after an additional cancer therapy. In some embodiments, the nanoparticle composition and the additional cancer therapy are administered in alternation. In some embodiments, this additional cancer therapy comprises an additional chemotherapy.

In some embodiments of the methods of treating cancer of the disclosure, the methods further comprise a standard of care for the cancer. A standard of care for a cancer is a generally accepted appropriate treatment for a given cancer indication based on scientific evidence, and represents the current consensus of the scientific and medical communities. Standards of care for cancers can include, but are not limited to, radiation treatment, surgery to resect tumors, either partially or fully, and additional cancer therapies such as combination therapies, small molecule inhibitors or immunotherapies. An appropriate standard of care for a cancer will be apparent to the person of ordinary skill in the art.

A cancer that is to be treated can be staged according to an American Joint Committee on Cancer (AJCC) classification as Stage I, Stage IIA, Stage IIB, Stage IIIA, Stage IIIB, Stage IIIC, or Stage IV. A cancer that is to be treated can be assigned a grade according to an AJCC classification as Grade GX (e.g., grade cannot be assessed), Grade 1, Grade 2, Grade 3 or Grade 4. A cancer that is to be treated can be staged according to an AJCC pathologic classification (pN) of pNX, pN0, PN0 (I−), PN0 (I+), PN0 (mol−), PN0 (mol+), PN1, PN1 (mi), PN1a, PN1b, PN1c, pN2, pN2a, pN2b, pN3, pN3a, pN3b, or pN3c. Alternatively, or in addition, a cancer can be staged according to the TNM staging system, which divides most types of cancers into 4 stages. Stage 1 usually means that a cancer is relatively small and contained within the organ of origin. Stage 2 cancers have usually not started to spread into surround tissues, but that the tumor is larger than stage 1. In some embodiments, stage 2 means that the cancer has spread into the lymph nodes close to the tumor. Stage 3 cancers are usually larger, and have started to spread into surrounding tissues and lymph nodes. Stage 4, or metastatic cancers, are typically cancers that have spread from the point of origin to other organ(s) in the body.

A cancer that is to be treated can be evaluated by DNA cytometry, flow cytometry, or image cytometry. A cancer that is to be treated can be typed as having 10%, 20%, 30%, 40%, 50%, 60%, 70%, 80%, or 90% of cells in the synthesis stage of cell division (e.g., in S phase of cell division). A cancer that is to be treated can be typed as having a low S-phase fraction or a high S-phase fraction.

As used herein, a “normal cell” is a cell that cannot be classified as part of a “cell proliferative disorder”. A normal cell lacks unregulated or abnormal growth, or both, that can lead to the development of an unwanted condition or disease. Preferably, a normal cell possesses normally functioning cell cycle checkpoint control mechanisms.

As used herein, “contacting a cell” refers to a condition in which a compound or other composition of matter is in direct contact with a cell, or is close enough to induce a desired biological effect in a cell.

As used herein, “monotherapy” refers to the administration of a single active or therapeutic agent to a subject in need thereof. Preferably, monotherapy will involve administration of a therapeutically effective amount of an active agent. For example, administering a nanoparticle comprising an siRNA targeting GPC2 to a subject in need of treatment of cancer. Monotherapy may be contrasted with combination therapy, in which a combination of multiple active agents is administered, preferably with each component of the combination present in a therapeutically effective amount.

As used herein, “treating” or “treat” describes the management and care of a subject for the purpose of combating a disease, condition, or disorder and includes the administration of a pharmaceutical composition of the disclosure to alleviate the symptoms or complications of cancer or to eliminate the cancer.

As used herein, the term “alleviate” is meant to describe a process by which the severity of a sign or symptom of cancer is decreased. Importantly, a sign or symptom can be alleviated without being eliminated. In a preferred embodiment, the administration of pharmaceutical compositions of the disclosure leads to the elimination of a sign or symptom, however, elimination is not required. Effective dosages are expected to decrease the severity of a sign or symptom. For instance, a sign or symptom of a disorder such as cancer, which can occur in multiple locations, is alleviated if the severity of the cancer is decreased within at least one of multiple locations.

As used herein, the term “severity” is meant to describe the potential of cancer to transform from a precancerous, or benign, state into a malignant state. Alternatively, or in addition, severity is meant to describe a cancer stage, for example, according to the TNM system (accepted by the International Union Against Cancer (UICC) and the American Joint Committee on Cancer (AJCC)) or by other art-recognized methods. Cancer stage refers to the extent or severity of the cancer, based on factors such as the location of the primary tumor, tumor size, number of tumors, and lymph node involvement (spread of cancer into lymph nodes). Alternatively, or in addition, severity is meant to describe the tumor grade by art-recognized methods (see, National Cancer Institute, www.cancer.gov). Tumor grade is a system used to classify cancer cells in terms of how abnormal they look under a microscope and how quickly the tumor is likely to grow and spread. Many factors are considered when determining tumor grade, including the structure and growth pattern of the cells. The specific factors used to determine tumor grade vary with each type of cancer. Severity also describes a histologic grade, also called differentiation, which refers to how much the tumor cells resemble normal cells of the same tissue type (see, National Cancer Institute, www.cancer.gov). Furthermore, severity describes a nuclear grade, which refers to the size and shape of the nucleus in tumor cells and the percentage of tumor cells that are dividing (see, National Cancer Institute, www.cancer.gov).

As used herein, the term “aggressive” indicates a cancer that can grow, form or spread quickly. Cancers termed aggressive may be susceptible to treatment, or they may resist treatment. An aggressive cancer can comprise any sort of cancer. Alternatively, or in addition, the term “aggressive” may describe a cancer that requires a more severe or intense than the usual form of treatment for that cancer.

As used herein, the term “refractory” describes a cancer that does not respond to an attempted form of treatment. Refractory cancers can also be termed resistant cancers.

In another aspect of the disclosure, severity describes the degree to which a tumor has secreted growth factors, degraded the extracellular matrix, become vascularized, lost adhesion to juxtaposed tissues, or metastasized. Moreover, severity describes the number of locations to which a primary tumor has metastasized. Finally, severity includes the difficulty of treating tumors of varying types and locations. For example, inoperable tumors, those cancers which have greater access to multiple body systems (hematological and immunological tumors), and those which are the most resistant to traditional treatments are considered most severe. In these situations, prolonging the life expectancy of the subject and/or reducing pain, decreasing the proportion of cancerous cells or restricting cells to one system, and improving cancer stage/tumor grade/histological grade/nuclear grade are considered alleviating a sign or symptom of the cancer.

As used herein the term “symptom” is defined as an indication of disease, illness, injury, or that something is not right in the body. Symptoms are felt or noticed by the individual experiencing the symptom, but may not easily be noticed by others. Others are defined as non-health-care professionals.

As used herein the term “sign” is also defined as an indication that something is not right in the body. But signs are defined as things that can be seen by a doctor, nurse, or other health care professional.

Cancer is a group of diseases that may cause almost any sign or symptom. The signs and symptoms will depend on where the cancer is, the size of the cancer, and how much it affects the nearby organs or structures. If a cancer spreads (metastasizes), then symptoms may appear in different parts of the body.

As a cancer grows, it begins to push on nearby organs, blood vessels, and nerves. This pressure creates some of the signs and symptoms of cancer. Cancers may form in places where it does not cause any symptoms until the cancer has grown quite large.

Cancer may also cause symptoms such as fever, fatigue, or weight loss. This may be because cancer cells use up much of the body's energy supply or release substances that change the body's metabolism. Or the cancer may cause the immune system to react in ways that produce these symptoms. While the signs and symptoms listed above are the more common ones seen with cancer, there are many others that are less common and are not listed here. However, all art-recognized signs and symptoms of cancer are contemplated and encompassed by the disclosure.

Treating cancer may result in a reduction in size of a tumor. A reduction in size of a tumor may also be referred to as “tumor regression”. Preferably, after treatment according to the methods of the disclosure, tumor size is reduced by 5% or greater relative to its size prior to treatment; more preferably, tumor size is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Size of a tumor may be measured by any reproducible means of measurement. The size of a tumor may be measured as a diameter of the tumor.

Treating cancer may result in a reduction in tumor volume. Preferably, after treatment according to the methods of the disclosure, tumor volume is reduced by 5% or greater relative to its size prior to treatment; more preferably, tumor volume is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75% or greater. Tumor volume may be measured by any reproducible means of measurement.

Treating cancer may result in a decrease in number of tumors. Preferably, after treatment, tumor number is reduced by 5% or greater relative to number prior to treatment; more preferably, tumor number is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. Number of tumors may be measured by any reproducible means of measurement. The number of tumors may be measured by counting tumors visible to the naked eye or at a specified magnification. Preferably, the specified magnification is 2×, 3×, 4×, 5×, 10×, or 50×.

Treating cancer may result in a decrease in number of metastatic lesions in other tissues or organs distant from the primary tumor site. Preferably, after treatment according to the methods of the disclosure, the number of metastatic lesions is reduced by 5% or greater relative to number prior to treatment; more preferably, the number of metastatic lesions is reduced by 10% or greater; more preferably, reduced by 20% or greater; more preferably, reduced by 30% or greater; more preferably, reduced by 40% or greater; even more preferably, reduced by 50% or greater; and most preferably, reduced by greater than 75%. The number of metastatic lesions may be measured by any reproducible means of measurement. The number of metastatic lesions may be measured by counting metastatic lesions visible to the naked eye or at a specified magnification. Preferably, the specified magnification is 2×, 3×, 4×, 5×, 10×, or 50×.

Treating cancer can result in stabilization of disease where tumors neither progress nor regress. Preferably, stabilization will be maintained by more than 30 days; more preferably, by more than 60 days; more preferably, by more than 90 days; and most preferably, by more than 120 days.

Treating cancer can result in an increase in average survival time of a population of treated subjects in comparison to a population receiving carrier alone. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active agent. An increase in average survival time of a population may also be measured, for example, by calculating for a population the average length of survival following completion of a first round of treatment with an active agent.

Treating cancer can result in an increase in average survival time of a population of treated subjects in comparison to a population of untreated subjects. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active agent. An increase in average survival time of a population may also be measured, for example, by calculating for a population the average length of survival following completion of a first round of treatment with an active agent.

Treating cancer can result in increase in average survival time of a population of treated subjects in comparison to a population receiving monotherapy with a drug or standard of care that is not a nanoparticle composition of the present invention. Preferably, the average survival time is increased by more than 30 days; more preferably, by more than 60 days; more preferably, by more than 90 days; and most preferably, by more than 120 days. An increase in average survival time of a population may be measured by any reproducible means. An increase in average survival time of a population may be measured, for example, by calculating for a population the average length of survival following initiation of treatment with an active agent. An increase in average survival time of a population may also be measured, for example, by calculating for a population the average length of survival following completion of a first round of treatment with an active agent.

Treating cancer can result in a decrease in the mortality rate of a population of treated subjects in comparison to a population receiving carrier alone. Treating cancer can result in a decrease in the mortality rate of a population of treated subjects in comparison to an untreated population. Treating cancer can result in a decrease in the mortality rate of a population of treated subjects in comparison to a population receiving monotherapy with a drug that is not a pharmaceutical composition of the present invention. Preferably, the mortality rate is decreased by more than 2%; more preferably, by more than 5%; more preferably, by more than 10%; and most preferably, by more than 25%. A decrease in the mortality rate of a population of treated subjects may be measured by any reproducible means. A decrease in the mortality rate of a population may be measured, for example, by calculating for a population the average number of disease-related deaths per unit time following initiation of treatment with an active agent. A decrease in the mortality rate of a population may also be measured, for example, by calculating for a population the average number of disease-related deaths per unit time following completion of a first round of treatment with an active agent.

Treating cancer can result in a decrease in tumor growth rate. Preferably, after treatment, tumor growth rate is reduced by at least 5% relative to number prior to treatment; more preferably, tumor growth rate is reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Tumor growth rate may be measured by any reproducible means of measurement. Tumor growth rate can be measured according to a change in tumor diameter per unit time.

Treating cancer can result in a decrease in tumor regrowth. Preferably, after treatment, tumor regrowth is less than 5%; more preferably, tumor regrowth is less than 10%; more preferably, less than 20%; more preferably, less than 30%; more preferably, less than 40%; more preferably, less than 50%; even more preferably, less than 50%; and most preferably, less than 75%. Tumor regrowth may be measured by any reproducible means of measurement. Tumor regrowth is measured, for example, by measuring an increase in the diameter of a tumor after a prior tumor shrinkage that followed treatment. A decrease in tumor regrowth is indicated by failure of tumors to reoccur after treatment has stopped.

Treating cancer can result in a reduction in the rate of cellular proliferation. Preferably, after treatment, the rate of cellular proliferation is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The rate of cellular proliferation may be measured by any reproducible means of measurement. The rate of cellular proliferation is measured, for example, by measuring the number of dividing cells in a tissue sample per unit time.

Treating cancer can result in a reduction in the proportion of proliferating cells. Preferably, after treatment, the proportion of proliferating cells is reduced by at least 5%; more preferably, by at least 10%; more preferably, by at least 20%; more preferably, by at least 30%; more preferably, by at least 40%; more preferably, by at least 50%; even more preferably, by at least 50%; and most preferably, by at least 75%. The proportion of proliferating cells may be measured by any reproducible means of measurement. Preferably, the proportion of proliferating cells is measured, for example, by quantifying the number of dividing cells relative to the number of nondividing cells in a tissue sample. The proportion of proliferating cells can be equivalent to the mitotic index.

Treating cancer can result in a decrease in size of an area or zone of cellular proliferation. Preferably, after treatment, size of an area or zone of cellular proliferation is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. Size of an area or zone of cellular proliferation may be measured by any reproducible means of measurement. The size of an area or zone of cellular proliferation may be measured as a diameter or width of an area or zone of cellular proliferation.

Treating cancer can result in a decrease in the number or proportion of cells having an abnormal appearance or morphology. Preferably, after treatment, the number of cells having an abnormal morphology is reduced by at least 5% relative to its size prior to treatment; more preferably, reduced by at least 10%; more preferably, reduced by at least 20%; more preferably, reduced by at least 30%; more preferably, reduced by at least 40%; more preferably, reduced by at least 50%; even more preferably, reduced by at least 50%; and most preferably, reduced by at least 75%. An abnormal cellular appearance or morphology may be measured by any reproducible means of measurement. An abnormal cellular morphology can be measured by microscopy, e.g., using an inverted tissue culture microscope. An abnormal cellular morphology can take the form of nuclear pleiomorphism.

Treating cancer can result in cell death, and preferably, cell death results in a decrease of at least 10% in number of cells in a population. More preferably, cell death means a decrease of at least 20%; more preferably, a decrease of at least 30%; more preferably, a decrease of at least 40%; more preferably, a decrease of at least 50%; most preferably, a decrease of at least 75%. Number of cells in a population may be measured by any reproducible means. A number of cells in a population can be measured by fluorescence activated cell sorting (FACS), immunofluorescence microscopy and light microscopy. Methods of measuring cell death are as shown in Li et al., Proc Natl Acad Sci USA. 100(5): 2674-8, 2003. In some aspects, cell death occurs by apoptosis.

### Routes of Administration

Nanoparticles comprising dsRNAs or siRNAs targeting GPC2 can be administered to a subject by many of the well-known methods currently used for therapeutic treatment. For example, for treatment of cancers, a compositions comprising siRNAs targeting GPC2 may be injected directly into tumors, injected into the blood stream or body cavities or taken orally or applied through the skin with patches. The dose chosen should be sufficient to constitute effective treatment but not so high as to cause unacceptable side effects. The state of the disease condition (e.g., cancer, precancer, and the like) and the health of the patient should preferably be closely monitored during and for a reasonable period after treatment.

The compositions comprising nanoparticles comprising dsRNAs or siRNAs targeting GPC2 can be administered orally, nasally, transdermally, pulmonary, inhalationally, buccally, sublingually, intraperintoneally, subcutaneously, intramuscularly, intravenously, rectally, intrapleurally, intrathecally and parenterally. In some embodiments, the parenteral administration comprises intramuscular, intraperitoneal, subcutaneous or intravenous administration. One skilled in the art will recognize the advantages of certain routes of administration.

Compositions of the disclosure comprising nanoparticles may be administered parenterally. Systemic administration of compositions comprising nanoparticles of the disclosure can also be by intravenous, transmucosal, subcutaneous, intraperitoneal, intramuscular or transdermal means. For intravenous parenteral administration, compositions comprising nanoparticles may be administered by injection or by infusion. For transmucosal or transdermal administration, penetrants appropriate to the barrier to be permeated are used in the formulation. Such penetrants are generally known in the art, and include, for example, for transmucosal administration, detergents, bile salts, and fusidic acid derivatives. Transmucosal administration can be accomplished through the use of nasal sprays or suppositories.

### Dosages

In therapeutic applications, the dosages of the pharmaceutical compositions used in accordance with the invention vary depending on the agent, the age, weight, and clinical condition of the recipient patient, and the experience and judgment of the clinician or practitioner administering the therapy, among other factors affecting the selected dosage. Generally, the dose should be sufficient to result in slowing, and preferably regressing, the growth of the tumors and also preferably causing complete regression of the cancer. Dosages may vary depending on the age and size of the subject and the type and severity of the cancer.

The term “therapeutically effective amount”, as used herein, refers to an amount of a pharmaceutical agent to treat, ameliorate, or prevent a cancer in a subject, or to exhibit a detectable therapeutic or inhibitory effect on said cancer in a subject. The effect can be detected by any assay method known in the art. The precise effective amount for a subject will depend upon the subject's body weight, size, and health; the nature and extent of the condition; and the therapeutic or combination of therapeutics selected for administration. Therapeutically effective amounts for a given situation can be determined by routine experimentation that is within the skill and judgment of the clinician.

For any dsRNA or siRNA, the therapeutically effective amount can be estimated initially either in cell culture assays, e.g., of neoplastic cells, or in animal models, usually rats, mice, rabbits, dogs, or pigs. The animal model may also be used to determine the appropriate concentration range and route of administration. In some embodiments, a standard xenograft or patient derived xenograft mouse model can be used to determine the effectiveness of GPC2 targeting dsRNAs or siRNAs on a cancer of the disclosure. Such information can then be used to determine useful doses and routes for administration in humans. Therapeutic/prophylactic efficacy and toxicity may be determined by standard pharmaceutical procedures in cell cultures or experimental animals, e.g., the maximum tolerated dose and no observable adverse effect dose. Pharmaceutical compositions that exhibit large therapeutic windows are preferred. The dosage may vary within this range depending upon the dosage form employed, sensitivity of the patient, and the route of administration.

Dosage and administration are adjusted to provide sufficient levels of the active agent(s) or to maintain the desired effect. Factors which may be taken into account include the severity of the disease state, general health of the subject, age, weight, and gender of the subject, diet, time and frequency of administration, drug combination(s), reaction sensitivities, and tolerance/response to therapy. Long-acting pharmaceutical compositions may be administered every 3 to 4 days, every week, or once every two weeks depending on half-life and clearance rate of the particular formulation.

The dosage of nanoparticles comprising dsRNAs or siRNAs required depends on the choice of the route of administration; the nature of the formulation; the nature of the patient's illness; the subject's size, weight, surface area, age, and sex; other drugs being administered; and the judgment of the attending physician. Wide variations in the needed dosage are to be expected in view of the differing efficiencies of various routes of administration. For example, oral administration would be expected to require higher dosages than administration by intravenous injection (e.g., 2-, 3-, 4-, 6-, 8-, 10-; 20-, 50-, 100-, 150-, or more fold). Variations in these dosage levels can be adjusted using standard empirical routines for optimization as is well understood in the art. Administrations can be single or multiple. Encapsulation of the inhibitor in a suitable delivery vehicle (e.g., capsules or implantable devices) may increase the efficiency of delivery, particularly for oral delivery.

A therapeutically effective dose of nanoparticles comprising GPC2 targeting dsRNAs or siRNAs described herein can optionally be combined with approved amounts of therapeutic agents, and described herein. For example, a therapeutically effective dose of the nanoparticles comprising dsRNAs or siRNAs described herein can be combined with a therapeutically effective amount of Cisplatin, Carboplatin, Cyclophosphamide, Doxorubicin, Topotecan or Etoposide. In some embodiments, a therapeutically effective dose of nanoparticles comprising GPC2 targeting dsRNAs or siRNAs described herein, and optionally, a therapeutically effective dose of an additional therapeutic agent, can be combined with a standard of care for a cancer.

### Kits and Articles of Manufacture

The invention provides kits comprising any one or more of the compositions described herein, including but not limited to compositions comprising nanoparticles comprising siRNAs targeting GPC2, the nanoparticles optionally comprising one or more chemotherapeutic agents. The kits are for use in the treatment of cancer.

Nanoparticles comprising siRNAs targeting GPC2 can be lyophilized before being packaged in the kit, or can be provided in solution with a pharmaceutically acceptable carrier, diluent of excipient.

In some embodiments of the kits of the disclosure, the kit comprises a therapeutically effective amount of the composition comprising nanoparticles comprising siRNAs targeting GPC2, and instructions for use in the treatment of cancer. In some embodiments, the kit further comprises at least one additional cancer therapeutic agent, such as Cisplatin, Carboplatin, Cyclophosphamide, Doxorubicin, Topotecan or Etoposide.

In some embodiments, the nanoparticle comprises PLGA polymers and an HA targeting agent.

Articles of manufacture include, but are not limited to, instructions for use of the kit in treating cancers, for example astrocytoma, breast cancer, colorectal cancer, Ewing's sarcoma, gastric cancer, leiomyosarcoma, liver cancer, lung cancer, mesothelioma, ovarian cancer, pancreatic cancer, renal cancer, rhabdomyosarcoma or neuroblastoma.

In some embodiments, the kits further comprise instructions for administering the nanoparticles and pharmaceutical compositions comprising same of the disclosure.

All percentages and ratios used herein, unless otherwise indicated, are by weight. Other features and advantages of the present invention are apparent from the different examples. The provided examples illustrate different components and methodology useful in practicing the present invention. The examples do not limit the claimed invention. Based on the present disclosure the skilled artisan can identify and employ other components and methodology useful for practicing the present invention.

### EXAMPLES

### Example 1: Expression of Glypican-2 Across Multiple Cancer Cell Lines and Multiple Cancer Indications

GPC2 mRNA expression levels were determined across multiple cell lines and multiple cancer indications from the Cancer Genome Atlas database, a publicly available resource. Reads Per Kilobase of transcript per Million (RPKM) values were tabulated. Levels greater than 1 represent an increased level of GPC2 mRNA expression. As shown, a significant degree of GPC2 mRNA expression is present across all of the cell lines examined in this analysis. Levels of Glypican-2 are shown in Table 6:

TABLE 6
Glypican-2 expression in multiple cell
lines and multiple cancer indications.

| | | Normalized GPC2 expression |
|---|---|---|
| Indication | Cell line | (RPKM values) |
| Astrocytoma | U138MG | 4.36 |
| | LN18 | 4.77 |
| | A172 | 4.83 |
| | U118MG | 4.91 |
| Breast cancer | BT549 | 4.74 |
| | SKBR3 | 4.78 |
| | MCF7 | 5.06 |
| Colorectal cancer | HCT116 | 4.25 |
| | T84 | 4.8 |
| | COLO205 | 4.87 |
| Ewing's sarcoma | TC-32 | 5.26 |
| | TC71 | 5.75 |
| Gastric Cancer | AGS | 4.58 |
| | NCIN87 | 4.59 |
| | KATOIII | 4.95 |
| | SNU16 | 5.27 |
| Leiomyosarcoma | SKUT1 | 4.67 |
| | SKLMS1 | 4.97 |
| Liver cancer | HEPG2 | 4.83 |
| | C3A | 5.01 |
| | HEP3B217 | 5.08 |
| | JHH5 | 5.11 |
| Lung cancer | SW1271 | 5.15 |
| | DMS114 | 5.34 |
| | SHP77 | 5.37 |
| Mesothelioma | MSTO211H | 4.74 |
| Ovarian cancer | COV362 | 4.95 |
| | TOV112D | 5.06 |
| | SKOV3 | 4.20 |
| | OV90 | 5.16 |
| Pancreatic cancer | HS766T | 4.24 |
| | BXPC3 | 4.33 |
| | CAPAN1 | 4.72 |
| Renal cancer | A704 | 4.35 |
| | 786O | 4.36 |
| | CAKI1 | 4.54 |
| | 769P | 4.55 |
| Rhabdomyosarcoma | RH18 | 4.77 |
| | RH30 | 5.16 |
| | RH41 | 5.74 |
| Neuroblastoma | MHHNB11 | 7.64 |
| | KPNRTBM1 | 7.77 |
| | SHSY5Y | 8.43 |
| | SKNAS | 5.01 |
| | CHP212 | 5.04 |
| | IMR32 | 6.53 |

As can be seen in Table 6, GPC-2 is expressed in multiple cancer cell lines from astrocytoma, breast cancer, colorectal cancer, Ewing's sarcoma, gastric cancer, leiomyosarcoma, liver cancer, lung cancer, ovarian cancer, pancreatic cancer, renal cancer, rhadomyosarcoma and neuroblastoma cells, as well as a mesothelioma cell line.

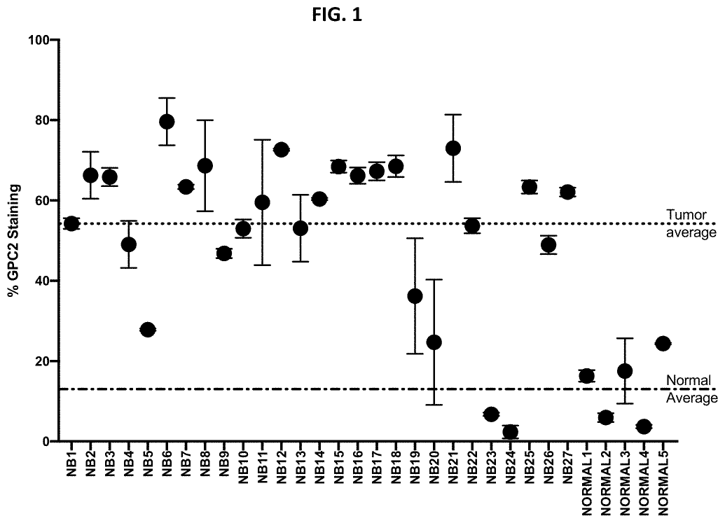

GPC2 protein levels were assayed in neuroblastoma tumors, and levels of GPC2 protein were compared to normal tissues (FIG. 1). GPC2 protein expression levels were determined across multiple tumor tissues and normal peripheral nerve tissues via immunohistochemistry using an antibody against GPC2. The degree of GPC2 staining was quantified using an automated image analyzer and plotted the percent GPC2 staining. Data show on average that significant expression of GPC2 can be detected in neuroblastoma tumors compared to normal tissues.

### Example 2: GPC2 mRNA Knockdown Using siRNAs in TC-32 Ewing's Sarcoma Cells

Several siRNA sequences (n=15) were screened to determine their ability to knockdown (KD) GPC2 expression in multiple cancer cell lines. siRNA sequences were identified using an open source online tool available from either Sigma (sequences 1-11; see www.sigmaaldrich.com/life-science/custom-oligos/sima-oligos/sima-design-service.html or from Dharmacon (sequences 12-15; see dharmacon.horizondiscovery.com/custom-sima). The listed sequences span the full length of the mature GPC2 mRNA. Sense and anti-sense strands for representative siRNAs are shown in Table 7.

TABLE 7
GPC2 siRNAs.

| Name | Sense | Anti-sense |
|---|---|---|
| GPC2 | CUCCUGAUCCUGGCUGAUA[dT][dT] | UAUCAGCCAGGAUCAGGAG[dT][dT] |
| siRNA 1: | (SEQ ID NO: 203) | (SEQ ID NO: 204) |
| GPC2 | CUCAUCUACCGAUGGCUCU[dT][dT] | AGAGCCAUCGGUAGAUGAG[dT][dT] |
| siRNA 2: | (SEQ ID NO: 205) | (SEQ ID NO: 206) |
| GPC2 | CCUGCUUGGACCUCGAUAA[dT][dT] | UUAUCGAGGUCCAAGCAGG[dT][dT] |
| siRNA 3: | (SEQ ID NO: 207) | (SEQ ID NO: 208) |
| GPC2 | GUGGUUCGUGGCUGUCUCA[dT][dT] | UGAGACAGCCACGAACCAC[dT][dT] |
| siRNA 4: | (SEQ ID NO: 209) | (SEQ ID NO: 210) |
| GPC2 | CUCAGUAGCCCAGCACUCU[dT][dT] | AGAGUGCUGGGCUACUGAG[dT][dT] |
| siRNA 5: | (SEQ ID NO: 211) | (SEQ ID NO: 212) |
| GPC2 | CUGCUGUUCCAGUGAGACA[dT][dT] | UGUCUCACUGGAACAGCAG[dT][dT] |
| siRNA 6: | (SEQ ID NO: 213) | (SEQ ID NO: 214) |
| GPC2 | CUCCUUUCUGGUUCACACA[dT][dT] | UGUGUGAACCAGAAAGGAG[dT][dT] |
| siRNA 7: | (SEQ ID NO: 215) | (SEQ ID NO: 216) |
| GPC2 | GAGUGUGGUUUCCUUAGAA[dT][dT] | UUCUAAGGAAACCACACUC[dT][dT] |
| siRNA 8: | (SEQ ID NO: 217) | (SEQ ID NO: 218 |
| GPC2 | GAGUACACCUGCUGUUCCA[dT][dT] | UGGAACAGCAGGUGUACUC[dT][dT] |
| siRNA 9: | (SEQ ID NO: 219) | (SEQ ID NO: 220) |
| GPC2 | GACACGACCUGGACGGGCA[dT][dT] | UGCCCGUCCAGGUCGUGUC[dT][dT] |
| siRNA 10: | (SEQ ID NO: 221) | (SEQ ID NO: 222) |
| GPC2 | CUGACUACCUGCUCUGCCU[dT][dT] | AGGCAGAGCAGGUAGUCAG[dT][dT] |
| siRNA 11: | (SEQ ID NO: 223) | (SEQ ID NO: 224) |
| GPC2 | GCGCUUAAGGUGCCGGUGU[dT][dT] | ACACCGGCACCUUAAGCGC[dT][dT] |
| siRNA 12: | (SEQ ID NO: 225) | (SEQ ID NO: 226) |
| GPC2 | CCUUUGAGCUGACGGCCGA[dT][dT] | UCGGCCGUCAGCUCAAAGG[dT][dT] |
| siRNA 13: | (SEQ ID NO: 227) | (SEQ ID NO: 228) |
| GPC2 | CCUGCUUCUGCUGCUGCCU[dT][dT] | AGGCAGCAGCAGAAGCAGG[dT][dT] |
| siRNA 14: | (SEQ ID NO: 229) | (SEQ ID NO: 230) |
| GPC2 | GAAGAAAUGUGGUCAGCGA[dT][dT] | UCGCUGACCACAUUUCUUC[dT][dT] |
| siRNA 15: | (SEQ ID NO: 231) | (SEQ ID NO: 232) |

Unless otherwise indicated, sequences in Table 7 refer to ribonucleic acids (RNAs). d[T] refers to deoxyribonucleic acids (DNA).

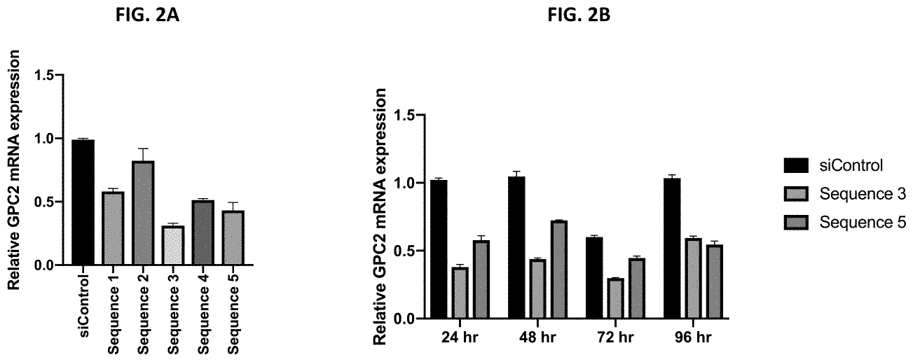

siRNAs 1-5 from Table 7 were diluted to 40 nM in OptiMEM media and mixed in a 1:3 ratio (weight by volume) with RNAiMAX. This was incubated for 15 minutes at room temperature and dispensed onto cells plated in a 6 well tissue culture plate. Total RNA samples were collected at either 72 hours post transfection to compare knockdown efficiency (FIG. 2A), or at 24, 48, 72 or 96 hours post transfection to determine the extent of GPC2 mRNA knockdown over time (FIG. 2B). Expression levels of mRNA of GPC2 were measured in these samples using primers specific for GPC2 obtained commercially from Thermofisher (Carlsbad, Calif.) and standard qPCR technique. Taken together these data indicate that the greatest degree of GPC2 knockdown was observed with sequences 3 and 5 and that knockdown lasts for at least 96 hours post transfection.

### Example 3: GPC2 mRNA Knockdown Affects Cell Viability in Multiple Cancer Cell Lines

GPC2 mRNA knockdown and changes in cell viability were assessed in multiple cancer cell lines. Cells were transfected with either a scrambled control siRNA or with GPC2 siRNA sequence 3 or sequence 5 from Table 7 using the RNAiMAX Lipofectamine kit (Invitrogen, Carlsbad, Calif.) as previously described in Example 2, either in 96 well format (for viability assays) or 6 well format (for knockdown assessment). Following a 96 hour incubation period, cells were washed and the viability measurement was assessed using Cell Titer Glo 2.0. Total RNA was isolated from the 6 well plate samples to determine knockdown efficiency.

As shown in Table 8, although GPC2 knockdown was observed in each cell line using both sequences 3 and 5, the degree of GPC2 knockdown was cell line dependent. In most instances, knockdown of GPC2 expression resulted in a decrease in cell viability.

TABLE 8
Effect of siRNA mediated GPC2 knockdown
across multiple cancer cell lines

| | | siRNA | % GPC2 | % |
|---|---|---|---|---|
| Cell Line | Indication | sequence | knockdown | Viability |
| MCF7 | Breast | Sequence 3 | 72 | 79.67 |
| | | Sequence 5 | 82 | 67.11 |
| SKNAS | Neuroblastoma | Sequence 3 | 25 | 84.58 |
| | | Sequence 5 | 44 | 56.14 |
| CHP212 | | Sequence 3 | 50 | 70.18 |
| | | Sequence 5 | 65 | 64.46 |
| IMR32 | | Sequence 3 | 36 | 89.05 |
| | | Sequence 5 | 25 | 63.62 |
| RH30 | Rhabdomyosarcoma | Sequence 3 | 78 | 79.02 |
| | | Sequence 5 | 83 | 46.03 |
| RH41 | | Sequence 3 | 37 | 131.49 |
| | | Sequence 5 | 60 | 31.32 |
| TC32 | Ewing's Sarcoma | Sequence 3 | 69 | 78.24 |
| | | Sequence 5 | 57 | 77.18 |
| TC71 | | Sequence 3 | 42 | 102.39 |
| | | Sequence 5 | 33 | 100.92 |
| TOV112D | Ovarian Carcinoma | Sequence 3 | 89 | 152.77 |
| | | Sequence 5 | 78 | 81.15 |
| COV362 | | Sequence 3 | 85 | 102.76 |
| | | Sequence 5 | 79 | 94.27 |
| SNU16 | Gastric | Sequence 3 | 53 | 92.74 |
| | | Sequence 5 | 25 | 89.01 |
| GIST-T1 | Gastrointestinal | Sequence 3 | 61 | 70.87 |
| | Stromal | Sequence 5 | 76 | 40.13 |

### Example 4: Comparison of the Activity of Multiple GPC2 siRNA Sequences in Neuroblastoma Cell Lines

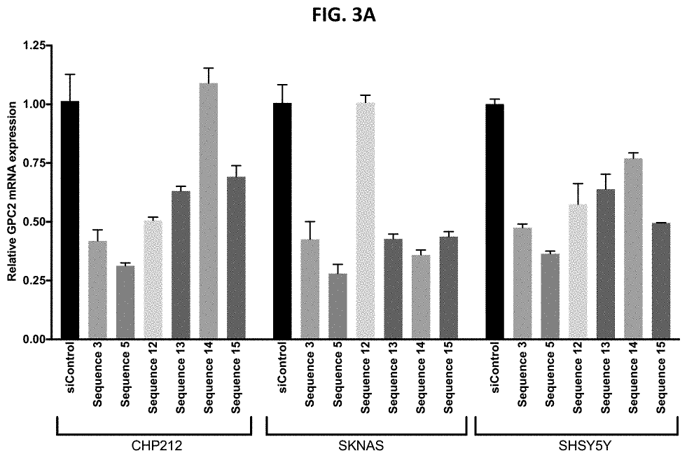

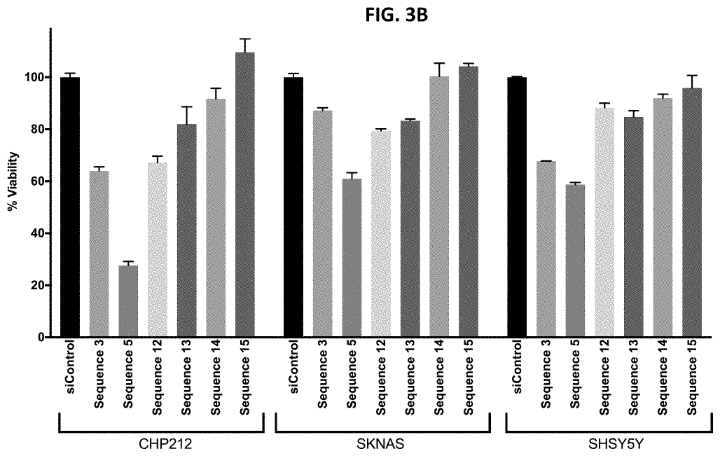

GPC2 mRNA knockdown was assessed in multiple neuroblastoma cell lines. Cells were transfected with either a scrambled control siRNA, with GPC2 siRNA sequences 3 or 5 from Table 7, or with siRNA sequences generated by Dharmacon (sequences 12-15 from Table 7) using the RNAiMAX Lipofectamine kit (Invitrogen, Carlsbad, Calif.) as previously described. Following a 96 hour incubation period, total RNA samples were collected to compare knockdown efficiency and viability was measured in parallel plated cells. As shown, although GPC2 knockdown was observed in each cell line using most siRNA sequences (FIG. 3A), the degree of GPC2 knockdown was greater with sequences 3 and 5 and corresponding greater viability decrease was observed (FIG. 3B).

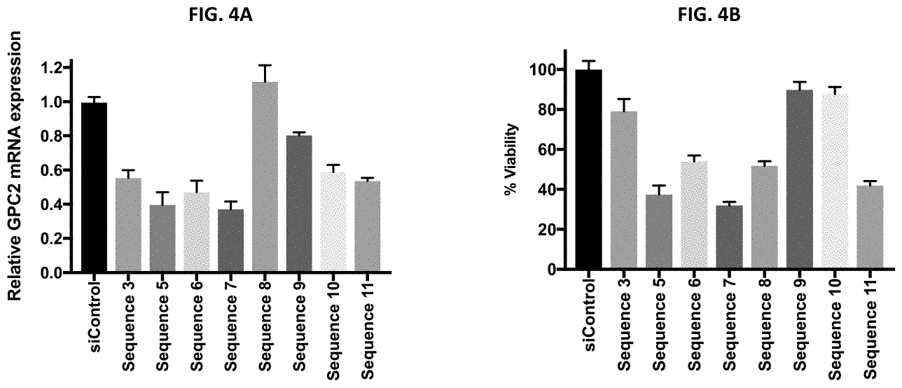

The extent of GPC2 mRNA reduction was assessed with additional GPC2 siRNA sequences in the CHP212 neuroblastoma cell line. Cells were transfected with either a scrambled control siRNA or with GPC2 siRNA sequences 3 or 5, or with siRNA sequences 6-11 (Sigma) using the RNAiMAX Lipofectamine kit (Invitrogen, Carlsbad, Calif.) as previously described. Following a 96 hour incubation period, total RNA samples were collected and viability was measured in parallel plated cells. As shown, although GPC2 knockdown was observed using most siRNA sequences (FIG. 4A), the degree of GPC2 knockdown was greater with sequences 5 and 7 and corresponding greater viability decrease was observed (FIG. 4B).

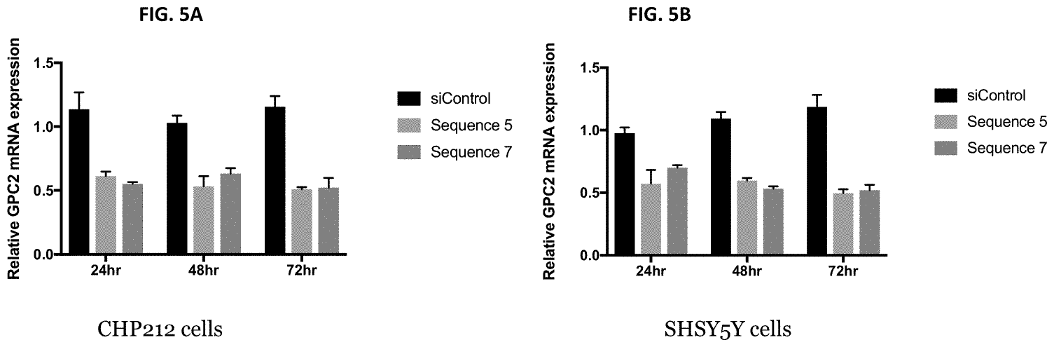

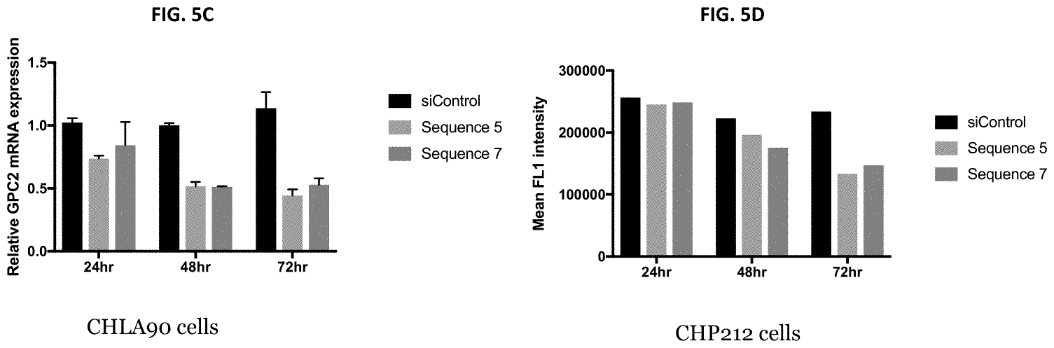

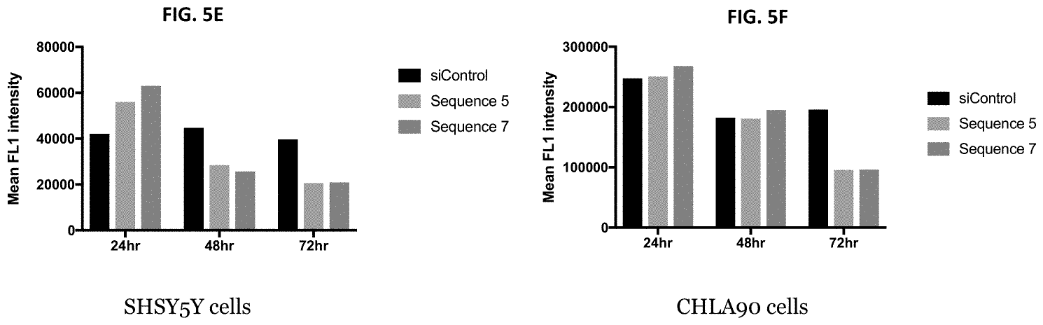

The extent of GPC2 mRNA reduction (knockdown) and corresponding protein level expression changes were assessed in three neuroblastoma cell lines following siRNA knockdown of GPC2. The CHP212 neuroblastoma cell line (FIG. 5A and FIG. 5D), the SH-SY5Y neuroblastoma cell line (FIG. 5B and FIG. 5E) and the CHLA90 neuroblastoma cell line (FIG. 5C and FIG. 5F) were transfected with GPC2 siRNA sequences 5 and 7 from Table 7 and a scrambled control siRNA using the RNAiMAX Lipofectamine kit (Invitrogen, Carlsbad, Calif.) as previously described. Following a 24, 48, or 72 hour incubation period, cells were washed and total RNA was isolated for measuring GPC2 mRNA expression. For protein analysis, cells were fixed, immunostained using an antibody against GPC2 protein, protein expression was measured via flow cytometry. As shown, GPC2 mRNA knockdown was observed beginning at the 24 hr time point and lasted for at least 72 hours. GPC2 protein expression started to decrease at the 48 hr time point.

### Example 5: Knockdown of GPC2 mRNA Results in an Increased Sensitivity to Cisplatin

GPC2 mRNA was reduced using the indicated siRNAs and changes in cell viability were assessed in multiple cancer cell lines, in combination with Cisplatin treatment. Cells were transfected with either a scrambled control siRNA or with GPC2 siRNA sequences 3 or 5 from Table 7 using the RNAiMAX Lipofectamine kit (Invitrogen, Carlsbad, Calif.), as previously described, in either in 96 well (for viability assays) or 6 well format (for total RNA). Cisplatin was added at increasing concentrations (0 to 30 uM) to transfected cells at 24 hours post transfection. Following a 72 hour incubation period, cell viability was measured using Cell Titer Glo 2.0. Total RNA samples were also isolated to measure knockdown efficiency. As shown in Table 9 below, siRNA mediated knockdown of GPC2 was associated with an increase in the sensitivity to Cisplatin in several cell lines, most notably in neuroblastoma cells.

TABLE 9
Effect of siRNA mediated GPC2 knockdown on sensitivity to Cisplatin in
multiple cell lines

| | | % GPC2 | | | | | |
|---|---|---|---|---|---|---|---|
| | | knockdown | | Cisplatin IC₅₀ (μM) | | | |
| Cell | | siRNA | siRNA | | siRNA | siRNA | |
| Line | Indication | #3¹ | #5² | Ctl³ | #3¹ | #5² | Data Summary |
| RH30 | Rhabdomyo- | 80 | 86 | 2.1 | 2.1 | 2.5 | KD⁴ with sequence 3 or 5 |
| | sarcoma | | | | | | does not increase |
| | | | | | | | sensitivity to cisplatin |
| RH41 | | 55 | 70 | 3.9 | 3.2 | 1 | KD with sequence 5 |
| | | | | | | | increases sensitivity to |
| | | | | | | | cisplatin |
| SNU16 | Gastric | 50 | 52 | 16 | 16 | 16 | KD with sequence 3 or 5 |
| | carcinoma | | | | | | does not increase |
| | | | | | | | sensitivity to cisplatin |
| GIST- | Gastrointestinal | 55 | 75 | 9 | 13 | 1 | KD with sequence 5 |
| T1 | Stromal | | | | | | increases sensitivity to |
| | | | | | | | cisplatin |
| TOV112D | Ovarian | 90 | 86 | 4 | 5 | 7 | KD with sequence 3 and 5 |
| | cancer | | | | | | does not increase |
| | | | | | | | sensitivity to cisplatin |
| COV362 | | 85 | 89 | 6.1 | 6.4 | 18 | KD with sequence 3 or 5 |
| | | | | | | | does not increase |
| | | | | | | | sensitivity to cisplatin |
| TC32 | Ewing's | 70 | 75 | 1.2 | 2.3 | 1.2 | KD with sequence 3 or 5 |
| | sarcoma | | | | | | does not increase |
| | | | | | | | sensitivity to cisplatin |
| TC71 | | 55 | 60 | 1.4 | 1.3 | 1.4 | KD with sequence 3 or 5 |
| | | | | | | | does not increase |
| | | | | | | | sensitivity to cisplatin |
| CHP212 | Neuroblastoma | 60 | 70 | 14.0 | 7.4 | 6.0 | KD with sequence 3 or 5 |
| | | | | | | | increases sensitivity to |
| | | | | | | | cisplatin |
| SKNAS | | 40 | 70 | 60 | 20 | 9 | KD with sequence 3 or 5 |
| | | | | | | | increases sensitivity to |
| | | | | | | | cisplatin |
| SH- | | 55 | 75 | 15.4 | 17.6 | 9.3 | KD with sequence 5 |
| SY5Y | | | | | | | increases sensitivity to |
| | | | | | | | cisplatin |
| KELLY | | 65 | 75 | 19.4 | 21.7 | 2.5 | KD with sequence 5 |
| | | | | | | | increases sensitivity to |
| | | | | | | | cisplatin |
| SK-N- | | 55 | 68 | 9 | 8 | 3 | KD with sequence 5 |
| BE2 | | | | | | | increases sensitivity to |
| | | | | | | | cisplatin |
| CHLA- | | 50 | 55 | 23 | 15 | 15 | KD with sequence 3 or 5 |
| 90 | | | | | | | increases sensitivity to |
| | | | | | | | cisplatin |
| CHL A- | | 25 | 35 | 24 | 11 | 6 | KD with sequence 3 or 5 |
| 136 | | | | | | | increases sensitivity to |
| | | | | | | | cisplatin |
| CHLA- | | 5 | 8 | 18 | 16 | 17 | KD with sequence 3 or 5 |
| 172 | | | | | | | does not increase |
| | | | | | | | sensitivity to cisplatin |

¹siRNA sequence #3 from Table 7
²siRNA sequence #5 from Table 7
³scrambled control siRNA
⁴KD; knockdown

### Example 6: Knockdown of GPC2 mRNA Results in an Increased Sensitivity to Carboplatin

GPC2 mRNA expression was reduced using the indicated siRNAs and changes in cell viability were assessed in multiple neuroblastoma cell lines. Cells were transfected with either a control siRNA or with GPC2 siRNA sequences 3, 5 or 7 from Table 7 using the RNAiMAX Lipofectamine kit (Invitrogen, Carlsbad, Calif.) as previously described in 96 wells for viability assays. Carboplatin was added at increasing concentrations (0 to 150 uM) to transfected cells at 24 hours post transfection. Following a 72 hour incubation period, viability measurements were made using Cell Titer Glo 2.0. Representative data for GPC2 knockdown with individual siRNAs generated from a separate experiment are shown in Table 10. As shown in Table 10 below, knockdown of GPC2 was associated with an increase in the sensitivity to carboplatin in several cell lines.

TABLE 10
Effect of siRNA mediated GPC2 knockdown on sensitivity to Carboplatin in
multiple cell lines

| Cell | Ctl¹ | siRNA #3² | | siRNA #5³ | | siRNA #7⁴ | | |
|---|---|---|---|---|---|---|---|---|
| Line | IC₅₀⁵ | % KD⁶ | IC₅₀⁵ | % KD⁶ | IC₅₀⁵ | % KD⁶ | IC₅₀⁵ | Data Summary |
| CHP212 | 23.19 | 60 | 19.89 | 70 | 10.41 | 70 | 8.14 | KD⁷ with sequence 3, 5 or 7 |
| | | | | | | | | increases sensitivity to |
| | | | | | | | | carboplatin |
| SKNAS | 220 | 40 | 40 | 70 | 15.3 | 64 | Not | KD with sequence 3 or 5 |
| | | | | | | | tested | increases sensitivity to |
| | | | | | | | | carboplatin |
| SH- | 1.89 | 55 | 2.3 | 75 | 1.55 | 70 | 0.47 | KD with sequence 5 or 7 |
| SY5Y | | | | | | | | increases sensitivity to |
| | | | | | | | | carboplatin |
| KELLY | 13 | 65 | 12 | 75 | 9 | 73 | Not | KD with sequence 5 increases |
| | | | | | | | tested | sensitivity to carboplatin |
| SK-N- | 30.99 | 55 | 27.9 | 68 | 11 | 69 | 2.5 | KD with sequence 3, 5 or 7 |
| BE2 | | | | | | | | increases sensitivity to |
| | | | | | | | | carboplatin |
| CHLA- | 150 | 50 | 100 | 55 | 100 | 65 | 15 | KD with sequence 3, 5 or 7 |
| 90 | | | | | | | | increases sensitivity to |
| | | | | | | | | carboplatin |
| CHLA- | 31 | 25 | 47 | 35 | 36 | 30 | Not | KD with sequence 3 or 5, does |
| 136 | | | | | | | tested | not increase sensitivity to |
| | | | | | | | | carboplatin |
| CHLA- | 110 | 5 | 100 | 8 | 100 | 20 | Not | KD with sequence 3 or 5 |
| 172 | | | | | | | tested | increases sensitivity to |
| | | | | | | | | carboplatin |

¹Ctl; scrambled control siRNA
²siRNA #3; siRNA with sequence 3 from Table 7
³siRNA #5; siRNA with sequence 5 from Table 7
⁴siRNA #7; siRNA with sequence 7 from Table 7
⁵IC₅₀; carboplatin IC₅₀ in cells transfected with the control siRNA or GPC2 siRNAs, as indicated, in μM
⁶% KD; percent GPC2 mRNA reduction (knockdown) observed with the indicated siRNA, compared to scrambled siRNA control
⁷KD; knockdown

### Example 7: Knockdown of GPC2 mRNA Results in an Increased Sensitivity to Cyclophosphamide

GPC2 mRNA expression was reduced with the indicated siRNAs and changes in cell viability were assessed in multiple neuroblastoma cell lines. Cells were transfected with either a control siRNA or with GPC2 siRNA sequences 3, 5 or 7 from Table 7 using the RNAiMAX Lipofectamine kit (Invitrogen, Carlsbad, Calif.) as previously described in 96 wells for viability assays. Cyclophosphamide was added at increasing concentrations (0-20 uM) to transfected cells at 24 hr post transfection. Following a 72 hour incubation period, viability measurements were made using Cell Titer Glo 2.0. Representative data for GPC2 knockdown with individual siRNAs generated from a separate experiment are shown in Table 11. As shown in Table 11 below, knockdown of GPC2 mRNA was associated with an increase in the sensitivity to Cyclophosphamide in several cell lines.

TABLE 11
Effect of siRNA mediated GPC2 knockdown on sensitivity to
Cyclophosphamide in multiple cell lines

| | Ctl¹ | siRNA #3² | | siRNA #5³ | | siRNA #7⁴ | | |
|---|---|---|---|---|---|---|---|---|
| Cell | IC₅₀ | % | IC₅₀ | % | IC₅₀ | % | IC₅₀ | |
| Line | (μM)⁵ | KD⁶ | (μM)⁵ | KD⁶ | (μM)⁵ | KD⁶ | (μM)⁵ | Data Summary |
| CHP212 | 1.36 | 60 | 0.87 | 70 | 0.45 | 70 | 1.2 | KD⁷ with sequence 3, 5 or 7 |
| | | | | | | | | increases sensitivity to |
| | | | | | | | | cyclophosphamide |
| SKNAS | 12 | 40 | 1.37 | 70 | 0.48 | 64 | Not | KD with sequence 3 or 5 |
| | | | | | | | tested | increases sensitivity to |
| | | | | | | | | cyclophosphamide |
| SH- | 2 | 55 | 1.5 | 75 | 0.3 | 70 | 0.94 | KD with sequence 3, 5 or 7 |
| SY5Y | | | | | | | | increases sensitivity to |
| | | | | | | | | cyclophosphamide |
| KELLY | 10 | 65 | 8 | 75 | 1 | 73 | Not | KD with sequence 3 or 5 |
| | | | | | | | tested | increases sensitivity to |
| | | | | | | | | cyclophosphamide |
| SK-N- | 4.17 | 55 | 2.41 | 68 | 1.8 | 69 | 0.41 | KD with sequence 3, 5 or 7 |
| BE2 | | | | | | | | increases sensitivity to |
| | | | | | | | | cyclophosphamide |
| CHLA- | 30 | 50 | 30 | 55 | 20 | 65 | 10 | KD with sequence 5 or 7 |
| 90 | | | | | | | | increases sensitivity to |
| | | | | | | | | cyclophosphamide |
| CHLA- | 6 | 25 | 11 | 35 | 5 | 30 | Not | KD sequence 3 or 5 does |
| 136 | | | | | | | tested | not increase sensitivity to |
| | | | | | | | | cyclophosphamide |
| CHLA- | 15 | 5 | 10 | 8 | 10 | 20 | Not | KD with sequence 3 or 5 |
| 172 | | | | | | | tested | increases sensitivity to |
| | | | | | | | | cyclophosphamide |

¹Ctl; scrambled control siRNA
²siRNA #3; siRNA with sequence 3 from Table 7
³siRNA #5; siRNA with sequence 5 from Table 7
⁴siRNA #7; siRNA with sequence 7 from Table 7
⁵IC₅₀ (μM); Cyclophosphamide IC₅₀ in microMolar concentration (μM), in cells transfected with the control siRNA or GPC2 siRNAs, as indicated
⁶% KD; percent GPC2 mRNA reduction (knockdown) observed with the indicated siRNA
⁷KD; knockdown

### Example 8: Knockdown of GPC2 mRNA Results in an Increased Sensitivity to Doxorubicin

GPC2 mRNA expression was reduced and changes in cell viability were assessed in multiple neuroblastoma cell lines. Cells were transfected with either a control siRNA or with GPC-2 siRNA sequences 3, 5 or 7 from Table 7 using the RNAiMAX Lipofectamine kit (Invitrogen, Carlsbad, Calif.) as previously described in 96 wells for viability assays. Doxorubicin was added at increasing concentrations (0-5 uM) to transfected cells at 24 hours post transfection. Following a 72 hour incubation period, viability measurements were made using Cell Titer Glo. Representative data for GPC2 knockdown with individual siRNAs generated from a separate experiment are shown in Table 12. As shown in Table 12 below, knockdown of GPC2 was associated with an increase in the sensitivity to Doxorubicin in several cell lines.

TABLE 12
Effect of siRNA mediated GPC2 knockdown on sensitivity to
Cyclophosphamide in multiple cell lines

| | Ctl¹ | siRNA #3² | | siRNA #5³ | | siRNA #7⁴ | | |
|---|---|---|---|---|---|---|---|---|
| Cell | IC₅₀ | % | IC₅₀ | % | IC₅₀ | % | IC₅₀ | |
| Line | (μM)⁵ | KD⁶ | (μM)⁵ | KD⁶ | (μM)⁵ | KD⁶ | (μM)⁵ | Data Summary |
| CHP212 | 0.03 | 60 | 0.03 | 70 | 0.02 | 70 | 0.02 | KD7 with sequence 3, 5 or 7 |
| | | | | | | | | does not increase sensitivity |
| | | | | | | | | to doxorubicin |
| SKNAS | 0.14 | 40 | 0.04 | 70 | 0.02 | 64 | Not | KD with sequence 3 or 5 |
| | | | | | | | tested | increases sensitivity to |
| | | | | | | | | doxorubicin |
| SH- | 0.06 | 55 | 0.08 | 75 | 0.05 | 70 | 0.03 | KD with sequence 7 increases |
| SY5Y | | | | | | | | sensitivity to doxorubicin |
| KELLY | 0.07 | 65 | 0.07 | 75 | 0.06 | 73 | Not | KD with sequence 3 or 5 does |
| | | | | | | | tested | not increase sensitivity to |
| | | | | | | | | doxorubicin |
| SK-N- | 1 | 55 | 1 | 68 | 0.02 | 69 | 0.2 | KD with sequence 5 or 7 |
| BE2 | | | | | | | | increases sensitivity to |
| | | | | | | | | doxorubicin |
| CHLA- | 0.36 | 50 | 0.35 | 55 | 0.25 | 65 | 0.13 | KD with sequence 5 or 7 |
| 90 | | | | | | | | increases sensitivity to |
| | | | | | | | | doxorubicin |
| CHLA- | 0.14 | 25 | 0.17 | 35 | 0.08 | 30 | Not | KD with sequence 5 increases |
| 136 | | | | | | | tested | sensitivity to doxorubicin |
| CHLA- | 2 | 5 | 2 | 8 | 0.9 | 20 | Not | KD with sequence 5 increases |
| 172 | | | | | | | tested | sensitivity to doxorubicin |

¹Ctl; scrambled control siRNA
²siRNA #3; siRNA with sequence 3 from Table 7
³siRNA #5; siRNA with sequence 5 from Table 7
⁴siRNA #7; siRNA with sequence 7 from Table 7
⁵IC₅₀ (μM); Doxorubicin IC₅₀ in microMolar concentration (μM), in cells transfected with the control siRNA or GPC2 siRNAs, as indicated
⁶% KD; percent GPC2 mRNA reduction (knockdown) observed with the indicated siRNA
⁷KD; knockdown

### Example 9: Knockdown of GPC2 mRNA Results in an Increased Sensitivity to Etoposide

GPC2 mRNA expression was reduced and changes in cell viability were assessed in multiple neuroblastoma cell lines. Cells were transfected with either a control siRNA or with GPC2 siRNA sequences 3, 5 or 7 from Table 7 using the RNAiMAX Lipofectamine kit (Invitrogen, Carlsbad, Calif.) as previously described in 96 wells for viability assays. Etoposide was added at increasing concentrations (0-15 uM) to transfected cells at 24 hours post transfection. Following a 72 hour incubation period, viability measurement were made using Cell Titer Glo 2.0. Representative data for GPC2 knockdown with individual siRNAs generated from a separate experiment are shown in Table 13. As shown in Table 13, knockdown of GPC2 was associated with an increase in the sensitivity to Etoposide in several cell lines.

TABLE 13
Effect of siRNA mediated GPC2 knockdown on sensitivity to Etoposide in
multiple cell lines

| | Ctl¹ | siRNA #3² | | siRNA #5³ | | siRNA #7⁴ | | |
|---|---|---|---|---|---|---|---|---|
| Cell | IC₅₀ | % | IC₅₀ | % | IC₅₀ | % | IC₅₀ | |
| Line | (μM)⁵ | KD⁶ | (μM)⁵ | KD⁶ | (μM)⁵ | KD⁶ | (μM)⁵ | Data Summary |
| CHP212 | 0.03 | 60 | 0.03 | 70 | 0.02 | 70 | 0.02 | Knockdown with sequence 5 |
| | | | | | | | | increases sensitivity to etoposide |
| SKNAS | 0.14 | 40 | 0.04 | 70 | 0.02 | 64 | Not | Knockdown with sequence 3 or 5 |
| | | | | | | | tested | increases sensitivity to etoposide |
| SH- | 0.06 | 55 | 0.08 | 75 | 0.05 | 70 | 0.03 | Knockdown with sequence 3, 5 or |
| SY5Y | | | | | | | | 7 increases sensitivity to etoposide |
| KELLY | 0.07 | 65 | 0.07 | 75 | 0.06 | 73 | Not | Knockdown with sequence 3 or 5 |
| | | | | | | | tested | increases sensitivity to etoposide |
| SK-N- | 1 | 55 | 1 | 68 | 0.02 | 69 | 0.2 | Knockdown with sequence 3, 5 or |
| BE2 | | | | | | | | 7 increases sensitivity to etoposide |
| CHLA- | 0.36 | 50 | 0.35 | 55 | 0.25 | 65 | 0.13 | Knockdown with sequence 5 or 7 |
| 90 | | | | | | | | increases sensitivity to etoposide |
| CHLA- | 0.14 | 25 | 0.17 | 35 | 0.08 | 30 | Not | Knockdown with sequence 3 |
| 136 | | | | | | | tested | increases sensitivity to etoposide |
| CHLA- | 2 | 5 | 2 | 8 | 0.9 | 20 | Not | Knockdown with sequence 3 or 5 |
| 172 | | | | | | | tested | increases sensitivity to etoposide |

¹Ctl; scrambled control siRNA
²siRNA #3; siRNA with sequence 3 from Table 7
³siRNA #5; siRNA with sequence 5 from Table 7
⁴siRNA #7; siRNA with sequence 7 from Table 7
⁵IC₅₀ (μM); Etoposide IC₅₀ in microMolar concentration (μM), in cells transfected with the control siRNA or GPC2 siRNAs, as indicated
⁶% KD; percent GPC2 mRNA reduction (knockdown) observed with the indicated siRNA

### Example 10: Knockdown of GPC2 mRNA Results in an Increased Sensitivity to Topotecan

GPC2 mRNA expression was reduced and changes in cell viability were assessed in multiple neuroblastoma cell lines. Cells were transfected with either a control siRNA or with GPC2 siRNA sequences 5 or 7 from Table 7 using the RNAiMAX Lipofectamine kit (Invitrogen, Carlsbad, Calif.) as previously described in 96 wells for viability assays. Topotecan was added at increasing concentrations (0-5 uM) to transfected cells at 24 hours post transfection. Following a 72 hour incubation period, viability measurements were made using Cell Titer Glo 2.0. Representative data for GPC2 knockdown with individual siRNAs generated from a separate experiment are shown in Table 14. As shown in Table 14, knockdown of GPC2 was associated with an increase in the sensitivity to Topotecan in several cell lines.

TABLE 14
Effect of siRNA mediated GPC2 knockdown on sensitivity to Topotecan in
multiple cell lines

| | Control | | | | | |
|---|---|---|---|---|---|---|
| | siRNA | siRNA #5¹ | | siRNA #7² | | |
| Cell | IC₅₀ | % | IC₅₀ | % | IC₅₀ | |
| Line | (μM)³ | KD⁴ | (μM)³ | KD⁴ | (μM)³ | Data Summary |
| CHP212 | 0.02 | 70 | 0.01 | 70 | 0.01 | Knockdown with sequence 5 or 7 does |
| | | | | | | not increase sensitivity to topotecan |
| SKNAS | 1.42 | 70 | 0.02 | 64 | 0.02 | Knockdown with sequence 5 or 7 |
| | | | | | | increases sensitivity to topotecan |
| SH- | 0.05 | 75 | 0.04 | 70 | 0.04 | Knockdown with sequence 5 or 7 does |
| SY5Y | | | | | | not increase sensitivity to topotecan |
| KELLY | 0.03 | 75 | 0.06 | 73 | 0.02 | Knockdown with sequence 5 or 7 does |
| | | | | | | not increase sensitivity to topotecan |
| SK-N- | 0.02 | 68 | 0.01 | 69 | 0.01 | Knockdown with sequence 5 or 7 does |
| BE2 | | | | | | not increase sensitivity to topotecan |
| CHLA- | 0.03 | 55 | 0.11 | 65 | 0.14 | Knockdown with sequence 5 or 7 does |
| 90 | | | | | | not increase sensitivity to topotecan |

¹siRNA #5; siRNA with sequence 5 from Table 7
²siRNA #7; siRNA with sequence 7 from Table 7
³IC₅₀ (μM); Etoposide IC₅₀ in microMolar concentration (μM), in cells transfected with the control siRNA or GPC2 siRNAs, as indicated
⁴% KD; percent GPC2 mRNA reduction (knockdown) observed with the indicated siRNA

### Example 11: Knockdown of GPC2 mRNA Affects Tumor Spheroid Formation

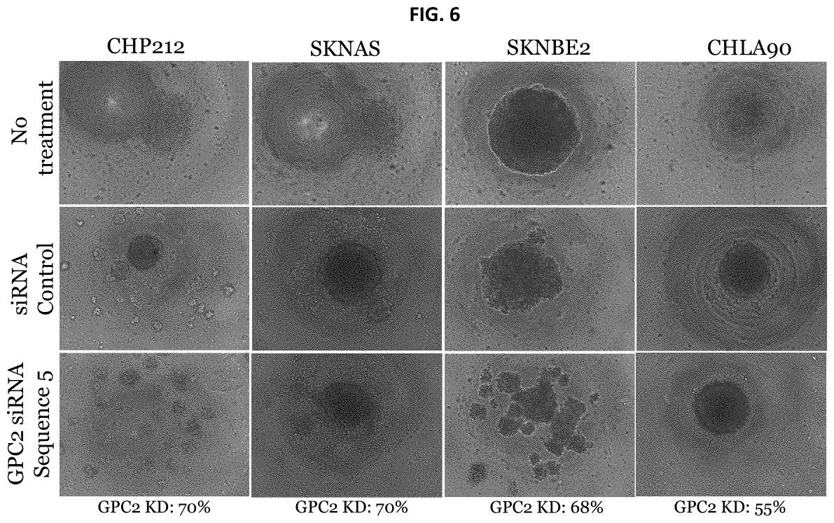

The effect of GPC2 knockdown on tumor spheroid formation was assessed. CHP212, SKNAS, SKNBE2 or CHLA90 neuroblastoma cells were transfected with either a scrambled control siRNA or with a GPC2 siRNA of sequence 5 from Table 7 using the RNAiMAX Lipofectamine kit (Invitrogen, Carlsbad, Calif.), as previously described. Following transfection, cells were plated in a low attachment 96 well plate for spheroid formation while under siRNA treatment. At 96 hours following transfection, spheroid images were taken using the Evos FL microscope ((Life Technologies, Carlsbad, Calif.) at 100× magnification. As shown in FIG. 6, cells that exhibited the highest degree of GPC2 mRNA knockdown showed the greatest inhibition in their ability to form spheroids.

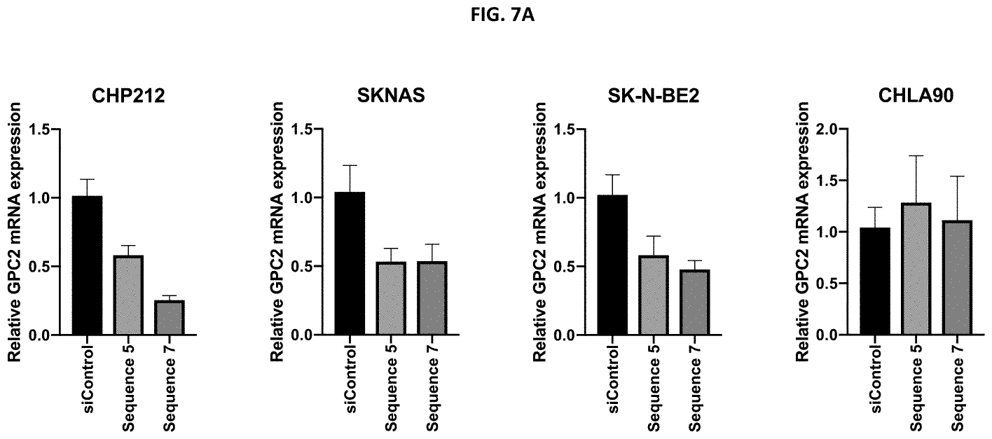

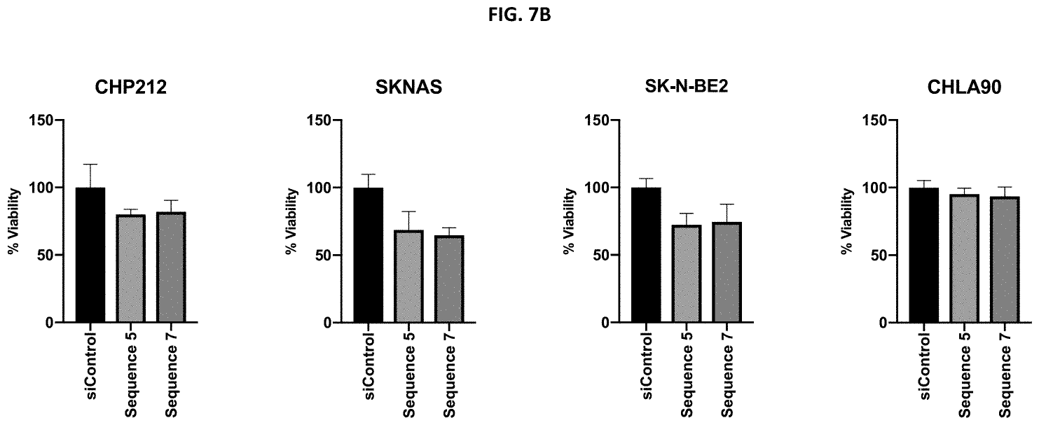

The effect of siRNA mediated GPC2 mRNA knockdown was also assessed on established spheroid cultures. Spheroids were established by seeding cells in low attachment plates and culturing for 96 hours. Spheroids were transfected with either a scrambled control siRNA or with GPC2 siRNA of sequence 5 or 7 from Table 7, using the RNAiMAX Lipofectamine kit (Invitrogen, Carlsbad, Calif.) as previously described. At the 96 hour time point following transfection, the degree of GPC2 mRNA knockdown (FIG. 7A) as well as spheroid viability (FIG. 7B) was measured. As shown in FIGS. 7A and 7B, where GPC2 knockdown was observed, a decrease in spheroid viability was also seen.

Testing the effect of GPC2 mRNA knockdown on spheroids showed similar cytotoxicity to that seen in two dimensional cultures. GPC2 knockdown not only inhibited the formation of tumor spheroids, it also decreased cell viability after spheroids had already formed. This suggests that targeting this pathway may affect both established primary tumors as well as formation of new metastatic lesions.

1. A nanoparticle comprising a small interfering RNA (siRNA), wherein the siRNA comprises a sense region and anti-sense region complementary to said sense region such that the sense region and the anti-sense region together form an RNA duplex, and wherein the sense region comprises a sequence at least 70% identical to a glypican-2 (GPC2) mRNA sequence.

2. The nanoparticle of claim 1, wherein the sense region comprises a sequence that is identical to the GPC2 mRNA sequence.

3. The nanoparticle of claim 1, wherein the siRNA is capable of inducing RNAi-mediated degradation of the GPC2 mRNA.

4. The nanoparticle of claim 1, wherein the sense region is encoded by a first single stranded RNA molecule and the anti-sense region is encoded by a second single stranded RNA molecule.

5. The nanoparticle of claim 4, wherein the first single stranded RNA molecule comprises a first 3′ overhang and the second single stranded RNA molecule comprises a second 3′ overhang.

6.-8. (canceled)

9. The nanoparticle of claim 1, wherein the RNA duplex is between 17 and 24 nucleotides in length.

10. (canceled)

11. The nanoparticle of claim 1, wherein the GPC2 mRNA sequence comprises SEQ ID NO: 1 or SEQ ID NO: 2.

12.-13. (canceled)

14. The nanoparticle of claim 1, wherein the siRNA comprises a sequence selected from the group consisting of SEQ ID NOs: 3-232.

15. The nanoparticle of claim 1, wherein the sense region comprises a sequence of CCUGCUUGGACCUCGAUAA (SEQ ID NO: 3), CUCAGUAGCCCAGCACUCU (SEQ ID NO: 4) or CUCCUUUCUGGUUCACACA (SEQ ID NO: 5).

16. The nanoparticle of claim 1, wherein the sense region comprises a sequence of CCUGCUUGGACCUCGAUAA (SEQ ID NO: 3) and the anti-sense region comprises a sequence of UUAUCGAGGUCCAAGCAGG (SEQ ID NO: 6).

17. The nanoparticle of claim 1, wherein the sense region comprises a sequence of CUCAGUAGCCCAGCACUCU (SEQ ID NO: 4) and the anti-sense region comprises a sequence of AGAGUGCUGGGCUACUGAG (SEQ ID NO: 7).

18. The nanoparticle of claim 1, wherein the sense region comprises a sequence of CUCCUUUCUGGUUCACACA (SEQ ID NO: 5) and the anti-sense region comprises a sequence of UGUGUGAACCAGAAAGGAG (SEQ ID NO: 8).

19. The nanoparticle of claim 1, wherein the siRNA comprises at least one modified nucleotide.

20. The nanoparticle of claim 19, wherein the at least one modified nucleotide increases stability of the RNA duplex.

21. The nanoparticle of claim 19, wherein the at least one modified nucleotide comprises a locked nucleic acid (LNA).

22. The nanoparticle of claim 1, wherein the nanoparticle comprises a liposome, a micelle, a polymer-based nanoparticle, a lipid-polymer based nanoparticle, a nanocrystal, a carbon nanotube based nanoparticle or a polymeric micelle.

23. The nanoparticle of claim 22, wherein the polymer-based nanoparticle comprises a multiblock copolymer, a diblock copolymer, a polymeric micelle or a hyperbranched macromolecule.

24. The nanoparticle of claim 22, wherein the polymer-based nanoparticle comprises a multiblock copolymer or a diblock copolymer.

25. The nanoparticle of claim 23, wherein the polymer-based nanoparticle comprises a poly(lactic-co-glycolic acid) PLGA polymer.

26. The nanoparticle of claim 1, wherein the nanoparticle comprises a targeting agent.

27. (canceled)

28. The nanoparticle of claim 26, wherein the targeting agent is hyaluronic acid.

29. (canceled)

30. The nanoparticle of claim 1, wherein the nanoparticle comprises a chemotherapeutic agent.

31. The nanoparticle of claim 30, wherein the chemotherapeutic agent comprises a platinum based antineoplastic agent, a DNA alkylating agent, a DNA intercalating agent, or a topoisomerase inhibitor.

32.-35. (canceled)

36. A pharmaceutical composition comprising the nanoparticle of claim 1, and one or more of a pharmaceutically acceptable carrier, a diluent or an excipient.

37.-47. (canceled)

48. A kit comprising the nanoparticle of claim 1.

49. A kit comprising the pharmaceutical composition of claim 36.

50. (canceled)

51. A method of treating a cancer in a subject, comprising administering to the subject a therapeutically effective amount of the nanoparticle of claim 1 to the subject.

52. A method of treating cancer in a subject, comprising administering to the subject a therapeutically effective amount of the pharmaceutical composition of claim 36 to the subject.

53.-56. (canceled)

57. A method of treating a cancer in a subject, comprising administering to the subject a therapeutically effective amount of a composition comprising a nanoparticle, the nanoparticle comprising a small interfering RNA (siRNA), wherein the siRNA comprises a sense region and anti-sense region complementary to said sense region such that the sense region and the anti-sense region together form an RNA duplex, wherein the sense region comprises a sequence at least 70% identical to a glypican-2 (GPC2) mRNA sequence of SEQ ID NO: 1 or SEQ ID NO: 2.

58.-103. (canceled)
